(19) United States
(12) Patent Application Publication
(10) Pub. No.: US 2022/0363681 A1
(43) Pub. Date: Nov. 17, 2022

(54) OXO SIX-MEMBERED CYCLOPYRIMIDINE COMPOUND, PREPARATION METHOD AND MEDICAL USE THEREOF

(71) Applicants: GenFleet Therapeutics (Shanghai) Inc., Shanghai (CN); Zhejiang Genfleet Therapeutics Co., Ltd., Shaoxing, Zhejiang (CN)

(72) Inventors: Fusheng ZHOU, Shanghai (CN); Tao JIANG, Shanghai (CN); Wan HE, Shanghai (CN); Zhubo LIU, Shanghai (CN); Leitao ZHANG, Shanghai (CN); Jiong LAN, Shanghai (CN)

(21) Appl. No.: 17/635,938

(22) PCT Filed: Aug. 12, 2020
(86) PCT No.: PCT/CN2020/108658

§ 371 Date: Feb. 16, 2022

(30) Foreign Application Priority Data
Aug. 16, 2019 (CN) 201910757297.9

(51) Int. Cl. C07D 471/10; C07D 401/14; C07D 405/14; C07D 403/14; C07D 471/04; C07D 471/08

(52) U.S. Cl. CPC C07D 471/10; C07D 401/14; C07D 405/14; C07D 403/14; C07D 471/04; C07D 471/08

(57) ABSTRACT

Provided is an oxygen-substituted six-membered ring pyrimidine compound with selective inhibitory effect on KRAS gene mutation and a pharmaceutically acceptable salt, a stereoisomer, a solvate, or a prodrug thereof, as represented by formula (I). The definition of each group or symbol in the formula is detailed in the specification. Moreover, a pharmaceutical composition containing the compound and an application thereof in the preparation of cancer drugs are also provided.

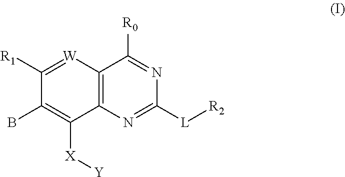

### TECHNICAL FIELD

The present invention relates to the technical field of medicine, and in particular relates to an oxygen-substituted six-membered cyclopyrimidine compound, its application as a selective inhibitor against KRAS gene mutation, and a pharmaceutical composition prepared therefrom.

### BACKGROUND

Lung cancer is the cancer with the highest incidence in the world. It ranks first among all cancers in China. It is also the cancer with the highest incidence and mortality in China. According to data released by the American Cancer Society in 2016, about 1.8 million people suffer from lung cancer, and nearly 80% of lung cancers are non-small cell lung cancer (NSCLC). RAS is a group of closely related monomeric globular proteins (21 kDa molecular weight), which have 188-189 amino acids and bind to guanosine diphosphate GDP or guanosine triphosphate GTP. Members of the RAS subfamily include HRAS, KRAS and NRAS. RAS acts as a molecular switch, and when RAS contains bound GDP, it is in a dormant or closed position and is “inactive”. When cells are exposed to certain growth-promoting stimuli, RAS is induced to convert the bound GDP into GTP. When combined with GTP, RAS is “on” and can interact with other downstream target proteins and activate these proteins. RAS protein itself has a very low inherent ability to hydrolyze GTP and restore it to GDP (thus turning itself into a closed state). The exogenous protein GTPase Activated Protein (GAP) is required to restore it to the closed state. The interaction between GAP and RAS greatly accelerates the conversion of GTP to GDP. Any mutation in RAS will affect the interaction between RAS and GAP and the ability of the conversion of GTP to GDP. Such mutation will lead to prolonged protein activation time, thereby prolonging cell signaling, which in turn will cause cells to continue to grow and divide. Since such signaling causes cell growth and division, over-activated RAS signaling can eventually lead to cancer. Among lung cancers, mutations in the RAS genes are confirmed in about 32% of lung cancers. Any one mutation in the three main subtypes of the RAS (HRAS, NRAS, or KRAS) genes can lead to the occurrence of human tumors. It has been reported that the KRAS gene has the highest mutation frequency in the RAS genes, and KRAS mutations are detected in 25-30% of tumors. In comparison, the rates of oncogenic mutations in NRAS and HRAS family members are much lower (8% and 3%, respectively). The most common KRAS mutations are found in residues G12 and G13 as well as residue Q61 in the P loop. The G12C mutation is a frequent mutation of the KRAS gene (mutation of glycine-12 to cysteine). This mutation has been found in about 13% of cancers, about 43% of lung cancers, and almost 100% of MYH-related polyposis (familial colon cancer syndrome). Therefore, it is a better direction to develop inhibitors that selectively inhibit KRAS mutations. In order to increase the inhibitory activity against KRAS mutations while reducing the inhibitory activity against wild-type KRAS, it has great significance to develop novel selective inhibitors of RAS mutants with higher activity, better selectivity, and lower toxicity.

### SUMMARY OF THE PRESENT INVENTION

The present invention provides an oxygen-substituted six-membered cyclopyrimidine compound, which, as a selective inhibitor against KRAS mutation, has the advantages of high activity, good selectivity, low toxicity and less side effects.

In one aspect of the present invention, it provides an oxygen-substituted six-membered cyclopyrimidine compound, or a pharmaceutically acceptable salt, stereoisomer, solvent or prodrug thereof, and the structure of the compound is as shown in formula (I):

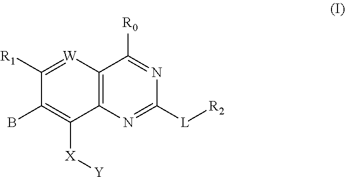

wherein,
R₀ is

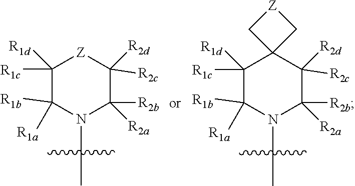

wherein [symbol] represents that the nitrogen atom is connected with other parts of the molecule; wherein,
R₁ₐ, R_{1b}, R_{1c}, R_{1d}, R₂ₐ, R_{2b}, R_{2c}, R_{2d} are the same or different, and are each independently hydrogen, halogen, C₁₋₃ alkyl, —C₁₋₃ alkyl-hydroxy, —C₁₋₃ alkyl-cyano, —C₁₋₃ alkyl-C₁₋₆ alkoxy, —C₁₋₃ alkyl-halo C₁₋₆ alkyl or —C₁₋₃ alkyl-halo C₁₋₆ alkoxy;
Z is N—C(O)—CR_{X3}═CR_{X1}R_{X2} or N—C(O)—C≡CR_{X4}; wherein, R_{X1}, R_{X2} are each independently hydrogen, halogen, cyano, NR^{a}R^{b}, C₁₋₃ alkyl, halo C₁₋₃ alkyl, —C₁₋₃ alkyl-hydroxy, —C₁₋₃ alkyl-cyano, —C₁₋₃ alkyl-C₁₋₃ alkoxy, —C₁₋₃ alkyl-NR^{a}R^{b}, —C₁₋₃ alkyl-3- to 6-membered heterocycloalkyl, —C₁₋₃ alkyl-5- or 6-membered monocyclic heteroaryl; wherein, R^{a}, R^{b} are each independently hydrogen or C₁₋₃ alkyl; R_{X3} is hydrogen, halogen, —O—C₁₋₃ alkyl or —O—C₃₋₆ cycloalkyl; R_{X4} is hydrogen, halo C₁₋₃ alkyl, —C₁₋₃ alkyl-hydroxy, —C₁₋₃ alkyl-cyano, —C₁₋₃ alkyl-C₁₋₃ alkoxy; and
when R₀ is

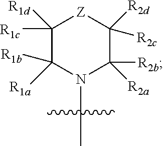

R₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁₁, —NH—R₁₁ or —N(R₁₁)R₁₂; wherein R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; or
when R₀ is

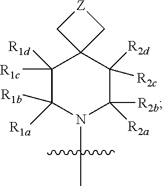

R₁ is halogen, cyano, substituted or unsubstituted C₁₋₆ alkyl, —O—R₁₁, —NH—R₁₁ or —N(R₁₁)R₁₂; wherein R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms;
L is a bond, —CR_{L1}R_{L2}—, —O—(CR_{L1}R_{L2})ₜ₁— or —NH—(CR_{L3}R_{L4})ₜ₂—; wherein, R_{L1}, R_{L2}, R_{L3}, R_{L4} are the same or different, and are each independently hydrogen, halogen, hydroxyl, hydroxymethyl, hydroxyethyl, C₁₋₃ alkyl or oxo group; t1, t2 are each independently 0, 1, 2, 3 or 4; among R_{L1} and R_{L2} or among R_{L3} and R_{L4}, when one of them is oxo group, the other does not exist;
R₂ is halogen, hydroxy, —SO₂C₁₋₆ alkyl, substituted or unsubstituted 3- to 20-membered heterocycloalkyl, substituted or unsubstituted C₃₋₂₀ cycloalkyl, substituted or unsubstituted 5- or 6-membered monocyclic heteroaryl or NR₂₁R₂₂; wherein R₂₁, R₂₂ are each independently hydrogen, substituted or unsubstituted C₁₋₆ alkyl, —SO₂C₁₋₆ alkyl, —SO₂C₃₋₆ cycloalkyl, —C(O)C₁₋₆ alkyl or —C(O) halo C₁₋₆ alkyl; or R₂₁, R₂₂ together with the nitrogen atom connected form a substituted or unsubstituted 3- to 20-membered heterocycloalkyl; wherein, the 3- to 20-membered heterocycloalkyl, the 5- or 6-membered monocyclic heteroaryl each independently have 1, 2 or 3 heteroatoms selected from N, O and S as ring atoms;
X is O, NR₃, S, S(O) or S(O)₂; wherein R₃ is hydrogen, C₁₋₆ alkyl, C₂₋₆ alkenyl, C₃₋₆ cycloalkyl or 3- to 6-membered heterocycloalkyl; wherein the 3- to 6-membered heterocycloalkyl has 1, 2 or 3 heteroatoms selected from N, O and S as ring atoms;
Y is substituted or unsubstituted C₃₋₂₀ cycloalkyl or substituted or unsubstituted 3- to 20-membered heterocycloalkyl; wherein the 3- to 20-membered heterocycloalkyl has 1, 2 or 3 heteroatoms selected from N, O and S as ring atoms;
W is CR₄ or N; wherein R₄ is hydrogen, halogen, cyano, hydroxyl, substituted or unsubstituted C₁₋₆ alkyl, substituted or unsubstituted C₁₋₆ alkoxy, substituted or unsubstituted C₃₋₂₀ cycloalkyl, substituted or unsubstituted C₃₋₂₀ cycloalkoxy, —NH—(C₁₋₄ alkyl) or —N(C₁₋₄ alkyl)₂;
the “substituted” mentioned above each independently means that 1, 2, 3, or 4 hydrogen atoms in the group are replaced by substituents independently selected from the group S; and the substituents in the group S are selected from the group consisting of hydroxyl, halogen, nitro, oxo, C₁₋₆ alkyl, C₁₋₆ alkyl substituted with hydroxy, benzyl, —(CH₂)ᵤ-cyano, —(CH₂)ᵤ—C₁₋₆ alkoxy, —(CH₂)ᵤ-halo C₁₋₆ alkoxy, —(CH₂)ᵤ-halo C₁₋₆ alkyl, —(CH₂)ᵤ-3- to 6-membered heterocycloalkyl, —(CH₂)ᵤ-5- or 6-membered monocyclic heteroaryl, —(CH₂)ᵤ—C₃₋₈ cycloalkyl, —(CH₂)ᵤ—O—(CH₂)ᵥ—C₃₋₈ cycloalkyl, —(CH₂)ᵤ—O—(CH₂)ᵥ—C₁₋₆ alkoxy, —(CH₂)ᵤ—O—(CH₂)ᵥOH, —(CH₂)ᵤ—SO₂C₁₋₆ alkyl, —(CH₂)ᵤ—NRₐ₀R_{b0}, —(CH₂)ᵤC(O)NRₐ₀R_{b0}, —(CH₂)ᵤC(O)C₁₋₆ alkyl, —C(O)OC₁₋₆ alkyl, —NRₐ₀C(O)—(CH₂)ᵤ—NRₐ₀R_{b0}, —NRₐ₀C(O)—(CH₂)ᵤOH, —NRₐ₀C(O) -halo C₁₋₆ alkyl; wherein the 3- to 6-membered heterocycloalkyl, the 5- or 6-membered monocyclic heteroaryl each independently have 1, 2 or 3 heteroatoms selected from N, O and S as ring atoms; the 3- to 6-membered heterocycloalkyl, the 5- or 6-membered monocyclic heteroaryl are optionally substituted with 1, 2 or 3 substituents selected from halogen, cyano, C₁₋₃ alkyl, C₁₋₃ alkoxy and C₃₋₆ cycloalkyl; u, v are each independently 0, 1, 2, 3 or 4; Rₐ₀, R_{b0} are each independently hydrogen or C₁₋₃ alkyl;
B is C₆₋₁₀ aryl, 5- or 6-membered monocyclic heteroaryl or 8- to 10-membered bicyclic heteroaryl; wherein the 5- or 6-membered monocyclic heteroaryl, the 8- to 10-membered bicyclic heteroaryl each independently have 1, 2 or 3 heteroatoms selected from N, O and S as ring atoms; the C₆₋₁₀ aryl, the 5- or 6-membered monocyclic heteroaryl, the 8- to 10-membered bicyclic heteroaryl are unsubstituted or substituted with 1, 2, 3 or 4 groups independently selected from Rₛ₁; or
B is the structure as shown in formula (B):

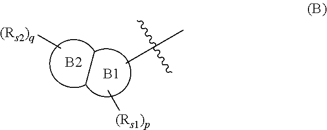

wherein, the B1 ring is a benzene ring or a 5- or 6-membered monocyclic heteroaryl ring; the B2 ring is a 5- or 6-membered heterocycloalkyl ring fused with the B1 ring or a 5- or 6-membered cycloalkyl ring fused with the B1 ring; wherein the 5- or 6-membered monocyclic heteroaryl ring, the 5- or 6-membered heterocycloalkyl ring each independently have 1, 2 or 3 heteroatoms selected from N, O and S as ring atoms;
(Rₛ₁)ₚ means that the hydrogens on the B1 ring are replaced by p of Rₛ₁, p is 0, 1, 2 or 3, and each Rₛ₁ is the same or different;
(Rₛ₂)_{q} means that the hydrogens on the B2 ring are replaced by q of Rₛ₂, q is 0, 1, 2 or 3, and each Rₛ₂ is the same or different;
Rₛ₁, Rₛ₂ are each independently hydroxy, halogen, nitro, oxo, C₁₋₆ alkyl, C₁₋₆ alkyl substituted with hydroxy, benzyl, —(CH₂)ᵤ₁-cyano, —(CH₂)ᵤ₁—C₁₋₆ alkoxy, —(CH₂)ᵤ₁-halo C₁₋₆ alkoxy, —(CH₂)ᵤ₁-halo C₁₋₆ alkyl, —(CH₂)ᵤ₁-3- to 6-membered heterocycloalkyl, —(CH₂)ᵤ₁-5- or 6-membered monocyclic heteroaryl, —(CH₂)ᵤ₁—C₃₋₈ cycloalkyl, —(CH₂)ᵤ₁—O—(CH₂)ᵥ₁—C₃₋₈ cycloalkyl, —(CH₂)ᵤ₁—O—(CH₂)ᵥ₁—C₁₋₆ alkoxy, —(CH₂)ᵤ₁—O—(CH₂)ᵥ₁OH, —(CH₂)ᵤ₁—SO₂C₁₋₆ alkyl, —(CH₂)ᵤ₁—NRₐ₀R_{b0}, —(CH₂)ᵤ₁—C(O)NRₐ₀R_{b0}, —(CH₂)ᵤ₁—C(O)C₁₋₆ alkyl, —C(O)OC₁-6 alkyl, —NRₐ₀C(O)—(CH₂)ᵤ₁—NRₐ₀R_{b0}, —NRₐ₀C(O)—(CH₂)ᵤ₁OH, —NRₐ₀C(O)-halo C₁₋₆ alkyl; wherein, the 3- to 6-membered heterocycloalkyl, the 5- or 6-membered monocyclic heteroaryl each independently have 1, 2 or 3 heteroatoms selected from N, O and S as ring atoms; the 3- to 6-membered heterocycloalkyl, the 5- or 6-membered monocyclic heteroaryl are optionally substituted with 1, 2 or 3 substituents selected from halogen, cyano, C₁₋₃ alkyl, C₁₋₃ alkoxy and C₃₋₆ cycloalkyl; u1, v1 are each independently 0, 1, 2, 3 or 4; Rₐ₀, R_{b0} are each independently hydrogen or C₁₋₃ alkyl.

In one embodiment, Z is —NC(O)—CR_{X3}═CR_{X1}R_{X2}; wherein R_{X1}, R_{X2} are each independently hydrogen, halogen, cyano, amino, NHCH₃, N(CH₃)₂, methyl, ethyl, n-propyl, isopropyl, monochloromethyl, dichloromethyl, trichloromethyl, monochloroethyl, 1,2-dichloroethyl, trichloroethyl, monobromoethyl, monofluoromethyl, difluoromethyl, trifluoromethyl, monofluoroethyl, difluoroethyl, trifluoroethyl, —CH₂-hydroxy, —CH₂-cyano, —CH₂-methoxy, —CH₂-ethoxy, —CH₂-propoxy, —CH₂-isopropoxy, —CH₂—NH₂, —CH₂—NHCH₃, —CH₂—N(CH₃)₂, —CH₂-3- to 6-membered heterocycloalkyl, —CH₂-5- or 6-membered monocyclic heteroaryl; the 3- to 6-membered heterocycloalkyl is selected from the group consisting of aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran; the 5- or 6-membered monocyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine; the 3- to 6-membered heterocycloalkyl, the 5- or 6-membered monocyclic heteroaryl are optionally substituted with 1 or 2 substituents selected from halogen and C₁₋₃ alkyl; R_{X3} is hydrogen, halogen, methoxy, ethoxy, propoxy or isopropoxy.

In one embodiment, Z is N—C(O)—CR_{X3}═CR_{X1}R_{X2}; wherein R_{X1}, R_{X2} are each independently hydrogen; R_{X3} is hydrogen.

In one embodiment, Z is N—C(O)—C≡CR_{X4}; wherein R_{X4} is hydrogen, monochloromethyl, dichloromethyl, trichloromethyl, monochloroethyl, 1,2-dichloroethyl, trichloroethyl, monobromoethyl, monofluoromethyl, difluoromethyl, trifluoromethyl, monofluoroethyl, difluoroethyl, trifluoroethyl, —CH₂-hydroxyl, —CH₂-cyano, —CH₂-methoxy, —CH₂-ethoxy, —CH₂-propoxy, —CH₂-isopropoxy.

In one embodiment, Z is N—C(O)—C≡CR_{X4}; wherein R_{X4} is hydrogen.

In one embodiment, when R₀ is

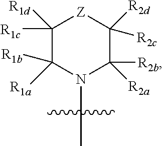

each group in R₀ is defined as before; R₁ is halogen, cyano, substituted or unsubstituted C₁₋₃ alkyl, substituted or unsubstituted C₂₋₄ alkenyl, substituted or unsubstituted C₃₋₆ cycloalkyl, —O—R₁₁, —NH—R₁ or —N(R₁₁)R₁₂; wherein, R₁₁, R₁₂ are each independently substituted or unsubstituted C₁₋₃ alkyl, substituted or unsubstituted C₃₋₆ cycloalkyl, substituted or unsubstituted 3- to 6-membered heterocycloalkyl, substituted or unsubstituted phenyl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, when R₀ is

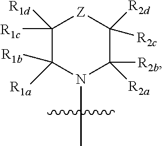

each group in R₀ is defined as before; R₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁₁, —NH—R₁₁ or —N(R₁₁)R₁₂; wherein, R₁₁, R₁₂ are each independently substituted or unsubstituted C₁₋₃ alkyl, substituted or unsubstituted C₃₋₆ cycloalkyl, substituted or unsubstituted 3- to 6-membered heterocycloalkyl, substituted or unsubstituted phenyl or substituted or unsubstituted 5- or 6-membered monocyclic heteroaryl; or Rn, R₁₂ together with the nitrogen atom connected form a substituted or unsubstituted 3- to 6-membered heterocycloalkyl; the “substituted” mentioned above each independently means that 1, 2, 3, or 4 hydrogen atoms in the group are replaced by substituents independently selected from the group S; the C₃₋₆ cycloalkyl is selected from cyclopropyl, cyclobutyl, cyclopentyl, cyclohexyl; the 3- to 6-membered heterocycloalkyl is selected from aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran; the 5- or 6-membered monocyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, when R₀ is

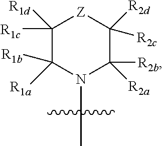

each group in R₀ is defined as before; R₁ is halogen, cyano, unsubstituted C₁₋₃ alkyl, unsubstituted C₂₋₄ alkenyl, unsubstituted C₃₋₆ cycloalkyl, —O—R₁₁, —NH—R₁ or —N(R₁₁)R₁₂; wherein, R₁₁, R₁₂ are each independently substituted or unsubstituted C₁₋₃ alkyl.

In one embodiment, when R₀ is

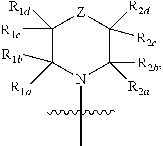

each group in R₀ is defined as before; R₁ is halogen, cyano, methyl, ethenyl, cyclopropyl —O-methyl or —NH-methyl.

In one embodiment, when R₀ is

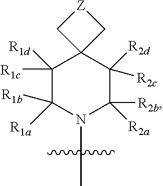

each group in R₀ is defined as before; R₁ is halogen, cyano, substituted or unsubstituted C₁₋₃ alkyl, —O—R₁₁, —NH—R₁₁ or —N(R₁₁)R₁₂; wherein, Rn, R₁₂ are each independently substituted or unsubstituted C₁₋₃ alkyl, substituted or unsubstituted C₃₋₆ cycloalkyl, substituted or unsubstituted 3- to 6-membered heterocycloalkyl, substituted or unsubstituted phenyl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, when R₀ is

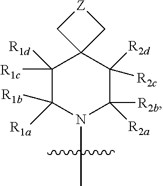

each group in R₀ is defined as before; R₁ is halogen, cyano, substituted or unsubstituted C₁₋₆ alkyl, —O—R₁₁, —NH—R₁₁ or —N(R₁₁)R₁₂; wherein, R₁₁, R₁₂ are each independently substituted or unsubstituted C₁₋₃ alkyl, substituted or unsubstituted C₃₋₆ cycloalkyl, substituted or unsubstituted 3- to 6-membered heterocycloalkyl, substituted or unsubstituted phenyl or substituted or unsubstituted 5- or 6-membered monocyclic heteroaryl; or Ru, R₁₂ together with the nitrogen atom connected form a substituted or unsubstituted 3- to 6-membered heterocycloalkyl; the “substituted” mentioned above each independently means that 1, 2, 3, or 4 hydrogen atoms in the group are replaced by substituents independently selected from the group S; the C₃₋₆ cycloalkyl is selected from cyclopropyl, cyclobutyl, cyclopentyl, cyclohexyl; the 3- to 6-membered heterocycloalkyl is selected from aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran; the 5- or 6-membered monocyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, when R₀ is

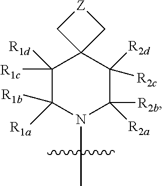

each group in R₀ is defined as before; R₁ is cyano, methyl, —O-methyl, or —NH-methyl.

In one embodiment, the compound represented by formula (I) is the compound represented by formula (II):

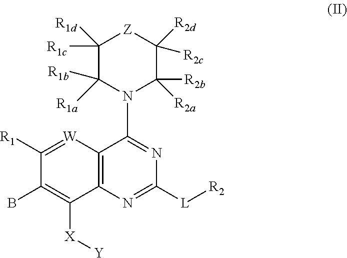

wherein, R₂, X, Y, B, W, Z, L are defined as before; R₁ₐ, R_{1b}, R_{1c}, R_{1d}, R₂ₐ, R_{2b}, R_{2c}, R_{2d} are the same or different, and are each independently hydrogen, halogen, C₁₋₃ alkyl, —C₁₋₃ alkyl-hydroxy, —C₁₋₃ alkyl-cyano, —C₁₋₃ alkyl-C₁₋₆ alkoxy, —C₁₋₃ alkyl-halo C₁₋₆ alkyl or —C₁₋₃ alkyl-halo C₁₋₆ alkoxy; R₁₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁₁, —NH—R₁₁ or —N(R₁₁)R₁₂; wherein, R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, the compound represented by formula (I) is the compound represented by formula (III):

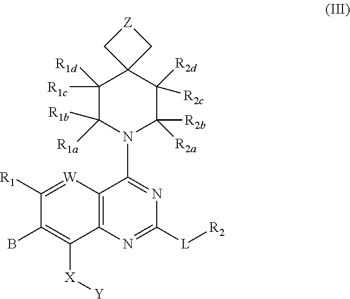

wherein, R₂, X, Y, B, W, Z, L are defined as before; R₁ₐ, R_{1b}, R_{1c}, R_{1d}, R₂ₐ, R_{2b}, R_{2c}, R_{2d} are the same or different, and are each independently hydrogen, halogen, C₁₋₃ alkyl, —C₁₋₃ alkyl-hydroxy, —C₁₋₃ alkyl-cyano, —C₁₋₃ alkyl-C₁₋₆ alkoxy, —C₁₋₃ alkyl-halo C₁₋₆ alkyl or —C₁₋₃ alkyl-halo C₁₋₆ alkoxy; R₁ is halogen, cyano, substituted or unsubstituted C₁₋₆ alkyl, —O—R₁₁, —NH—R₁ or —N(R₁₁)R₁₂; wherein, R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, R₂ is halogen, hydroxy, —SO₂C₁₋₃ alkyl, substituted or unsubstituted 3- to 6-membered heterocycloalkyl, substituted or unsubstituted C₃₋₆ cycloalkyl, substituted or unsubstituted 5- or 6-membered monocyclic heteroaryl, or NR₂₁R₂₂; wherein, R₂₁, R₂₂ are each independently hydrogen, substituted or unsubstituted C₁₋₃ alkyl, —SO₂C₁₋₃ alkyl, —SO₂C₃₋₆ cycloalkyl, —C(O)C₁₋₃ alkyl or —C(O)halo C₁₋₃ alkyl; or R₂₁ and R₂₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, R₂ is halogen, hydroxy, —SO₂C₁₋₃ alkyl, substituted or unsubstituted 3- to 6-membered heterocycloalkyl, substituted or unsubstituted C₃₋₆ cycloalkyl, substituted or unsubstituted 5- or 6-membered monocyclic heteroaryl, or NR₂₁R₂₂; wherein, R₂₁, R₂₂ are each independently hydrogen, substituted or unsubstituted C₁₋₃ alkyl, —SO₂C₁₋₃ alkyl, —SO₂C₃₋₆ cycloalkyl, —C(O)C₁₋₃ alkyl or —C(O)halo C₁₋₃ alkyl; or R₂₁ and R₂₂ together with the nitrogen atom connected form a substituted or unsubstituted 3- to 6-membered heterocycloalkyl; wherein, the “substituted” mentioned above each independently means that 1, 2, 3, or 4 hydrogen atoms in the group are replaced by substituents independently selected from the group S; the C₃₋₆ cycloalkyl is selected from cyclopropyl, cyclobutyl, cyclopentyl, cyclohexyl; the 3- to 6-membered heterocycloalkyl is selected from aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran; the 5- or 6-membered monocyclic heteroaryl is selected from: thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, W is CR₄ or N; wherein, R₄ is hydrogen, halogen, cyano, hydroxy, substituted or unsubstituted C₁₋₃ alkyl, substituted or unsubstituted C₁₋₃ alkoxy, substituted or unsubstituted C₃₋₆ cycloalkyl, substituted or unsubstituted C₃₋₆ cycloalkoxy, —NH—(C₁₋₃ alkyl) or —N(C₁₋₃ alkyl)₂; the “substituted” mentioned above each independently means that 1, 2, 3, or 4 hydrogen atoms in the group are replaced by substituents independently selected from the group S.

In one embodiment, W is CR₄ or N; wherein, R₄ is hydrogen, halogen, cyano, hydroxy, substituted or unsubstituted C₁₋₃ alkyl, substituted or unsubstituted C₁₋₃ alkoxy, substituted or unsubstituted C₃₋₆ cycloalkyl, substituted or unsubstituted C₃₋₆ cycloalkoxy, —NH—(C₁₋₃ alkyl) or —N(C₁₋₃ alkyl)₂; the “substituted” mentioned above each independently means that 1, 2, 3, or 4 hydrogen atoms in the group are replaced by substituents independently selected from the group S; the C₃₋₆ cycloalkyl is selected from cyclopropyl, cyclobutyl, cyclopentyl, cyclohexyl; the C₃₋₆ cycloalkoxy is selected from cyclopropoxy, cyclobutoxy, cyclopentyloxy, cyclohexyloxy.

In one embodiment, W is CH or N.

In one embodiment, the substituents in the group S are selected from the group consisting of hydroxy, halogen, nitro, oxo, C₁₋₃ alkyl, hydroxy substituted C₁₋₃ alkyl, benzyl, —(CH₂)ᵤ-cyano, —(CH₂)ᵤ—C₁₋₃ alkoxy, —(CH₂)ᵤ-halo C₁₋₃ alkoxy, —(CH₂)ᵤ-halo C₁₋₃ alkyl, —(CH₂)ᵤ-3- to 6-membered heterocycloalkyl, —(CH₂)ᵤ-5- or 6-membered monocyclic heteroaryl, —(CH₂)ᵤ—C₃₋₆ cycloalkyl, —(CH₂)ᵤ—O—(CH₂)ᵥ—C₃₋₆ cycloalkyl, —(CH₂)ᵤ—O—(CH₂)ᵥ—C₁₋₃ alkoxy, —(CH₂)ᵤ—O—(CH₂)ᵥOH, —(CH₂)ᵤ—SO₂C₁₋₃ alkyl, —(CH₂)ᵤ—NRₐ₀R_{b0}, —(CH₂)ᵤ—C(O)NRₐ₀R_{b0}, —(CH₂)ᵤ—C(O)C₁₋₃ alkyl, —C(O)OC₁₋₃ alkyl, —NRₐ₀C(O)—(CH₂)ᵤ—NRₐ₀R_{b0}, —NRₐ₀C(O)—(CH₂)ᵤOH, —NRₐ₀C(O)-halo C₁₋₃ alkyl; wherein, the 3- to 6-membered heterocycloalkyl, the 5- or 6-membered monocyclic heteroaryl each independently have 1, 2 or 3 heteroatoms selected from N, O and S as ring atoms; the 3- to 6-membered heterocycloalkyl and the 5- or 6-membered monocyclic heteroaryl are optionally substituted with 1, 2 or 3 substituents selected from halogen, cyano, C₁₋₃ alkyl, C₁₋₃ alkoxy and C₃₋₆ cycloalky; u, v are each independently 0, 1, 2, 3 or 4; Rₐ₀, R_{b0} are each independently hydrogen or C₁₋₃ alkyl.

In one embodiment, the substituent in the group S is halogen.

In one embodiment, the substituents in the group S are selected from the group consisting of C₁₋₃ alkyl, —(CH₂)ᵤ-3- to 6-membered heterocycloalkyl, —(CH₂)ᵤ—SO₂C₁₋₃ alkyl, —(CH₂)ᵤ—NRₐ₀R_{b0}; wherein, the 3- to 6-membered heterocycloalkyl has 1, 2 or 3 heteroatoms selected from N, O and S as ring atoms; the 3- to 6-membered heterocycloalkyl is optionally substituted with 1, 2 or 3 substituents selected from halogen, cyano, C₁₋₃ alkyl, C₁₋₃ alkoxy and C₃₋₆ cycloalky; u is 0, 1, 2, 3 or 4; Rₐ₀, R_{b0} are each independently hydrogen or C₁₋₃ alkyl.

In one embodiment, in the substituents in the group S, the 3- to 6-membered heterocycloalkyl is selected from aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran.

In one embodiment, in the substituents in the group S, the C₃₋₆ cycloalkyl is selected from cyclopropyl, cyclobutyl, cyclopentyl, cyclohexyl.

In one embodiment, in the substituents in the group S, the 5- or 6-membered monocyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, the Rₛ₁, Rₛ₂ are each independently selected from hydroxy, halogen, nitro, oxo, C₁₋₃ alkyl, hydroxy substituted C₁₋₃ alkyl, benzyl, —(CH₂)ᵤ₁-cyano, —(CH₂)ᵤ₁—C₁₋₃ alkoxy, —(CH₂)ᵤ₁-halo C₁₋₃ alkoxy, —(CH₂)ᵤ₁-halo C₁₋₃ alkyl, —(CH₂)ᵤ₁-3- to 6-membered heterocycloalkyl, —(CH₂)ᵤ₁-5- or 6-membered monocyclic heteroaryl, —(CH₂)ᵤ₁—C₃₋₆ cycloalkyl, —(CH₂)ᵤ₁—O—(CH₂)ᵥ₁—C₃₋₆ cycloalkyl, —(CH₂)ᵤ₁—O—(CH₂)ᵥ₁—C₁₋₃ alkoxy, —(CH₂)ᵤ₁—O—(CH₂)ᵥ₁OH, —(CH₂)ᵤ₁—SO₂C₁₋₃ alkyl, —(CH₂)ᵤ₁—NRₐ₀R_{b0}, —(CH₂)ᵤ₁—C(O)NRₐ₀R_{b0}, —(CH₂)ᵤ₁—C(O)C₁₋₃ alkyl, —C(O)OC₁-3 alkyl, —NRₐ₀C(O)—(CH₂)ᵤ₁—NRₐ₀R_{b0}, —NRₐ₀C(O)—(CH₂)ᵤ₁OH, —NRₐ₀C(O)-halo C₁₋₃ alkyl; wherein, the 3- to 6-membered heterocycloalkyl, the 5- or 6-membered monocyclic heteroaryl each independently have 1, 2 or 3 heteroatoms selected from N, O and S as ring atoms; the 3- to 6-membered heterocycloalkyl, the 5- or 6-membered monocyclic heteroaryl are optionally substituted with 1, 2 or 3 substituents selected from halogen, cyano, C₁₋₃ alkyl, C₁₋₃ alkoxy and C₃₋₆ cycloalky; u1, v1 are each independently 0, 1, 2, 3 or 4; Rₐ₀, R_{b0} are each independently hydrogen or C₁₋₃ alkyl.

In one embodiment, the Rₛ₁, Rₛ₂ are each independently halogen.

In one embodiment, the Rₛ₁, Rₛ₂ are each independently selected from C₁₋₃ alkyl, —(CH₂)ᵤ₁-3- to 6-membered heterocycloalkyl, —(CH₂)ᵤ₁—SO₂C₁₋₃ alkyl, —(CH₂)ᵤ₁—NRₐ₀R_{b0}; wherein, the 3- to 6-membered heterocycloalkyl has 1, 2 or 3 heteroatoms selected from N, O and S as ring atoms; the 3- to 6-membered heterocycloalkyl is optionally substituted with 1, 2 or 3 substituents selected from halogen, cyano, C₁₋₃ alkyl, C₁₋₃ alkoxy and C₃₋₆ cycloalky; u1 is 0, 1, 2, 3 or 4; Rₐ₀, R_{b0} are each independently hydrogen or C₁₋₃ alkyl.

In one embodiment, in Rₛ₁, Rₛ₂, the 3- to 6-membered heterocycloalkyl is selected from aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran.

In one embodiment, in Rₛ₁, Rₛ₂, the C₃₋₆ cycloalkyl is selected from cyclopropyl, cyclobutyl, cyclopentyl, cyclohexyl.

In one embodiment, in Rₛ₁, Rₛ₂, the 5- or 6-membered monocyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, the compound represented by formula (I) is the compound represented by formula (II-1) or formula (III-1):

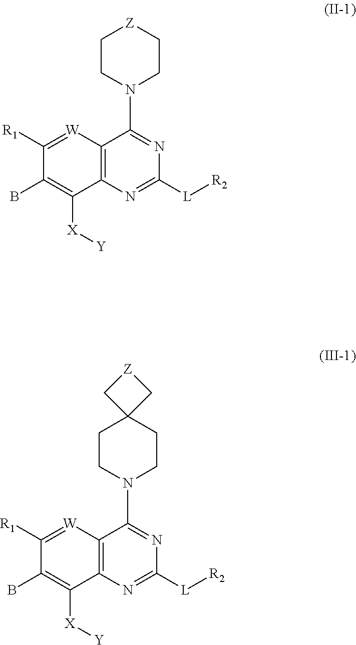

in each formula, R₂, X, Y, B, W, Z, L are defined as before; in formula (II-1), R₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁₁, —NH—R₁ or —N(R₁₁)R₁₂; wherein, R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; in formula (III-1), R₁ is halogen, cyano, substituted or unsubstituted C₁₋₆ alkyl, —O—R₁₁, —NH—R₁₁ or —N(R₁₁)R₁₂; wherein, R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, R₁₁ is substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; wherein, 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, R₁₁ is substituted or unsubstituted C₁₋₃ alkyl, substituted or unsubstituted C₃₋₆ cycloalkyl, substituted or unsubstituted 3- to 6-membered heterocycloalkyl, substituted or unsubstituted phenyl, substituted or unsubstituted 5- or 6-membered monocyclic heteroaryl; the “substituted” mentioned above each independently means that 1, 2, 3, or 4 hydrogen atoms in the group are replaced by substituents independently selected from the group S; the C₃₋₆ cycloalkyl is selected from cyclopropyl, cyclobutyl, cyclopentyl, cyclohexyl; the 3- to 6-membered heterocycloalkyl is selected from aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran; the 5- or 6-membered monocyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, in the formula (II-1) or formula (II), R₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁ or —NH—R₁₁; wherein, R₁₁ is substituted or unsubstituted C₁₋₆ alkyl or substituted or unsubstituted C₃₋₆ cycloalkyl; the “substituted” mentioned above each independently means that 1, 2, 3, or 4 hydrogen atoms in the group are replaced by substituents independently selected from the group S.

In one embodiment, in the formula (III-1) or formula (III), R₁ is halogen, cyano, substituted or unsubstituted C₁₋₆ alkyl, —O—R₁₁ or —NH—R₁₁; wherein, R₁₁ is substituted or unsubstituted C₁₋₆ alkyl or substituted or unsubstituted C₃₋₆ cycloalkyl; the “substituted” mentioned above each independently means that 1, 2, 3, or 4 hydrogen atoms in the group are replaced by substituents independently selected from the group S.

In one embodiment, W is CR₄; wherein, R₄ is hydrogen, halogen, cyano, hydroxy, substituted or unsubstituted C₁₋₃ alkyl, substituted or unsubstituted C₁₋₃ alkoxy, substituted or unsubstituted C₃₋₆ cycloalkyl, substituted or unsubstituted C₃₋₆ cycloalkoxy, —NH—(C₁₋₃ alkyl) or —N(C₁₋₃ alkyl)₂; the “substituted” mentioned above each independently means that 1, 2, 3, or 4 hydrogen atoms in the group are replaced by substituents independently selected from the group S.

In one embodiment, R₄ is hydrogen.

In one embodiment, W is N.

In one embodiment, X is O, NH, S, S(O) or S(O)_{2.}

In one embodiment, Y is substituted or unsubstituted C₃₋₆ cycloalkyl or substituted or unsubstituted 3- to 6-membered heterocycloalkyl; wherein, the 3- to 6-membered heterocycloalkyl has 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, X—Y is selected from the group consisting of: O-substituted or unsubstituted C₃₋₆ cycloalkyl, O-substituted or unsubstituted 3- to 6-membered heterocycloalkyl; wherein, the 3- to 6-membered heterocycloalkyl has 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, X—Y is selected from the group consisting of O-substituted or unsubstituted C₃₋₆ cycloalkyl, O-substituted or unsubstituted 3- to 6-membered heterocycloalkyl; wherein, the 3- to 6-membered heterocycloalkyl has one atom O as the ring atom; the “substituted” mentioned above each independently means that 1 or 2 hydrogen atoms in the group are replaced by halogen.

In one embodiment, L is a bond; R₂ is NR₂₁R₂₂; wherein, R₂₁ and R₂₂ together with the nitrogen atom connected form a substituted or unsubstituted 3- to 6-membered heterocycloalkyl, substituted or unsubstituted 6- to 10-membered fused heterocycloalkyl, or substituted or unsubstituted 7- to 11-membered spiro heterocycloalkyl; wherein, the 3- to 6-membered heterocycloalkyl, the 6- to 10-membered fused heterocycloalkyl, the 7- to 11-membered spiro heterocycloalkyl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, L is —CR_{L1}R_{L2}—, —O—(CR_{L1}R_{L2})ₜ₁— or —NH—(CR_{L3}R_{L4})ₜ₂—; R₂ is halogen, hydroxy, —SO₂C₁₋₆ alkyl, substituted or unsubstituted 3- to 6-membered heterocycloalkyl, substituted or unsubstituted 6- to 10-membered fused heterocycloalkyl, substituted or unsubstituted 7- to 11-membered spiro heterocycloalkyl, substituted or unsubstituted C₃₋₈ cycloalkyl, substituted or unsubstituted 5- or 6-membered monocyclic heteroaryl, or NR₂₁R₂₂; wherein, R_{L1}, R_{L2}, R_{L3}, R_{L4} are the same or different, and are each independently hydrogen, halogen, hydroxy, hydroxymethyl, hydroxyethyl, C₁₋₃ alkyl or oxo; among R_{L1} and R_{L2} or among R_{L3} and R_{L4}, when one of them is oxo group, the other does not exist; t1, t2 are each independently 0, 1, 2, 3 or 4; R₂₁, R₂₂ are each independently hydrogen, substituted or unsubstituted C₁₋₆ alkyl, —SO₂C₁₋₆ alkyl, —SO₂C₃₋₆ cycloalkyl, —C(O)C₁₋₆ alkyl, —C(O)halo C₁₋₆ alkyl; or R₂₁ and R₂₂ together with the nitrogen atom connected form a substituted or unsubstituted 3- to 6-membered heterocycloalkyl, a substituted or unsubstituted 6- to 10-membered fused heterocycloalkyl, or a substituted or unsubstituted 7- to 11-membered spiro heterocycloalkyl; wherein, the 3- to 6-membered heterocycloalkyl, the 6- to 10-membered fused heterocycloalkyl, the 7- to 11-membered spiro heterocycloalkyl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

In one embodiment, in the groups (such as R₁, B), the C₆₋₁₀ aryl are each independently phenyl or naphthyl.

In one embodiment, when the C₆₋₁₀ aryl is phenyl, B is selected from the following structure:

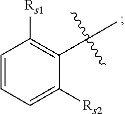

wherein, Rₛ₁, Rₛ₂ are defined as before.

In one embodiment, in the groups (such as R₁, R₂, B), the 5- or 6-membered monocyclic heteroaryls are each independently selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, in the groups (such as R₁, B), the 8- to 10-membered bicyclic heteroaryls are each independently a 9- or 10-membered bicyclic heteroaryl ring in which a phenyl ring is fused with a 5- or 6-membered monocyclic heteroaryl ring, or a 8- to 10-membered bicyclic heteroaryl ring in which a 5- or 6-membered monocyclic heteroaryl ring is fused with a 5- or 6-membered monocyclic heteroaryl ring; wherein, the 9- or 10-membered bicyclic heteroaryl ring in which a phenyl ring is fused with a 5- or 6-membered monocyclic heteroaryl ring, the 8- to 10-membered bicyclic heteroaryl ring in which a 5- or 6-membered monocyclic heteroaryl ring is fused with a 5- or 6-membered monocyclic heteroarylring each independently have 1, 2 or 3 heteroatoms selected from N, O and S as ring atoms.

In one embodiment, the 5- or 6-membered monocyclic heteroaryl ring to form the bicyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, the 5- or 6-membered monocyclic heteroaryl ring to form the bicyclic heteroaryl is selected from the following structures:

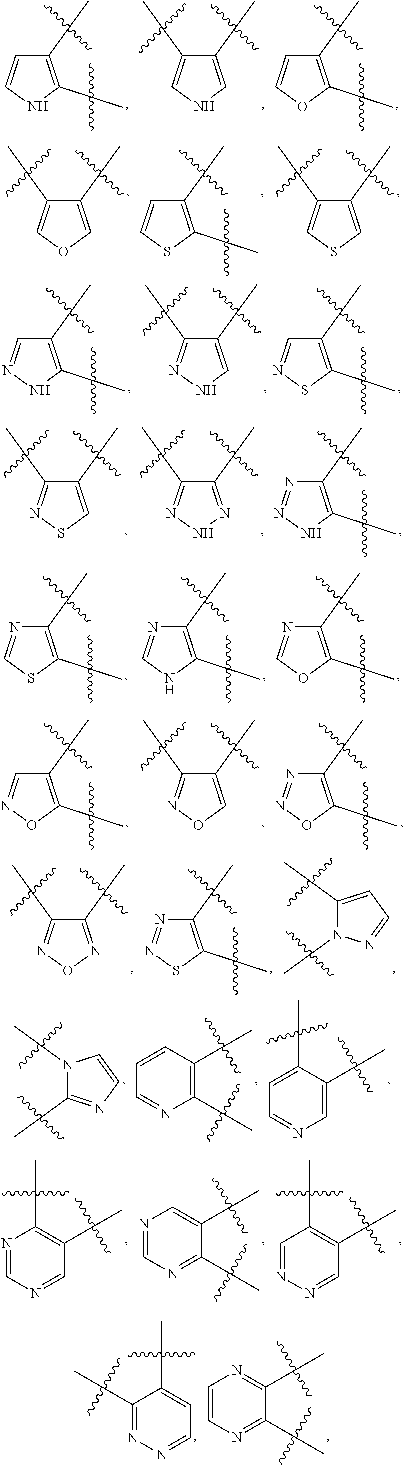

wherein, the two ring atoms connected represented by “ [symbol] ” are the pair of adjacent atoms that are shared with other rings when they are fused.

In one embodiment, in the groups (such as R₁, B), the 8- to 10-membered bicyclic heteroaryls are each independently selected from benzoxazole, benzisoxazole, benzimidazole, benzothiazole, benzisothiazole, benzotriazole, benzofuran, benzothiophene, indole, indazole, isoindole, quinoline, isoquinoline, quinazoline, quinoxaline, cinnoline, pyridopyrimidine and naphthyridine.

In one embodiment, in ring B1, the 5- or 6-membered monocyclic heteroaryl ring is selected from thiophene ring, furan ring, thiazole ring, isothiazole ring, imidazole ring, oxazole ring, pyrrole ring, pyrazole ring, triazole ring, 1,2,3-triazole ring, 1,2,4-triazole ring, 1,2,5-triazole ring, 1,3,4-triazole ring, tetrazole ring, isoxazole ring, oxadiazole ring, 1,2,3-oxadiazole ring, 1,2,4-oxadiazole ring, 1,2,5-oxadiazole ring, 1,3,4-oxadiazole ring, thiadiazole ring, pyridine ring, pyridazine ring, pyrimidine ring, pyrazine ring.

In one embodiment, in ring B1, the 5- or 6-membered monocyclic heteroaryl ring is selected from the following structures:

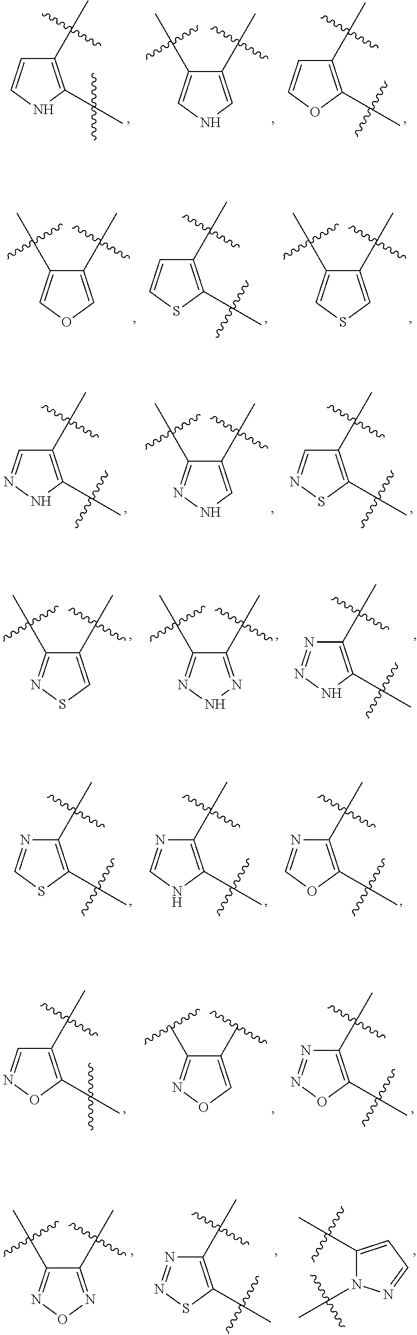

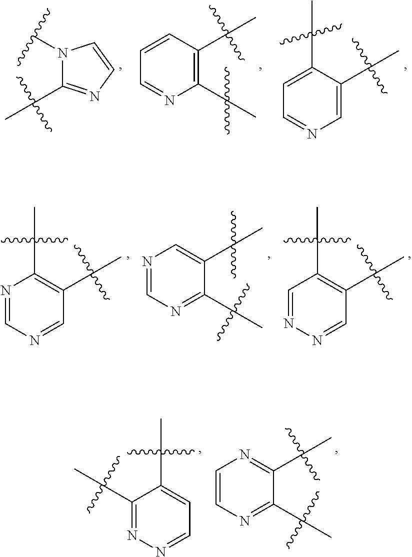

wherein, the two ring atoms connected represented by “ [symbol] ” are the pair of adjacent atoms that are shared with other rings when they are fused.

In one embodiment, in ring B2, the 5- or 6-membered cycloalkyl ring fused with ring B1 is selected from cyclopentyl ring, cyclopentenyl ring, cyclohexyl ring, cyclohexenyl ring, cyclohexadienyl ring, cyclopentanone, cyclopentane-1,3-dione, cyclohexanone, cyclohexane-1,3-dione.

In one embodiment, in ring B2, the 5- or 6-membered heterocycloalkyl ring fused with ring B1 is selected from oxazolidine, pyrrolidine-2-one, pyrrolidine-2,5-dione, 1,3-dioxolane, dihydrofuran-2 (3H)-one, dihydrofuran-2,5-dione, piperidine-2-one, piperidine-2,6-dione, tetrahydro-2H-pyran-2-one, imidazolidine, tetrahydrofuran, tetrahydrothiophene, tetrahydropyrrole, 1,3-dioxolane-2-one, oxazolidine-2-one, imidazolidine-2-one, piperidine, piperazine, piperazine-2-one, morpholine, morpholine-3-one, morpholine-2-one, thiomorpholine-3-one 1,1-dioxide, thiomorpholine, thiomorpholine-1,1-dioxide, tetrahydropyran, 1,2-dihydroazetidine, 1,2-dihydrooxetadiene, 2,5-dihydro-TH-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazinane, hexahydropyrimidine, 1,4-dioxane, tetrahydropyrimidine-2 (1H)-one, 1,4-dioxane-2-one, 5,6-dihydro-2H-pyran-2-one, 5,6-dihydropyrimidine-4 (3H)-one, 3,4-dihydropyridine-2 (1H)-one, 5,6-dihydropyridine-2 (1H)-one, 5,6-dihydropyrimidine-4 (1H)-one, pyrimidine-4 (3H)-one, pyrimidine-4 (1H)-one, 4,5-dihydro-TH-imidazole, 2,3-dihydro-1H-imidazole, 2,3-dihydroxazole, 1,3-dioxolene, 2,3-dihydrothiophene, 2,5-dihydrothiophene, 3,4-dihydro-2H-1,4-oxazine, 3,4-dihydro-2H-1,4-thiazide 1,1-dioxide, 1,2,3,4-tetrahydropyrazine, 1,3-dihydro-2H-pyrrole-2-one, 1,5-dihydro-2H-pyrrole-2-one, 1H-pyrrole-2,5-dione, furan-2 (3H)-one, furan-2 (5H)-one, 1,3-dioxolene-2-one, oxazole-2 (3H)-one, 1,3-dihydro-2H-imidazole-2-one, furan-2,5-dione, 3,6-dihydropyridine-2 (1H)-one, pyridine-2,6-(1H, 3H)-dione, 5,6-dihydro-2H-pyran-2-one, 3,6-dihydro-2H-pyran-2-one, 3,4-dihydro-2H-1,3-oxazine, 3,6-dihydro-2H-1,3-oxazine, 1,2,3,4-tetrahydropyrimidine.

In one embodiment B is selected from the following structures:

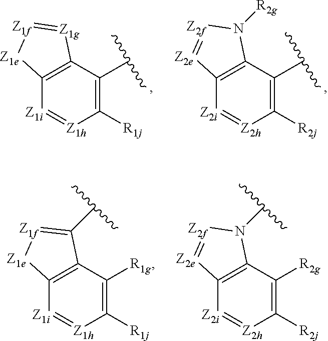

wherein, Z₁ₑ is NR₁ₑ, O or S; Z_{1f} is N or CR_{1f}; Z_{1g} is N or CR_{1g}; Z₁ₕ is N or CR₁ₕ; Z₁ᵢ is N or CR₁ᵢ; Z₂ₑ is N or CR₂ₑ; Z_{2f} is N or CR_{2f}; Z₂ₕ is N or CR₂ₕ; Z₂ᵢ is N or CR₂ᵢ; R₁ₑ, R_{1f}, R_{1g}, R₁ₕ, R₁ᵢ, R₁ⱼ, R₂ₑ, R_{2f}, R_{2g}, R₂ₕ, R₂ᵢ, R₂ⱼ are each independently hydrogen, halogen, C₁₋₃ alkyl, —CONH₂, —CONHC₁₋₃ alkyl, —CON(C₁₋₃ alkyl)₂, cyano, nitro, cyclopropyl, cyclobutyl, cyclopentyl, cyclohexyl, hydroxy, acetyl, hydroxymethyl, hydroxyethyl, carboxyl, NH₂, NHC₁₋₃ alkyl, N(C₁₋₃ alkyl)₂, halo C₁₋₃ alkyl, C₁₋₃ alkoxy, C₃₋₆ cycloalkoxy, C₂₋₄ alkenyl, C₂₋₄alkynyl, —C(O)OC₁₋₃ alkyl, —CHO, —OC(O)C₁₋₃ alkyl, —SO₂C₁₋₃ alkyl, —SO₂-phenyl or —CO-phenyl.

In one embodiment, in the groups (such as R₁, B), the 8- to 10-membered bicyclic heteroaryl is selected from the following structures:

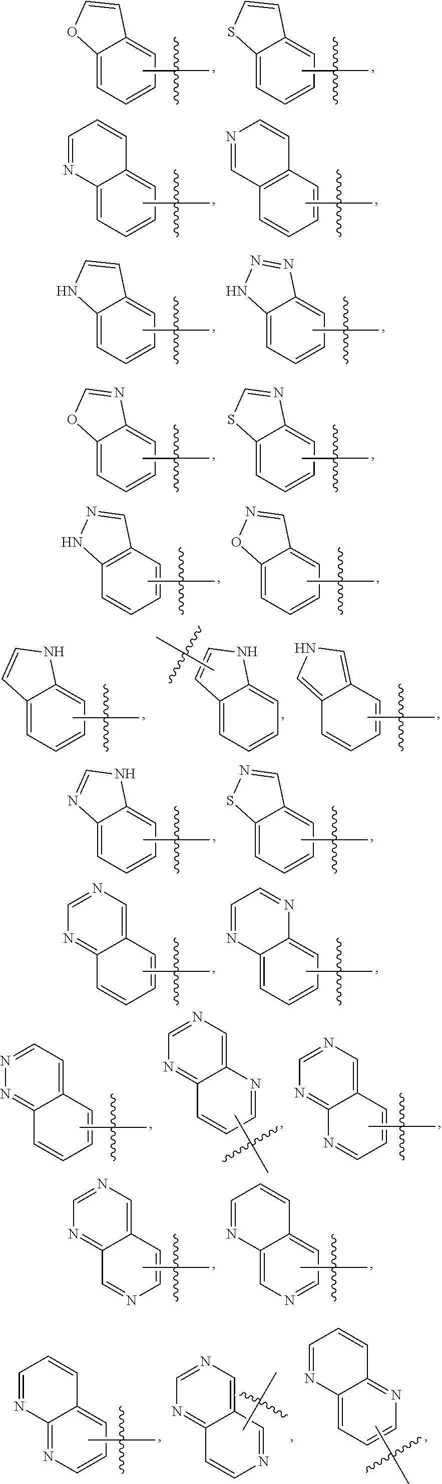

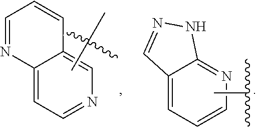

In one embodiment, in the groups (such as R₁, B), the 8- to 10-membered bicyclic heteroaryl is selected from the following structures:

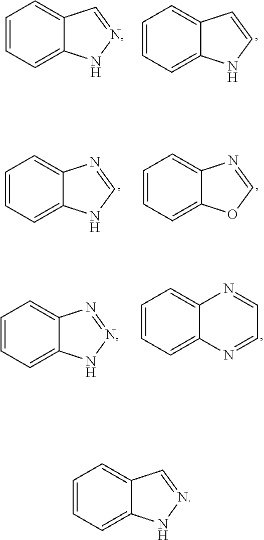

In one embodiment, in the groups (such as R₁, B), the 8- to 10-membered bicyclic heteroaryl is selected from the following structures:

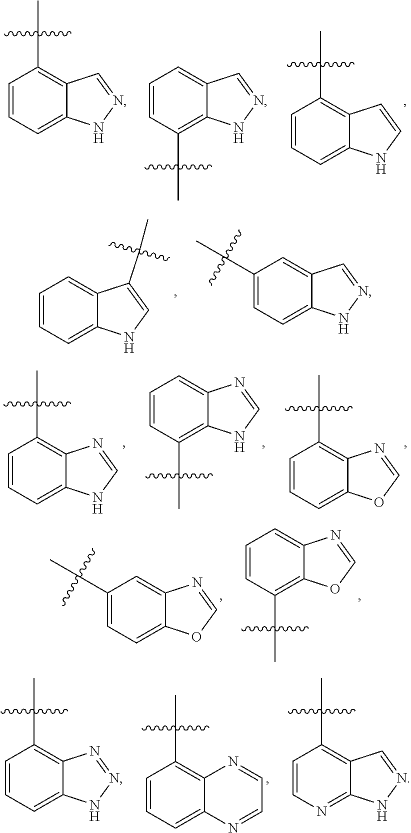

In one embodiment, B is the structure as shown in formula (B), and in formula (B),

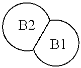

is selected from the following structures:

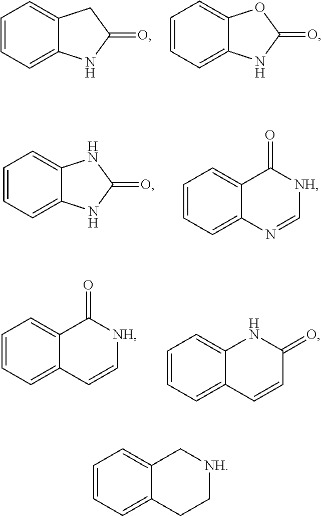

In one embodiment B is the structure as shown in formula (B) which is selected from the following structures:

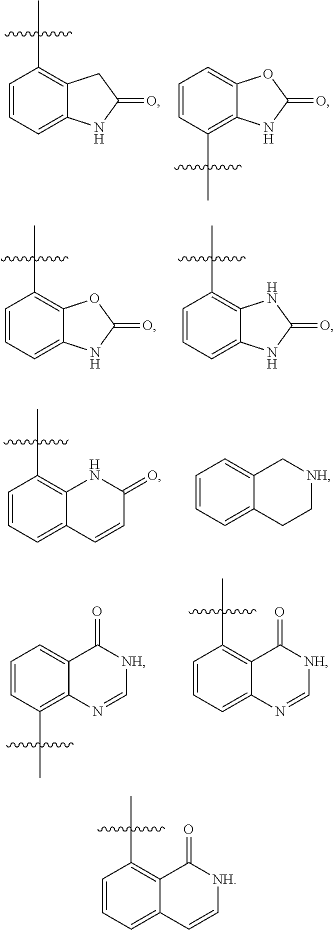

In one embodiment B is selected from the following structures:

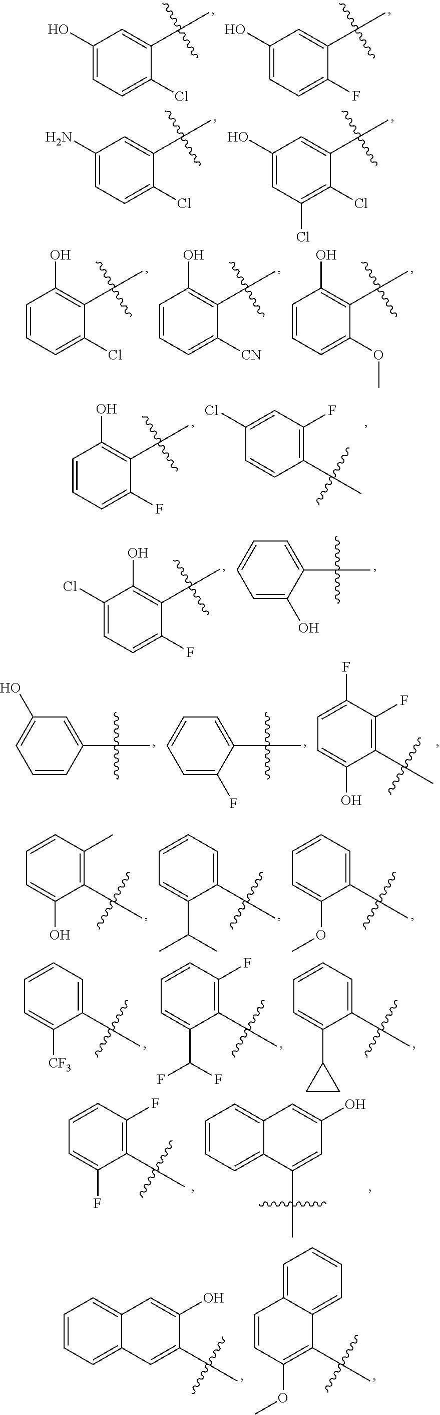

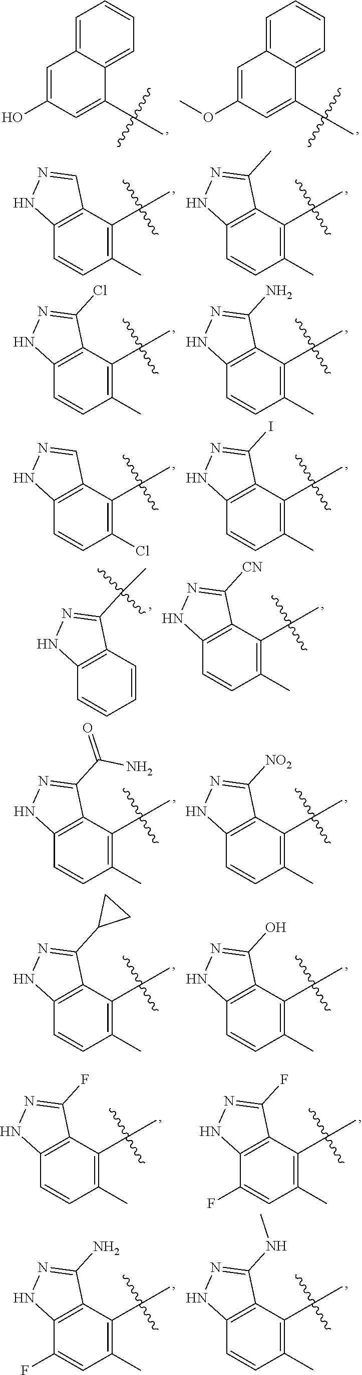

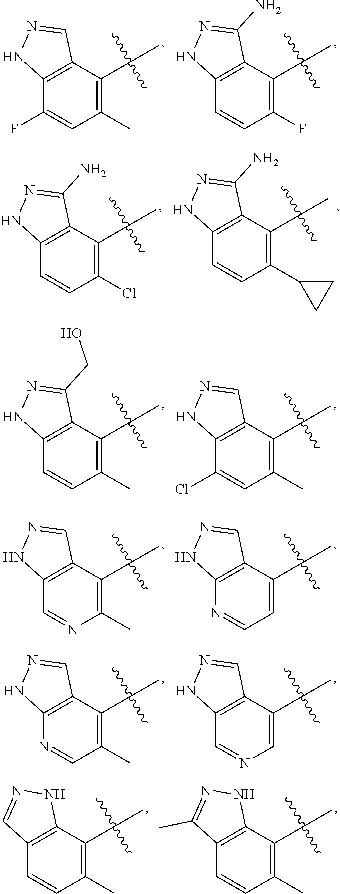

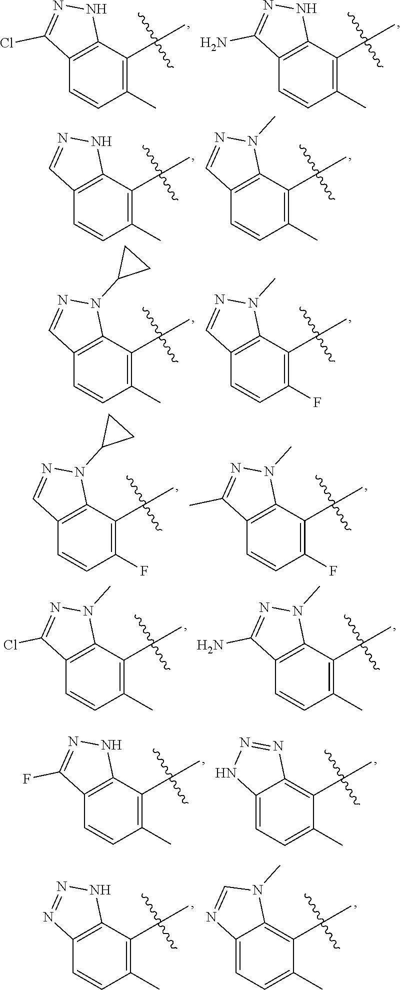

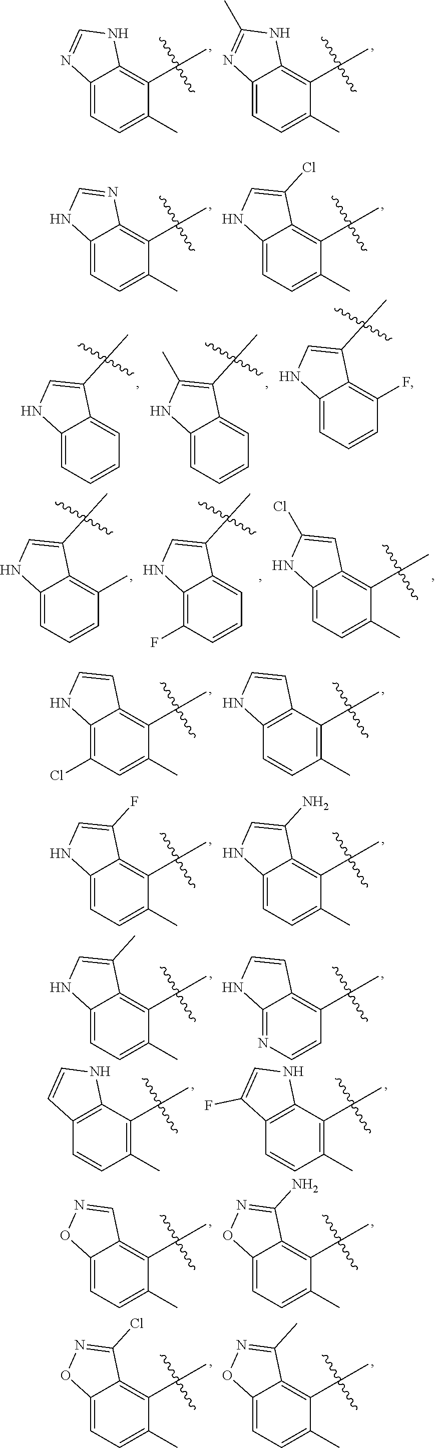

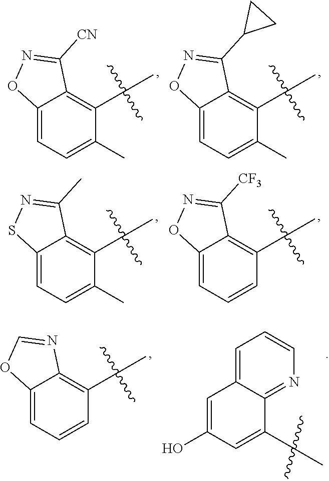

In one embodiment, -L-R₂ in the formula (I) is selected from the following structures:

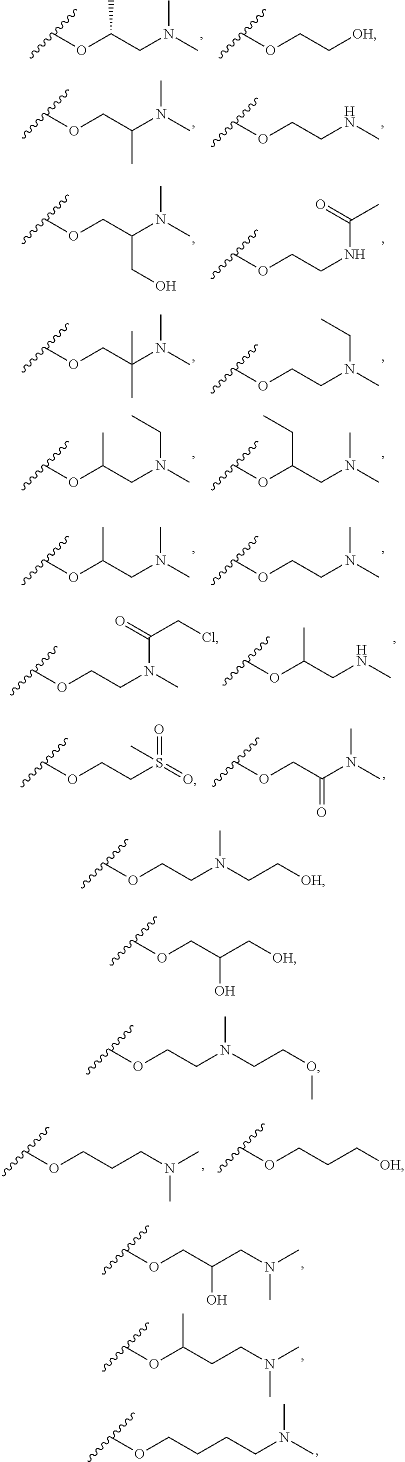

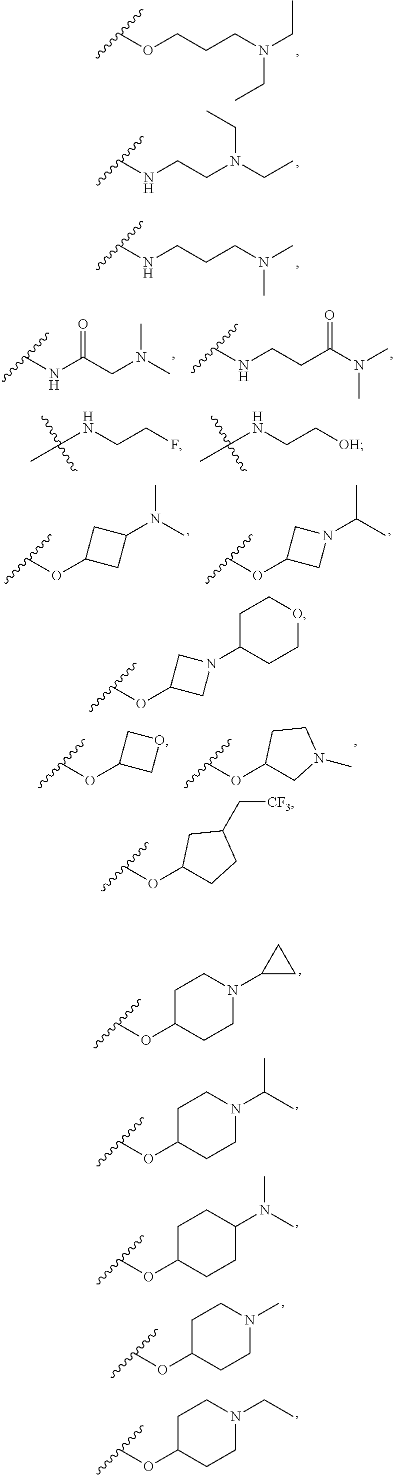

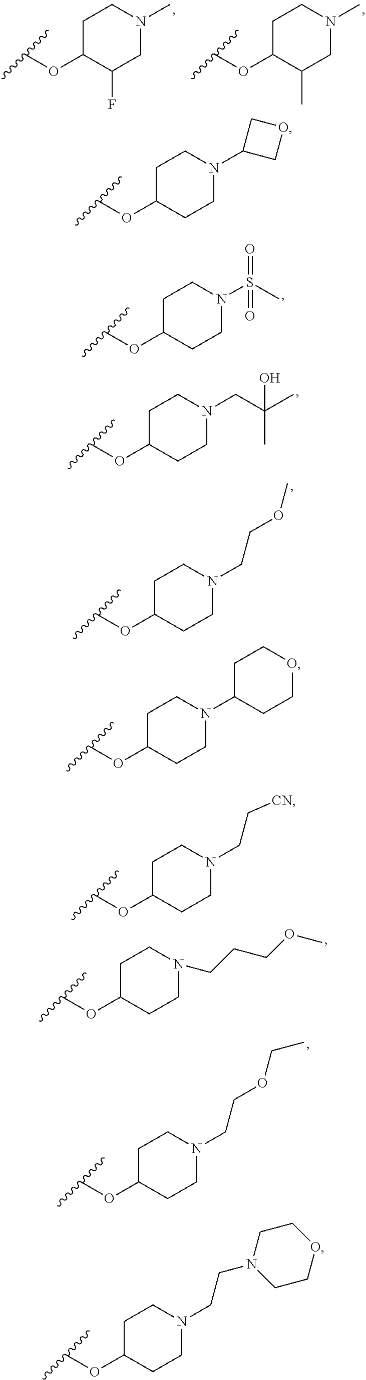

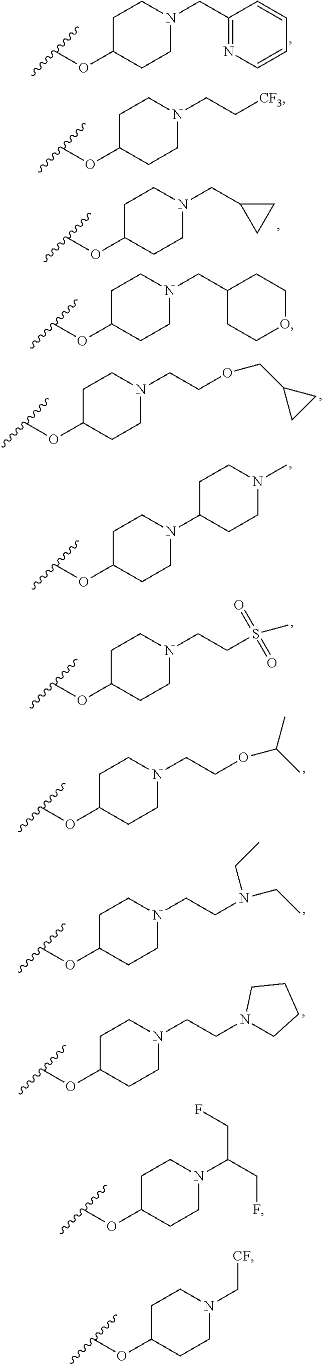

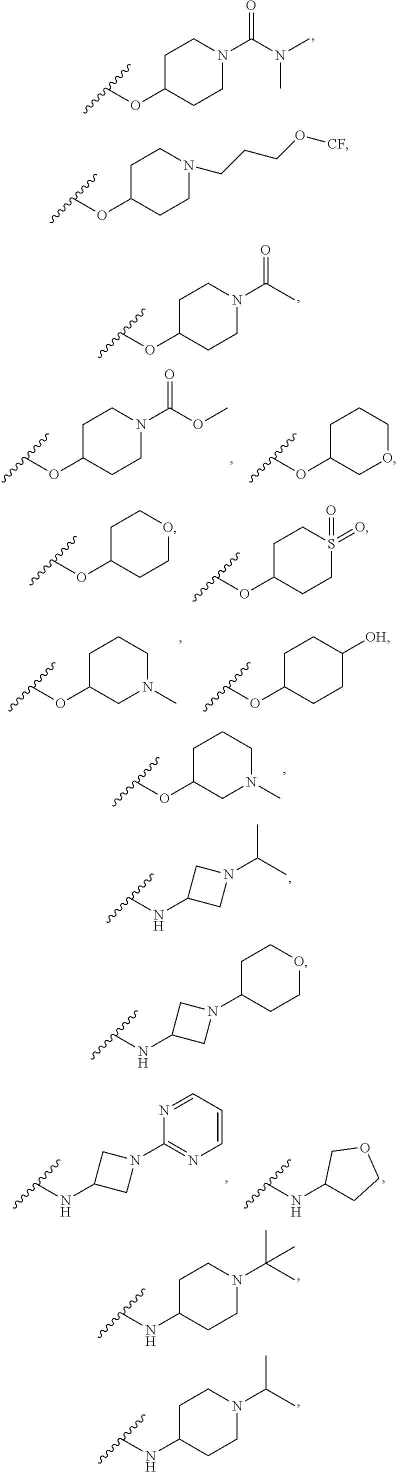

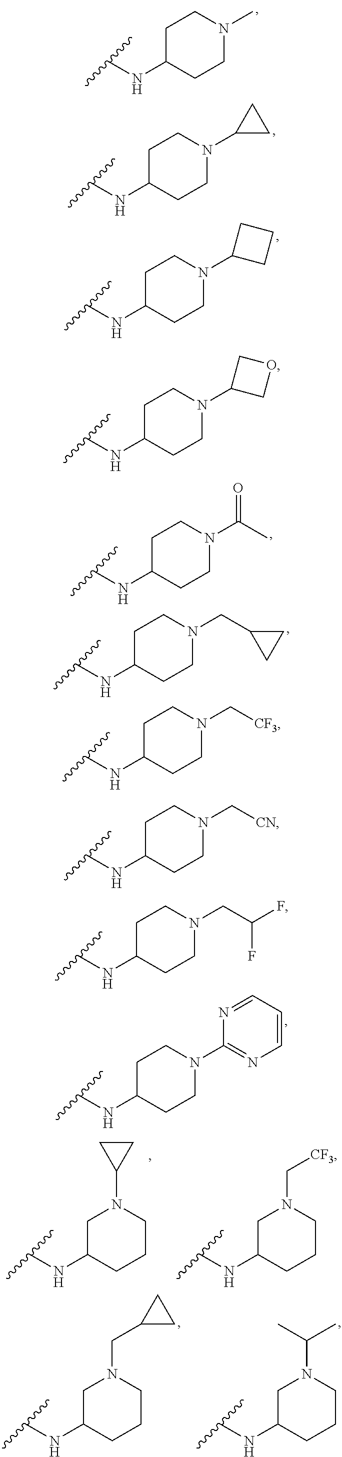

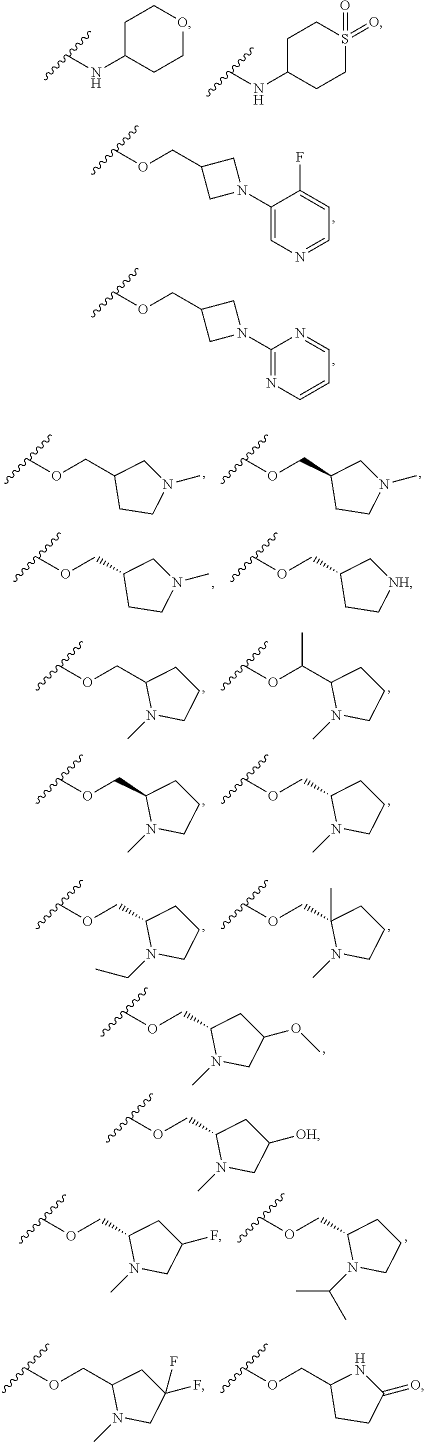

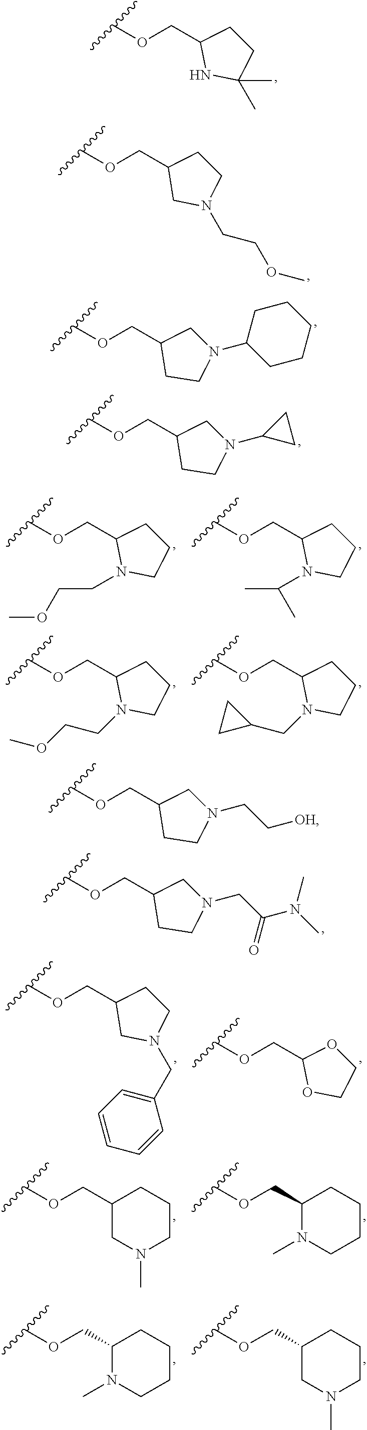

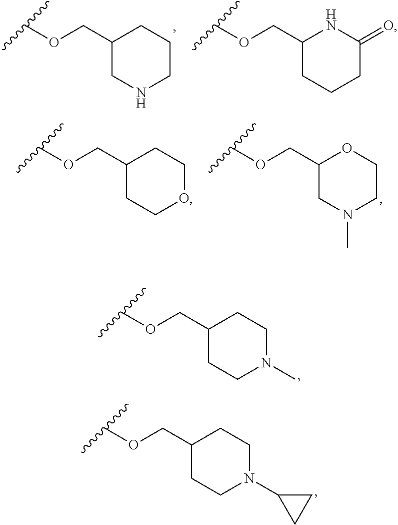

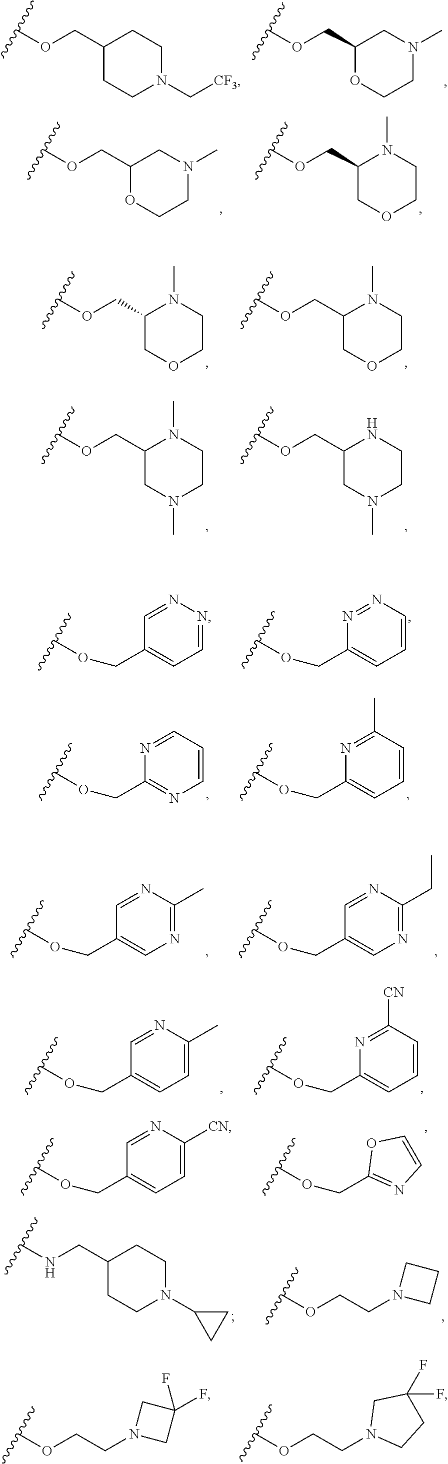

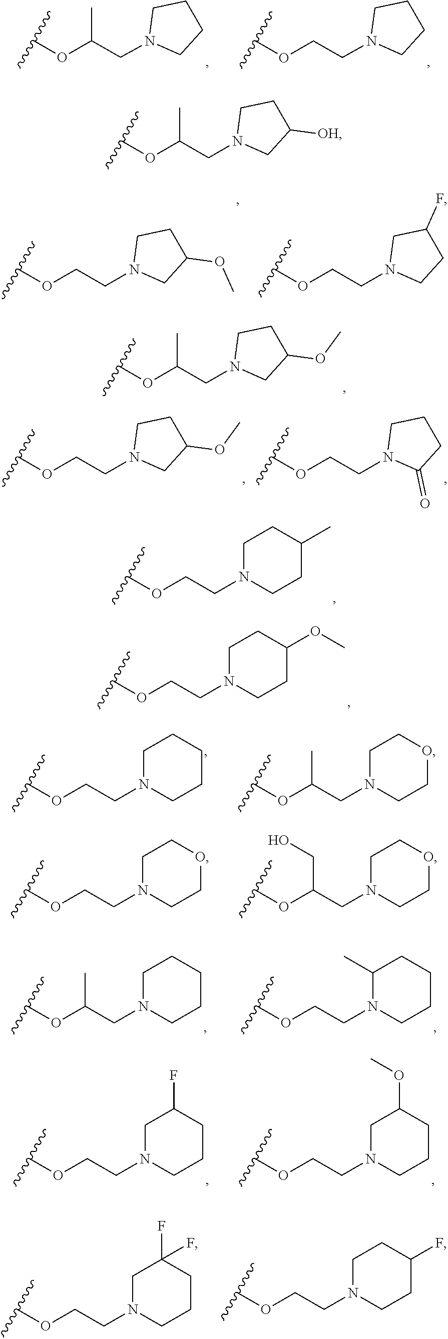

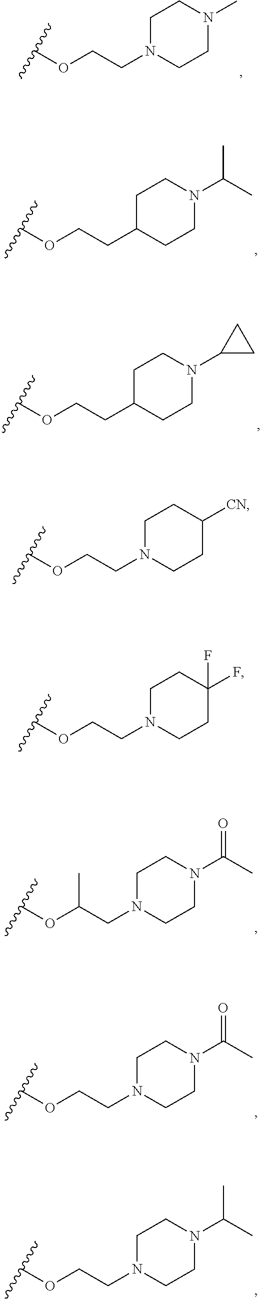

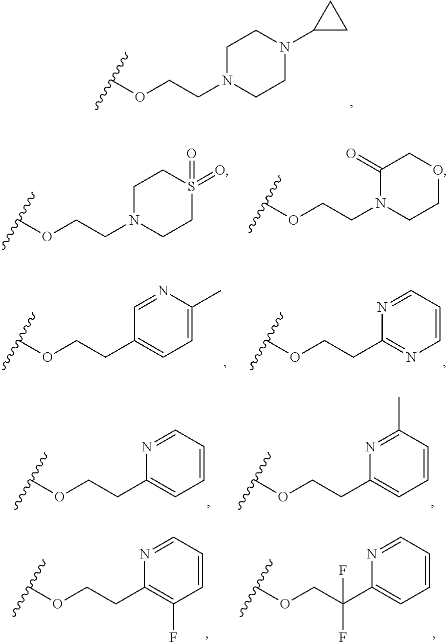

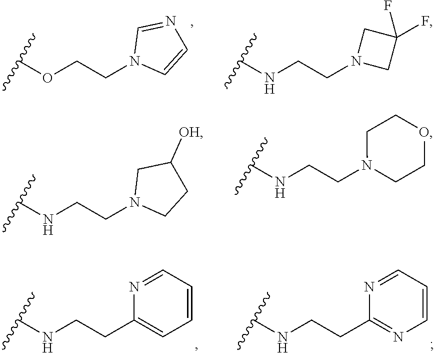

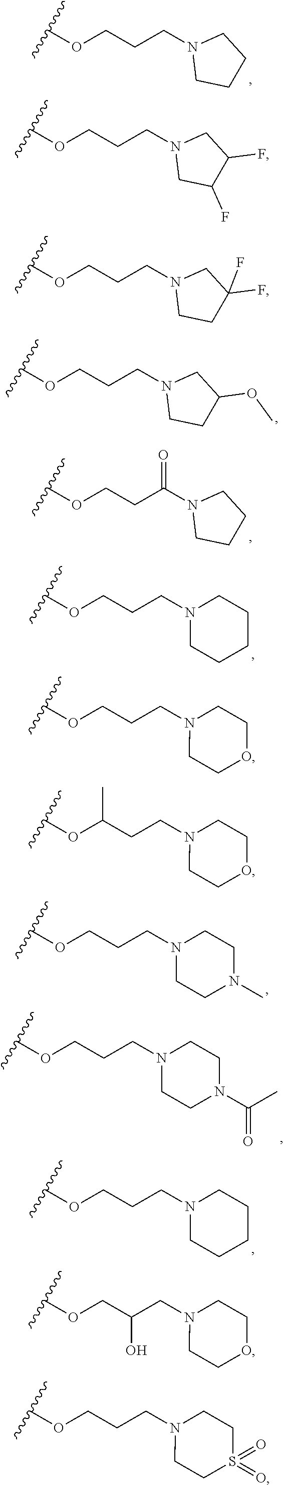

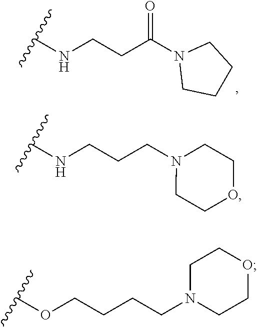

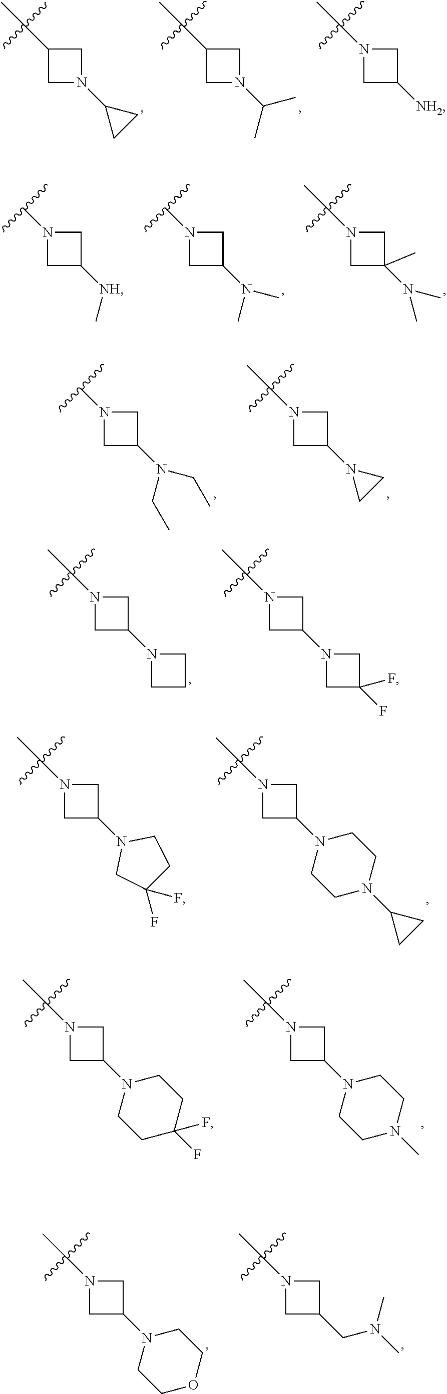

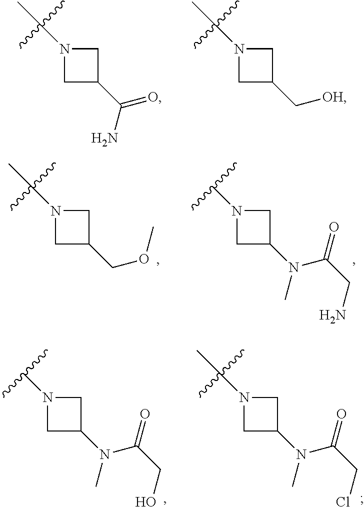

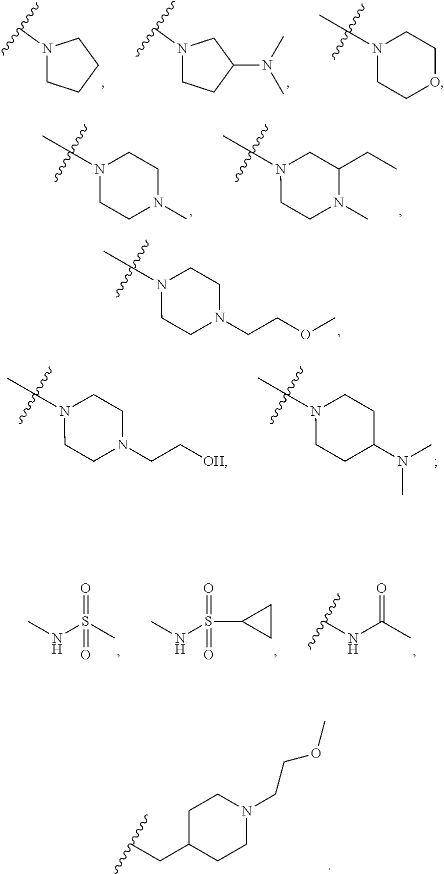

In one embodiment, in the groups (such as R₂), the 5- or 6-membered monocyclic heteroaryl is selected from:

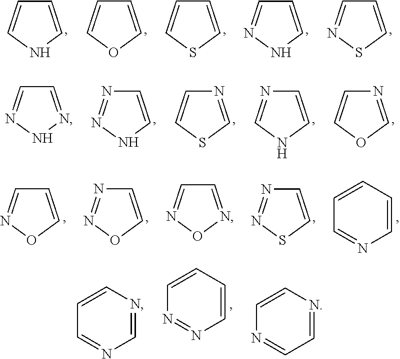

In one embodiment, in the groups (such as R₂), the 3- to 20-membered heterocycloalkyl is 3- to 6-membered heterocycloalkyl, 6- to 10-membered fused heterocycloalkyl, 7- to 11-membered spiro heterocycloalkyl, or 7- to 10-membered bridged heterocycloalkyl.

In one embodiment, in the groups (such as R₂), the C₃₋₂₀ cycloalkyl is C₃₋₈ cycloalkyl, and is selected from cyclopropyl, cyclobutyl, cyclopentyl, cyclopentenyl, cyclohexyl, cyclohexenyl, cyclohexadienyl, cycloheptyl, cycloheptatrienyl, cyclooctyl, cyclobutanone, cyclobutane-1,2-dione, cyclopentanone, cyclopentane-1,3-dione, cyclohexanone, cyclohexane-1,3-dione; the above C₃₋₈ cycloalkyl is unsubstituted or substituted with 1, 2, 3 or 4 substituents independently selected from the group S.

In one embodiment, the 3- to 20-membered heterocycloalkyl formed by R₂₁ and R₂₂ together with the nitrogen atom connected is 3- to 6-membered nitrogen-containing heterocycloalkyl, 6- to 10-membered nitrogen-containing fused heterocycloalkyl, or 7- to 11-membered nitrogen-containing spiro heterocycloalkyl.

In one embodiment, the 3- to 6-membered nitrogen-containing heterocycloalkyl formed by R₂₁ and R₂₂ together with the nitrogen atom connected is selected from the following structures:

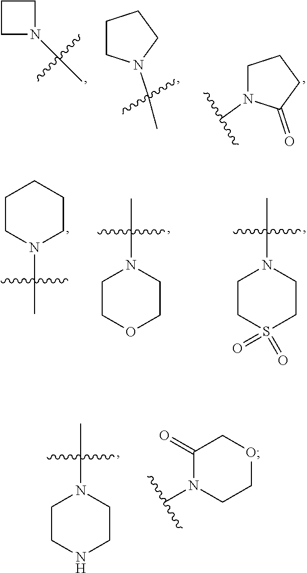

the 3- to 6-membered nitrogen-containing heterocycloalkyl is unsubstituted or substituted with 1, 2, 3 or 4 substituents independently selected from the group S.

In one embodiment, the X, Y, R₀, R₁, B, W, L, R₂ are each independently the corresponding groups in each specific compound in the examples.

In one embodiment, the compound of formula (I) or formula (II) or formula (III) is selected from each specific compound prepared in the examples.

In one embodiment, the compound of formula (I) formula (II) or formula (III) is selected from the compounds Z1-Z26 in the examples.

In another aspect, the present invention provides a pharmaceutical composition, comprising the compound as described above or a pharmaceutically acceptable salt, stereoisomer, solvate or prodrug thereof, and a pharmaceutically acceptable carrier.

As used herein, the term “pharmaceutically acceptable carrier” means any formulation or carrier medium capable of delivering an effective amount of the active substance of the invention without interfering with the biological activity of the active substance and without causing adverse effects to the host or subject. It is a non-toxic, inert, solid, semi-solid substance or liquid filling agent, diluent, packaging material or auxiliary preparation or any type of excipient. Representative carriers include water, oil, vegetables and minerals, cream base, lotion base, ointment base and the like. These bases include suspension agents, viscosifiers, transdermal promoters and the like. Their formulations are known to those skilled in the field of cosmetic or topical medicine.

In an embodiment of the present invention, the pharmaceutical composition may be administered in any form of oral, spray inhalation, rectal administration, nasal administration, buccal administration, topical administration, parenteral administration, such as, subcutaneous, intravenous, intramuscular, intraperitoneal, intrathecal, intraventricular, intrasternal and intracranial injection or infusion, or administered by means of an explanted reservoir. When administered orally, the compound of the present invention may be formulated into any orally acceptable dosage form, including but not limited to tablets, capsules, aqueous solutions or aqueous suspensions. Carriers used in tablets typically include lactose and cornstarch. Lubricants such as magnesium stearate may also be added. Diluents used in capsules typically include lactose and dried cornstarch. Aqueous suspensions are typically formulated by mixing an active ingredient with appropriate emulsifiers and suspension agents. Sweeteners, fragrances or colorants may be added to the oral dosage form as required. When topically administered, especially to the affected surface or organ readily accessible by topical application, such as eye, skin, or lower intestinal neuropathy, the compound of the present invention may be formulated into different topical dosage forms depending on the surface or organs. When topically administered to eyes, the compound of the present invention may be formulated into a dosage form of micronized suspension or solution using an isotonic sterile saline of a certain pH as the carrier, in which preservatives such as benzyl alkoxide chloride may or may not be added. For ocular administration, the compound may be formulated into a form of cream, such as, Vaseline cream.

When administered topically to skin, the compound of the present invention may be formulated into a suitable dosage form of ointment, lotion or cream, in which an active ingredient is suspended or dissolved in one or more carriers. The carriers useful in an ointment formulation include but not limited to: mineral oils, liquid vaseline, white vaseline, propylene glycol, polyoxyethylene, polypropylene oxide, emulsified wax and water. The carriers useful in a lotion or cream include but not limited to: mineral oils, sorbitan monostearate, cetyl ester wax, hexadecenyl aryl alcohol, 2-octyldodecanol, benzyl alcohol and water. The compound of the present invention may be administered in a dosage form of sterile injections, including sterial aqueous injection or oil suspension or sterile injection solution. Useful carriers and solvents include water, Ringer's solution and isotonic sodium chloride solution. Further, sterilized non-volatile oils can also be used as solvents or suspension media, such as monotriglycerides or diglycerides.

In another aspect, the present invention provides use of the compound as described above, or a pharmaceutically acceptable salt, stereoisomer, solvate or prodrug thereof or the pharmaceutical composition as described above in the manufacture of a medicament for preventing and/or treating cancer.

In one embodiment, the cancer is pancreatic ductal cancer, colorectal cancer, multiple myeloma, lung cancer, skin melanoma, endometrioid carcinoma, uterine carcinosarcoma, thyroid cancer, acute myeloid leukemia, bladder urothelial cancer, stomach cancer, cervical cancer, head and neck squamous cell carcinoma, diffuse large B-cell lymphoma, esophageal cancer, chronic lymphocytic leukemia, lung squamous cell carcinoma, small cell lung cancer, renal papillary cell carcinoma, adenoid cystic carcinoma, chromophobe renal cell carcinoma, liver cancer, breast invasive carcinoma, cervical squamous cell carcinoma, ovarian serous adenocarcinoma, adrenal cortex carcinoma, prostate cancer, neuroblastoma, brain low-grade glioma, glue Plasmoblastoma, medulloblastoma, esophageal squamous cell carcinoma, renal clear cell carcinoma, osteosarcoma, ovarian small cell carcinoma, rhabdoid tumor, sarcoma, small intestinal neuroendocrine tumor, T-cell prolymphocytic leukemia. In one embodiment, the cancer is lung cancer, preferably non-small cell lung cancer.

In another aspect, the present invention provides use of the compound as described above, or a pharmaceutically acceptable salt, stereoisomer, solvate or prodrug thereof or the pharmaceutical composition as described above in the manufacture of an inhibitor against KRAS mutation (preferably, the KRAS mutation is KRAS G12C mutation).

As used therein, the term “pharmaceutically acceptable salts” refers to salts of the compound of the present invention which are pharmaceutically acceptable, and can retain the biological effectiveness of the free base without other side effects. The type of pharmaceutical acceptable salts includes: acid addition salts formed with inorganic acids (such as hydrochloric acid, hydrobromic acid, sulfuric acid, nitric acid, phosphoric acid, and the like) or organic acids (such as acetic acid, propionic acid, hexanoic acid, cyclopentanepropionic acid, glycolic acid, pyruvic acid, trifluoroacetic acid, formic acid, lactic acid, malonic acid, succinic acid, malic acid, maleic acid, fumaric acid, tartaric acid, citric acid, benzoic acid, cinnamic acid, mandelic acid, methanesulfonic acid, ethanesulfonic acid, benzenesulfonic acid, naphthalene sulfonic acid, camphor sulfonic acid, gluconic acid, glutamic acid, salicylic acid, stearic acid, muconic acid and the like); or salts formed when an acidic proton present in the parent compound either is replaced by a metal ion such as an alkali metal ion or alkaline earth ion, such as sodium salt, potassium salt, calcium salt and magnesium salt, and the like; or coordinates with an organic base such as ethanolamine, diethanolamine, triethanolamine, N-methylglucamine, and the like. The pharmaceutically acceptable salts of the present invention can be synthesized from the parent compound containing acid radicals or base radicals by conventional chemical methods. In general, such salts are prepared by the reaction of these compounds in a form of free acid or base with a stoichiometric amount of the appropriate base or acid in water or an organic solvent or a mixture of the both. In general, non-aqueous media such as ether, ethyl acetate, ethanol, isopropanol or acetonitrile are preferred. In addition to salt forms, the compounds provided herein also exist in prodrug forms. The prodrugs of the compounds described herein are readily chemically altered under physiological conditions to be converted into the compounds of the invention. In addition, prodrugs can be converted to the compounds of the present invention by chemical or biochemical methods in an in vivo environment.

As used herein, the term “solvate” refers to a substance formed by combining the compound of the present invention with a pharmaceutically acceptable solvent. Pharmaceutical acceptable solvates include water, ethanol, acetic acid and the like. The solvates include stoichiometric solvates and non-stoichiometric solvates, preferably hydrates. Certain compounds of the present invention may be present in unsolvated or solvated forms, including hydrated forms. In general, solvated forms are equivalent to unsolvated forms and both are included within the scope of the present invention.

As used herein, the compound as represented by formula (I) of the present invention may contain one or more chiral centers and exist in different optically active forms. When the compound contains one chiral center, the compound contain enantiomers. The present invention includes these two isomers and mixtures thereof, such as racemic mixtures. Enantiomers can be resolved by methods known in the art, such as crystallization and chiral chromatography and other methods. When the compound of formula (I) contains more than one chiral center, diastereomers may exist. The present invention includes resolved optically pure specific isomers and mixtures of diastereomers. Diastereomers can be resolved by methods known in the art, such as crystallization and preparative chromatography. As used herein, the term “stereoisomers” include both conformational and configurational isomers, wherein configurational isomers mainly include cis-trans isomers and optical isomers. The compound of the present invention may be present in a stereoisomeric form, and thereby cover all possible stereoisomeric forms, including but not limited to cis-trans isomers, tautomers, enantiomers, diastereomers, atropisomers, and the like. The compound of the present invention can also be present in forms such as any combination or any mixture of the aforementioned stereoisomers, such as a mixture of mesoisomer, racemate, atropisomer in equal amounts, and the like. For example, a single enantiomer, a single diastereomers, or a mixture thereof, or a single atropisomer, or a mixture thereof. When the compound of the present invention contains an olefinic double bond, it includes a cis-isomer and trans-isomer, and any combination thereof, unless otherwise specified. The atropisomers of the present invention are stereoisomers with axial or planar chirality based on the restriction of intramolecular rotation. The compound of the present invention has two atropisomers derived from axial asymmetry, which is produced by restricting the rotation of bond between the substituents B which is a cyclic group such as C₆₋₁₀ aryl, a 5- or 6-membered monocycloheteroaryl, a 8- to 10-membered bicycloheteroaryl (especially when the adjacent positions of two ends of the bond have substituents at the ortho positions) and the rings such as substituted (hetero)aromatic pyrimidines, and forming a steric hindrance. Regarding the atropisomer of the present invention, the compound has a structure of formula (I) or the compound of formula (I) has an isomer produced by asymmetric carbon, which represents any one of a pair of atropisomers present in each isomeric compound. And as a medicine, an atropisomer with excellent activity is preferred. The compound of formula (I) has optical isomers derived from asymmetric carbon, axial asymmetry, and the like, if necessary, a single isomer can be obtained by methods known in the art, such as crystallization or chiral chromatography and other methods. The atropisomers of the compound of the present invention can be labeled in P or M configuration, and can also be labeled in other commonly used ways known in the art.

As used herein, the term “heteroatom” is selected from nitrogen, oxygen and sulfur, wherein the nitrogen can be optionally substituted, and the sulfur is also optionally substituted, for example oxidized, i.e. forming S(O)ₜ₃ (wherein t3 is an integer of 0 to 2).

As used herein, the term “alkyl” refers to a linear or branched saturated aliphatic hydrocarbon group having 1 to 20 carbon atoms. The term “C₁₋₁₀ alkyl” refers to a linear or branched alkyl group having 1 to 10 carbon atoms, more preferably 1, 2, 3, 4, 5 or 6 carbon atoms, i.e., C₁₋₆ alkyl, more preferably, C₁₋₄ alkyl, most preferably, C₁₋₃ alkyl. Specific examples include, but are not limited to, methyl, ethyl, n-propyl, iso-propyl, n-butyl, iso-butyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl and various branched isomers thereof.

As used herein, the term “alkoxy” refers to a group having a structure of —O-alkyl, wherein the alkyl is as defined above. The term “C₁₋₁₀ alkoxy” refers to an alkoxy group having 1 to 10 carbon atoms, preferably, C₁₋₆ alkoxy, more preferably, C₁₋₄ alkoxy, most preferably, C₁₋₃ alkoxy.

Specific examples include, but are not limited to, methoxy, ethoxy, n-propoxy, iso-propoxy, n-butoxy, tert-butoxy, iso-butoxy, n-pentoxy and the like.

As used herein, the term “alkenyl” refers to an alkyl as defined above having one or more carbon-carbon double bond at any position of the chain. The term “C₂₋₈ alkenyl” refers to an alkenyl group having 2 to 8 carbon atoms and at least one carbon-carbon double bond, preferably, an alkenyl group having 2 to 6 carbon atoms and 1 to 2 carbon-carbon double bonds, i.e., C₂₋₆ alkenyl, more preferably, an alkenyl group having 2 to 4 carbon atoms and 1 to 2 carbon-carbon double bonds, i.e., C₂₋₄ alkenyl. Specific examples include, but are not limited to, ethenyl, 1-propenyl, 2-propenyl, 1-, 2- or 3-butenyl, pentenyl, hexenyl, butadienyl, and the like.

As used herein, the term “alkynyl” refers to an alkyl as defined above having one or more carbon-carbon triple bond at any position of the chain. The term “C₂₋₈ alkynyl” refers to an alkynyl group having 2 to 8 carbon atoms and at least one carbon-carbon triple bond, preferably, an alkynyl group having 2 to 6 carbon atoms and 1 to 2 carbon-carbon triple bonds, i.e., C₂₋₆ alkynyl, more preferably, an alkynyl group having 2 to 4 carbon atoms and 1 to 2 carbon-carbon triple bonds, i.e., C₂₋₄ alkynyl. Specific examples include, but are not limited to, ethynyl, 1-propynyl, 2-propynyl, 1-, 2- or 3-butynyl, and the like.

As used herein, the term “halogen” refers to fluoro, chloro, bromo and iodine.

As used herein, the term “haloalkyl” refers to an alkyl as defined above which is substituted with one or more (such as 1, 2, 3, 4 or 5) halogens. The term “halo C₁₋₁₀ alkyl” refers to a haloalkyl having 1 to 10 carbon atoms, preferably, halo C₁₋₆ alkyl, more preferably, halo C₁₋₄ alkyl, most preferably, halo C₁₋₃ alkyl. Specific examples include, but are not limited to, chloromethyl, dichloromethyl, trichloromethyl, chloroethyl, 1,2-dichloroethyl, trichloroethyl, bromoethyl, fluoromethyl, difluoromethyl, trifluoromethyl, fluoroethyl, difluoroethyl, trifluoroethyl, and the like.

As used herein, the term “haloalkoxy” refers to an alkoxy as defined above which is substituted with one or more (such as 1, 2, 3, 4 or 5) halogens. The term “halo C₁₋₁₀ alkoxy” refers to a haloalkoxy having 1 to 10 carbon atoms, preferably, halo C₁₋₆ alkoxy, more preferably, halo C₁₋₄ alkoxy, most preferably, halo C₁₋₃ alkoxy. Specific examples include, but are not limited to, trifluoromethoxy, trifluoroethoxy, fluoromethoxy, fluoroethoxy, difluoromethoxy, difluoroethoxy, and the like.

As used herein, the terms “cycloalkyl” and “cycloalkyl ring” can be used interchangeably, and refer to a saturated or partially unsaturated monocyclic or polycyclic cyclic hydrocarbon group. The term “cycloalkyl” may be cycloalkyl containing 3 to 20 carbon atoms (C₃₋₂₀ cycloalkyl), preferably cycloalkyl containing 3 to 12 carbon atoms (C₃₋₁₂cycloalkyl), more preferably cycloalkyl containing 3 to 10 carbon atoms (C₃₋₁₀cycloalkyl), and more preferably cycloalkyl containing 3 to 6 carbon atoms (C₃₋₆cycloalkyl). The ring carbon atoms of the cycloalkyl may be optionally substituted with 1, 2, or 3 oxo groups to form a cyclic ketone structure.

When the cycloalkyl is monocyclic cycloalkyl, monocyclic cycloalkyl containing 3 to 8 ring carbon atoms (i.e., 3- to 8-membered or C₃₋₈) is preferred; “C₃₋₈monocyclic cycloalkyl” and “C₃₋₈cycloalkyl” can be used interchangeably herein; monocyclic cycloalkyl containing 3 to 6 ring carbon atoms (i.e., 3- to 6-membered or C₃₋₆) is more preferred; non-limiting examples of monocyclic cycloalkyl (or C₃₋₆ monocyclic cycloalkyl) include cyclopropyl, cyclobutyl, cyclopentyl, cyclopentenyl, cyclohexyl, cyclohexenyl, cyclohexadienyl, cycloheptyl, cycloheptatrienyl, cyclooctyl, cyclobutanone, cyclobutane-1,2-dione, cyclopentanone, cyclopentane-1,3-dione, cyclohexanone, cyclohexane-1,3-dione, etc.

Typically, cycloalkyl containing 3 to 6 carbon atoms (C₃₋₆cycloalkyl) refers to monocyclic cycloalkyl (C₃₋₆monocyclic cycloalkyl). As used herein, “3- to 6-membered monocyclic”, “3- to 6-membered monocyclic cycloalkyl”, “C₃₋₆monocyclic cycloalkyl” and “C₃₋₆cycloalkyl” can be used interchangeably, and refer to a saturated or partially unsaturated all-carbon monocyclic ring containing 3 to 6 ring atoms. The ring carbon atoms of the monocyclic ring may be optionally substituted with 1, 2, or 3 oxo groups to form a cyclic ketone structure. Examples of 3- to 6-membered monocyclic ring include (but are not limited to): cyclopropyl ring, cyclobutyl ring, cyclopentyl ring, cyclopentenyl ring, cyclohexyl ring, cyclohexenyl ring, cyclohexadienyl ring, cyclobutanone, cyclobutane-1,2-dione, cyclopentanone, cyclopentane-1,3-dione, cyclohexanone, cyclohexane-1,3-dione, etc.

In the case of polycyclic cycloalkyl, the polycyclic cycloalkyl includes spirocycloalkyl, fused cycloalkyl and bridged cycloalkyl.

The cycloalkyl ring can be fused to an aryl, heteroaryl or heterocycloalkyl ring, wherein the ring linked to the parent structure is a cycloalkyl ring, non-limiting examples include indanyl, tetrahydronaphthyl, benzocycloheptyl, etc. Cycloalkyl groups can be optionally substituted or unsubstituted, and when substituted, the substituents are preferably one or more of the groups described in the present application.

As used herein, the term “heterocycloalkyl” and “heterocycloalkyl ring” can be used interchangeably, and refer to a saturated or partially unsaturated monocyclic or polycyclic cyclic hydrocarbon group, wherein one or more (preferably 1 to 4, 1 to 3, or 1 to 2) ring atoms are the heteroatoms selected from nitrogen, oxygen or S(O)ₜ₃ (wherein t3 is an integer of 0 to 2), with the ring part of —O—O—, —O—S— or —S—S— being excluded, and the rest of the ring atoms are carbon atoms. The term “heterocycloalkyl” may be heterocycloalkyl containing 3 to 20 ring atoms (i.e., 3- to 20-membered), preferably 3- to 12-membered heterocycloalkyl, more preferably 3- to 10-membered heterocycloalkyl, and more preferably 3- to 6-membered heterocycloalkyl, wherein one or more (preferably 1 to 4) ring atoms are the heteroatoms selected from nitrogen, oxygen or S(O)ₜ₃ (wherein t3 is an integer of 0 to 2), with the ring part of —O—O—, —O—S— or —S—S— being excluded, and the rest of the ring atoms are carbon atoms. Nitrogen atoms may be substituted or unsubstituted (i.e., N or NR, R is hydrogen or any of the substituents already defined herein). The ring carbon atoms of the heterocycloalkyl may be optionally substituted with 1, 2, or 3 oxo groups to form a cyclic ketone, cyclic lactone or cyclolactam structure.

As used herein, with regard to “3- to 20-membered heterocycloalkyl”, “3- to 12-membered heterocycloalkyl”, “3- to 10-membered heterocycloalkyl” or “3- to 6-membered heterocycloalkyl”, when these heterocycloalkyl groups are 3-membered heterocycloalkyl groups and contain only one heteroatom as a ring atom, the heteroatom is not a nitrogen atom.

In one embodiment, “heterocycloalkyl” refers to monocyclic heterocycloalkyl, wherein the monocyclic heterocycloalkyl is saturated or partially unsaturated, preferably monocyclic heterocycloalkyl containing 3 to 8 ring atoms (i.e., 3- to 8-membered), of which 1, 2, or 3 are heteroatoms, more preferably monocyclic heterocycloalkyl containing 3 to 6 ring atoms (i.e., 3- to 6-membered), of which 1, 2, or 3 are heteroatoms, and most preferably monocyclic heterocycloalkyl containing 5 or 6 ring atoms (i.e., 5- or 6-membered), of which 1, 2, or 3 are heteroatoms. As used herein, the term “3- to 6-membered heterocycloalkyl” and “3- to 6-membered monocyclic heterocycloalkyl” can be used interchangeably, and the term “5- or 6-membered heterocycloalkyl” and “5- or 6-membered monocyclic heterocycloalkyl” can be used interchangeably. When the heteroatom is a nitrogen atom, the nitrogen atom may be substituted or unsubstituted (i.e., N or NR, R is hydrogen or other substituents already defined herein). When the heteroatom is a sulfur atom, the sulfur atom may be optionally oxidized (i.e., S(O)ₜ₃, t3 is an integer of 0 to 2). The ring carbon atoms of the monocyclic heterocycloalkyl may be optionally substituted with 1, 2, or 3 oxo groups to form a cyclic ketone, cyclic lactone or cyclolactam structure. Non-limiting examples of monocyclic heterocycloalkyl include: aziridine, ethylene oxide, azetidine, azetidine-2-one, oxetane, oxetane-2-one, oxazolidine, pyrrolidine-2-one, pyrrolidine-2,5-dione, 1,3-dioxolane, dihydrofuran-2(3H)-one, dihydrofuran-2,5-dione, piperidine-2-one, piperidine-2,6-dione, tetrahydro-2H-pyran-2-one, imidazolidine, tetrahydrofuran, tetrahydrothiophene, tetrahydropyrrole, 1,3-dioxolane-2-one, oxazolidine-2-one, imidazolidine-2-one, piperidine, piperazine, piperazine-2-one, morpholine, morpholine-3-one, morpholine-2-one, thiomorpholine-3-one 1,1-dioxide, thiomorpholine, thiomorpholine-1,1-dioxide, tetrahydropyran, 1,2-dihydroazetidine, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, 1,3-oxazinane, hexahydropyrimidine, 1,4-dioxane, tetrahydropyrimidine-2(1H)-one, 1,4-dioxane-2-one, 5,6-dihydro-2H-pyran-2-one, 5,6-dihydropyrimidine-4(3H)-one, 3,4-dihydropyridine-2(1H)-one, 5,6-dihydropyridine-2 (1H)-one, 5,6-dihydropyrimidine-4(1H)-one, pyrimidine-4(3H)-one, pyrimidine-4(1H)-one, 4,5-dihydro-1H-imidazole, 2,3-dihydro-1H-imidazole, 2,3-dihydroxazole, 1,3-dioxolene, 2,3-dihydrothiophene, 2,5-dihydrothiophene, 3,4-dihydro-2H-1,4-oxazine, 3,4-dihydro-2H-1,4-thiazide 1,1-dioxide, 1,2,3,4-tetrahydropyrazine, 1,3-dihydro-2H-pyrrole-2-one, 1,5-dihydro-2H-pyrrole-2-one, 1H-pyrrole-2,5-dione, furan-2(3H)-one, furan-2(5H)-one, 1,3-dioxolene-2-one, oxazole-2(3H)-one, 1,3-dihydro-2H-imidazole-2-one, furan-2,5-dione, 3,6-dihydropyridine-2(1H)-one, pyridine-2,6-(1H,3H)-dione, 5,6-dihydro-2H-pyran-2-one, 3,6-dihydro-2H-pyran-2-one, 3,4-dihydro-2H-1,3-oxazine, 3,6-dihydro-2H-1,3-oxazine, 1,2,3,4-tetrahydropyrimidine, etc.

Typically, 3- to 6-membered heterocycloalkyl refers to 3- to 6-membered monocyclic heterocycloalkyl. As used herein, “3- to 6-membered monocyclic heterocyclic ring” or “3- to 6-membered monocyclic heterocycloalkyl” can be used interchangeably, and refers to 3- to 6-membered, preferably 4- to 6-membered, and more preferably 5- to 6-membered, saturated or partially unsaturated monocyclic ring, in which 1, 2 or 3 carbon atoms are substituted with heteroatoms selected from nitrogen, oxygen or S(O)ₜ₅ (wherein t5 is an integer of 0 to 2), with the ring part of —O—O—, —O—S— or —S—S— being excluded, and the rest of the ring atoms are carbon atoms.

The ring carbon atoms of the monocyclic heterocyclic ring may be optionally substituted with 1, 2, or 3 oxo groups to form a cyclic ketone, cyclic lactone or cyclolactam structure. Examples of 3- to 6-membered monocyclic heterocyclic ring include (but are not limited to) aziridine, ethylene oxide, azetidine, oxetane, tetrahydrofuran, tetrahydrothiophene, tetrahydropyrrole, piperidine, pyrroline, oxazolidine, piperazine, dioxolane, dioxane, morpholine, thiomorpholine, thiomorpholine-1,1-dioxide, tetrahydropyran, 1,2-dihydroazetidine, 1,2-dihydrooxetadiene, 2,5-dihydro-1H-pyrrole, 2,5-dihydrofuran, 2,3-dihydrofuran, 2,3-dihydro-1H-pyrrole, 3,4-dihydro-2H-pyran, 1,2,3,4-tetrahydropyridine, 3,6-dihydro-2H-pyran, 1,2,3,6-tetrahydropyridine, etc.

Two ring atoms connected in the above-mentioned monocyclic heterocycloalkyl, including C—C and N—C, can be optionally fused with cycloalkyl, heterocycloalkyl, aryl or heteroaryl as defined in the present invention, such as monocyclic cycloalkyl ring, monocyclic heterocycloalkyl ring, monoaryl ring, and 5- or 6-membered monocyclic heteroaryl ring, to form a fused polycyclic ring, and the two ring atoms connected in the monocyclic heterocycloalkyl, which are together with other rings to form fused rings, are preferably C—C.

In one embodiment, “heterocycloalkyl” refers to polycyclic heterocycloalkyl, including spiroheterocycloalkyl, fused heterocycloalkyl and bridged heterocycloalkyl.

As used herein, the term “spiroheterocycloalkyl” refers to saturated or partially unsaturated polycyclic heterocycloalkyl, wherein one atom (called a spiro atom) is shared between the monocyclic rings in the system, and one or more (for example, 1 to 4, or 1 to 3, or 1 to 2) of the ring atoms are heteroatoms selected from nitrogen, oxygen or S(O)ₜ₄ (wherein t4 is an integer of 0 to 2), and the rest of the ring atoms are carbon. The term “saturated spiroheterocycloalkyl” refers to a spiroheterocycloalkyl system containing no unsaturated bonds. The term “partially unsaturated spiroheterocycloalkyl” means that one or more rings in the spiroheterocycloalkyl system may contain one or more double bonds, but none of the rings has a fully conjugated π-electron system. The term “spiroheterocycloalkyl” may be a spiroheterocycloalkyl containing 5 to 20 ring atoms (i.e., 5- to 20-membered), wherein one atom (called a spiro atom) is shared between 3- to 8-membered monocyclic rings (i.e., containing 3 to 8 ring atoms), and the spiroheterocycloalkyl is preferably 6- to 14-membered spiroheterocycloalkyl, and more preferably 7- to II-membered spiroheterocycloalkyl; and one or more of the ring atoms are heteroatoms selected from nitrogen, oxygen or S(O)ₜ₄ (wherein t4 is an integer of 0 to 2), and the rest of the ring atoms are carbon. When the heteroatom is a nitrogen atom, the nitrogen atom may be substituted or unsubstituted (i.e., N or NR, R is hydrogen or other substituents already defined herein). Each monocyclic ring can contain one or more double bonds, but none of the rings has a fully conjugated π-electron system. According to the number of shared spiro atoms between the rings, the spiroheterocycloalkyl is divided into monospiroheterocycloalkyl, bisspiroheterocycloalkyl or polyspiroheterocycloalkyl, preferably monospiroheterocycloalkyl and bisspiroheterocycloalkyl. More preferably, 7-membered (4-membered monocyclic ring/4-membered monocyclic ring), 8-membered (4-membered monocyclic ring/5-membered monocyclic ring), 9-membered (4-membered monocyclic ring/6-membered monocyclic ring, 5-membered monocyclic ring/5-membered monocyclic ring), 10-membered (5-membered monocyclic ring/6-membered monocyclic ring), or 11-membered (6-membered monocyclic ring/6-membered monocyclic ring) monospiroheterocycloalkyl. The non-limiting examples of spiroheterocycloalkyl include:

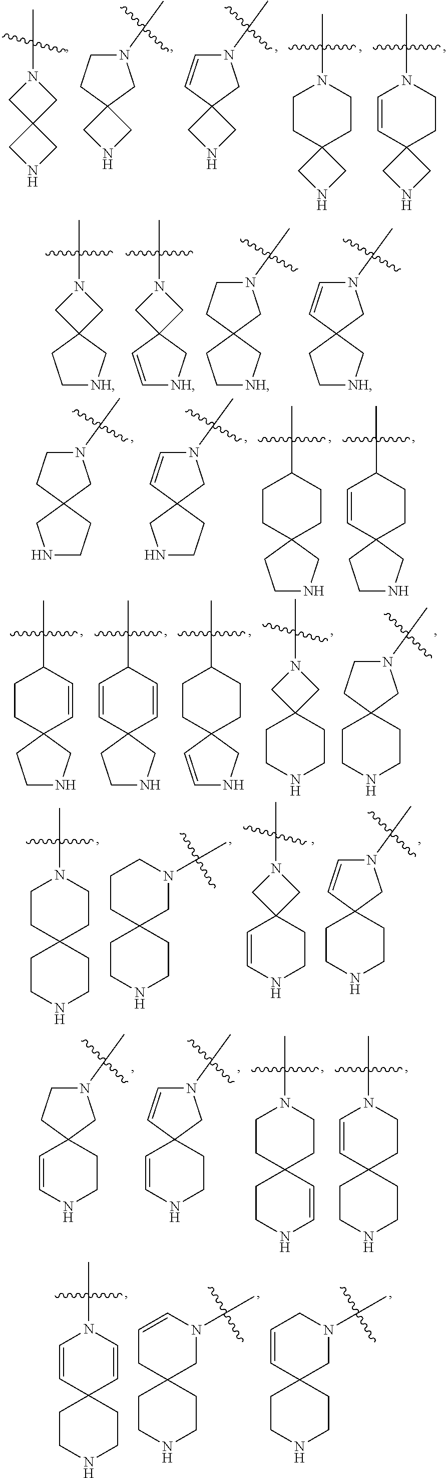

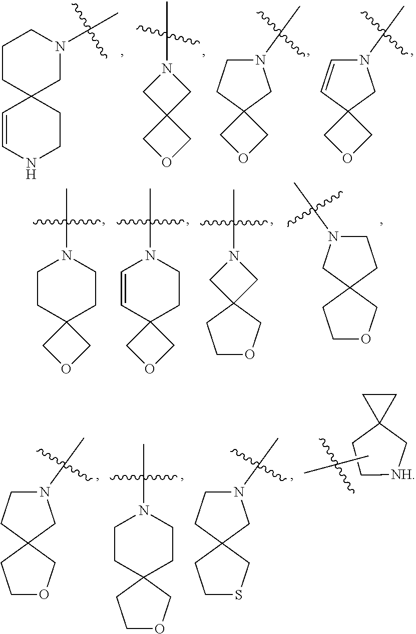

As used herein, the term “fused heterocycloalkyl” refers to saturated or partially unsaturated polycyclic heterocycloalkyl, wherein each ring in the system shares an adjacent pair of atoms with other rings in the system, and one or more (for example, 1 to 4, or 1 to 3, or 1 to 2) of the ring atoms in the system are heteroatoms selected from nitrogen, oxygen or S(O)ₜ₄ (wherein t4 is an integer of 0 to 2), and the rest of the ring atoms are carbon. The term “saturated fused heterocycloalkyl” refers to a fused heterocycloalkyl system containing no unsaturated bonds. The term “partially unsaturated fused heterocycloalkyl” means that one or more rings in the fused heterocycloalkyl system may contain one or more double bonds, but none of the rings has a fully conjugated π-electron system. The term “fused heterocycloalkyl” may be a fused heterocycloalkyl containing 5 to 20 ring atoms (i.e., 5- to 20-membered), preferably 6- to 14-membered fused heterocycloalkyl, more preferably 6- to 10-membered fused heterocycloalkyl, and more preferably 8- to 10-membered fused heterocycloalkyl; and one or more of the ring atoms in the system are heteroatoms selected from nitrogen, oxygen or S(O)ₜ₄ (wherein t4 is an integer of 0 to 2), and the rest of the ring atoms are carbon. When the heteroatom is a nitrogen atom, the nitrogen atom may be substituted or unsubstituted (i.e., N or NR, R is hydrogen or other substituents already defined herein). According to the number of constituent rings, it can be divided into bicyclic, tricyclic, tetracyclic or polycyclic fused heterocycloalkyl, preferably bicyclic or tricyclic, and more preferably 8-membered (5-membered monocyclic ring fused with 5-membered monocyclic ring), 9-membered (5-membered monocyclic ring fused with 6-membered monocyclic ring) or 10-membered (6-membered monocyclic ring fused with 6-membered monocyclic ring) bicyclic fused heterocycloalkyl. The non-limiting examples of fused heterocycloalkyl include:

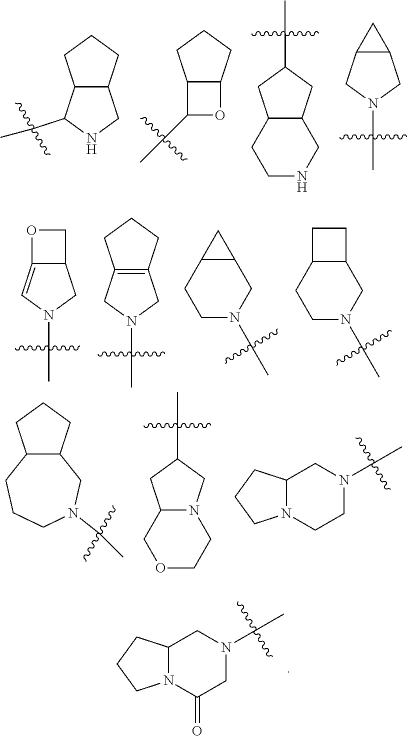

As used herein, the term “bridged heterocycloalkyl” refers to saturated or partially unsaturated polycyclic heterocycloalkyl, wherein any two rings in the system share two atoms that are not directly connected, and one or more (for example, 1 to 4, or 1 to 3, or 1 to 2) of the ring atoms are heteroatoms selected from nitrogen, oxygen or S(O)ₜ₃ (wherein t3 is an integer of 0 to 2), and the rest of the ring atoms are carbon. The term “saturated bridged heterocycloalkyl” refers to a bridged heterocycloalkyl system containing no unsaturated bonds. The term “partially unsaturated bridged heterocycloalkyl” means that one or more rings in the bridged heterocycloalkyl system may contain one or more double bonds, but none of the rings has a fully conjugated π-electron system. The term “bridged heterocycloalkyl” may be a bridged heterocycloalkyl containing 5 to 20 ring atoms (i.e., 5- to 20-membered), preferably 6- to 14-membered bridged heterocycloalkyl, and more preferably 7- to 10-membered bridged heterocycloalkyl; and one or more (for example, 1 to 4, or 1 to 3, or 1 to 2) of the ring atoms are heteroatoms selected from nitrogen, oxygen or S(O)ₜ₃ (wherein t3 is an integer of 0 to 2), and the rest of the ring atoms are carbon. According to the number of constituent rings, it can be divided into bicyclic, tricyclic, tetracyclic or polycyclic bridged heterocycloalkyl, preferably bicyclic, tricyclic or tetracyclic, and more preferably bicyclic or tricyclic. The non-limiting examples of bridged heterocycloalkyl include:

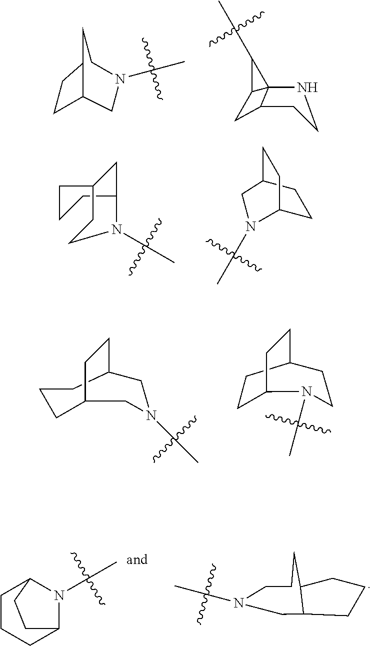

In the present invention, the above-mentioned various heterocycloalkyl groups may be optionally substituted or unsubstituted; when substituted, the substituents are preferably one or more groups as described in the present application.

As used herein, in “spiroheterocycloalkyl”, “bridged heterocycloalkyl” or “fused heterocycloalkyl”, when the ring containing a heteroatom is a 3-membered ring and contains only one heteroatom as a ring atom, the heteroatom is not a nitrogen atom.

As used herein, the terms “aryl”, “aryl ring” and “aromatic ring” are used interchangeably and refer to fully unsaturated aliphatic hydrocarbon groups. It can be an all-carbon monocyclic ring containing 6 to 14 ring atoms (i.e., 6- to 14-membered or C₆₋₁₄), an all-carbon polycyclic ring (the rings are connected via covalent bonds, not by means of fusion) or an all-carbon fused polycyclic ring (that is, a ring that shares adjacent pairs of carbon atoms) group, wherein at least one ring in the ring system is aromatic, that is, it has a conjugated 7-electron system. Preferably, aryl containing 6 to 10 ring carbon atoms (i.e., 6- to 10-membered or C₆₋₁₀). Each ring in the ring system contains 5 or 6 ring atoms.

In one embodiment, “aryl” refers to a monoaryl or polyaryl ring, non-limiting examples of which include: phenyl, biphenyl, etc.

In one embodiment, “aryl” refers to an aromatic fused polycyclic ring, which is a polycyclic group in which monoaryl ring is fused with one or more monoaryl rings, non-limiting examples of which include: naphthyl, anthracenyl, etc.

In one embodiment, aryl ring described herein (e.g., monoaryl ring, preferably phenyl) may be fused with one or more non-aromatic rings to form a polycyclic group, wherein the ring connected to the parent structure is an aromatic ring or a non-aromatic ring, and the non-aromatic ring includes but is not limited to: a 3- to 6-membered monocyclic heterocycloalkyl ring, preferably a 5- or 6-membered monocyclic heterocycloalkyl ring (the ring carbon atoms of the monocyclic heterocycloalkyl ring may be substituted with 1 to 2 oxo groups to form a cyclolactam or cyclic lactone structure); a 3- to 6-membered monocyclic cycloalkyl ring, preferably a 5- or 6-membered monocyclic cycloalkyl ring (the ring carbon atoms of the monocyclic cycloalkyl ring may be substituted with 1 or 2 oxo groups to form a cyclic ketone structure), etc. The polycyclic group in which the above-mentioned monoaryl ring is fused with one or more non-aromatic rings can be connected to other groups or the parent structure via a nitrogen atom or carbon atom, and the ring connected to the parent structure is a monoaryl ring or non-aromatic ring. The non-limiting examples include:

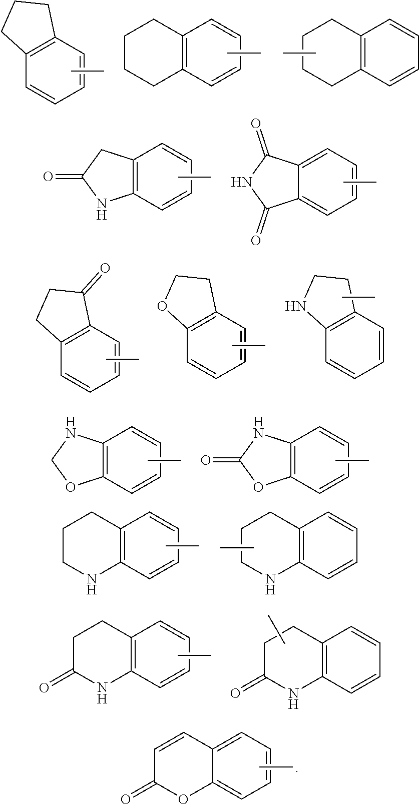

In the present invention, the above-mentioned various aryl groups may be optionally substituted or unsubstituted; when substituted, the substituents are preferably one or more groups as described in the present application.

As used herein, the term “heteroaryl”, “heteroaryl ring” and “heteroaromatic ring” are used interchangeably and refer to fully unsaturated aliphatic hydrocarbon groups containing heteroatoms. It may be a monocyclic or fused polycyclic ring (that is, a ring that shares adjacent pairs of carbon atoms or heteroatoms) group which may contain 5 to 14 ring atoms (i.e., 5- to 14-membered), preferably 5 to 10 ring atoms (i.e., 5- to 10-membered), more preferably 5, 6, 8, 9 or 10 ring atoms, in which 1 to 4 heteroatoms are contained therein as ring atoms, and the heteroatoms are selected from oxygen, sulfur and nitrogen. The nitrogen and sulfur atoms can be optionally oxidized, and the nitrogen atoms can be optionally quaternized. The heteroaryl preferably has 6, 10 or 14 π electrons shared in the ring system. At least one ring in the ring system is aromatic.

In one embodiment, “heteroaryl” refers to monocyclic heteroaryl ring (preferably 5- or 6-membered monocyclic heteroaryl ring), non-limiting examples of which include: thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine, etc.

In one embodiment, “heteroaryl” refers to fused polyheteroaryl ring (preferably 8- to 10-membered bicyclic heteroaryl ring). The fused polyheteroaryl ring includes both a polycyclic group (preferably a 9- or 10-membered bicyclic heteroaryl ring) in which a monoaryl ring (preferably phenyl) is fused with a monocyclic heteroaryl ring (preferably a 5- or 6-membered monocyclic heteroaryl ring), and a polycyclic group (preferably a 8- to 10-membered bicyclic heteroaryl ring) in which a monocyclic heteroaryl (preferably a 5- or 6-membered monocyclic heteroaryl) is fused with a monocyclic heteroaryl (preferably a 5- or 6-membered monocyclic heteroaryl).

Any two ring atoms connected in the above-mentioned monocyclic heteroaryl ring, including C—C, N—C and N—N, can be fused with cycloalkyl, heterocycloalkyl, aryl or heteroaryl as defined in the present invention, such as monocyclic cycloalkyl ring, monocyclic heterocycloalkyl ring, monoaryl ring, and 5- or 6-membered monocyclic heteroaryl ring, to form a fused polycyclic ring. The two ring atoms connected in the monocyclic heteroaryl ring forming a fused ring with other rings are preferably C—C, and The non-limiting examples include the following forms:

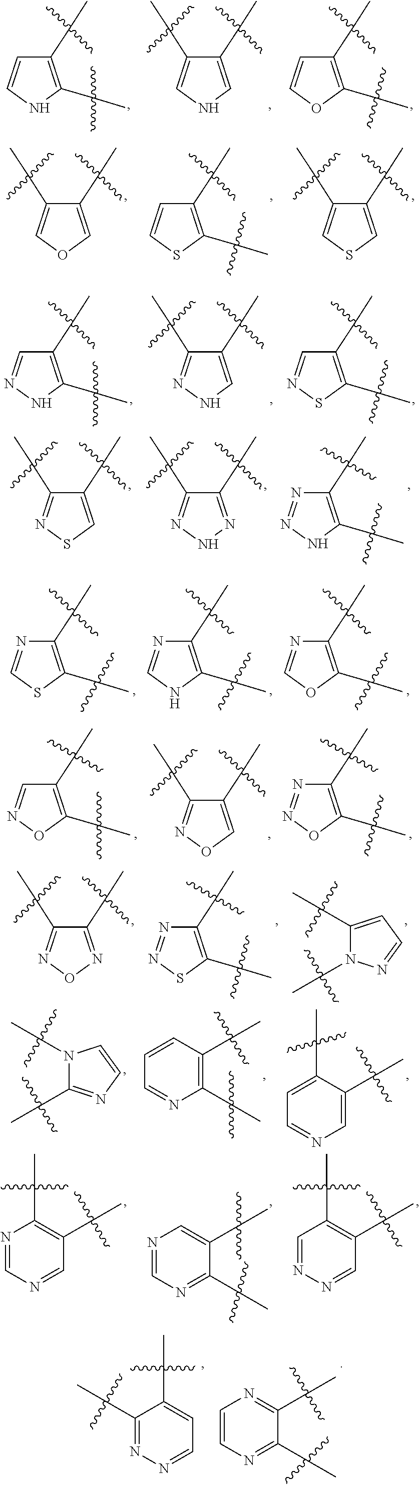

Non-limiting examples of fused polyheteroaryl rings include: benzo[d]isoxazole, 1H-indole, isoindole, 1H-benzo [d]imidazole, benzo[d]isothiazole, 1H-benzo[d][1,2,3]triazole, benzo[d]oxazole, benzo[d]thiazole, indazole, benzofuran, benzo[b]thiophene, quinoline, isoquinoline, quinazoline, quinoxaline, cinnoline, pyrido[3,2-d]pyrimidine, pyrido[2,3-d]pyrimidine, pyrido[3,4-d]pyrimidine, pyrido[4,3-d]pyrimidine, 1,8-naphthyridine, 1,7-naphthyridine, 1,6-naphthyridine, 1,5-naphthyridine, pyrazolo[1,5-a]pyrimidine, imidazolo[1,2-b]pyridazine, etc.

The above-mentioned monocyclic heteroaryl, or a polycyclic group in which a monoaryl ring is fused with a monocyclic heteroaryl ring, or a polycyclic group in which a monocyclic heteroaryl is fused with a monocyclic heteroaryl may be connected to other groups or the parent structure via a nitrogen atom or carbon atom. In the case of a polycyclic group, the ring connected to the parent structure is a heteroaryl ring, an aryl ring, a monocyclic cycloalkyl ring or a monocyclic heterocycloalkyl ring, non-limiting examples of which include:

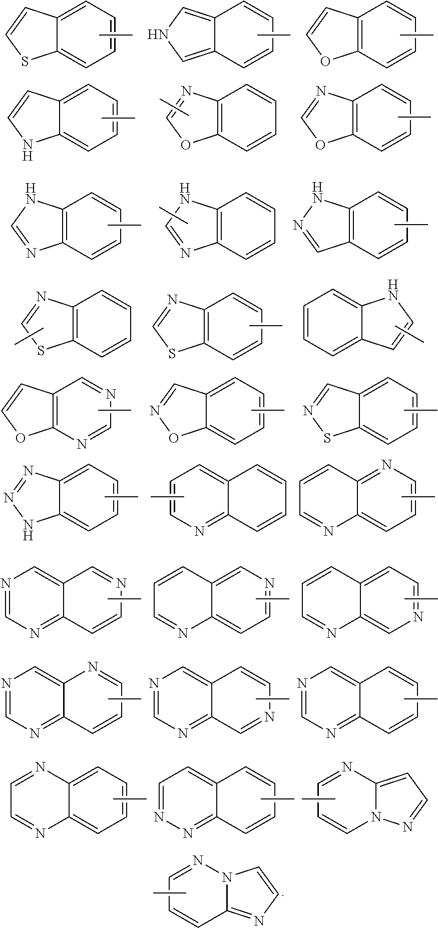

In one embodiment, the heteroaryl ring as described in the present invention (e.g., monocyclic heteroaryl ring, preferably 5- or 6-membered monocyclic heteroaryl ring) may be fused with one or more non-aromatic rings to form a polycyclic group, wherein the ring connected to the parent structure is a heteroaryl ring or a non-aromatic ring, and the non-aromatic ring includes but is not limited to: a 3- to 6-membered (preferably a 5- or 6-membered) monocyclic heterocycloalkyl ring (the ring carbon atoms of the monocyclic heterocycloalkyl ring may be substituted with 1 to 2 oxo groups to form a cyclolactam or cyclic lactone structure); a 3- to 6-membered (preferably a 5- or 6-membered) monocyclic cycloalkyl ring (the ring carbon atoms of the monocyclic cycloalkyl ring may be substituted with 1 or 2 oxo groups to form a cyclic ketone structure), etc.

The polycyclic group in which the above-mentioned monocyclic heteroaryl ring is fused with one or more non-aromatic rings can be connected to other groups or the parent structure via a nitrogen atom or carbon atom, and the ring connected to the parent structure is a heteroaryl ring or a non-aromatic ring. Non-limiting examples thereof include:

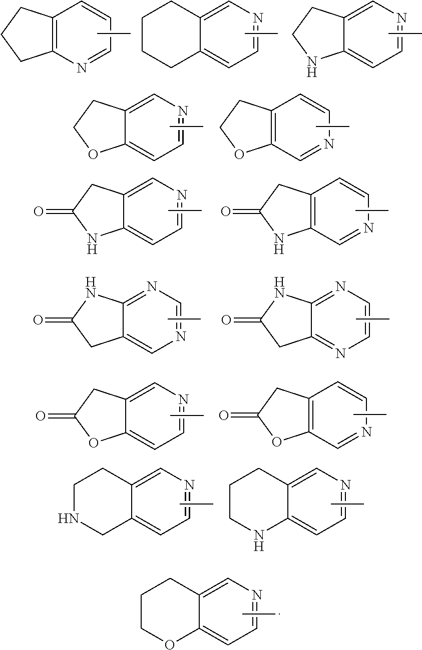

In the present invention, the above-mentioned various heteroaryl groups may be substituted or unsubstituted; when substituted, the substituents are preferably one or more groups as described in the present application.

As used herein, the term “hydroxyl” refers to —OH group.

As used herein, the term “hydroxymethyl” refers to —CH₂OH, and “hydroxyethyl” refers to —CH₂CH₂OH or —CHOHCH_{3.}

As used herein, the term “cyanomethyl” refers to —CH₂CN, “cyanoethyl” refers to —CH₂CH₂CN or —CHCNCH_{3.}

As used herein, the term “amino” refers to —NH_{2.}

As used herein, the term “cyano” refers to —CN.

As used herein, the term “nitro” refers to —NO_{2.}

As used herein, the term “benzyl” refers to —CH₂-phenyl.

As used herein, the term “oxo” refers to ═O.

As used herein, the term “carboxyl” refers to —C(O)OH.

As used herein, the term “carboxylate group” refers to —C(O)O (alkyl) or —C(O)O (cycloalkyl).

As used herein, the term “acetyl” refers to —COCH_{3.}

As used herein, the term “substituted” refers to the substitution of any one or more hydrogen atoms on a specified atom with substituents, which may include deuterium and hydrogen variants, so long as the valence of the specified atom is normal and the substituted compounds are stable. When the substituent is oxo (i.e. ═O), it means that two hydrogen atoms are substituted. Oxo substitution does not occur on aryl groups. The term “optionally substituted” means that it may or may not be substituted, and unless otherwise specified, the type and number of substituents may be arbitrary on a chemically achievable basis.

When any variable (such as R) occurs more than once in the composition or structure of a compound, its definition in each case is independent. Thus, for example, if a group is substituted with 0 to 2R, the group may optionally be substituted with up to two R, with independent options for R in each case. Furthermore, combinations of substituents and/or variants thereof are permissible only if such combinations result in stable compounds.

As used herein, C₁₋₁₀ may preferably be C₁₋₆; more preferably C₁₋₄; more preferably C₁₋₃. For example, C₁₋₁₀ alkyl may preferably be C₁₋₆ alkyl; more preferably C₁₋₄ alkyl; more preferably C₁₋₃ alkyl. For example, C₁₋₁₀ alkoxy may preferably be C₁₋₆ alkoxy; more preferably C₁₋₄ alkoxy; more preferably C₁₋₃ alkoxy.

As used herein, C₃₋₂₀ may preferably be C₃₋₁₀; more preferably C₃₋₈; more preferably C₃₋₆; more preferably C₃₋₅. For example, C₃₋₂₀ cycloalkyl may preferably be C₃₋₈ cycloalkyl; more preferably C₃₋₆ cycloalkyl; more preferably C₃₋₆ cycloalkyl.

In one embodiment, in any group, the 3- to 20-membered heterocycloalkyl is 3- to 6-membered heterocycloalkyl, 6- to 10-membered fused heterocycloalkyl, 7- to 11-membered spiroheterocycloalkyl or 7- to 10-membered bridged heterocycloalkyl, wherein, the 3- to 6-membered heterocycloalkyl, 6- to 10-membered fused heterocycloalkyl, 7- to 11-membered spiroheterocycloalkyl, and 7- to 10-membered bridged heterocycloalkyl each independently have 1, 2, or 3 heteroatoms selected from N, O and S as ring atoms.

In one embodiment, in any group, the C₃₋₆ cycloalkyl is selected from cyclopropyl, cyclobutyl, cyclopentyl, cyclohexyl.

In one embodiment, in any group, the 3- to 6-membered heterocycloalkyl is selected from aziridine, ethylene oxide, azetidine, oxetane, tetrahydrofuran, tetrahydrothiophene, tetrahydropyrrole, piperidine, piperazine, morpholine, thiomorpholine, thiomorpholine-1,1-dioxide, tetrahydropyran.

In one embodiment, in any group, the 5- or 6-membered monocyclic heteroaryl is selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

In one embodiment, in any group, the 8- to 10-membered bicyclic heteroaryl is selected from benzoxazole, benzisoxazole, benzimidazole, benzothiazole, benzisothiazole, benzotriazole, benzofuran, benzothiophene, indole, indazole, isoindole, quinoline, isoquinoline, quinazoline, quinoxaline, cinnoline, pyridopyrimidine and naphthyridine.

### DETAILED DESCRIPTION OF THE INVENTION

The compound of the present invention can be prepared by a variety of synthetic methods well known to those skilled in the art, including the specific embodiments listed below, the embodiments obtained by combining those listed below with other chemical synthesis methods, and the equivalent alternatives well known to those skilled in the art. Preferred embodiments include, but are not limited to, the examples of the present invention.

The present invention is illustrated in details by means of the examples, but is not meant to be unfavorably limited thereto. In the present invention, the present invention has been described in details, wherein specific embodiments thereof are also disclosed. It will be apparent to those skilled in the art that various changes and modifications can be made to the specific embodiments of the present invention without departing from the spirit and scope of the present invention. If specific conditions are not indicated in the examples, it shall be carried out in accordance with conventional conditions or those recommended by the manufacturer. The reagents or instruments without their manufacturers indicated are all conventional products that can be purchased commercially. As used herein, room temperature refers to 20-25° C.

### Example 1 Preparation of 1-(4-(6-chloro-8-(3,3-difluorocyclobutoxy)-7-(5-methyl-1H-indazol-4-yl)-2-((1-methylpiperidin-4-yl)oxy)quinazolin-4-yl)piperazin-1-yl)prop-2-en-1-one (Z1)

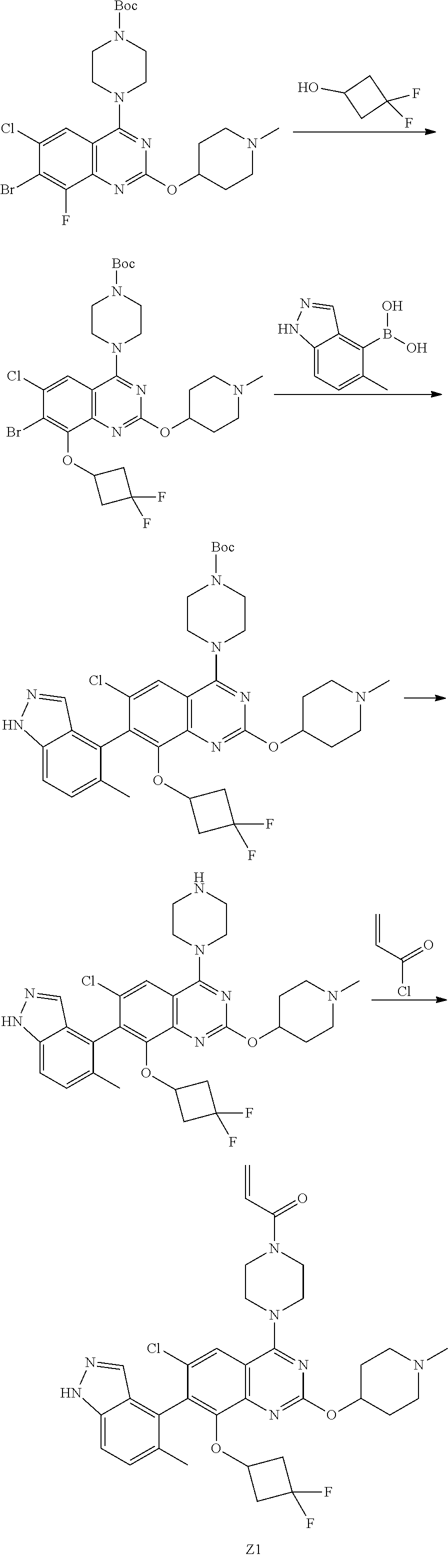

step 1: 3,3-difluorocyclobutanol (52 mg, 0.478 mmoL) was added into 5 mL of dry tetrahydrofuran, then NaH (38 mg, 0.957 mmoL) was added under ice-water bath conditions, after stirring for 10 min, tert-butyl 4-(7-bromo-6-chloro-8-fluoro-2-((1-methylpiperidin-4-yl)oxy) quinazolin-4-yl)piperazin-1-carboxylate (178 mg, 0.319 mmoL) was added. After the mixture was reacted at room temperature for 1 h, it was quenched with ice water and extracted with ethyl acetate 3 times. The organic phases were combined, concentrated under reduced pressure, purified by column chromatography (methanol/dichloromethane: 0˜10%) to obtain tert-butyl 4-(7-bromo-6-chloro-8-(3,3-difluorocyclobutoxy)-2-((1-methylpiperidin-4-yl)oxy)quinazolin-4-yl)piperazin-1-carboxylate (145 mg, Y70%). ES-API: [M+H]⁺=646.1.

step 2: tert-butyl 4-(7-bromo-6-chloro-8-(3,3-difluorocyclobutoxy)-2-((1-methylpiperidin-4-yl)oxy)quinazolin-4-yl)piperazin-1-carboxylate (145 mg, 0.224 mmoL), 5-methyl-1H-indazole-4-boronic acid (71 mg, 0.403 mmoL), Pd₂(dba)₃ (20 mg, 0.022 mmoL), 2-Biscyclohexylphosphine-2′,6′-dimethoxybiphenyl Sphos (18 mg, 0.044 mmoL) and potassium phosphate (95 mg, 0.448 mmoL) were added into a 5 mL microwave tube, then water (0.2 mL) and 1,4-dioxane (2 mL) were added. The system atmosphere was replaced with nitrogen for 1 min, and the reaction was heated at 120° C. for 50 min in the microwave. It was detected by LCMS till the reaction was complete. The mixture was poured into water, extracted with ethyl acetate, which was dried over anhydrous sodium sulfate, concentrated, and then purified by column chromatography (methanol/dichloromethane: 0˜10%) to obtain tert-butyl 4-(6-chloro-8-(3,3-difluorocyclobutoxy)-7-(5-methyl-1H-indazol-4-yl)-2-((1-methylpiperidin-4-yl)oxy))quinazolin-4-yl)piperazin-1-carboxylate (60 mg, Y50%). ES-API: [M+H]⁺=698.3.

step 3: tert-butyl 4-(6-chloro-8-(3,3-difluorocyclobutoxy)-7-(5-methyl-1H-indazol-4-yl)-2-((1-methylpiperidin-4-yl)oxy))quinazolin-4-yl)piperazin-1-carboxylate (60 mg, 0.57 mmoL) was added into 2 mL of anhydrous methanol, and 4M HCl (a 1,4-dioxane solution, 2 mL) was added dropwise slowly under ice bath conditions. The reaction was warmed up to room temperature, and then reacted for 2 h. After the reaction was complete, the mixture was concentrated under reduced pressure to obtain 6-chloro-8-(3,3-difluorocyclobutoxy)-7-(5-methyl-1H-indazol-4-yl)-2-((1-methylpiperidin-4-yl)oxy)-4-(piperazin-1-yl)quinazoline (55 mg, crude). ES-API: [M+H]⁺=598.2.

step 4: 6-chloro-8-(3,3-difluorocyclobutoxy)-7-(5-methyl-1H-indazol-4-yl)-2-((1-methylpiperidin-4-yl)oxy)-4-(piperazin-1-yl)quinazoline (51 mg, 0.085 mmoL) and triethylamine (43 mg, 0.425 mmoL) were added into 3 mL of tetrahydrofuran and 1 mL of water, and acryloyl chloride (7.7 mg, 0.085 mmoL) was then added dropwise slowly under ice-water bath conditions. The mixture was stirred for 10 min, and then extracted with dichloromethane. The organic layer was dried over anhydrous sodium sulfate, concentrated under reduced pressure and purified by preparative HPLC to obtain 1-(4-(6-chloro-8-(3,3-difluorocyclobutoxy)-7-(5-methyl-1H-indazol-4-yl)-2-((1-methylpiperidin-4-yl)oxy)quinazolin-4-yl)piperazin-1-yl)pro p-2-en-1-one (Z1, 8.5 mg, two-step reaction yield is 15%). ES-API: [M+H]⁺=652.2. ¹HNMR (400 MHz, DMSO-d)δ 8.20 (s, 1H), 7.89 (s, 1H), 7.52 (d, J=8.6 Hz, 1H), 7.44 (s, 1H), 7.33 (d, J=8.6 Hz, 1H), 6.80 (dd, J=16.7, 10.4 Hz, 1H), 6.21 (dd, J=16.7, 2.4 Hz, 1H), 5.74 (dd, J=10.4, 2.4 Hz, 1H), 5.03 (m, 1H), 4.74 (s, 1H), 4.29 (t, J=5.1 Hz, 3H), 3.86 (s, 5H), 3.48 (m, 5H), 2.73 (m, 3H), 2.24 (s, 3H), 2.15 (s, 3H), 2.06 (d, J=11.1 Hz, 2H), 1.79 (m, 2H).

### Example 2 Preparation of 1-(4-(6-chloro-7-(5-methyl-1H-indazol-4-yl)-2-((1-methylpiperidin-4-yl)oxy)-8-((tetrahydro-2H-pyran-4-yl)oxy)quinazolin-4-yl)piperazin-1-yl)prop-2-en-1-one (Z2)

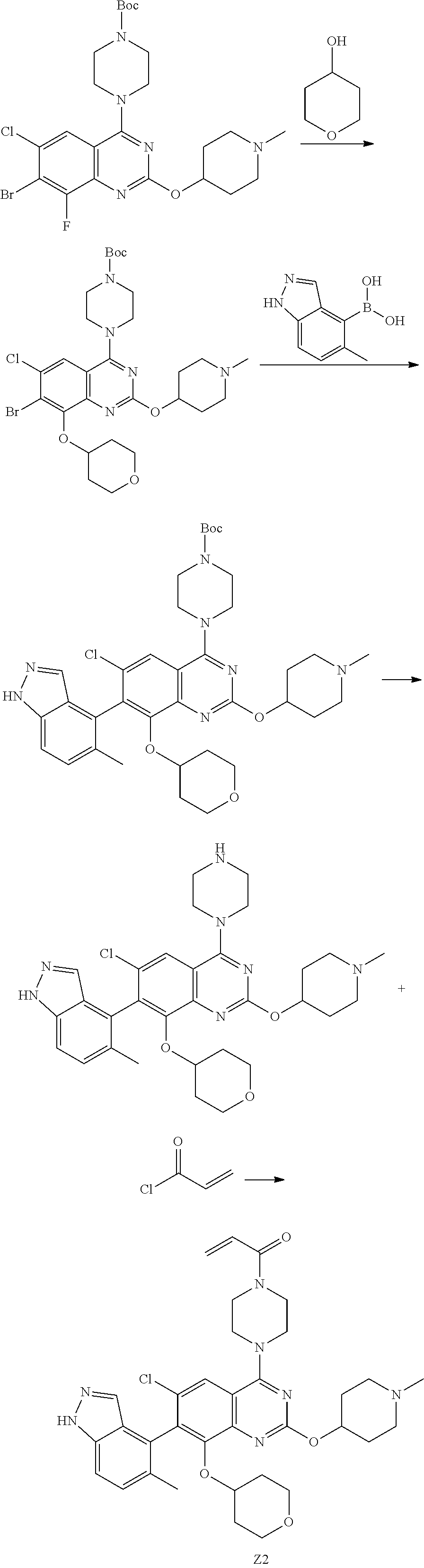

step 1: sodium hydride (43.2 mg, 1.08 mmol) was added into to a 50 mL round bottom flask and then anhydrous tetrahydrofuran (5 ml) was added. After that under ice-water bath a solution of tetrahydro-2H-pyran-4-ol (83 mg, 0.81 mmol) in tetrahydrofuran (2 ml) was added dropwise and then reacted for 10 min. After that a solution of tert-butyl 4-(7-bromo-6-chloro-8-fluoro-2-((1-methylpiperidin-4-yl)oxy)quinazolin-4-yl)piperazin-1-carboxylate (300 mg, 0.54 mmol) in tetrahydrofuran (5 ml) was added, reacted for 10 min under ice-water bath and then reacted for 2 h at 70° C. Then 30 mL of water was added into the reaction solution, which was extracted with 30 mL of ethyl acetate three times. The organic phase was dried and concentrated, and then purified by column chromatography (methanol/dichloromethane: 0˜10%) to obtain tert-butyl 4-(7-bromo-6-chloro-2-((1-methylpiperidin-4-yl)oxy)-8-((tetrahydro-2H-pyran-4-yl) oxy)quinazolin-4-yl)piperazin-1-carboxylate (245 mg, Y: 71%).

step 2: to a 50 mL round bottom flask, tert-butyl 4-(7-bromo-6-chloro-2-((1-methylpiperidin-4-yl)oxy)-8-((tetrahydro-2H-pyran-4-yl)oxy)quinazolin-4-yl)piperazin-1-carboxylate (245 mg, 0.38 mmol), (5-methyl-TH-indazol-4-yl) boronic acid (100 mg, 0.57 mmol), SPhos (16 mg, 0.038 mmol), Tris (dibenzylideneacetone)dipalladium (35 mg, 0.038 mmol) and potassium phosphate (241 mg, 1.14 mmol) were added to dioxane (10 ml) and water (2 ml). After the system atmosphere was replaced with nitrogen three times, and the oil bath was heated to 120° C., the reaction was stirred for 16 h. After the reaction was cooled to room temperature, 30 mL of water was added, and the mixture was extracted with 30 mL of dichloromethane two times. The organic phase was dried and concentrated, and then purified by column chromatography (methanol/dichloromethane: 0˜10%) to obtain tert-butyl 4-(6-chloro-7-(5-methyl-TH-indazol-4-yl)-2-((1-methylpiperidin-4-yl)oxy)-8-((tetrahydro-2H-pyran)-4-yl)oxy)quinazolin-4-yl)piperazin-1-carboxylate (131 mg, Y: 51%).

step 3: to a 50 mL round bottom flask, tert-butyl 4-(6-chloro-7-(5-methyl-TH-indazol-4-yl)-2-((1-methylpiperidin-4-yl)oxy)-8-((tetrahydro-2H-pyran)-4-yl)oxy) quinazolin-4-yl)piperazin-1-carboxylate (131 mg, 0.19 mmol), dichloromethane (5 ml) and trifluoroacetic acid (2 ml) were added. The reaction was conducted at room temperature for 0.5 h. The organic phase was dried and concentrated to dryness. Then adding dichloromethane (30 ml) and concentrating were repeated three times to obtain a crude of 6-chloro-7-(5-methyl-1H-indazol-4-yl)-2-((1-methylpiperidin-4-yl)oxy)-4-(piperazin-1-yl)-8-((tetrahydro-2H-pyran-4-yl)oxy)quinazoline (151 mg, Y: 100%).

step 4: to a 50 mL round bottom flask, 6-chloro-7-(5-methyl-1H-indazol-4-yl)-2-((1-methylpiperidin-4-yl)oxy)-4-(piperazin-1-yl)-8-((tetrahydro-2H-pyran-4-yl)oxy)quinazoline (151 mg, 0.26 mmol) and dichloromethane (10 ml) were added, then triethylamine was added to adjust the pH of the reaction system to about 8. After that under ice-water bath a solution of acryloyl chloride (26 ml, 0.21 mmol) in dichloromethane (3 ml) was added dropwise and reacted for 5 min. Then 30 ml of a saturated sodium bicarbonate solution was added, and the mixture was extracted with 30 ml of dichloromethane three times. The organic phase was dried and concentrated to dryness, then purified by preparative HPLC to obtain 1-(4-(6-chloro-7-(5-methyl-1H-indazol-4-yl)-2-((1-methylpiperidin-4-yl)oxy)-8-((tetrahydro-2H-pyran-4-yl)oxy)quinazolin-4-yl)piperazin-1-yl)prop-2-en-1-one (Z2, 20 mg, Y: 12%). ES-API: [M+H]⁺=646.2.

### Example 3 Preparation of 1-(4-(6-chloro-8-cyclobutoxy-7-(5-methyl-1H-indazol-4-yl)-2-((1-methylpiperidin-4-yl)oxy)quinazolin-4-yl)piperazin-1-yl)prop-2-en-1-one (Z3)

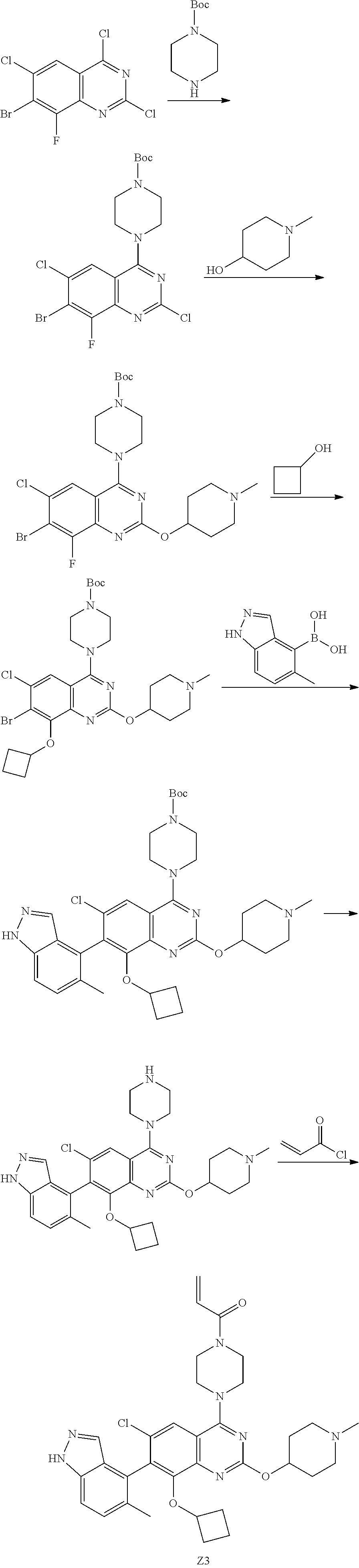

step 1: 7-bromo-2,4,6-trichloro-8-fluoro quinazoline (600 mg, 1.830 mmol, 1.0 eq) was added to a mixed solution of N,N-diisopropylethylamine (2.365 g, 18.30 mmol, 10.0 eq) and acetonitrile (20 mL), and then cooled to 0-5° C. under ice-water bath. Under vigorous stirring, compound tert-butyl piperazin-1-carboxylate (344.04 mg, 1.848 mmol, 1.01 eq) was added in batches. Under nitrogen protection the system was allowed to be warmed from 0° C. to room temperature. The reaction was monitored by TLC [PE:EA=4:1, v/v] till the raw materials disappeared and the reaction was stopped. 80 mL of ethyl acetate was added to the system, which was washed with saturated brine three times (3*60 mL). The ethyl acetate phase was dried over anhydrous sodium sulfate and filtered. The filtrate was dried by rotary evaporation under reduced pressure to obtain tert-butyl 4-(7-bromo-2,6-dichloro-8-fluoroquinazolin-4-yl)piperazin-1-carboxylate 800 mg (Y: 90%) as a pale yellow solid, ES-API: [M+H]⁺=479.1.

step 2: to 20 mL of DMSO, tert-butyl 4-(7-bromo-2,6-dichloro-8-fluoroquinazolin-4-yl)piperazin-1-carboxylate (800 mg, 1.672 mmol, 1.0 eq), 1-methyl-4-piperidinol (1.925 g, 16.72 mmol, 10.0 eq) and potassium fluoride (971.43 mg, 16.72 mmol, 10.0 eq) were added in sequence. Under nitrogen protection, the reaction was slowly warmed up to 120° C. and reacted for 2 h. The reaction was monitored by TLC [PE:EA=4:1, v/v] till the raw materials disappeared and the reaction was stopped. The reaction system was cooled to room temperature, then 800 mL of ethyl acetate was added, and the mixture was washed with saturated brine five times (5*50 mL). The ethyl acetate phase was dried over anhydrous sodium sulfate and filtered. The filtrate was dried by rotary evaporation under reduced pressure, and purified by P-TLC [DCM:MeOH=10:1, v/v] to obtain tert-butyl 4-(7-bromo-6-chloro-8-fluoro-2-((1-methylpiperidin-4-yl)oxy)quinazolin-4-yl)piperazin-1-carboxylate 400 mg (Y: 45%) as a pale yellow solid, ES-API: [M+H]⁺=558.1.

step 3: to 30 mL of dry THF, cyclobutanol (37.35 mg, 0.5183 mmol, 1.05 eq) and potassium tert-butoxide (110.77 mg, 0.9872 mmol, 2.0 eq) were added in sequence. After being stirred at room temperature for 0.5 h, tert-butyl 4-(7-bromo-6-chloro-8-fluoro-2-((1-methylpiperidin-4-yl)oxy)quinazolin-4-yl)piperazin-1-carboxylate (275 mg, 0.4936 mmol, 1.0 eq) was added. Under nitrogen protection, the system was warmed up to 70° C. and reacted for 1.5 h. The reaction was monitored by TLC [DCM:MeOH=10:1, v/v] till the raw materials disappeared and the reaction was stopped. The reaction system was cooled to room temperature, then 30 mL of saturated ammonium chloride aqueous solution was added and then 80 mL of ethyl acetate was added, and the mixture was washed with saturated brine five times (5*50 mL). The ethyl acetate phase was dried over anhydrous sodium sulfate and filtered. The filtrate was dried by rotary evaporation under reduced pressure and purified by P-TLC [DCM:MeOH=10:1, v/v] to obtain pale yellow tert-butyl 4-(7-bromo-6-chloro-8-cyclobutoxy-2-((1-methylpiperidin-4-yl)oxy)quinazolin-4-yl)piperazin-1-carboxylate 130 mg (Y: 44%), ES-API: [M+H]⁺=610.1.

step 4: tert-butyl 4-(7-bromo-6-chloro-8-cyclobutoxy-2-((1-methylpiperidin-4-yl)oxy) quinazolin-4-yl)piperazin-1-carboxylate (130 mg, 0.2134 mmol, 1.0 eq), 5-methyl-1H-indazole-4-boronic acid (75.15 mg, 0.4268 mmol, 2.0 eq), tris (dibenzylideneacetone)dipalladium (29.32 mg, 0.03201 mmol, 0.15 eq), 2-dicyclohexylphosphine-2′,6′-dimethoxy-biphenyl (13.14 mg, 0.03201 mmol, 0.15 eq) and potassium phosphate (88.35 mg, 0.6402 mmol, 3.0 eq) were added to a mixed solution of 12 mL of dioxane and 2.5 mL of water. The system was charged with nitrogen for 3 min and then reacted under microwave at 120° C. for 50 min. The reaction system was cooled to room temperature, then 50 mL of ethyl acetate was added and washed with saturated brine three times (3*30 mL). The ethyl acetate phase was dried over anhydrous sodium sulfate and filtered. The filtrate was dried by rotary evaporation under reduced pressure and purified by PTLC [DCM:MeOH=10:1, v/v] to obtain 190 mg of tert-butyl 4-(6-chloro-8-cyclobutoxy-7-(5-methyl-1H-indazol-4-yl)-2-((1-methylpiperidin-4-yl)oxy)quinazolin-4-yl))piperazin-1-carboxylate (crude) as a light brown solid, ES-API: [M+H]⁺=662.3.

step 5: tert-butyl 4-(6-chloro-8-cyclobutoxy-7-(5-methyl-1H-indazol-4-yl)-2-((1-methylpiperidin-4-yl)oxy)quinazolin-4-yl))piperazin-1-carboxylate (190 mg, 0.2134 mmol, 1.0 eq) was dissolved in 8 mL of dichloromethane, and finally 2 mL of trifluoroacetic acid was added. The reaction was conducted at room temperature overnight. The reaction was monitored by LC-MS till the reaction was complete. The solvent was dried by rotary evaporation under reduced pressure to obtain 210 mg of crude 6-chloro-8-cyclobutoxy-7-(5-methyl-1H-indazol-4-yl)-2-((1-methylpiperidin-4-yl)oxy)-4-(piperazin-1-yl)quinazoline trifluoroacetate as brown oily viscous liquid, which was directly used in the next step. ES-API: [M+H]⁺=562.3.

step 6: 6-chloro-8-cyclobutoxy-7-(5-methyl-1H-indazol-4-yl)-2-((1-methylpiperidin-4-yl)oxy)-4-(piperazin-1-yl)quinazoline trifluoroacetate (210 mg, 0.2134 mmol, 1.0 eq) was added to 30 mL of anhydrous tetrahydrofuran. After cooling to 0-5° C. under ice-water bath, triethylamine (215.5 mg, 2.134 mmol, 10.0 eq) was added. After stirring for 10 min a solution of acryloyl chloride (20.28 mg, 0.2241 mmol, 1.05 eq) in tetrahydrofuran (2.0 mL) was added dropwise. Under ice-water bath, the mixture was stirred for 30 min. The reaction was monitored by TLC [DCM:MeOH=10:1, v/v] till the raw materials disappeared and the reaction was stopped. 50 mL of ethyl acetate was added to the system and then washed with saturated brine (3*30 mL). The ethyl acetate phase was dried over anhydrous sodium sulfate and filtered. The filtrate was dried by rotary evaporation under reduced pressure (≤40° C.) and the crude was purified by preparative liquid chromatography to obtain 32 mg of 1-(4-(6-chloro-8-cyclobutoxy-7-(5-methyl-1H-indazol-4-yl)-2-((1-methylpiperidin-4-yl)oxy)quinazolin-4-yl)piperazin-1-yl)prop-2-en-1-one (Z3, Y: 24.38%) as a white solid, ES-API: [M+H]⁺⁼616.3. ¹HNMR (400 MHz, dmso)δ 9.77 (d, J=41.0 Hz, 1H), 7.86 (s, 1H), 7.48 (d, J=8.4 Hz, 1H), 7.41 (d, J=2.7 Hz, 1H), 7.31 (d, J=8.6 Hz, 1H), 6.80 (dd, J=15.0, 10.5 Hz, 1H), 6.15 (d, J=16.7 Hz, 1H), 5.72 (d, J=11.1 Hz, 1H), 5.23 (d, J=79.6 Hz, 1H), 4.61 (dt, J=14.0, 7.1 Hz, 1H), 3.83 (s, 7H), 3.51 (d, J=12.2 Hz, 2H), 3.36 (d, J=11.1 Hz, 2H), 3.15 (s, 3H), 2.85-2.73 (m, 3H), 2.35 (d, J=11.8 Hz, 1H), 2.23 (d, J=13.3 Hz, 1H), 2.08 (s, 3H), 1.90 (s, 2H), 1.53 (dd, J=17.9, 8.7 Hz, 2H), 1.27 (dd, J=20.0, 15.4 Hz, 2H).

### Example 4 Preparation of 1-(4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-1-yl)prop-2-en-1-one (Z4)

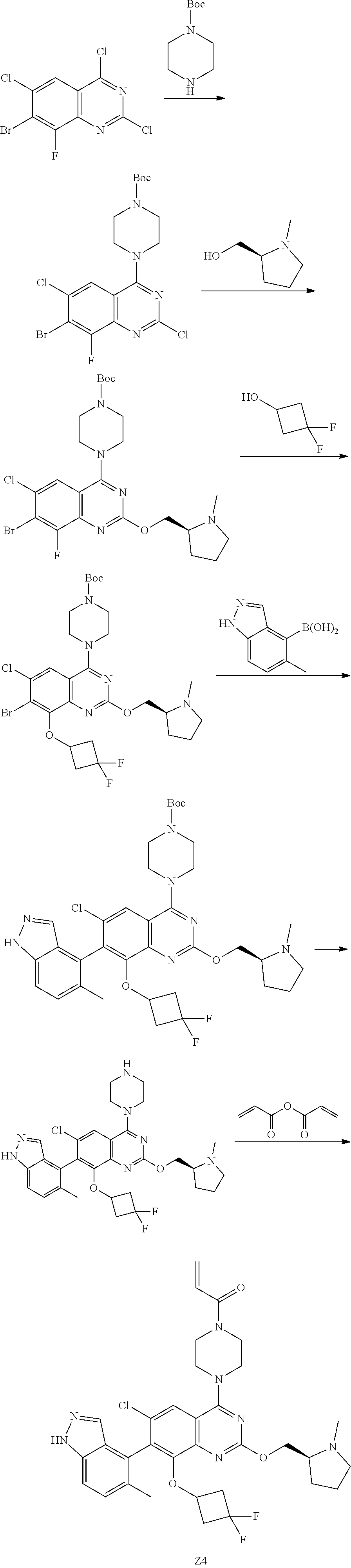

step 1: 7-bromo-2,4,6-trichloro-8-fluoro quinazoline (28 g, 84.8 mmol) and triethylamine (26 g, 254.4 mmol) were dissolved in acetonitrile and dichloromethane (250 mL/250 mL). After cooling in an ice bath, tert-butyl piperazin-1-carboxylate acetate (23.7 g, 127 mmol) was added. After the addition, the reaction was conducted for 2 h while keeping the temperature. After the reaction was complete, it was extracted with dichloromethane (300 mL×3), washed with water and washed with saturated brine, dried and then dried by rotary evaporation to obtain tert-butyl 4-(7-bromo-2,6-dichloro-8-fluoroquinazolin-4-yl)piperazin-1-carboxylate (37 g, Y: 100%) as a pale yellow solid. ES-API: [M+1]⁺=479.0.

step 2: tert-butyl 4-(7-bromo-2,6-dichloro-8-fluoroquinazolin-4-yl)piperazin-1-carboxylate (24 g, 50 mmol) was added into (S)-(1-methylpyrrolidin-2-yl) methanol (115 g, 1000 mmol), and then potassium fluoride (8.7 g, 150 mmol) was added. The reaction was conducted at 100° C. for 2 h. After cooling, the reaction solution was poured into water (300 mL) and extracted with ethyl acetate (200 mL×3). The organic phase was washed with water and washed with brine and then dried by rotary evaporation, and purified by column chromatography (methanol:dichloromethane=0-5%) to obtain tert-butyl (S)-4-(7-bromo-6-chloro-8-fluoro-2-((1-methylpyrrolidin-2-yl) methoxy)quinazolin-4-yl)piperazin-1-carboxylate (11 g, Y: 39%) as a yellow solid. ES-API: [M+1]⁺=558.1.

step 3: 3,3-difluorocyclobutanol (1.16 g, 10.75 mmol) was dissolved in tetrahydrofuran (30 mL), and sodium hydrogen (860 mg, 35.85 mmol) was added at 0° C. The reaction was stirred for 0.5 h while keeping the temperature. tert-butyl (S)-4-(7-bromo-6-chloro-8-fluoro-2-((1-methylpyrrolidin-2-yl)methoxy)quinazolin-4-yl)piperazin-1-carboxylate (4 g, 7.17 mmol) was then added into the reaction solution. The reaction was conducted for 1 h while keeping the temperature. The reaction solution was poured into ethyl acetate (100 mL), and the organic phase was washed with water (20 mL×2) and washed with brine (20 mL), dried over anhydrous sodium sulfate and filtered. The filtration was dried by rotary evaporation to obtain tert-butyl (S)-4-(7-bromo-6-chloro-8-(3,3-difluorocyclobutoxy)-2-((1-methylpyrrolidin-2-yl)methoxy)quinazolin-4-yl)piperazin-4-1-carboxylate (4.1 g, Y: 80%) as a yellow oil. ES-API: [M+1]⁺=646.1.

step 4: tert-butyl (S)-4-(7-bromo-6-chloro-8-(3,3-difluorocyclobutoxy)-2-((1-methylpyrrolidin-2-yl)methoxy)quinazolin-4-yl)piperazin-4-1-carboxylate (1.8 g, 2.83 mmol), (5-methyl-1H-indazol-4-yl) boronic acid (1.5 g, 8.49 mmol), tripotassium phosphate (3 g, 14.15 mmol), tris (dibenzylideneindenacetone)dipalladium (260 mg), 2-dicyclohexylphosphine-2′,4′,6′-triisopropylbiphenyl (130 mg) were added in sequence into dioxane (20 mL)/water (5 mL). The system atmosphere was replaced with nitrogen three times and then the temperature was warmed up to 105° C. and the reaction was conducted for 6 h. After cooling to room temperature, the reaction solution was poured into ethyl acetate (100 mL) and after being washed with brine once, it was mixed with silica gel and purified (methanol:dichloromethane=0˜1:20) to obtain tert-butyl 4-(6-chloro-8-(3,3-difluorocyclobutoxy)-7-(5-methyl-1H-indazol-4-yl)-2-((S)-1-methylpyrrolidin)-2-yl)methoxy)quinazolin-4-yl)piperazin-1-carboxylate (1.3 g, Y: 68%) as a yellow solid. ES-API: [M+1]⁺=698.2.

step 5: tert-butyl 4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-1-carboxylate (1.1 g, 1.57 mmol) was dissolved in dichloromethane (20 mL), and then trifluoroacetic acid (2 mL) was added. The reaction was conducted at room temperature for 0.5 h, and then dried by rotary evaporation to obtain crude 6-chloro-8-(3,3-difluorocyclobutoxy)-7-(5-methyl-1H-indazol-4-yl)-2-(((S)-1-methylpyrrolidin-2-yl)methoxy)-4-(piperazin-1-yl)quinazoline (1.2 g, Y: 100%) as a yellow oil.

step 6: 6-chloro-8-(3,3-difluorocyclobutoxy)-7-(5-methyl-1H-indazol-4-yl)-2-(((S)-1-methylpyrrolidin-2-yl)methoxy)-4-(piperazin-1-yl)quinazoline (1.1 g, 1.83 mmol) and triethylamine (2 mL) were dissolved in dichloromethane (20 mL). At 0° C. acrylic anhydride (208 mg, 1.65 mmol) was added and the reaction was conducted for 1 h. The reaction solution was dried by rotary evaporation and purified by flash column chromatography (dichloromethane:methanol=0˜20/1) to obtain crude 1-(4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-1-yl)prop-2-en-1-one (640 mg, the purity is 95%). The crude was lyophilized to obtain 1-(4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-1-yl)prop-2-en-1-one (Z4, 402 mg, Y: 33%). ES-API: [M+1]⁺=652.2. ¹HNMR (400 MHz, chloroform-d)δ 8.21 (d, J=140.7 Hz, 4H), 7.78 (s, 1H), 7.57-7.46 (m, 2H), 7.35 (d, J=8.6 Hz, 1H), 6.61 (dd, J=16.7, 10.7 Hz, 1H), 6.37 (d, J=16.7 Hz, 1H), 5.78 (d, J=10.5 Hz, 1H), 4.77 (dd, J=12.8, 6.5 Hz, 1H), 4.71-4.54 (m, 2H), 3.86 (d, J=18.7 Hz, 8H), 3.60 (d, J=8.7 Hz, 1H), 3.35 (s, 1H), 2.81 (d, J=4.3 Hz, 3H), 2.69 (dt, J=29.5, 8.6 Hz, 2H), 2.48-2.29 (m, 2H), 2.24 (s, 1H), 2.20 (s, 3H), 2.09 (d, J=12.9 Hz, 1H), 1.97 (d, J=10.4 Hz, 3H).

### Example 5 Preparation of 1-((3S)-4-(6-chloro-8-(3,3-difluorocyclobutanyl)-7-(5-methyl-1H-indazol-4-yl)-2-(((S)-1-meth ylpyrrolidin-2-yl)methoxy)quinazolin-4-yl)-3-methylpiperazin-1-yl)prop-2-en-1-one (Z5)

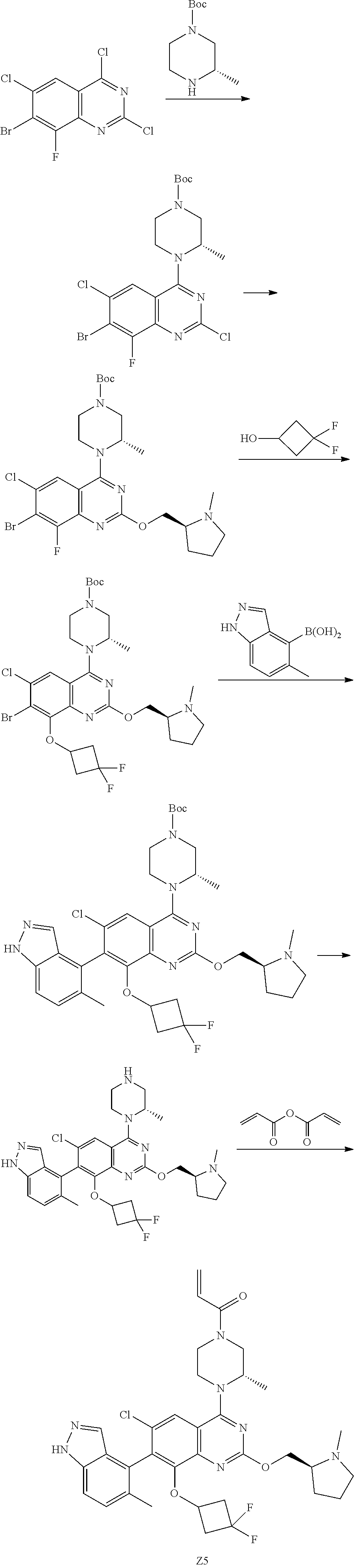

step 1: at 0° C., to a mixed solution of 7-bromo-2,4,6-trichloro-8-fluoroquinazoline (5.0 g, 15.13 mmol) and N,N′-diisopropylethylamine (5.87 g, 45.40 mmol) in dichloromethane (30 mL) and acetonitrile (30 mL) was added tert-butyl (S)-3-methylpiperazin-1-carboxylate (3.03 g, 15.13 mmol). Then the mixture was stirred at room temperature for 2 h. Then water (50 mL) was added and the mixture was extracted with dichloromethane (3×50 mL). The combined organic phase was washed with saturated brine (3×50 mL) and dried over anhydrous sodium sulfate. The solvent was removed by rotary evaporation. The residue was triturated with dichloromethane and petroleum ether and filtered with suction to obtain tert-butyl (S)-4-(7-bromo-2,6-dichloro-8-fluoroquinazolin-4-yl)-3-methylpiperazin-1-carboxylate (5.0 g, Y: 66.9%) as a yellow solid, which was used directly in the next step without further purification. ES-API: [M+1]⁺=495.0.

step 2: to a mixed solution of tert-butyl (S)-4-(7-bromo-2,6-dichloro-8-fluoroquinazolin-4-yl)-3-methylpiperazin-1-carboxylate (4.0 g, 8.09 mmol), triethylene diamine (454 mg, 4.05 mmol) and N-methyl-L-prolinol (2.80 g, 24.28 mmol) in N, N′-dimethylformamide (20 mL) was added cesium carbonate (7.91 g, 24.28 mmol). The mixture was heated to 80° C. under an argon atmosphere and kept for 3 h. The reaction solution was diluted with water (200 mL), and extracted with ethyl acetate (3×60 mL). The combined organic phase was washed with water (3×30 mL) and washed with saturated brine (3×50 mL), and dried over anhydrous sodium sulfate. The solvent was removed by rotary evaporation. The residue was purified by flash chromatography, eluting with dichloromethane and methanol (3%) to obtain tert-butyl (S)-4-(7-bromo-6-chloro-8-fluoro-2-(((S)-1-methylpyrrolidin-2-yl)methoxy)quinazolin-4-yl)-3-methylpiperazin-1-carboxylate (2.0 g, Y: 43.1%) as a yellow oil. ES-API: [M+1]⁺=572.2.

step 3: at 0° C., to a mixed solution of 3,3′-difluorocyclobutanol (396 mg, 3.67 mmol) in tetrahydrofuran (20 mL) was added sodium hydride (418.9 mg, 10.47 mmol, the purity is 60%). The obtained mixture was stirred at 0° C. for 1 h. Then a solution of tert-butyl (S)-4-(7-bromo-6-chloro-8-fluoro-2-(((S)-1-methylpyrrolidin-2-yl)methoxy)quinazolin-4-yl)-3-methylpiperazin-1-carboxylate (2.0 g, 3.49 mmol) in tetrahydrofuran (10 mL) was added into the reaction mixture. Then the reaction was conducted at room temperature for 1 h. The reaction mixture was poured into ice water (100 mL) and extracted with dichloromethane (3×100 mL). The combined organic phase was washed with saturated ammonium chloride (2×30 mL) and washed with saturated brine (3×50 mL). The solvent was removed by rotary evaporation. The residue was purified by flash chromatography, eluting with dichloromethane and methanol (3%), to obtain tert-butyl (S)-4-(7-bromo-6-chloro-8-(3,3′-difluorocyclobutoxy)-2-(((S)-1-methylpyrrolidin-2-yl)methyl)quinazolin-pyridin-4-yl)-3-methylpiperazin-1-carboxylate (1.0 g, Y: 43.3%) as a red oil. ES-API: [M+1]⁺=660.2

step 4: to a suspension of tert-butyl (S)-4-(7-bromo-6-chloro-8-(3,3′-difluorocyclobutoxy)-2-(((S)-1-methylpyrrolidin-2-yl)methyl)quinazolin-pyridin-4-yl)-3-methylpiperazin-1-carboxylate (1 g, 1.51 mmol), (5-methyl-1H-indazol-4-yl) boronic acid (532.5 mg, 3.03 mmol) and potassium phosphate (642.3 mg, 3.03 mmol) in 1,4-dioxane (10 mL) and deionized water (2.5 mL) were added tris (dibenzylideneindenacetone)dipalladium (138.5 mg, 0.15 mmol) and dicyclohexylphosphorus-2,6-dimethoxy 1,1′-biphenyl (124.2 mg, 0.30 mmol). The obtained mixture was heated to 100° C. and stirred overnight under an argon atmosphere. After cooling to room temperature, the reaction mixture was extracted with dichloromethane (3×150 mL). The combined organic phase was washed with water (1×50 mL) and washed with saturated brine (3×50 mL) and dried over anhydrous sodium sulfate. The solvent was removed by rotary evaporation. The residue was purified by flash chromatography, eluting with dichloromethane and methanol (3%), to obtain tert-butyl (3S)-4-(6-chloro-8-(3,3′-difluorocyclobutoxy)-7-(5-methyl-1H-indazol-4-yl)-2-(((S)-1-)methylpyrrolidin-2-yl)methoxy)quinazolin-4-yl)-3-methylpiperazin-1-carboxylate (450 mg, Y: 41.8%) as a yellow oil. ES-API: [M+1]⁺=712.3

step 5: to a solution of tert-butyl (3S)-4-(6-chloro-8-(3,3′-difluorocyclobutoxy)-7-(5-methyl-1H-indazol-4-yl)-2-(((S)-1-)methylpyrrolidin-2-yl)methoxy)quinazolin-4-yl)-3-methylpiperazin-1-carboxylate (450 mg, 0.63 mmol) in dichloromethane (4 mL), trifluoroacetic acid (2 mL) was added. The obtained mixture was stirred at room temperature for 30 min. The pH of the reaction mixture was adjusted to 8 with 1M sodium bicarbonate. The reaction mixture was extracted with dichloromethane (3×30 mL). The combined organic phase was washed with saturated brine (3×30 mL), and dried over anhydrous sodium sulfate. The solvent was removed by rotary evaporation to obtain 6-chloro-8-(3,3′-difluorocyclobutoxy)-7-(5-methyl-1H-indazol-4-yl)-4-((S)-2-methylpiperazin-1-yl)-2-(((S)-1-methylpyrrolidin-2-yl)methoxy)quinazoline (354 mg, Y: 91.5%) as a yellow oil, which was used directly in the next step without further purification. ES-API: [M+1]⁺=612.3

step 6: to a solution of 6-chloro-8-(3,3′-difluorocyclobutoxy)-7-(5-methyl-1H-indazol-4-yl)-4-((S)-2-methylpiperazin-1-yl)-2-(((S)-1-methylpyrrolidin-2-yl)methoxy)quinazoline (354 mg, 0.58 mmol) in dichloromethane (5 mL) was added triethylamine (87.8 mg, 0.87 mmol). The mixture was cooled to 0° C. At 0° C. acrylic anhydride (87.5 mg, 0.69 mmol) was added and stirred for 2 h. Then the solvent was removed under reduced pressure. The residue was purified by Pre-HPLC to obtain 1-((3S)-4-(6-chloro-8-(3,3-difluorocyclobutanyl)-7-(5-methyl-1H-indazol-4-yl)-2-(((S)-1-methylpyrrolidin-2-yl)methoxy)quinazolin-4-yl)-3-methylpiperazin-1-yl)prop-2-en-1-one (Z5, 87.5 mg, Y: 22.7%) as a white solid. ¹HNMR (400 MHz, chloroform-d)δ 7.74 (s, 1H), 7.54 (d, J=20.8 Hz, 2H), 7.38 (d, J 9.2 Hz, 1H), 6.39 (d, J=16.5 Hz, 1H), 5.79 (d, J=10.3 Hz, 1H), 4.67 (s, 3H), 3.65 (s, 3H), 2.96 (s, 4H), 2.15 (d, J=57.2 Hz, 14H), 1.44 (s, 7H). HPLC purity: 97.42% (214 nm); 97.87% (254 nm); ES-API: [M+1]⁺=666.3.

### Example 6 Preparation of 2-((2S)-1-acryloyl-4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-2-yl) acetonitrile (Z6)

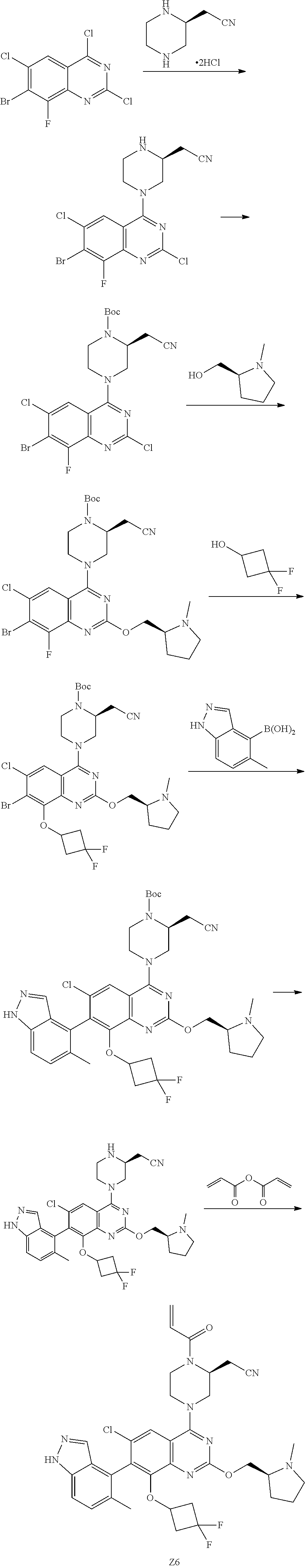

step 1: 7-bromo-2,4,6-trichloro-8-fluoroquinazoline (5 g, 0.015 mol) and triethylamine (7.7 g, 0.076 mol) were dissolved in acetonitrile (100 mL). After cooling in an ice bath, (S)-2-(piperazin-2-yl) acetonitrile (bishydrochloride) (3.6 g, 0.018 mol) was added. After the addition, the reaction was conducted for 1 h while keeping the temperature. The reaction solution was directly used in the next step (Y: 100%). ES-API: [M+1]⁺=420.1.

step 2: to the reaction solution of step 1 triethylamine (4.6 g, 0.045 mol) and 4-dimethylaminopyridine (183 mg, 1.5 mmol) were added, and di-tert-butyl dicarbonate (6.5 g, 0.03 mol) was added dropwise. After the addition, the reaction was conducted at room temperature overnight. The reaction solution was poured into water (200 mL) and extracted with ethyl acetate (100 mL×3). The organic phase was washed with water and washed with brine and then dried by rotary evaporation, and purified by column chromatography (petroleum ether:ethyl acetate=10-30%) to obtain tert-butyl (S)-4-(7-bromo-2,6-dichloro-8-fluoroquinazolin-4-yl)-2-(cyanomethyl)piperazin-1-carboxylate (3.6 g, Y: 46%) as a yellow oil. ES-API: [M+1]⁺=520.1.

step 3: tert-butyl (S)-4-(7-bromo-2,6-dichloro-8-fluoroquinazolin-4-yl)-2-(cyanomethyl)piperazin-1-carboxylate (3.6 g, 6.93 mmol) was added to (S)-(1-methylpyrrolidin-2-yl) methanol (12 g, 0.1 mol), and then potassium fluoride (1.2 g, 20.8 mmol) was added. The reaction was conducted at 100° C. for 2 h. After cooling, the reaction solution was poured into water (200 mL) and extracted with ethyl acetate (100 mL×3). The organic phase was washed with water and washed with brine and then dried by rotary evaporation, and purified by column chromatography (methanol:dichloromethane=0˜10%) to obtain tert-butyl (S)-4-(7-bromo-6-chloro-8-fluoro-2-(((S)-1-methylpyrrolidin-2-yl)methoxy) quinazolin-4-yl)-2-(4-yl)cyanomethyl)piperazin-1-carboxylate (3.4 g, Y: 82%) as a yellow oil. ES-API: [M+1]⁺=599.1. ¹HNMR (400 MHz, CDCl₃)δ 7.71 (s, 1H), 4.64 (s, 1H), 4.53 (dd, J=10.7, 4.4 Hz, 1H), 4.34 (dd, J=10.6, 6.3 Hz, 1H), 4.13 (ddd, J=22.0, 21.2, 12.1 Hz, 4H), 3.62 (dd, J=11.5, 8.6 Hz, 1H), 3.39 (d, J=8.8 Hz, 2H), 3.16-3.05 (m, 1H), 2.82-2.62 (m, 3H), 2.50 (s, 3H), 2.21 (d, J=16.1 Hz, 1H), 2.05 (d, J=4.2 Hz, 1H), 1.78 (dd, J=19.9, 8.7 Hz, 3H), 1.50 (d, J=10.9 Hz, 9H).

step 4: 3,3-difluorocyclobutanol (1.5 g, 14.05 mmol) was dissolved in tetrahydrofuran (30 mL), and at 0° C. sodium hydrogen (375 mg, 9.36 mmol) was added. The reaction was stirred for 0.5 h while keeping the temperature. tert-butyl (S)-4-(7-bromo-6-chloro-8-fluoro-2-(((S)-1-methylpyrrolidin-2-yl)methoxy)quinazolin-4-yl)-2-(4-yl)cyanomethyl)piperazin-1-carboxylate (2.8 g, 4.68 mmol) was then added into the reaction solution. The reaction was conducted for 1 h while keeping the temperature. The reaction solution was poured into ethyl acetate (100 mL), and the organic phase was washed with water (20 mL×2) and then washed with brine (20 mL), dried over anhydrous sodium sulfate and filtered. The filtration was dried by rotary evaporation to obtain tert-butyl ((S)-4-(7-bromo-6-chloro-8-(3,3-difluorocyclobutoxy)-2-(((S)-1-methylpyrrolidin-2-yl)methoxy)quinazolin-4-yl)-2-(cyanomethyl)piperazin-1-carboxylate (3 g, Y: 78%) as a yellow oil. ES-API: [M/2+1]⁺=344.1.

step 5: tert-butyl ((S)-4-(7-bromo-6-chloro-8-(3,3-difluorocyclobutoxy)-2-(((S)-1-methylpyrrolidin-2-yl)methoxy)quinazolin-4-yl)-2-(cyanomethyl)piperazin-1-carboxylate (3 g, 4.37 mmol), (5-methyl-1H-indazol-4-yl) boronic acid (2.3 g, 13.12 mmol), tripotassium phosphate (4.6 g, 21.87 mmol), tris (dibenzylideneindenacetone)dipalladium (300 mg), 2-dicyclohexylphosphine-2′,4′,6′-triisopropylbiphenyl (300 mg) were added in sequence into dioxane (60 mL) and water (10 mL). The system atmosphere was replaced with nitrogen three times and then the temperature was warmed up to 105° C. and the reaction was conducted for 5 h. After cooling to room temperature, the reaction solution was poured into ethyl acetate (100 mL) and after being washed with brine once it was purified by column on silica gel (methanol:dichloromethane=0˜1:10) to obtain tert-butyl (2S)-4-(6-chloro-8-(3,3-difluorocyclobutoxy)-7-(5-methyl-1H-indazol-4-yl)-2-(((S)-1-)methylpyrrolidin-2-yl)methoxy)quinazolin-4-yl)-2-(cyanomethyl)piperazin-1-carboxylate (1.6 g, Y: 49%) as a yellow solid. ES-API: [M/2+1]⁺=369.2.

step 6: tert-butyl (2S)-4-(6-chloro-8-(3,3-difluorocyclobutoxy)-7-(5-methyl-1H-indazol-4-yl)-2-(((S)-1-)methylpyrrolidin-2-yl)methoxy)quinazolin-4-yl)-2-(cyano methyl)piperazin-1-carboxylate (1.6 g, 2.17 mmol) was dissolved in dichloromethane (5 mL), and then trifluoroacetic acid (5 mL) was added. The reaction was conducted at room temperature for 0.5 h, and then the reaction mixture was dried by rotary evaporation to obtain crude 2-((2S)-4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-2-yl) acetonitrile (1.8 g, Y: 100%) as a yellow oil.

step 7: 2-((2S)-4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-2-yl) acetonitrile (1.4 g, 2.17 mmol) and triethylamine (659 mg, 6.51 mmol) were dissolved in dichloromethane (20 mL). At 0° C. acrylic anhydride (301 mg, 2.39 mmol) was added, and the reaction was conducted at 0° C. for 1 h. The reaction solution was dried by rotary evaporation and purified by flash column chromatography (dichloromethane:methanol:ammonium hydroxide=95:4:1) to obtain 2-((2S)-1-acryloyl-4-(6-chloro-8-(3,3-difluorocyclobutoxy)-7-(5-methyl-1H-indazol-4-yl)-2-(((S)-1-methylpyrrolidin-2-yl)methoxy)quinazolin-4-yl)piperazin-2-yl) acetonitrile (Z6, 200 mg, Y: 13%). ES-API: [M/2+1]⁺=346.2. ¹HNMR (400 MHz, CDCl₃)δ 10.46 (s, 1H), 7.78 (s, 1H), 7.58-7.46 (m, 2H), 7.37 (d, J=8.0 Hz, 1H), 6.60 (s, 1H), 6.41 (d, J=16.7 Hz, 1H), 5.84 (d, J=9.8 Hz, 1H), 5.12 (s, 1H), 4.74 (s, 1H), 4.52 (s, 1H), 4.35 (s, 3H), 4.04 (s, 1H), 3.72 (s, 2H), 3.46 (s, 1H), 3.16 (s, 1H), 2.95 (s, 1H), 2.75 (s, 2H), 2.52 (s, 3H), 2.34 (s, 3H), 2.21 (s, 3H), 2.07 (s, 2H), 1.82 (d, J=27.4 Hz, 4H).

### Example 7 Preparation of 1-(4-(6-chloro-8-cyclopropoxy-7-(5-methyl-1H-indazol-4-yl)-2-(((S)-1-methylpyrrolidin-2-yl) methoxy)quinazolinpyridin-4-yl)piperazin-1-yl)prop-2-en-1-one (Z7)

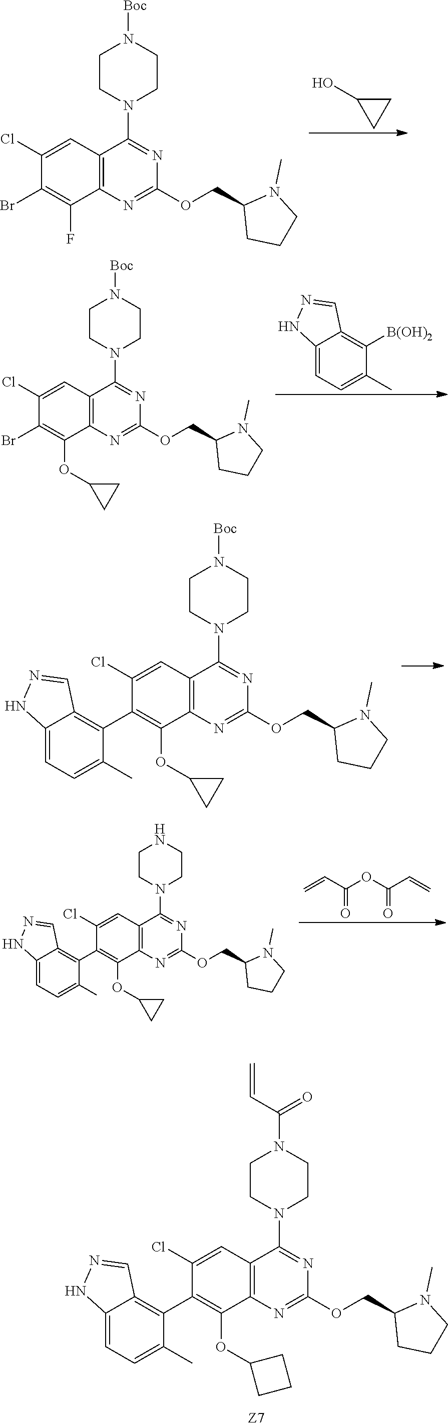

step 1: cyclopropanol (624 mg, 10.75 mmol) was dissolved in tetrahydrofuran (30 mL). At 0° C. sodium hydrogen (1.7 g, 71.7 mmol) was added. The reaction was stirred for 0.5 h while keeping the temperature. tert-butyl (S)-4-(7-bromo-6-chloro-8-fluoro-2-((1-methylpyrrolidin-2-yl)methoxy)quinazolin-4-yl)piperazin-1-carboxylate (4 g, 7.17 mmol) was then added into the reaction solution. The reaction was conducted for 0.5 h while keeping the temperature. The reaction solution was poured into ethyl acetate (100 mL), and the organic phase was washed with water (20 mL×2) and washed with brine (20 mL), dried over anhydrous sodium sulfate and filtered. The filtration was dried by rotary evaporation to obtain tert-butyl (S)-4-(7-bromo-6-chloro-8-cyclopropoxy-2-((1-methylpyrrolidin-2-yl)methoxy)quinazolin-4-yl)piperazin-1-carboxylate (3.1 g, Y: 74%) as a yellow oil. ES-API: [M+1]⁺=596.1.

step 2: tert-butyl (S)-4-(7-bromo-6-chloro-8-cyclopropoxy-2-((1-methylpyrrolidin-2-yl) methoxy)quinazolin-4-yl)piperazin-1-carboxylate (1.8 g, 3.1 mmol), (5-methyl-1H-indazol-4-yl) boronic acid (1.6 g, 9.3 mmol), tripotassium phosphate (3.3 g, 15.5 mmol), tris (dibenzylideneindenacetone)dipalladium (284 mg), 2-dicyclohexylphosphine-2′,4′,6′-triisopropylbiphenyl (145 mg) were added in sequence into dioxane (20 mL) and water (5 mL).

The system atmosphere was replaced with nitrogen three times, then the temperature was warmed up to 105° C. and the reaction was conducted for 6 h. After cooling to room temperature, the reaction solution was poured into ethyl acetate (100 mL) and after being washed with brine once it was purified by column on silica gel (methanol:dichloromethane=0˜1:20) to obtain tert-butyl 4-(6-chloro-8-cyclopropoxy-7-(5-methyl-1H-indazol-4-yl)-2-(((S)-1-methylpyrrolidin-2-yl)methoxy) quinazolin-4-yl)piperazin-1-carboxylate (900 mg, Y: 36%) as a yellow solid. ES-API: [M+1]+=648.3.

step 3: tert-butyl 4-(6-chloro-8-cyclopropoxy-7-(5-methyl-1H-indazol-4-yl)-2-(((S)-1-methylpyrrolidin-2-yl)methoxy)quinazolin-4-yl)piperazin-1-carboxylate (0.8 g, 1.24 mmol) was dissolved in dichloromethane (20 mL), and then trifluoroacetic acid (2 mL) was added. The reaction was conducted at room temperature for 0.5 h and the reaction mixture was dried by rotary evaporation to obtain crude 6-chloro-8-cyclopropoxy-7-(5-methyl-1H-indazol-4-yl)-2-(((S)-1-methylpyrrolidin-2-yl)methoxy)-4-(piperazin-1-yl)quinazoline (800 mg, Y: 100%) as a yellow oil.

step 4: 6-chloro-8-cyclopropoxy-7-(5-methyl-1H-indazol-4-yl)-2-(((S)-1-methylpyrrolidin-2-yl)methoxy)-4-(piperazin-1-yl)quinazoline (660 mg, 1.2 mmol) and triethylamine (2 mL) were dissolved in dichloromethane (20 mL). At 0° C. acrylic anhydride (137 mg, 1.08 mmol) was added, and the reaction was conducted at 0° C. for 1 h. The reaction solution was dried by rotary evaporation and purified by flash column chromatography (dichloromethane:methanol=0˜20/1) to obtain crude 1-(4-(6-chloro-8-cyclopropoxy-7-(5-methyl-1H-indazol-4-yl)-2-(((S)-1-methylpyrrolidin-2-yl)methoxy)quinazolinpyridin-4-yl)piperazin-1-yl)prop-2-en-1-one (400 mg, the purity is 93%), which was purified by preparative silica gel plate to obtain 1-(4-(6-chloro-8-cyclopropoxy-7-(5-methyl-1H-indazol-4-yl)-2-(((S)-1-methylpyrrolidin-2-yl)methoxy) quinazolinpyridin-4-yl)piperazin-1-yl)prop-2-en-1-one (Z7, 202 mg, Y: 20%). ES-API: [M+1]⁺=602.2. ¹HNMR (400 MHz, chloroform-d)δ 7.75 (s, 1H), 7.58 (s, 1H), 7.47 (d, J=8.4 Hz, 1H), 7.33 (d, J=8.7 Hz, 1H), 6.62 (dd, J=16.6, 10.8 Hz, 1H), 6.37 (d, J=16.6 Hz, 1H), 5.78 (d, J=10.5 Hz, 1H), 4.64 (s, 1H), 4.40 (d, J=17.3 Hz, 2H), 3.90 (d, J=31.4 Hz, 8H), 3.21 (s, 1H), 2.88 (s, 1H), 2.57 (s, 3H), 2.37 (d, J=12.5 Hz, 1H), 2.20 (s, 3H), 2.11 (s, 1H), 1.85 (q, J=16.5, 13.7 Hz, 3H), 0.31-0.10 (m, 4H).

step 5: Preparation of Z7A and Z7B

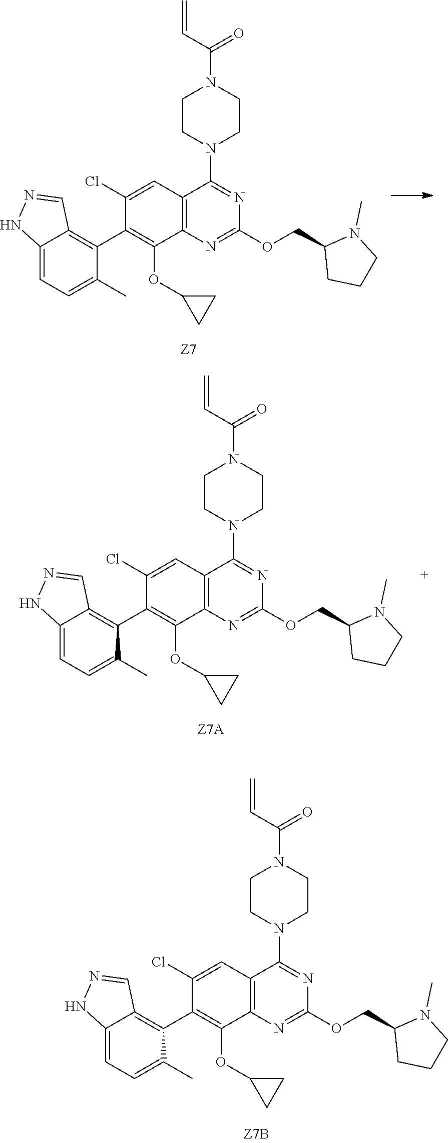

Z7 (850 mg) was separated through chiral resolution (mobile phase:n-hexane:ethanol ammonia methanol=75:25:0.2; column: IB 250 mm*4.6 mm 5 um; flow rate: 1.0 ml/min; column temperature: 30.0° C.) to obtain one isomer, arbitrarily designated as Z7A, 1-(4-((R)-6-chloro-8-cyclopropoxy-7-(5-methyl-1H-indazol-4-yl)-2-((((S)-1-methylpyrrolidin-2-yl))methoxy)quinazolin-4-yl)piperazin-1-yl)prop-2-en-1-one (retention time: 7.394 min; 401 mg; purity: 100%, de value: 99.1%). ES-API: [M+H]⁺=602.2; and the other isomer, arbitrarily designated as Z7B, 1-(4-((S)-6-chloro-8-cyclopropoxy-7-(5-methyl-1H-indazol-4-yl)-2-((((S)-1-methylpyrrolidin-2-yl))methoxy)quinazolin-4-yl)piperazin-1-yl)prop-2-en-1-one (retention time: 8.351 min; 395 mg, purity: 100%, de value: 99%). ES-API: [M+H]⁺=602.2.

### Example 8 Preparation of 1-(4-(6-chloro-8-cyclobutoxy-7-(5-methyl-1H-indazol-4-yl)-2-(((S)-1-methylpyrrolidin-2-yl)methoxy) quinazolinpyridin-4-yl)piperazin-1-yl)prop-2-en-1-one (Z8)

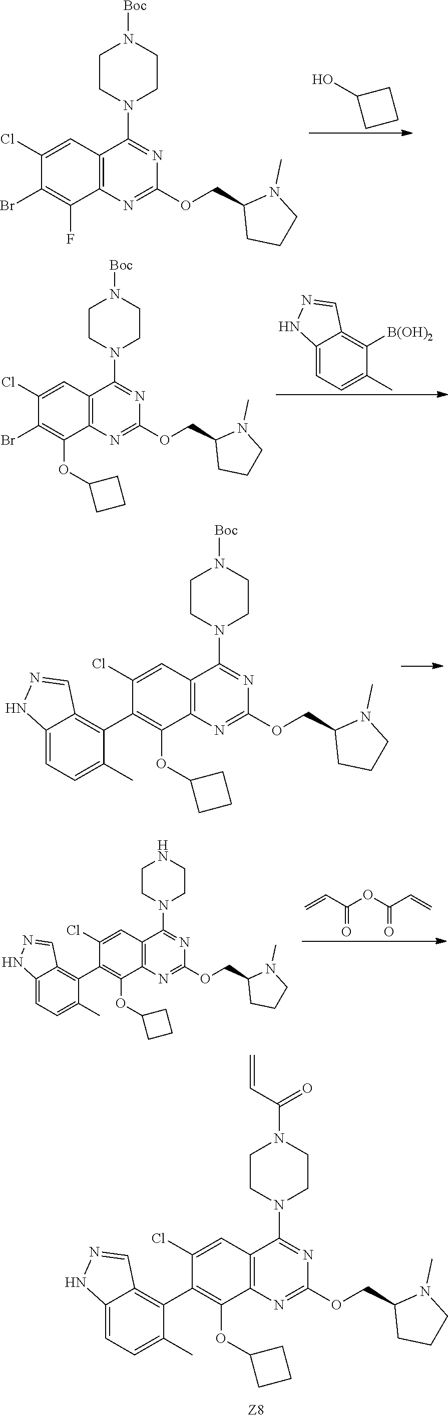

step 1: cyclobutanol (774 mg, 10.75 mmol) was dissolved in tetrahydrofuran (30 mL) and then at 0° C. sodium hydrogen (1.7 g, 71.7 mmol) was added. The reaction was stirred for 0.5 h while keeping the temperature. tert-butyl (S)-4-(7-bromo-6-chloro-8-fluoro-2-((1-methylpyrrolidin-2-yl)methoxy)quinazolin-4-yl)piperazin-1-carboxylate (4 g, 7.17 mmol) was then added into the reaction solution. The reaction was conducted for 0.5 h while keeping the temperature. The reaction solution was poured into ethyl acetate (100 mL), and the organic phase was washed with water (20 mL×2) and washed with brine (20 mL), dried over anhydrous sodium sulfate and filtered. The filtration was dried by rotary evaporation to obtain tert-butyl (S)-4-(7-bromo-6-chloro-8-cyclobutoxy-2-((1-methylpyrrolidin-2-yl)methoxy)quinazolin-4-yl)piperazin-1-carboxylate (2.8 g, Y: 65%) as a yellow oil. ES-API: [M+1]⁺=610.1.

step 2: tert-butyl (S)-4-(7-bromo-6-chloro-8-cyclobutoxy-2-((1-methylpyrrolidin-2-yl) methoxy)quinazolin-4-yl)piperazin-1-carboxylate (1.4 g, 2.3 mmol), (5-methyl-1H-indazol-4-yl) boronic acid (810 mg, 4.6 mmol), tripotassium phosphate (2.4 g, 11.5 mmol), tris (dibenzylideneindenacetone)dipalladium (210 mg), 2-dicyclohexylphosphine-2′,4′,6′-triisopropylbiphenyl (110 mg) were added in sequence into dioxane (20 mL) and water (5 mL). The system atmosphere was replaced with nitrogen three times, and then the temperature was warmed up to 105° C. and the reaction was conducted for 6 h. After cooling to room temperature, the reaction solution was poured into ethyl acetate (100 mL) and after being washed with brine once it was purified by column on silica gel (methanol:dichloromethane=0˜1:20) to obtain tert-butyl 4-(6-chloro-8-cyclobutoxy-7-(5-methyl-1H-indazol-4-yl)-2-(((S)-1-methylpyrrolidin-2-yl)methoxy)quinazolin-4-pyridin-4-yl)piperazin-1-carboxylate (500 g, Y: 30%) as a yellow solid. ES-API: [M+1]⁺=662.2.

step 3: tert-butyl 4-(6-chloro-8-cyclobutoxy-7-(5-methyl-1H-indazol-4-yl)-2-(((S)-1-methylpyrrolidin-2-yl)methoxy) quinazolin-4-pyridin-4-yl)piperazin-1-carboxylate (400 mg, 0.605 mmol) was dissolved in dichloromethane (10 mL), and then trifluoroacetic acid (2 mL) was added. The reaction was conducted at room temperature for 1 h and the reaction mixture was dried by rotary evaporation to obtain crude 6-chloro-8-cyclobutoxy-7-(5-methyl-1H-indazol-4-yl)-2-(((S)-1-methylpyrrolidin-2-yl)methoxy)-4-(piperazin-1-yl)quinazoline (400 mg, Y: 100%) as a yellow oil.

step 4: 6-chloro-8-cyclobutoxy-7-(5-methyl-1H-indazol-4-yl)-2-(((S)-1-methylpyrrolidin-2-yl)methoxy)-4-(piperazin-1-yl)quinazoline (300 mg, 0.535 mmol) and triethylamine (2 mL) were dissolved in dichloromethane (10 mL). At 0° C. acrylic anhydride (61 mg, 0.48 mmol) was added, and the reaction was conducted at 0° C. for 1 h. The reaction solution was dried by rotary evaporation and then purified by a silica gel plate (methanol:dichloromethane=0˜1:10) and lyophilized to obtain 1-(4-(6-chloro-8-cyclobutoxy-7-(5-methyl-1H-indazol-4-yl)-2-(((S)-1-methylpyrrolidin-2-yl)methoxy)quinazolinpyridin-4-yl)piperazin-1-yl)prop-2-en-1-one (Z8, 115 mg, Y: 25%). ES-API: [M+1]⁺=616.2. ¹HNMR (400 MHz, chloroform-d)δ 7.74 (s, 1H), 7.56 (s, 1H), 7.49 (d, J=8.6 Hz, 1H), 7.36 (d, J=8.6 Hz, 1H), 6.68-6.56 (m, 1H), 6.37 (d, J=16.7 Hz, 1H), 5.78 (d, J=10.5 Hz, 1H), 4.97 (s, 1H), 4.66 (d, J=30.7 Hz, 2H), 3.90 (d, J=25.0 Hz, 8H), 3.68 (d, J=23.3 Hz, 1H), 2.89 (s, 2H), 2.21 (s, 3H), 2.04 (s, 4H), 1.68 (d, J=65.0 Hz, 6H), 1.37 (d, J=35.0 Hz, 2H).

The following compounds were prepared according to the above-mentioned similar methods (with the difference that different raw materials were used instead) or conventional synthetic methods.

| Number | Structure | MS (M + H⁺) |
|---|---|---|
| Z9 | 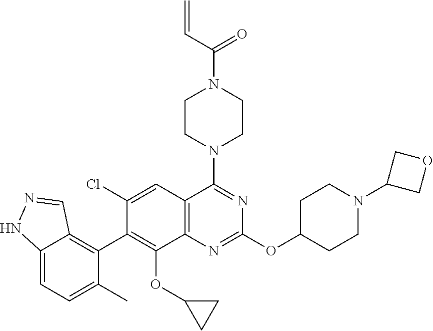 [SMILES: C=CC(=O)N1CCN(c2nc(OC3CCN(C4COC4)CC3)nc3c(OC4CC4)c(-c4c(C)ccc5[nH]ncc45)c(Cl)cc23)CC1] | 644.3 |
| Z14 | 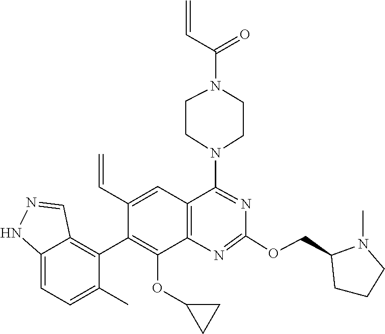 [SMILES: C=CC(=O)N1CCN(c2nc(OC[C@@H]3CCCN3C)nc3c(OC4CC4)c(-c4c(C)ccc5[nH]ncc45)c(C=C)cc23)CC1] | 594.3 |
| Z15 | 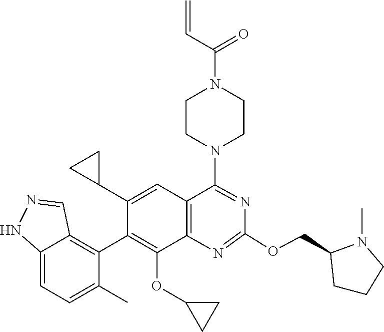 [SMILES: C=CC(=O)N1CCN(c2nc(OC[C@@H]3CCCN3C)nc3c(OC4CC4)c(-c4c(C)ccc5[nH]ncc45)c(C4CC4)cc23)CC1] | 608.3 |
| Z16 | 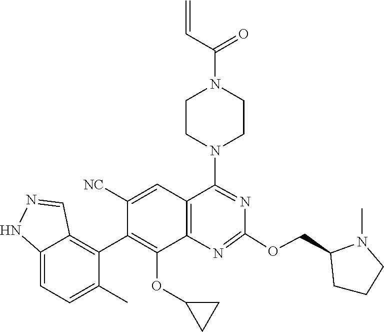 [SMILES: C=CC(=O)N1CCN(c2nc(OC[C@@H]3CCCN3C)nc3c(OC4CC4)c(-c4c(C)ccc5[nH]ncc45)c(C#N)cc23)CC1] | 593.3 |
| Z17 | 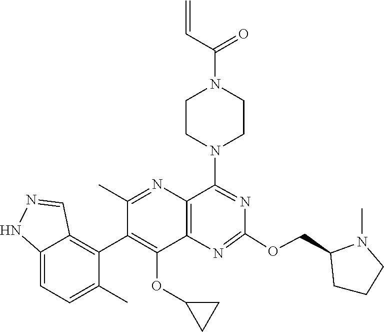 [SMILES: C=CC(=O)N1CCN(c2nc(OC[C@@H]3CCCN3C)nc3c(OC4CC4)c(-c4c(C)ccc5[nH]ncc45)c(C)nc23)CC1] | 583.3 |
| Z18 | 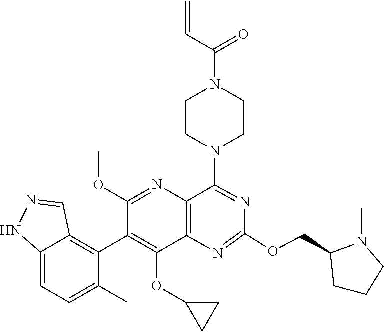 [SMILES: C=CC(=O)N1CCN(c2nc(OC[C@@H]3CCCN3C)nc3c(OC4CC4)c(-c4c(C)ccc5[nH]ncc45)c(OC)nc23)CC1] | 599.3 |
| Z19 | 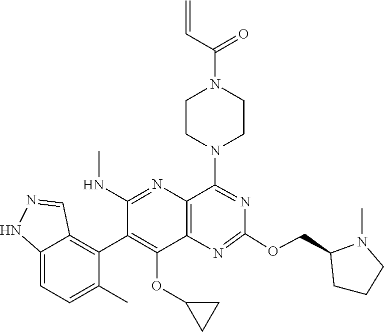 [SMILES: C=CC(=O)N1CCN(c2nc(OC[C@@H]3CCCN3C)nc3c(OC4CC4)c(-c4c(C)ccc5[nH]ncc45)c(NC)nc23)CC1] | 598.3 |
| Z20 | 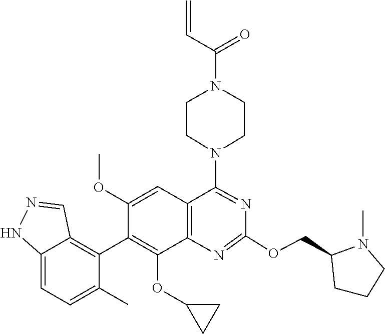 [SMILES: C=CC(=O)N1CCN(c2nc(OC[C@@H]3CCCN3C)nc3c(OC4CC4)c(-c4c(C)ccc5[nH]ncc45)c(OC)cc23)CC1] | 598.3 |
| Z21 | 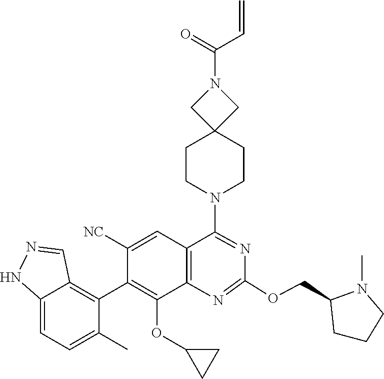 [SMILES: C=CC(=O)N1CC2(CCN(c3nc(OC[C@@H]4CCCN4C)nc4c(OC5CC5)c(-c5c(C)ccc6[nH]ncc56)c(C#N)cc34)CC2)C1] | 633.3 |
| Z22 | 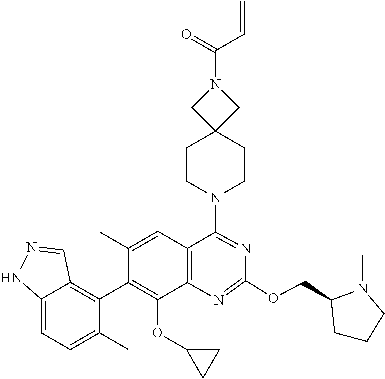 [SMILES: C=CC(=O)N1CC2(CCN(c3nc(OC[C@@H]4CCCN4C)nc4c(OC5CC5)c(-c5c(C)ccc6[nH]ncc56)c(C)cc34)CC2)C1] | 622.3 |
| Z23 | 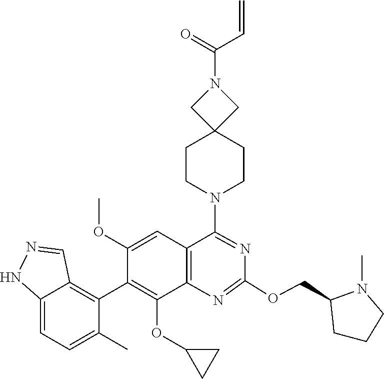 [SMILES: C=CC(=O)N1CC2(CCN(c3nc(OC[C@@H]4CCCN4C)nc4c(OC5CC5)c(-c5c(C)ccc6[nH]ncc56)c(OC)cc34)CC2)C1] | 638.3 |
| Z24 | 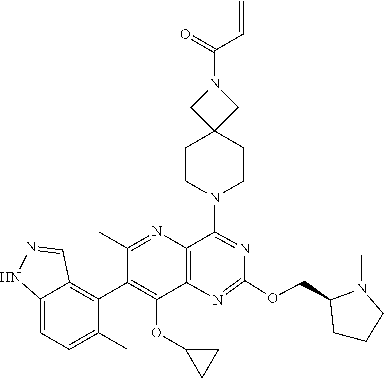 [SMILES: C=CC(=O)N1CC2(CCN(c3nc(OC[C@@H]4CCCN4C)nc4c(OC5CC5)c(-c5c(C)ccc6[nH]ncc56)c(C)nc34)CC2)C1] | 623.3 |
| Z25 | 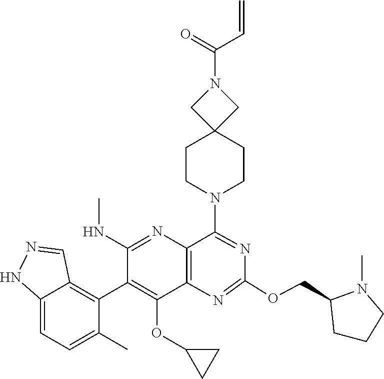 [SMILES: C=CC(=O)N1CC2(CCN(c3nc(OC[C@@H]4CCCN4C)nc4c(OC5CC5)c(-c5c(C)ccc6[nH]ncc56)c(NC)nc34)CC2)C1] | 638.3 |
| Z26 | 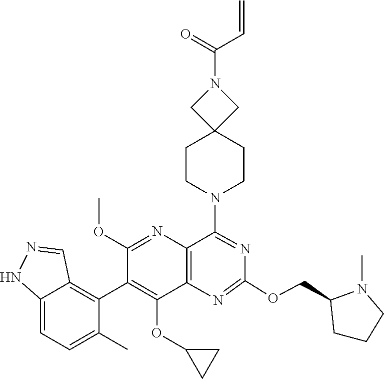 [SMILES: C=CC(=O)N1CC2(CCN(c3nc(OC[C@@H]4CCCN4C)nc4c(OC5CC5)c(-c5c(C)ccc6[nH]ncc56)c(OC)nc34)CC2)C1] | 639.3 |

Example 10 Preparation of Z10A

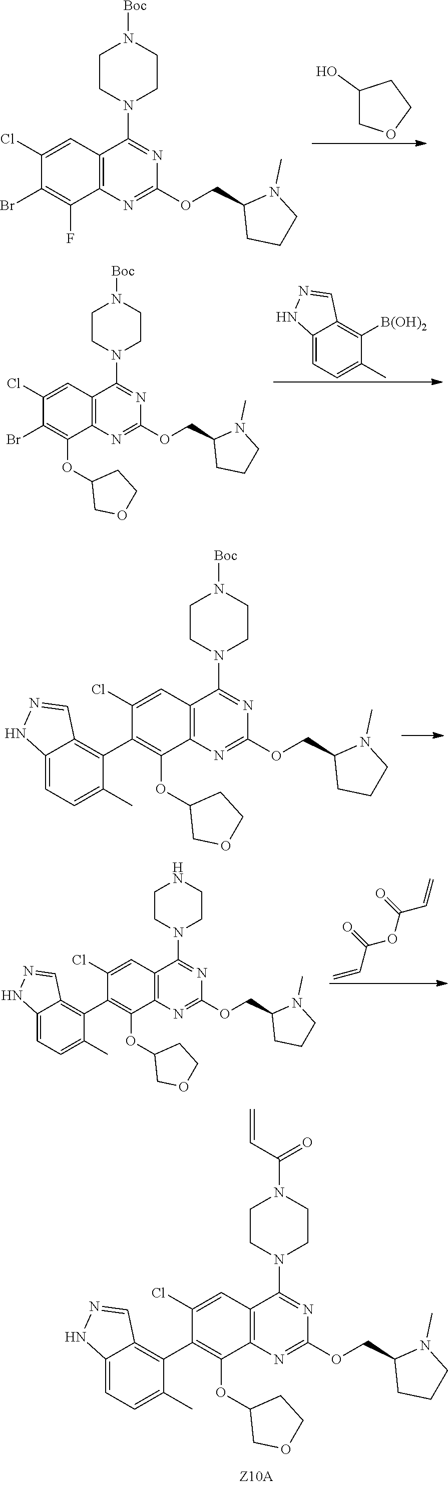

step 1: tetrahydrofuran-3-ol (238 mg, 2.7 mmol) was dissolved in tetrahydrofuran (10 ML) and at 0° C. sodium hydrogen (432 mg, 18 mmol) was added. The reaction was stirred for 0.5 h while keeping the temperature. tert-butyl (S)-4-(7-bromo-6-chloro-8-fluoro-2-((1-methylpyrrolidin-2-yl)methoxy)quinazolin-4-yl)piperazin-1-carboxylate (1 g, 1.8 mmol) was then added into the reaction solution. The reaction was conducted for 1 h while keeping the temperature. The reaction solution was poured into ethyl acetate (20 mL), and the organic phase was washed with water (20 mL×2) and washed with brine (20 mL), dried over anhydrous sodium sulfate and filtered. The filtration was dried by rotary evaporation to obtain tert-butyl 4-(7-bromo-6-chloro-2-(((S)-1-methylpyrrolidin-2-yl) methoxy)-8-((tetrahydrofuran-3-yl)oxy)quinazolin-4-yl)piperazin-1-carboxylate (630 mg, Y: 51%) as a yellow oil. ES-API: [M+1]⁺=626.1.

step 2: tert-butyl 4-(7-bromo-6-chloro-2-(((S)-1-methylpyrrolidin-2-yl)methoxy)-8-((tetrahydrofuran-3-yl)oxy)quinazolin-4-yl)piperazin-1-carboxylate (400 mg, 0.64 mmol), (5-methyl-1H-indazol-4-yl) boronic acid (225 mg, 1.28 mmol), tripotassium phosphate (678 mg, 3.2 mmol), tris (dibenzylideneindenacetone)dipalladium (59 mg, 0.064 mmol), 2-dicyclohexylphosphine-2′,4′,6′-triisopropylbiphenyl (30 mg, 0.064 mmol) were added in sequence into dioxane (4 mL) and water (1 mL). The system atmosphere was replaced with nitrogen three times, then the temperature was warmed up to 100° C. and the reaction was conducted for 16 h. After cooling to room temperature, the reaction solution was poured into ethyl acetate (10 mL) and after being washed with brine once it was purified by column on silica gel (methanol:dichloromethane=0˜1:20) to obtain tert-butyl 4-(6-chloro-7-(5-methyl-1H-indazol-4-yl)-2-(((S)-1-methylpyrrolidin-2-yl)methoxy)-8-((tetrahydrofuran)-3-yl)oxy)quinazolin-4-yl)piperazin-1-carboxylate (214 mg, Y: 22%) as a yellow solid. ES-API: [M+1]⁺=648.3.

step 3: tert-butyl 4-(6-chloro-7-(5-methyl-1H-indazol-4-yl)-2-(((S)-1-methylpyrrolidin-2-yl)methoxy)-8-((tetrahydrofuran)-3-yl)oxy)quinazolin-4-yl)piperazin-1-carboxylate (214 mg, 0.32 mmol) was dissolved in dichloromethane (5 mL) and then trifluoroacetic acid (2 mL) was added. The reaction was conducted at room temperature for 1 h and the reaction mixture was dried by rotary evaporation to obtain crude 6-chloro-7-(5-methyl-1H-indazol-4-yl)-2-(((S)-1-methylpyrrolidin-2-yl) methoxy)-4-(piperazin-1-yl)-8-((tetrahydrofuran-3-yl)oxy)quinazoline (180 mg, Y: 100%) as a yellow oil.

step 4: 6-chloro-7-(5-methyl-1H-indazol-4-yl)-2-(((S)-1-methylpyrrolidin-2-yl)methoxy)-4-(piperazin-1-yl)-8-((tetrahydrofuran-3-yl)oxy)quinazoline (180 mg, 0.31 mmol) and triethylamine (2 mL) were dissolved in dichloromethane (5 mL). At 0° C. acrylic anhydride was added (30 mg, 0.25 mmol), and the reaction was conducted at 0° C. for 0.5 h. To the reaction solution 20 mL of water and DCM (20 mL*3) were added, which was extracted and the organic phase was dried by rotary evaporation to obtain a crude, which was purified by preparation to obtain 1-(4-(6-chloro-7-(5-methyl-1H-indazol-4-yl)-2-(((S)-1-methylpyrrolidin-2-yl) methoxy)-8-((tetrahydrofuran-3-yl)oxy)quinazolin-4-yl)piperazin-1-yl)prop-2-en-1-one (20 mg, Y: 14.5%). Preparation and separation conditions: Elite P270, column: Ultimate XB-C18, 50*250 mm, 10 um; mobile phase: A: purified water B: purified acetonitrile; flow rate: 80 ml/min, gradient: within 40 min, B/A=20%-90%, wavelength: 214 nm, column temperature: room temperature. ES-API: [M+1]⁺=632.2. ¹HNMR (400 MHz, CDCl₃)δ 7.75 (s, 1H), 7.59 (s, 1H), 7.47 (d, J=8.8 Hz, 1H), 7.35 (t, J=6.7 Hz, 1H), 6.62 (dd, J=16.7, 10.8 Hz, 1H), 6.37 (d, J=17.0 Hz, 1H), 5.78 (d, J=10.6 Hz, 1H), 5.27 (d, J=78.3 Hz, 1H), 4.58 (s, 1H), 4.38 (s, 1H), 3.90 (d, J=28.0 Hz, 1OH), 3.58-3.37 (m, 2H), 3.20 (s, 1H), 2.86 (s, 1H), 2.56 (s, 3H), 2.37 (s, 1H), 2.23 (s, 3H), 2.11 (s, 1H), 1.82 (s, 3H).

### Example 12 Preparation of Z12

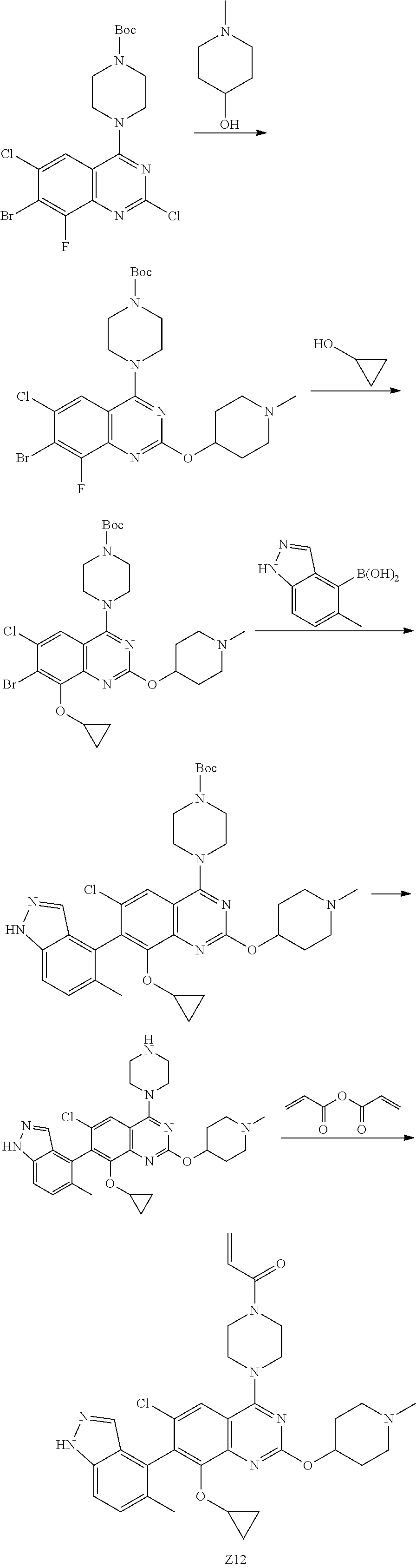

step 1: tert-butyl 4-(7-bromo-2,6-dichloro-8-fluoroquinazolin-4-yl)piperazin-1-carboxylate (1.5 g, 3.12 mmol) was added to (S)-(1-methylpyrrolidin-2-yl) methanol (5.38 g, 46.8 mmol), and then KF (362 mg, 6.24 mmol) was added. After warming up to 100° C. the reaction was conducted for 2 h. It was detected by LCMS till the reaction was complete. After the reaction solution was cooled to room temperature, 20 mL of water was added, and the mixture was extracted with ethyl acetate (30 mL*3). The organic phase was dried and dried by rotary evaporation to obtain a crude which was purified by flash column on silica gel (DCM/MeOH=100/1) to obtain 1.0 g of product tert-butyl 4-(7-bromo-6-chloro-8-fluoro-2-((1-methylpiperidin-4-yl)oxy)quinazolin-4-yl) piperazin-1-carboxylate. (Y: 43.5%). ES-API: [M+1]⁺=560.1.

step 2: cyclopropanol (207 mg, 3.58 mmol) was dissolved in tetrahydrofuran (15 mL) and at 0° C. sodium hydrogen (716 mg, 17.9 mmol) was added. The reaction was stirred for 0.5 h while keeping the temperature. tert-butyl 4-(7-bromo-6-chloro-8-fluoro-2-((1-methylpiperidin-4-yl)oxy) quinazolin-4-yl)piperazin-1-carboxylate (1.0 g, 1.79 mmol) was then added into the reaction solution. The reaction was conducted for 1 h while keeping the temperature. The reaction solution was poured into ethyl acetate (100 mL), and the organic phase was washed with water (15 mL×2), dried over anhydrous sodium sulfate and filtered. The filtration was dried by rotary evaporation to obtain tert-butyl 4-(7-bromo-6-chloro-8-cyclopropoxy-2-((1-methylpiperidin-4-yl)oxy)quinazolin-4-yl)piperazin-1-carboxylate (1.0 g, Y: 93.6%) as a yellow oil. ES-API: [M+1]⁺=596.1

step 3: tert-butyl (4-(7-bromo-6-chloro-8-cyclopropoxy-2-((1-methylpiperidin-4-yl)oxy) quinazolin-4-yl)piperazin-1-carboxylate (900 mg, 1.5 mmol), (5-methyl-1H-indazol-4-yl) boronic acid (528 mg, 3.0 mmol), tripotassium phosphate (1.59 g, 7.5 mmol), tris (dibenzylideneindenacetone)dipalladium (137 mg, 0.15 mmol), 2-dicyclohexylphosphine-2′,4′,6′-triisopropylbiphenyl (140 mg, 0.3 mmol) were added in sequence into dioxane (20 mL) and water (4 mL). The system atmosphere was replaced with nitrogen three times, then the temperature was warmed up to 100° C. and the reaction was conducted for 8 h. After cooling to room temperature, the reaction solution was poured into ethyl acetate (100 mL) and after being washed with brine once it was purified by column on silica gel (methanol dichloromethane=0˜1:20) to obtain tert-butyl 4-(6-chloro-8-cyclopropoxy-7-(5-methyl-1H-indazol-4-yl)-2-((1-methylpiperidin-4-yl)oxy)quinazolin-4-yl)-1-carboxylate-piperazine (670 mg, Y: 62%) as a yellow solid. ES-API: [M+1]⁺=648.3.

step 4: tert-butyl 4-(6-chloro-8-cyclopropoxy-7-(5-methyl-1H-indazol-4-yl)-2-((1-methylpiperidin-4-yl)oxy)quinazolin-4-yl)-1-carboxylate-piperazine (670 mg, 1.03 mmol) was dissolved in dichloromethane (10 mL) and then trifluoroacetic acid (2.5 mL) was added. The reaction was conducted at room temperature for 1 h and the reaction mixture was dried by rotary evaporation to obtain crude 6-chloro-8-cyclopropoxy-7-(5-methyl-1H-indazol-4-yl)-2-((1-methylpiperidin-4-yl)oxy)-4-(piperazin-1-yl)quinazoline (620 mg, Y: 100%) as a yellow oil.

step 5: 6-chloro-8-cyclopropoxy-7-(5-methyl-1H-indazol-4-yl)-2-((1-methylpiperidin-4-yl) oxy)-4-(piperazin-1-yl)quinazoline (564 mg, 1.03 mmol) and triethylamine (510 mg, 5.15 mmol) were dissolved in dichloromethane (5 mL). At 0° C. acrylic anhydride (194 mg, 1.54 mmol) was added, and the reaction was conducted at 0° C. for 0.5 h. 20 mL of water and DCM (30 mL*3) were added to the reaction solution which was extracted and the organic phase was dried by rotary evaporation to obtain a crude which was purified by preparation (Elite P270, column: Ultimate XB-C18, 50*250 mm, 10 um, mobile phase: A: purified water B: purified acetonitrile, flow rate: 80 ml/min, gradient: within 40 min, B/A=20%-90%, wavelength: 214 nm, column temperature: room temperature) and lyophilized to obtain 1-(4-(6-chloro-8-cyclopropoxy-7-(5-methyl-1H-indazol-4-yl)-2-((1-methylpiperidin-4-yl)oxy)quinazolin-4-yl)piperazin-1-yl)prop-2-en-1-one (133 mg, Y: 21.4%). ES-API: [M+1]⁺=602.2. ¹HNMR (400 MHz, CDCl₃)δ 10.39 (s, 1H), 7.76 (s, 1H), 7.59 (s, 1H), 7.48 (d, J=8.8 Hz, 1H), 7.35 (d, J=8.5 Hz, 1H), 6.62 (dd, J=16.8, 10.7 Hz, 1H), 6.37 (d, J=16.9 Hz, 1H), 5.78 (d, J=10.6 Hz, 1H), 5.17 (s, 1H), 4.20 (s, 1H), 3.90 (d, J=37.2 Hz, 8H), 2.84 (d, J=10.6 Hz, 2H), 2.31 (s, 3H), 2.18 (d, J=24.9 Hz, 7H), 1.97 (t, J=11.0 Hz, 3H), 0.24 (h, J=8.9, 8.2 Hz, 4H).

step 6: Preparation of Z12A and Z12B

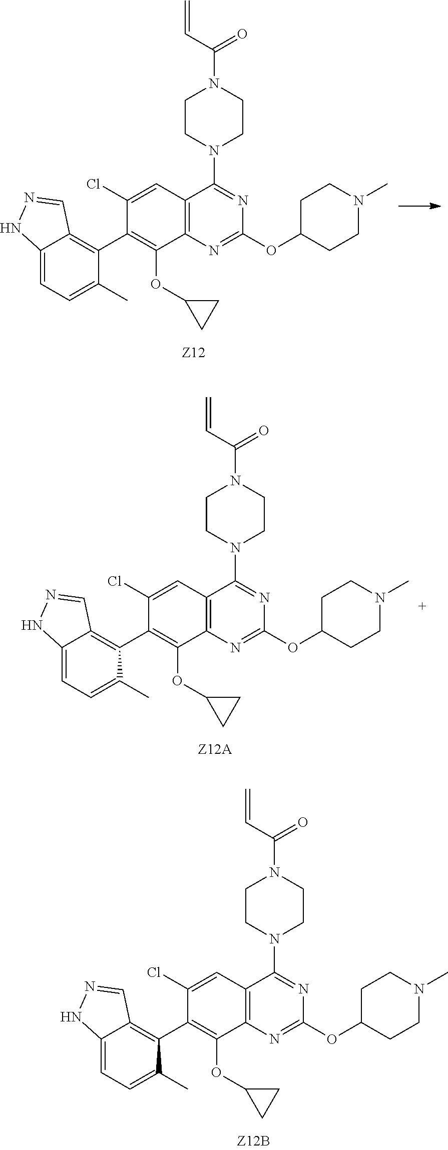

The above compound Z12 (70 mg) was separated through chiral resolution (mobile phase:acetonitrile:isopropanol:ammonia methanol=80:20:0.2); column: IC 250 mm*4.6 mm 5 um); flow rate: 1.0 ml/min; column temperature: 30.0° C.) to obtain: one isomer, arbitrarily designated as Z12A: (R)-1-(4-(6-chloro-8-cyclopropoxy-7-(5-methyl-1H-indazol-4-yl)-2-((1-methylpiperidin-4-yl)oxy)quinazolin-4-yl)piperazin-1-yl)prop-2-en-1-one (retention time: 9.829 min; 31 mg; purity: 100%, ee value: 99.3%). ES-API: [M+H]⁺=602.2; and the other isomer, arbitrarily designated as Z12B: (S)-1-(4-(6-chloro-8-cyclopropoxy-7-(5-methyl-1H-indazol-4-yl)-2-((1-methylpiperidin-4-yl)oxy)quinazolin-4-yl)piperazin-1-yl)prop-2-en-1-one (retention time: 12.528 min; 32 mg; purity: 100%, ee value: 99.2%). ES-API: [M+H]⁺=602.2.

### Example 13 Preparation of Z13

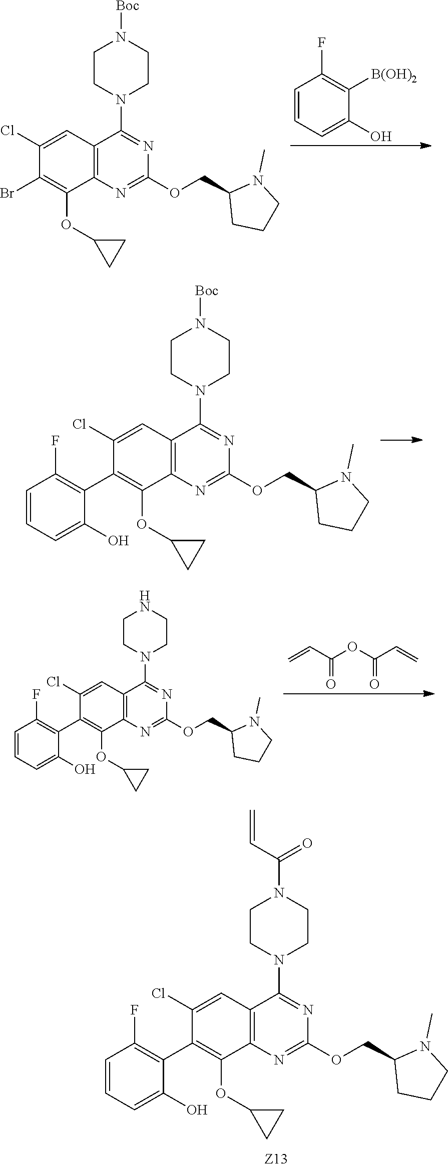

step 1: tert-butyl (S)-4-(7-bromo-6-chloro-8-cyclopropoxy-2-((1-methylpyrrolidin-2-yl) methoxy)quinazolin-4-yl)piperazin-1-carboxylate (1 g, 1.68 mmol), (2-fluoro-6-hydroxyphenyl) boronic acid (524 mg, 3.36 mmol), tripotassium Phosphate (1.78 g, 8.4 mmol), chloro(2-dicyclohexylphosphino-2′,6′-dimethoxy-1,1′-biphenyl)(2′-amino-1,1′-biphenyl-2-yl)palladium (II) (121 mg, 0.168 mmol), 2-dicyclohexylphosphine-2′,4′,6′-triisopropylbiphenyl (78 mg, 0.168 mmol) were added in sequence into dioxane (6 mL) and water (1.5 mL). The system atmosphere was replaced with nitrogen three times, then the temperature was warmed up to 100° C. and the reaction was conducted for 8 h. After cooling to room temperature, the reaction solution was poured into ethyl acetate (100 mL) and after being washed with brine once it was purified by column on silica gel (methanol:dichloromethane=0˜1:20) to obtain tert-butyl 4-(6-chloro-8-cyclopropoxy-7-(2-fluoro-6-hydroxyphenyl)-2-(((S)-1-methylpyrrolidin-2-yl)methoxy) quinazolin-4-yl))piperazin-1-carboxylate (370 mg, Y: 37%) as a yellow solid. ES-API: [M+1]+=628.3.

step 2: tert-butyl 4-(6-chloro-8-cyclopropoxy-7-(2-fluoro-6-hydroxyphenyl)-2-(((S)-1-methylpyrrolidin-2-yl)methoxy)quinazolin-4-yl))piperazin-1-carboxylate (370 mg, 0.59 mmol) was dissolved in dichloromethane (5 mL) and then trifluoroacetic acid (2 mL) was added. The reaction was conducted at room temperature for 1 h and the reaction mixture was dried by rotary evaporation to obtain crude 2-(6-chloro-8-cyclopropyl-2-(((S)-1-methylpyrrolidin-2-yl) methoxy)-4-(piperazin-1-yl)quinazolin-7-yl)-3-fluorophenol (300 mg, Y: 100%) as a yellow oil.

step 3: 2-(6-chloro-8-cyclopropyl-2-(((S)-1-methylpyrrolidin-2-yl)methoxy)-4-(piperazin-1-yl)quinazolin-7-yl)-3-fluorophenol (300 mg, 0.568 mmol) and triethylamine (2 mL) were dissolved in dichloromethane (5 mL). At 0° C. acrylic anhydride (64 mg, 0.51 mmol) was added, and the reaction was conducted at 0° C. for 0.5 h. 20 mL of water and DCM (20 mL*3) were added to the reaction solution which was extracted and the organic phase was dried by rotary evaporation to obtain a crude, which was purified by preparation to obtain 1-(4-(6-chloro-8-cyclopropyl-7-(2-fluoro-6-hydroxyphenyl)-2-(((S)-1-methylpyrrolidin-2-yl)methoxy) quinazolin-4-yl)piperazin-1-yl)prop-2-en-1-one (82.6 mg, Y: 26.5%). The preparation and separation conditions were the same as in Example 10. ES-API: [M+1]⁺=582.2. ¹HNMR (400 MHz, DMSO-d6)δ 9.94 (s, 1H), 7.80 (s, 1H), 7.25 (d, J=8.1 Hz, 1H), 6.90-6.62 (m, 3H), 6.15 (d, J=16.5 Hz, 1H), 5.72 (d, J=10.4 Hz, 1H), 4.67 (s, 1H), 4.42 (s, 1H), 4.25 (s, 1H), 3.78 (d, J=30.9 Hz, 9H), 3.04 (s, 1H), 2.43 (s, 4H), 1.99 (s, 1H), 1.70 (s, 3H), 0.46-0.20 (m, 4H).

### Example 27 Preparation of Z27

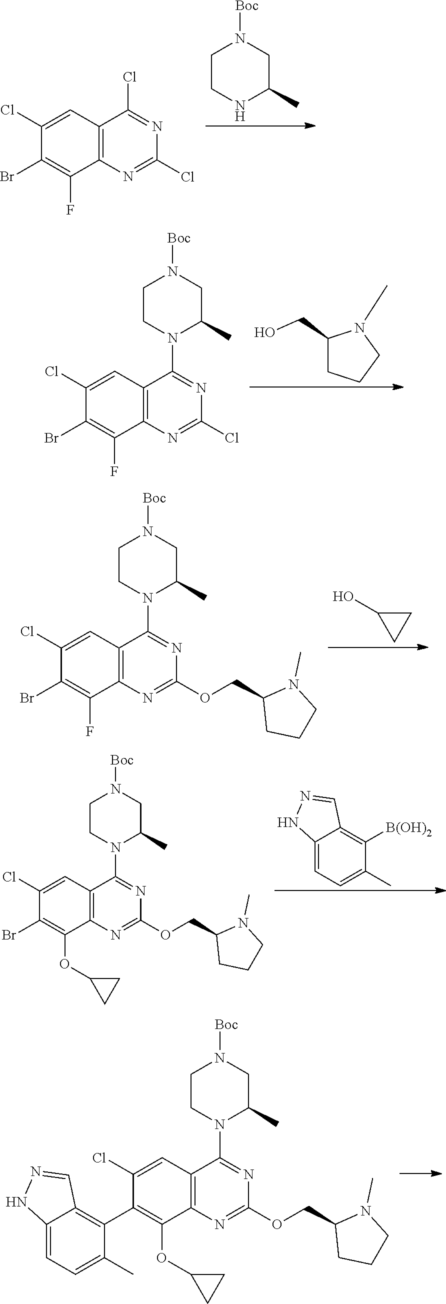

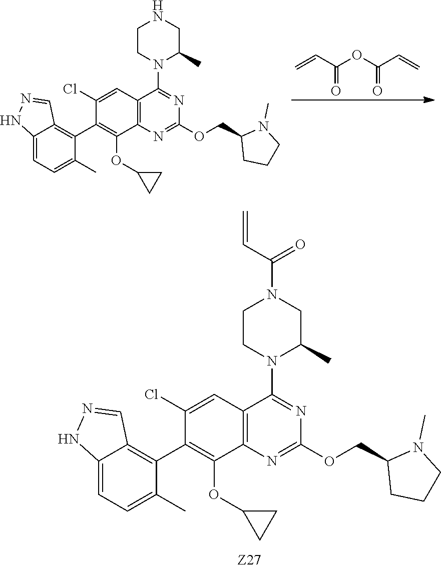

step 1: 7-bromo2,4,6-trichloro-8-fluoroquinazoline (1.5 g, 4.55 mmol) was added into DCM/ACN(15/15 mL). The reaction solution was cooled to 0° C. and then TEA (2.3 g, 22.8 mmol) was added slowly. After that tert-butyl (R)-3-methylpiperazin-1-carboxylate (1.0 g, 5.0 mmol) was added. The reaction solution was slowly warmed up to room temperature and then stirred for 2 h. It was detected by LCMS till the reaction was complete. DCM (30 mL*3) and water (30 mL) were added which was extracted and the organic phase was dried and dried by rotary evaporation to obtain 2.0 g tert-butyl (R)-4-(7-bromo-2,6-dichloro-8-fluoroquinazolin-4-yl)-3-methylpiperazin-1-carboxylate (Y: 100%) as a reddish brown solid. ES-API: [M+1]⁺=493.0.

step 2: tert-butyl (R)-4-(7-bromo-2,6-dichloro-8-fluoroquinazolin-4-yl)-3-methylpiperazin-1-carboxylate (2.9 g, 5.87 mmol) was added to (S)-(1-methylpyrrolidin-2-yl) methanol (10.1 g, 88 mmol) and then KF (1 g, 17.6 mmol) was added. The mixture was warmed up to 100° C. and the reaction was conducted for 2 h. It was detected by LCMS till the reaction was complete. After the reaction solution was cooled to room temperature 30 mL of water was added, which was extracted with ethyl acetate (30 mL*3). The organic phase was dried and dried by rotary evaporation to obtain a crude which was purified by flash column on silica gel (DCM/MeOH=50/1) to obtain 2.7 g of product tert-butyl (R)-4-(7-bromo-6-chloro-8-fluoro-2-(((S)-1-methylpyrrolidin-2-yl)methoxy)quinazolin-4-yl)-3-methylpiperazin-1-carboxylate (Y: 81%). ES-API: [M+1]⁺=572.1.

step 3: cyclopropanol (411 mg, 7.1 mmol) was dissolved in tetrahydrofuran (30 mL) and at 0° C. sodium hydrogen (1.13 g, 47.2 mmol) was added. The reaction was stirred for 0.5 h while keeping the temperature. tert-butyl (R)-4-(7-bromo-6-chloro-8-fluoro-2-(((S)-1-methylpyrrolidin-2-yl)methoxy)quinazolin-4-yl)-3-methylpiperazin-1-carboxylate (2.7 g, 4.72 mmol) was then added into the reaction solution. The reaction was conducted for 1 h while keeping the temperature. The reaction solution was poured into ethyl acetate (30 mL), the organic phase was washed with water (30 mL×2) and washed with brine (30 mL), dried over anhydrous sodium sulfate and filtered. The filtration was dried by rotary evaporation to obtain a yellow oil, which was purified by flash column on silica gel (DCM/MeOH=100/1) to obtain tert-butyl (R)-4-(7-bromo-6-chloro-8-cyclopropoxy-2-(((S)-1-methylpyrrolidin-2-yl)methoxy)quinazolin-4-yl)-3-methylpiperazin-1-carboxylate (1.6 g, Y: 57%). ES-API: [M+1]⁺=610.1.

step 4: tert-butyl (R)-4-(7-bromo-6-chloro-8-cyclopropoxy-2-(((S)-1-methylpyrrolidin-2-yl) methoxy)quinazolin-4-yl)-3-methylpiperazin-1-carboxylate (800 mg, 1.3 mmol), (5-methyl-1H-indazol-4-yl) boronic acid (463 mg, 2.6 mmol), tripotassium phosphate (1.4 g, 6.5 mmol), tris (dibenzylideneindenacetone)dipalladium (120 mg, 0.13 mmol), 2-dicyclohexylphosphine-2′,4′,6′-triisopropylbiphenyl (61 mg, 0.13 mmol) were added in sequence into dioxane (8 mL) and water (2 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once it was purified by column on silica gel (methanol:dichloromethane=0˜1:20) to obtain tert-butyl (3R)-4-(6-chloro-8-cyclopropoxy-7-(5-methyl-1H-indazol-4-yl)-2-(((S)-1-methylpyrrolidin-2-yl))methoxy)quinazolin-4-yl)-3-methylpiperazin-1-carboxylate (455 mg, Y: 52%) as a yellow solid. ES-API: [M+1]⁺=662.1 step 5: tert-butyl (3R)-4-(6-chloro-8-cyclopropoxy-7-(5-methyl-1H-indazol-4-yl)-2-(((S)-1-methylpyrrolidin-2-yl))methoxy)quinazolin-4-yl)-3-methylpiperazin-1-carboxylate (455 mg, 0.687 mmol) was dissolved in dichloromethane (8 mL) and then trifluoroacetic acid (3 mL) was added. The reaction was conducted at room temperature for 1 h and the reaction mixture was dried by rotary evaporation to obtain crude 6-chloro-8-cyclopropoxy-7-(5-methyl-TH-indazol-4-yl)-4-((R)-2-methylpiperazin-1-yl)-2-(((S)-1-methylpyrrolidin-2-yl)methoxy) quinazoline (500 mg, Y: 100%) as a yellow oil.

step 6: 6-chloro-8-cyclopropoxy-7-(5-methyl-TH-indazol-4-yl)-4-((R)-2-methylpiperazin-1-yl)-2-(((S)-1-methylpyrrolidin-2-yl)methoxy)quinazoline (500 mg, 0.89 mmol) and triethylamine (3 mL) were dissolved in dichloromethane (8 mL). At 0° C. acrylic anhydride (101 mg, 0.8 mmol) was added, and the reaction was conducted at 0° C. for 0.5 h. 20 mL of water and DCM (20 mL*3) were added to the reaction solution, which was extracted and the organic phase was dried by rotary evaporation to obtain 780 mg of a crude, 150 mg of which was purified by preparation to obtain 1-((3R)-4-(6-chloro-8-cyclopropoxy-7-(5-methyl-1H-indazol-4-yl)-2-(((S)-1-methylpyrrolidin-2-yl) methoxy)quinazolin-4-yl)-3-methylpiperazin-1-yl)prop-2-en-1-one (15.2 mg, Y: 10%). The preparation and separation conditions were the same as in Example 10. ES-API: [M+1]⁺=616.2. ¹HNMR (400 MHz, CDCl₃)δ 10.55 (s, 1H), 7.72 (s, 1H), 7.57 (s, 1H), 7.49 (d, J=8.5 Hz, 1H), 7.33 (d, J=8.8 Hz, 1H), 6.58 (d, J=25.7 Hz, 1H), 6.38 (d, J=16.8 Hz, 1H), 5.78 (d, J=10.5 Hz, 1H), 4.92 (d, J=132.9 Hz, 4H), 4.51-3.98 (m, 4H), 3.76 (d, J=71.0 Hz, 4H), 2.98 (s, 3H), 2.31 (s, 1H), 2.20 (s, 3H), 2.10 (s, 3H), 1.44 (t, J=7.8 Hz, 3H), 0.86 (d, J=15.7 Hz, 1H), 0.16 (dd, J=48.4, 21.9 Hz, 4H).

### Example 28 Preparation of Z28

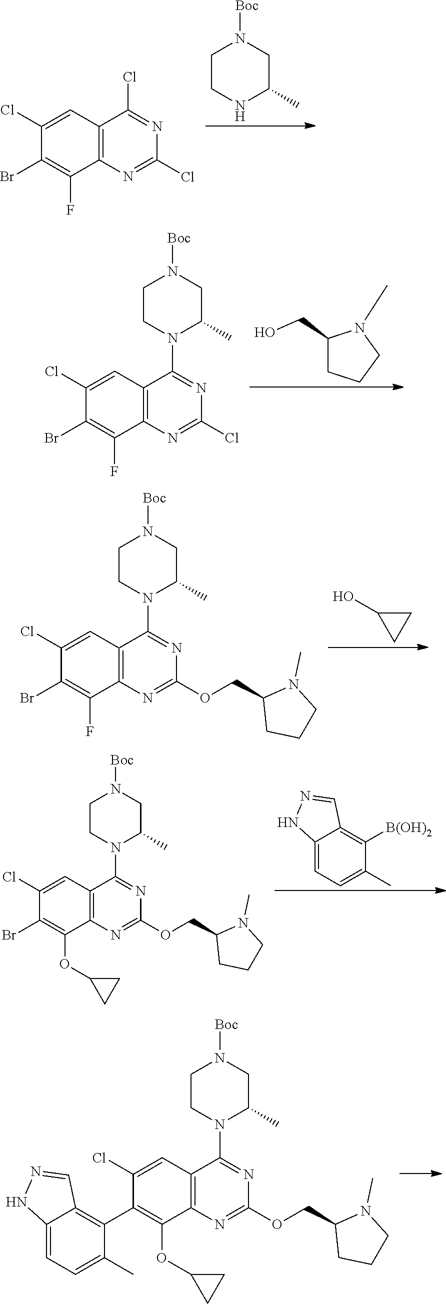

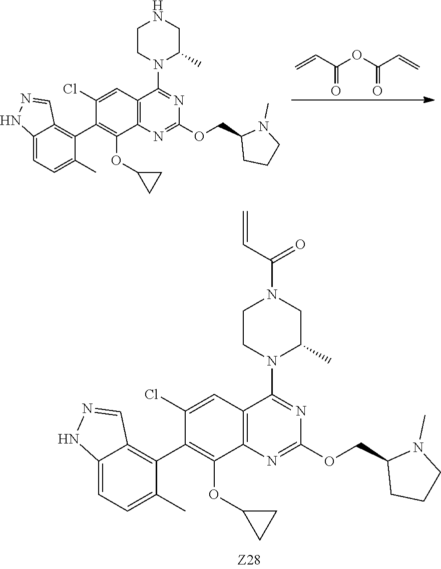

step 1: 7-bromo-2,4,6-trichloro-8-fluoroquinazoline (2 g, 6 mmol) was added into DCM/ACN(15/15 mL). The reaction solution was cooled to 0° C. and then TEA (3 g, 30 mmol) was added slowly. After that tert-butyl (S)-3-methylpiperazin-1-carboxylate (1.3 g, 6.6 mmol) was added. The reaction solution was slowly warmed up to room temperature and then stirred for 2 h. It was detected by LCMS till the reaction was complete. DCM (30 mL*3) and water (30 mL) were added which was extracted and the organic phase was dried and dried by rotary evaporation to obtain 2.9 g of tert-butyl (S)-4-(7-bromo-2,6-dichloro-8-fluoroquinazolin-4-yl)-3-methylpiperazin-1-carboxylate (Y: 100%) as a reddish brown solid. ES-API: [M+1]⁺=493.0.

step 2: tert-butyl (S)-4-(7-bromo-2,6-dichloro-8-fluoroquinazolin-4-yl)-3-methylpiperazin-1-carboxylate (2.9 g, 5.87 mmol) was added to (S)-(1-methylpyrrolidin-2-yl) methanol (10.1 g, 88 mmol) and then KF (1 g, 17.6 mmol) was added. The mixture was warmed up to 100° C. and the reaction was conducted for 2 h. It was detected by LCMS till the reaction was complete. After the reaction solution was cooled to room temperature 30 mL of water was added, which was extracted with ethyl acetate (30 mL*3). The organic phase was dried and dried by rotary evaporation to obtain a crude which was purified by flash column on silica gel (DCM/MeOH=50/1) to obtain 3.0 g of product tert-butyl (S)-4-(7-bromo-6-chloro-8-fluoro-2-(((S)-1-methylpyrrolidin-2-yl)methoxy)quinazolin-4-yl)-3-m ethylpiperazin-1-carboxylate (Y: 90%). ES-API: [M+1]⁺=572.1

step 3: cyclopropanol (456 mg, 7.86 mmol) was dissolved in tetrahydrofuran (30 mL) and at 0° C. sodium hydrogen (1.25 g, 52 mmol) was added. The reaction was stirred for 0.5 h while keeping the temperature. tert-butyl (S)-4-(7-bromo-6-chloro-8-fluoro-2-(((S)-1-methylpyrrolidin-2-yl)methoxy)quinazolin-4-yl)-3-methylpiperazin-1-carboxylate (3.0 g, 5.2 mmol) was then added into the reaction solution. The reaction was conducted for 1 h while keeping the temperature. The reaction solution was poured into ethyl acetate (30 mL), and the organic phase was washed with water (30 mL×2) and washed with brine (30 mL), dried over anhydrous sodium sulfate and filtered. The filtration was dried by rotary evaporation to obtain a yellow oil. The crude was purified by flash column on silica gel (DCM/MeOH=50/1) to obtain tert-butyl (S)-4-(7-bromo-6-chloro-8-cyclopropoxy-2-(((S)-1-methylpyrrolidin-2-yl)methoxy) quinazolin-4-yl)-3-methylpiperazin-1-carboxylate (1.7 g, Y: 54.8%). ES-API: [M+1]⁺=610.1

step 4: tert-butyl (S)-4-(7-bromo-6-chloro-8-cyclopropoxy-2-(((S)-1-methylpyrrolidin-2-yl) methoxy)quinazolin-4-yl)-3-methylpiperazin-1-carboxylate (800 mg, 1.3 mmol), (5-methyl-1H-indazol-4-yl) boronic acid (463 mg, 2.6 mmol), tripotassium phosphate (1.4 g, 6.5 mmol), tris (dibenzylideneindenacetone)dipalladium (120 mg, 0.13 mmol), 2-dicyclohexylphosphine-2′,4′,6′-triisopropylbiphenyl (61 mg, 0.13 mmol) were added in sequence into dioxane (8 mL) and water (2 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once it was purified by column on silica gel (methanol dichloromethane=0˜1:20) to obtain tert-butyl (3S)-4-(6-chloro-8-cyclopropoxy-7-(5-methyl-1H-indazol-4-yl)-2-(((S)-1-methylpyrrolidin-2-yl)) methoxy)quinazolin-4-yl)-3-methylpiperazin-1-carboxylate (600 mg, Y: 69%) as a yellow solid. ES-API: [M+1]⁺=662.1

step 5: tert-butyl (3S)-4-(6-chloro-8-cyclopropoxy-7-(5-methyl-1H-indazol-4-yl)-2-(((S)-1-methylpyrrolidin-2-yl))methoxy)quinazolin-4-yl)-3-methylpiperazin-1-carboxylate (600 mg, 0.9 mmol) was dissolved in dichloromethane (8 mL) and then trifluoroacetic acid (3 mL) was added. The reaction was conducted at room temperature for 1 h and the reaction mixture was dried by rotary evaporation to obtain crude 6-chloro-8-cyclopropoxy-7-(5-methyl-1H-indazol-4-yl)-4-((S)-2-methylpiperazin-1-yl)-2-(((S)-1-methylpyrrolidin-2-yl)methoxy)quinazoline (600 mg, Y: 100%) as a yellow oil.

step 6: 6-chloro-8-cyclopropoxy-7-(5-methyl-1H-indazol-4-yl)-4-((S)-2-methylpiperazin-1-yl)-2-(((S)-1-methylpyrrolidin-2-yl)methoxy)quinazoline (600 mg, 1.06 mmol) and triethylamine (3 mL) were dissolved in dichloromethane (8 mL). At 0° C. acrylic anhydride (106 mg, 0.84 mmol) was added, and the reaction was conducted at 0° C. for 0.5 h. 20 mL of water and DCM (20 mL*3) were added to the reaction solution, which was extracted and the organic phase was dried by rotary evaporation to obtain 780 mg of crude, which was purified to obtain 1-((3S)-4-(6-chloro-8-cyclopropoxy-7-(5-methyl-1H-indazol-4-yl)-2-(((S)-1-methylpyrrolidin-2-yl)methoxy)quinazolin-4-yl)-3-methylpiperazin-1-yl)prop-2-en-1-one (16.5 mg, Y: 2.5%). The preparation and separation conditions were the same as in Example 10. ES-API: [M+1]⁺=616.2. ¹HNMR (400 MHz, CDCl₃)δ 10.53 (s, 1H), 7.71 (s, 1H), 7.57 (d, J=8.3 Hz, 1H), 7.49 (s, 1H), 7.33 (d, J=8.6 Hz, 1H), 6.59 (d, J=17.3 Hz, 1H), 6.38 (d, J=16.7 Hz, 1H), 5.78 (d, J=10.5 Hz, 1H), 4.89-4.42 (m, 4H), 4.32 (s, 1H), 4.23-3.88 (m, 2H), 3.82-3.60 (m, 2H), 3.48-3.10 (m, 3H), 2.73 (s, 3H), 2.60 (s, 1H), 2.20 (s, 3H), 2.11-1.84 (m, 3H), 1.43 (d, J=7.6 Hz, 3H), 0.89-0.80 (m, 1H), 0.31-0.11 (m, 4H).

step 7: Preparation of Z28A and Z28B

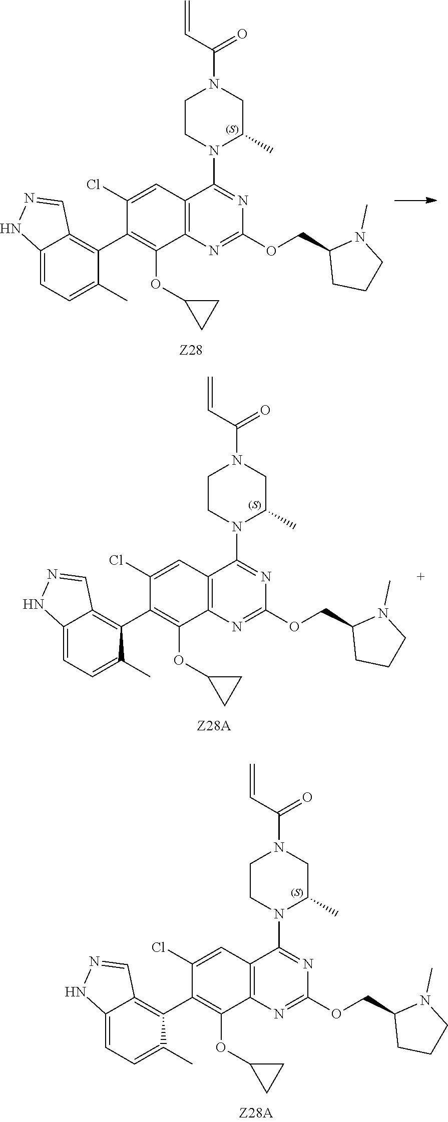

Z28 (440 mg) was separated through chiral resolution (mobile phase:HEP:isopropanol (0.1% DEA)=60:40; column: IG-H 0.46 cm I.D.×15 cm L; flow rate: 0.5 ml/min; column temperature: 25.0° C.) to obtain: one isomer, arbitrarily designated as Z28A: 1-((S)-4-((S)-6-chloro-8-cyclopropoxy-7-(5-methyl-1H-indazol-4-yl)-2-((((S)-1-methylpyrrolidone-pyridin-2-yl)methoxy)quinazolin-4-yl)-3-methylpiperazin-1-yl)prop-2-en-1-one (retention time: 1.977 min; 192 mg; purity: 100%, de value: 100%). ES-API: [M+H]⁺=616.2; and the other isomer, arbitrarily designated as Z28B: 1-((S)-4-((R)-6-chloro-8-cyclopropoxy-7-(5-methyl-1H-indazol-4-yl)-2-((((S)-1-methylpyrrolidone-pyridin-2-yl)methoxy)quinazolin-4-yl)-3-methylpiperazin-1-yl)prop-2-en-1-one (retention time: 1.886 min; 239 mg; purity: 100%, de value: 100%). ES-API: [M+H]⁺=616.2.

### Example 29 Preparation of Z29

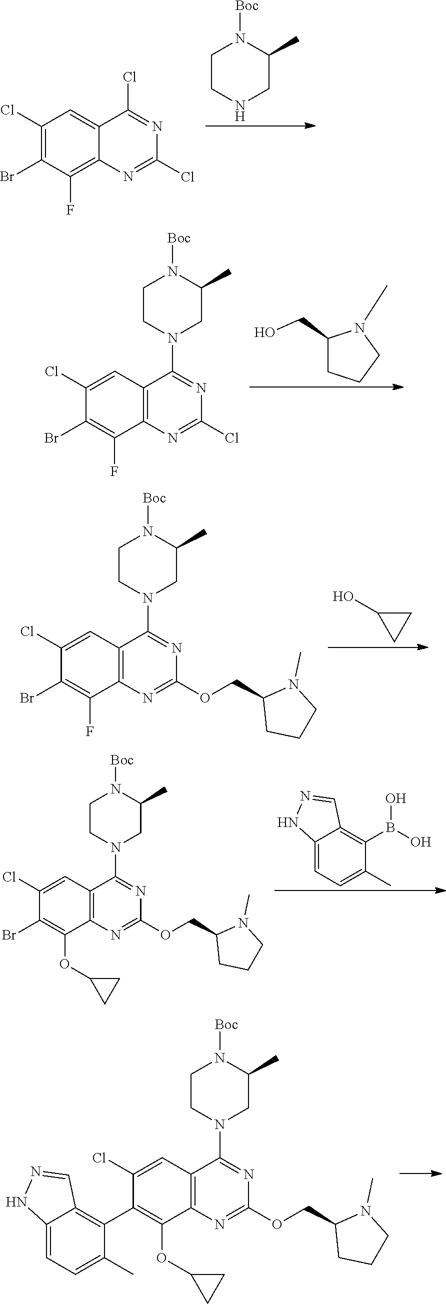

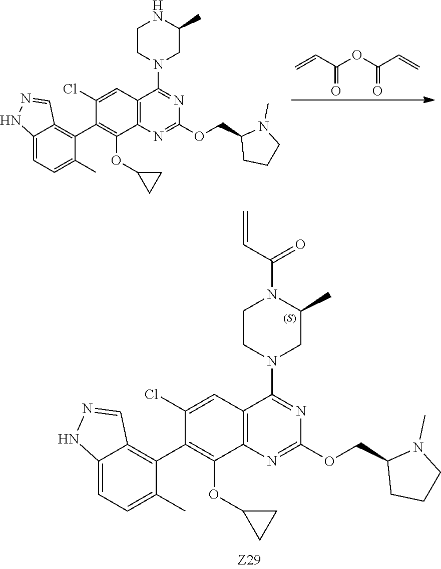

step 1: 7-bromo-2,4,6-trichloro-8-fluoroquinazoline (2 g, 6.06 mmol) was suspended in dichloromethane/acetonitrile (25/25 mL). The reaction solution was cooled to 0° C. and then triethylamine (1.84 g, 18.2 mmol) and tert-butyl (S)-2-methylpiperazin-1-carboxylate (1.50 g, 7.3 mmol) were added slowly. After the temperature was slowly warmed up to room temperature the reaction was stirred for 1 h. The reaction solution was extracted with dichloromethane and concentrated under reduced pressure to obtain crude tert-butyl (S)-4-(7-bromo-2,6-dichloro-8-fluoroquinazolin-4-yl)-2-methylpiperazin-1-carboxylate (3.1 g, as a reddish brown solid, Y: 100%), which was used in the next step. ES-API: [M+1]=493.0.

step 2: tert-butyl (S)-4-(7-bromo-2,6-dichloro-8-fluoroquinazolin-4-yl)-2-methylpiperazin-1-carboxylate (3.1 g, 6.28 mmol) was dissolved in DMF (40 mL), then (S)-(1-methylpyrrolidin-2-yl) methanol (2.6 g, 18.8 mmol), cesium carbonate (6.1 g, 18.8 mmol) and 1,4-diazabicyclo[2.2.2]octane (126 mg, 1.13 mmol) were added. Under nitrogen protection, the reaction was stirred at room temperature for 2 h. The reaction solution was extracted with ethyl acetate and concentrated under reduced pressure to give a crude which was purified by column chromatography (dichloromethane/methanol=20/1) to obtain tert-butyl (S)-4-(7-bromo-6-chloro-8-fluoro-2-((((S)-1-methylpyrrolidin-2-yl)methoxy)quinazolin-4-yl)-2-methylpiperazin-1-carboxylate (1.32 g, Y: 38, yellow liquid). ES-API: [M+1]=572.2.

step 3: sodium hydride (553 mg, 23.0 mmol) was suspended in dry tetrahydrofuran (10 mL) and after cooling to 0° C. cyclopropanol (200 mg, 3.46 mmol) was added. At 0° C. the reaction was stirred for 30 min, then a solution of tert-butyl (S)-4-(7-bromo-6-chloro-8-fluoro-2-((((S)-1-methylpyrrolidin-2-yl)methoxy)quinazolin-4-yl)-2-methylpiperazin-1-carboxylate (1.32 g, 2.30 mmol) in tetrahydrofuran (10 mL) was added dropwise. After warming up to room temperature, the reaction was stirred for 1 h. The reaction solution was quenched with saturated ammonium chloride solution, extracted with ethyl acetate and concentrated under reduced pressure to give a crude which was purified by column chromatography (dichloromethane/methanol=20/1) to obtain tert-butyl (S)-4-(7-bromo-6-chloro-8-cyclopropoxy-2-((((S)-1-methylpyrrolidin-2-yl)methoxy)quinazolin-4-yl)-2-methylpiperazin-1-carboxylate (714 mg, Y: 51%, yellow liquid). ES-API: [M+1]⁺=610.2.

step 4: tert-butyl (S)-4-(7-bromo-6-chloro-8-cyclopropoxy-2-((((S)-1-methylpyrrolidin-2-yl) methoxy)quinazolin-4-yl)-2-methylpiperazin-1-carboxylate (230 mg, 1.30 mmol), tris (dibenzylideneindenacetone)dipalladium (60 mg, 0.065 mmol), potassium phosphate (689 mg, 3.25 mmol) and 2-dicyclohexylphosphorus-2′,6′-diisopropoxy-1,1′-biphenyl (30 mg, 0.065 mmol) were dissolved in dioxane/water (6 mL/1 mL). Under nitrogen protection the reaction was stirred at 100° C. for 16 h. The reaction solution was extracted with ethyl acetate and concentrated under reduced pressure to give a crude which was purified by column chromatography (dichloromethane/methanol=20/1) to obtain tert-butyl (2S)-4-(6-chloro-8-cyclopropoxy-7-(5-methyl-1H-indazol-4-yl)-2-((((S)-1-methylpyrrolidin-2-yl)) methoxy)quinazolin-4-yl)-2-methylpiperazin-1-carboxylate (335 mg, Y: 68%, as a pale yellow solid). ES-API: [M+1]⁺=662.3

step 5: tert-butyl (2S)-4-(6-chloro-8-cyclopropoxy-7-(5-methyl-1H-indazol-4-yl)-2-((((S)-1-methylpyrrolidin-2-yl)) methoxy)quinazolin-4-yl)-2-methylpiperazin-1-carboxylate (335 mg, 0.5 mmol) was dissolved in dichloromethane (5 mL) and then trifluoroacetic acid (2.5 mL) was added. The reaction was stirred at room temperature for 30 min. The reaction solution was directly concentrated under reduced pressure to obtain crude 6-chloro-8-cyclopropoxy-7-(5-methyl-1H-indazol-4-yl)-4-((S)-3-methylpiperazin-1-yl)-2-((((S)-1-methylpyrrolidin-2-yl)methoxy)quinazoline (brown liquid, 335 mg), which was used directly in the next step. ES-API: [M+1]⁺=562.3.

step 6: 6-chloro-8-cyclopropoxy-7-(5-methyl-TH-indazol-4-yl)-4-((S)-3-methylpiperazin-1-yl)-2-((((S)-1-methylpyrrolidin-2-yl)methoxy)quinazoline (335 mg, 0.6 mmol) was dissolved in dichloromethane (5 mL) and after cooling to 0° C. triethylamine (3 mL) and acrylic anhydride (64 mg, 0.51 mmol) were added dropwise. The reaction was stirred at 0° C. for 30 min. The reaction solution was extracted with dichloromethane and concentrated under reduced pressure to give a crude which was purified by preparative plate (dichloromethane/methanol=12/1) to obtain 1-((2S)-4-(6-chloro-8-cyclopropoxy-7-(5-methyl-1H-indazol-4-yl)-2-((((S)-1-methylpyrrolidin-2-yl))methoxy)quinazolin-4-yl)-2-methylpiperazin-1-yl)prop-2-en-1-one (16.0 mg, Y: 8%, a white solid). ES-API: [M+1]⁺=616.3. ¹HNMR (400 MHz, DMSO-d6)δ 13.04 (s, 1H), 7.93 (s, 1H), 7.54-7.39 (m, 2H), 7.30 (d, J=8.5 Hz, 1H), 6.80 (dd, J=16.7, 10.5 Hz, 1H), 6.15 (d, J=16.5 Hz, 1H), 5.72 (d, J=10.4 Hz, 1H), 4.58-4.17 (m, 5H), 4.17-3.88 (m, 3H), 3.78-3.53 (m, 2H), δ 2.46 (s, 2H), 2.18-1.91 (m, 5H), 1.70 (dp, J=22.6, 7.1 Hz, 4H), 1.36-1.15 (m, 5H), 0.28-0.10 (m, 2H), 0.07 (d, J=3.4 Hz, 2H).

### Example 30 Preparation of Z30

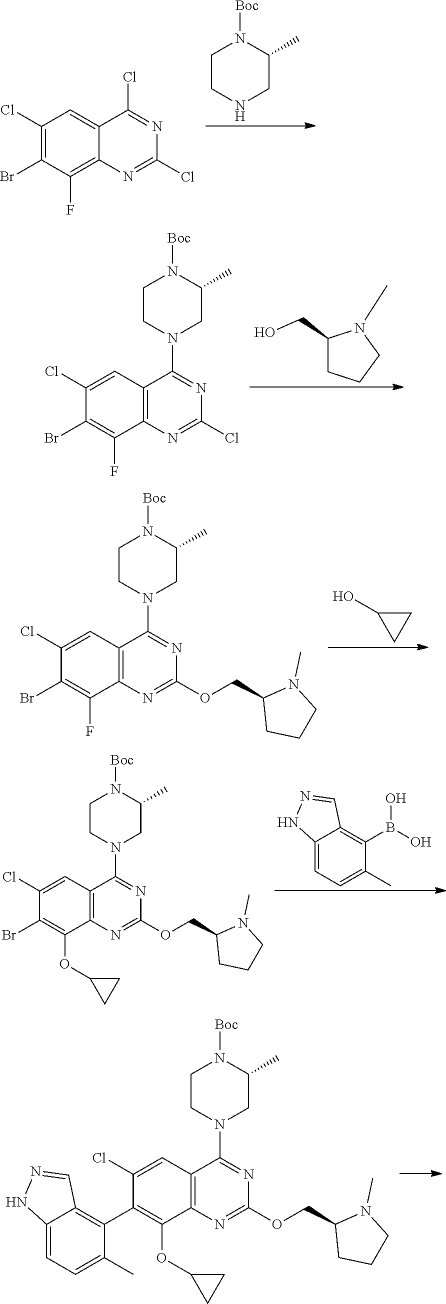

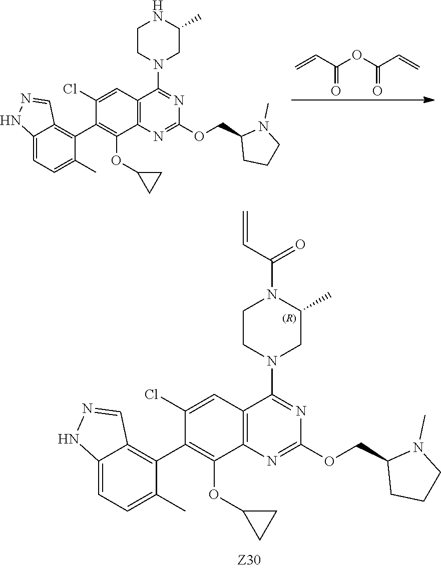

step 1: 7-bromo-2,4,6-trichloro-8-fluoroquinazoline (2 g, 6.06 mmol) was suspended in dichloromethane/acetonitrile (25 mL/25 mL). The reaction solution was cooled to 0° C. and then triethylamine (1.84 g, 18.2 mmol) and tert-butyl (R)-2-methylpiperazin-1-carboxylate (1.82 g, 9.09 mmol) were added slowly. After the temperature was slowly warmed up to room temperature the reaction was stirred for 1 h. The reaction solution was extracted with dichloromethane and concentrated under reduced pressure to obtain crude tert-butyl (R)-4-(7-bromo-2,6-dichloro-8-fluoroquinazolin-4-yl)-2-methylpiperazin-1-carboxylate (2.9 g, as a reddish brown solid, Y: 100%), which was used in the next step. ES-API: [M+1]⁺=493.0

step 2: tert-butyl (R)4-(7-bromo-2,6-dichloro-8-fluoroquinazolin-4-yl)-2-methylpiperazin-1-carboxylate (2.9 g, 5.87 mmol), (S)-(1-methylpyrrolidin-2-yl) methanol (10.0 g, 88.0 mmol), and potassium fluoride (1.02 g, 17.6 mmol) were heated to 100° C. and the reaction was conducted for 1 h. The reaction solution was extracted with dichloromethane and concentrated under reduced pressure to give a crude which was purified by column chromatography (dichloromethane/methanol=20/1) to obtain tert-butyl (R)-4-(7-bromo-6-chloro-8-fluoro-2-((((S)-1-methylpyrrolidin-2-yl)methoxy)quinazolin-4-yl)-2-methylpiperazin-1-carboxylate (1.5 g, Y: 43%, orange liquid). ES-API: [M+1]⁺=572.2

step 3: Under nitrogen protection sodium hydride (587 mg, 24.5 mmol) was suspended in dry tetrahydrofuran (10 mL) and after cooling to 0° C. cyclopropanol (213 mg, 3.67 mmol) was added. The reaction was stirred at 0° C. for 30 min then a solution of tert-butyl (R)-4-(7-bromo-6-chloro-8-fluoro-2-((((S)-1-methylpyrrolidin-2-yl)methoxy)quinazolin-4-yl)-2-methylpiperazin-1-carboxylate (1.50 g, 2.45 mmol) in tetrahydrofuran (10 mL) was added dropwise. After slowly warming up to room temperature, the reaction was stirred for 1 h. The reaction solution was quenched with saturated ammonium chloride solution, extracted with ethyl acetate and concentrated under reduced pressure to give a crude which was purified by column chromatography (dichloromethane/methanol=20/1) to obtain tert-butyl (R)-4-(7-bromo-6-chloro-8-cyclopropoxy-2-((((S)-1-methylpyrrolidin-2-yl)methoxy)quinazolin-4-yl)-2-methylpiperazin-1-carboxylate (250 mg, Y: 15%, pale yellow liquid). ES-API: [M+1]⁺=610.2

step 4: tert-butyl (R)-4-(7-bromo-6-chloro-8-cyclopropoxy-2-((((S)-1-methylpyrrolidin-2-yl) methoxy)quinazolin-4-yl)-2-methylpiperazin-1-carboxylate (144 mg, 0.82 mmol), Tris (dibenzylideneacetone)dipalladium (37 mg, 0.04 mmol), potassium phosphate (435 mg, 2.05 mmol) and 2-dicyclohexylphosphorus-2′,6′-diisopropoxy-1,1′-biphenyl (20 mg, 0.04 mmol) were dissolved in dioxane/water (4 mL/1 mL). Under nitrogen protection the reaction was stirred at 100° C. for 16 h. The reaction solution was extracted with ethyl acetate and concentrated under reduced pressure to give a crude which was purified by column chromatography (dichloromethane/methanol=20/1) to obtain tert-butyl (2R)-4-(6-chloro-8-cyclopropoxy-7-(5-methyl-1H-indazol-4-yl)-2-((((S)-1-methylpyrrolidin-2-yl))methoxy)quinazolin-4-yl)-2-methylpiperazin-1-carboxylate (86 mg, Y: 32%, brown liquid). ES-API: [M+1]⁺=662.3

step 5: tert-butyl (2R)-4-(6-chloro-8-cyclopropoxy-7-(5-methyl-1H-indazol-4-yl)-2-((((S)-1-methylpyrrolidin-2-yl))methoxy)quinazolin-4-yl)-2-methylpiperazin-1-carboxylate (86 mg, 0.13 mmol) was dissolved in dichloromethane (2 mL) and then trifluoroacetic acid (2 mL) was added. The reaction was stirred at room temperature for 30 min. The reaction solution was directly concentrated under reduced pressure to obtain crude 6-chloro-8-cyclopropoxy-7-(5-methyl-1H-indazol-4-yl)-4-((R)-3-methylpiperazin-1-yl)-2-((((S)-1-methylpyrrolidin-2-yl)methoxy)quinazoline (86 mg), which was used directly in the next step. ES-API: [M+1]⁺=562.3

step 6: 6-chloro-8-cyclopropoxy-7-(5-methyl-1H-indazol-4-yl)-4-((R)-3-methylpiperazin-1-yl)-2-((((S)-1-methylpyrrolidin-2-yl)methoxy)quinazoline (86 mg, 0.15 mmol) was dissolved in dichloromethane (2 mL) and after cooling to 0° C. triethylamine (2 mL) and acrylic anhydride (16 mg, 0.13 mmol) were added dropwise. The reaction was stirred at 0° C. for 30 min. The reaction solution was extracted with dichloromethane and concentrated under reduced pressure to give a crude which was purified by preparative plate (dichloromethane/methanol=12/1) to obtain 1-((2R)-4-(6-chloro-8-cyclopropoxy-7-(5-methyl-1H-indazol-4-yl)-2-((((S)-1-methylpyrrolidin-2-yl))methoxy)quinazolin-4-yl)-2-methylpiperazin-1-yl)prop-2-en-1-one (3.8 mg, Y: 4%, an off-white solid). ES-API: [M+1]⁺=616.3. ¹HNMR (400 MHz, CDCl₃)δ 7.81 (d, J=3.0 Hz, 1H), 7.59 (d, J=6.9 Hz, 1H), 7.50-7.43 (m, 1H), 7.37-7.30 (m, 1H), 6.61 (dd, J=16.8, 10.5 Hz, 1H), 6.37 (d, J=16.6 Hz, 1H), 5.77 (dd, J=10.5, 1.9 Hz, 1H), 4.63 (s, 1H), 4.50-4.23 (m, 3H), 4.14 (t, J=12.3 Hz, 1H), 3.63 (d, J=59.3 Hz, 1H), 3.56 (s, 2H), 3.35 (s, 1H), 3.20 (s, 1H), 2.86 (s, 1H), 2.55 (s, 3H), 2.37 (s, 1H), 2.21 (d, J=5.6 Hz, 3H), 2.10 (s, 2H), 1.46 (t, J=6.6 Hz, 3H), 1.32 (d, J=34.0 Hz, 3H), 0.99-0.79 (m, 1H), 0.29-0.11 (m, 4H).

### Example 31 Preparation of Z31

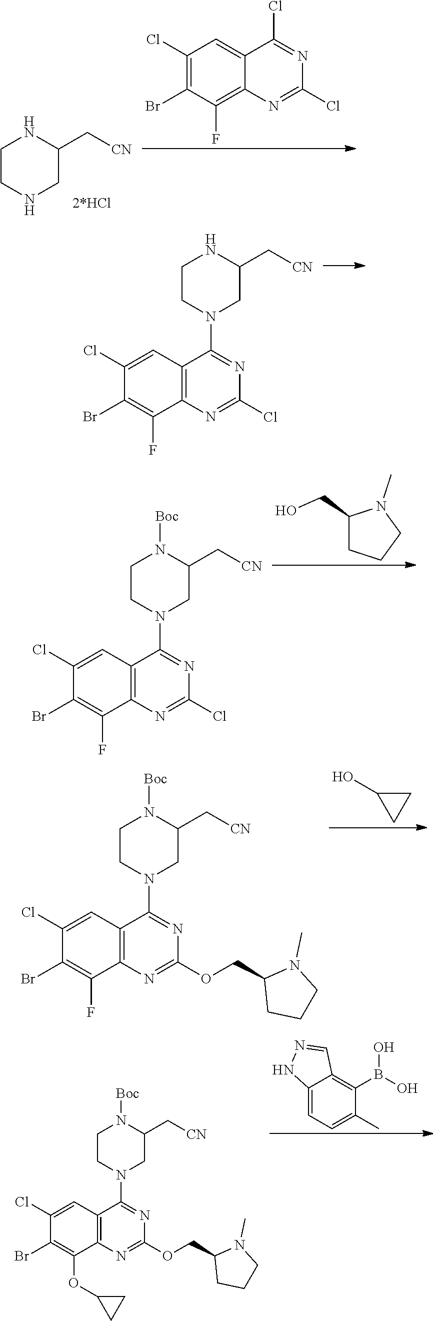

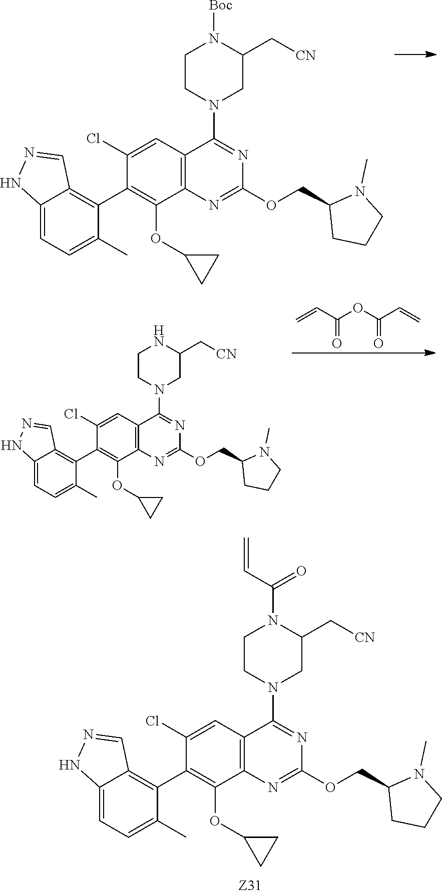

step 1: 7-bromo-2,4,6-trichloro-8-fluoroquinazoline (993 mg, 3.03 mmol), 2-(piperazin-2-yl) acetonitrile (600 mg, 3.03 mmol) was added into dichloromethane/acetonitrile (10/10 mL). The reaction solution was cooled to 0° C. and then triethylamine (1.53 g, 15.15 mmol) was added slowly. The reaction was stirred for 0.5 h. It was detected by LCMS till the reaction was complete. The reaction solution was directly used into the next step. ES-API: [M+1]⁺=418.0

step 2: To the reaction solution obtained in the previous step di-tert-butyl dicarbonate (660 mg, 3.03 mmol) and 4-dimethylaminopyridine (37 mg, 0.303 mmol) were added. The reaction was conducted at room temperature for 2 h. The mixture was separated by adding water and dichloromethane and the organic phase was washed with brine, dried over sodium sulfate, and dried by rotary evaporation to obtain crude tert-butyl 4-(7-bromo-2,6-dichloro-8-fluoroquinazolin-4-yl)-2-(cyanomethyl)piperazin-1-carboxylate (2.0 g, Y: 100%). ES-API: [M+1]⁺=518.0

step 3: tert-butyl 4-(7-bromo-2,6-dichloro-8-fluoroquinazolin-4-yl)-2-(cyanomethyl) piperazin-1-carboxylate (2.0 g, 3.03 mmol) was added to (S)-(1-methylpyrrolidin-2-yl) methanol (10 mL) and then potassium fluoride (527 mg, 9.09 mmol) was added. The mixture was heated to 100° C. and the reaction was conducted for 2 h. It was detected by LCMS till the reaction was complete. After the reaction solution was cooled to room temperature 30 mL of water was added, and the mixture was extracted with ethyl acetate (30 mL*3). The organic phase was dried and dried by rotary evaporation to obtain a crude which was purified by flash column on silica gel (dichloromethane/methanol=100/1) to obtain tert-butyl 4-(7-bromo-6-chloro-8-fluoro-2-((((S)-1-methylpyrrolidin-2-yl)methoxy)quinazolin-4-yl)-2-(cyanomethyl)piperazin-1-carboxylate (1.3 g,Y: 72%). ES-API: [M+1]⁺=597.1

step 4: tert-butyl 4-(7-bromo-6-chloro-8-fluoro-2-((((S)-1-methylpyrrolidin-2-yl) methoxy)quinazolin-4-yl)-2-(cyanomethyl)piperazin-1-carboxylate (1.3 g, 2.18 mmol), and cyclopropanol (190 mg, 3.27 mmol) were dissolved in tetrahydrofuran (15 mL). At 0° C. sodium hydrogen (872 mg, 21.8 mmol) was added in batches. The reaction was stirred for 0.5 h while keeping the temperature. The reaction was quenched by adding water, and the mixture was extracted with ethyl acetate. The organic phase was washed with brine, which was dried over anhydrous sodium sulfate, dried by rotary evaporation under reduced pressure. The crude was purified by flash column on silica gel (dichloromethane/methanol=100/1) to obtain tert-butyl 4-(7-bromo-6-chloro-8-cyclopropoxy-2-(((S)-1-methylpyrrolidin-2-yl)methoxy)quinazolin-4-yl)-2-(cyanomethyl)piperazin-1-carboxylate (350 mg, Y: 25.3%). ES-API: [M+1]⁺=635.2

step 5: tert-butyl 4-(7-bromo-6-chloro-8-cyclopropoxy-2-(((S)-1-methylpyrrolidin-2-yl) methoxy)quinazolin-4-yl)-2-(cyanomethyl)piperazin-1-carboxylate (250 mg, 0.39 mmol), (5-methyl-1H-indazol-4-yl) boronic acid (208 mg, 1.18 mmol), potassium carbonate (269 mg, 1.95 mmol) and Sphos Pd G2 (28 mg, 0.039 mmol) were added in sequence to dioxane (2.5 mL) and water (0.5 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once, it was dried over anhydrous sodium sulfate, dried by rotary evaporation under reduced pressure, and purified by thin layer chromatography (dichloromethane/methanol=10/1) to obtain tert-butyl 4-(6-chloro-8-cyclopropoxy-7-(5-methyl-1H-indazol-4-yl)-2-((((S)-1-methylpyrrolidin-2-yl)methoxy)quinazolin-4-yl)-2-(cyanomethyl)piperazin-1-carboxylate (50 mg, Y: 15%). ES-API: [M/2+1]⁺=344.1.

step 6: tert-butyl 4-(6-chloro-8-cyclopropoxy-7-(5-methyl-1H-indazol-4-yl)-2-((((S)-1-methylpyrrolidin-2-yl)methoxy)quinazolin-4-yl)-2-(cyanomethyl)piperazin-1-carboxylate (50 mg, 0.073 mmol) was dissolved in dichloromethane (0.5 mL) and then trifluoroacetic acid (0.5 mL) was added. The reaction was conducted at room temperature for 0.5 h and the reaction mixture was dried by rotary evaporation to obtain crude 2-(4-(6-chloro-8-cyclopropoxy-7-(5-methyl-1H-indazol-4-yl)-2-(((S)-1-methylpyrrolidin-2-yl)methoxy) quinazolin-4-yl)piperazin-2-yl) acetonitrile (50 mg, Y: 100%). ES-API: [M/2+1]⁺=294.1

step 7: 2-(4-(6-chloro-8-cyclopropoxy-7-(5-methyl-1H-indazol-4-yl)-2-(((S)-1-methylpyrrolidin-2-yl)methoxy)quinazolin-4-yl)piperazin-2-yl) acetonitrile (50 mg, 0.073 mmol) and triethylamine (0.5 mL) were dissolved in dichloromethane (1 mL). At 0° C. acrylic anhydride was added (7.4 mg, 0.058 mmol), and the reaction was conducted at 0° C. for 0.5 h. To the reaction solution 20 mL of water and dichloromethane (20 mL*3) were added which was extracted and the organic phase was washed with brine, dried over anhydrous sodium sulfate, dried by rotary evaporation under reduced pressure and purified by thin layer chromatography (dichloromethane/methanol=10/1) to obtain 2-(1-acryloyl-4-(6-chloro-8-cyclopropoxy-7-(5-methyl-1H-indazol-4-yl)-2-(((S)-1-methylpyrrolidin-2-yl))methoxy)quinazolin-4-yl)piperazin-2-yl) acetonitrile (2.2 mg, Y: 4.71%). ES-API: [M/2+1]⁺=321.1. ¹HNMR (400 MHz, CDCl₃)δ7.77 (s, 1H), 7.58 (s, 1H), 7.48 (d, J=8.5 Hz, 1H), 7.35 (d, J=8.5 Hz, 1H), 6.60 (s, 0H), 6.41 (d, J=16.7 Hz, 1H), 5.85 (d, J=10.6 Hz, 1H), 4.91 (s, 0H), 4.63 (s, 1H), 4.34 (s, 3H), 3.75-3.63 (m, 3H), 3.54 (s, 2H), 3.05 (dd, J=16.6, 8.2 Hz, 1H), 2.84 (s, 5H), 2.21 (d, J=1.8 Hz, 5H), 2.00 (s, 3H), 1.33 (s, 3H), 0.22 (dd, J=10.1, 5.7 Hz, 4H).

### Example 32 Preparation of Z32

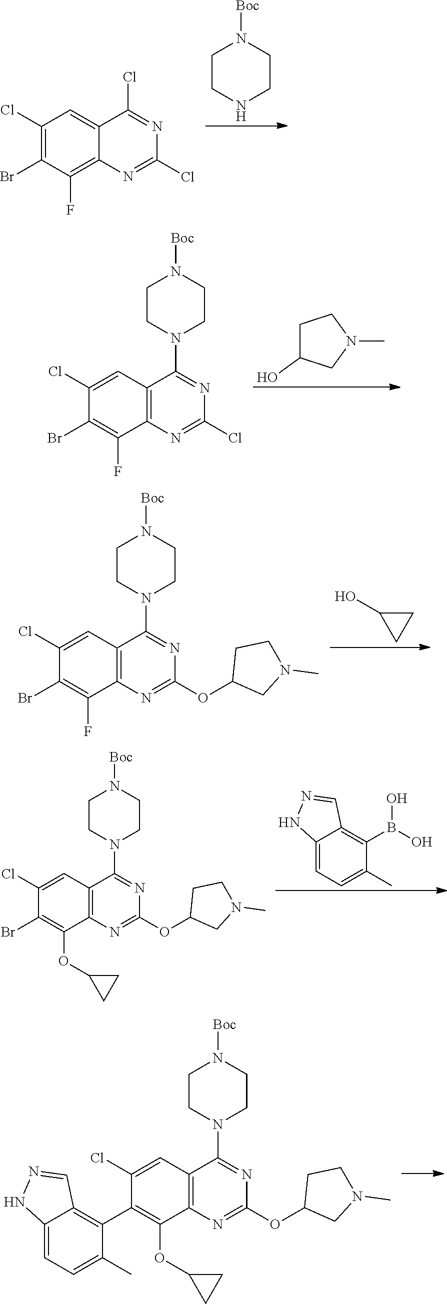

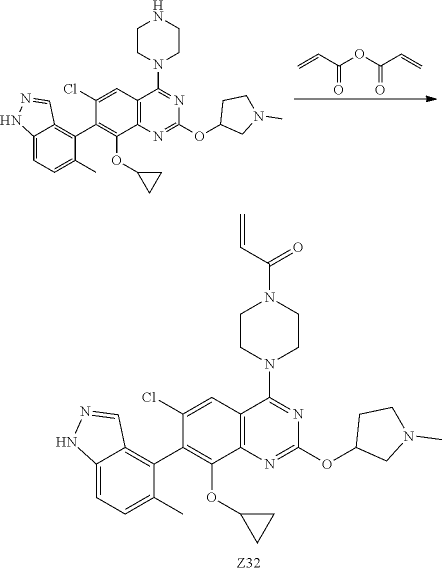

step 1: at ° C., to a mixed solution of 7-bromo-2,4,6-trichloro-8-fluoroquinazoline (5.0 g, 15.13 mmol) and N,N′-diisopropylethylamine (5.87 g, 45.40 mmol) in dichloromethane (50 mL) and acetonitrile (50 mL) were added tert-butyl piperazin-1-carboxylate (3.10 g, 16.65 mmol). Then the mixture was stirred at room temperature for 2 h. Then water (200 mL) was added and the mixture was extracted with dichloromethane (3×300 mL). The combined organic phase was washed with saturated brine (3×100 mL) and dried over anhydrous sodium sulfate. The solvent was removed by rotary evaporation. The residue was triturated with dichloromethane and petroleum ether and filtered with suction to obtain tert-butyl 4-(7-bromo-2,6-dichloro-8-fluoroquinazolin-4-yl)piperazin-1-carboxylate (5.0 g, 68.80%) as a yellow solid, which was used directly in the next step without further purification. ES-API: [M+1]⁺=479.0.

step 2: to a mixed solution of tert-butyl 4-(7-bromo-2,6-dichloro-8-fluoroquinazolin-4-yl)piperazin-1-carboxylate (4.7 g, 9.79 mmol) in N,N′-dimethylformamide (90 mL) were added cesium carbonate (7.97 g, 24.47 mmol), triethylene diamine (198 mg, 1.76 mmol) and 3-hydroxy-1-methyltetrahydropyrrole (1.19 g, 11.75 mmol). Then the mixture was stirred at room temperature for 4 h. The reaction mixture was poured into ice water (900 mL) and extracted with ethyl acetate (3×300 mL). The combined organic phase was washed with water (3×200 mL) and washed with saturated brine (3×150 mL) and dried over anhydrous sodium sulfate. The solvent was removed by rotary evaporation. The residue was purified by flash chromatography, eluting with dichloromethane and methanol (9%) to obtain tert-butyl 4-(7-bromo-6-chloro-8-fluoro-2-((1-methylpyrrolidin-3-yl)oxy)quinazolin-4-yl) piperazin-1-carboxylate (4.5 g, 84.38%) as a yellow oil. ES-API: [M+1]⁺=544.1.

step 3: at 0° C., to a mixed solution of cyclopropanol (799 mg, 13.77 mmol) in tetrahydrofuran (30 mL) sodium hydride (918 mg, 13.77 mmol, the purity is 60%) was added. The obtained mixture was stirred at 0° C. for 1 h. tert-butyl 4-(7-bromo-6-chloro-8-fluoro-2-((1-methylpyrrolidin-3-yl)oxy)quinazolin-4-yl)piperazin-1-carboxylate (2.5 g, 4.59 mmol) was added to the reaction mixture. Then the reaction was conducted at room temperature for 4 h. The reaction mixture was poured into ice water (200 mL) and extracted with dichloromethane (3×200 mL). The combined organic phase was washed with saturated ammonium chloride (3×100 mL) and washed with saturated brine (3×100 mL). The solvent was removed by rotary evaporation. The residue was purified by flash chromatography, eluting with dichloromethane and methanol (3%), to obtain tert-butyl 4-(7-bromo-6-chloro-8-cyclopropoxy-2-((1-methylpyrrolidin-3-yl)oxy)quinazolin-4-yl)piperazin-1-carboxylate (2.3 g, 85.99%) as a white solid. ES-API: [M+1]⁺=582.1.

step 4: to a suspension of tert-butyl 4-(7-bromo-6-chloro-8-cyclopropoxy-2-((1-methylpyrrolidin-3-yl)oxy)quinazolin-4-yl)piperazin-1-carboxylate (1.2 g, 2.06 mmol), (5-methyl-1H-indazol-4-yl) boronic acid (725 mg, 4.12 mmol), potassium phosphate (1.31 g, 6.18 mmol) in 1,4-dioxane (12 mL) and deionized water (4 mL) tris (dibenzylideneindenacetone)dipalladium (377 mg, 0.41 mmol) and dicyclohexylphosphorus-2,6-diisopropoxy 1,1″-biphenyl (192 mg, 0.41 mmol) were added. The obtained mixture was heated to 100° C. and stirred overnight under an argon atmosphere. After cooling to room temperature, the reaction mixture was extracted with dichloromethane (3×150 mL). The combined organic phase was washed with water (3×50 mL) and washed with saturated brine (3×50 mL) and dried over anhydrous sodium sulfate. The solvent was removed by rotary evaporation. The residue was purified by flash chromatography, eluting with dichloromethane and methanol (3%), to obtain tert-butyl 4-(6-chloro-8-cyclopropoxy-7-(5-methyl-1H-indazol-4-yl)-2-((1-methylpyrrolidin-3-yl)oxy)quinazolin-4-yl-1-carboxylate piperazine (530 mg, 40.60%) as a yellow solid. ES-API: [M+1]⁺=634.3.

step 5: to a solution of tert-butyl 4-(6-chloro-8-cyclopropoxy-7-(5-methyl-1H-indazol-4-yl)-2-((1-methylpyrrolidin-3-yl)oxy)quinazolin-4-yl-1-carboxylate piperazine (530 mg, 0.84 mmol) in dichloromethane (5 mL) trifluoroacetic acid (2 mL) was added. The obtained mixture was stirred at room temperature for 30 min. The pH of the reaction mixture was adjusted to 8 with 1M sodium bicarbonate. The reaction mixture was extracted with dichloromethane (3×30 mL). The combined organic phase was washed with saturated brine (3×30 mL) and dried over anhydrous sodium sulfate. The solvent was removed by rotary evaporation to obtain 6-chloro-8-cyclopropoxy-7-(5-methyl-1H-indazol-4-yl)-2-((1-methylpyrrolidin-3-yl)oxy)-4-(piperazin-1-yl)quinazoline (400 mg, 89.62%) as a yellow oil, which was used directly in the next step without further purification. ES-API: [M+1]⁺=534.2.

step 6: to a solution of 6-chloro-8-cyclopropoxy-7-(5-methyl-1H-indazol-4-yl)-2-((1-methylpyrrolidin-3-yl)oxy)-4-(piperazin-1-yl)quinazoline (380 mg, 0.71 mmol) in dichloromethane (5 mL) triethylamine (720 mg, 7.1 mmol) was added. The mixture was cooled to 0° C. At 0° C. acrylic anhydride (89 mg, 0.71 mmol) was added and stirred for 1 h. Then the solvent was removed under reduced pressure. The residue was purified by Pre-HPLC to obtain Z32 (20.2 mg, 4.83%) as a white solid. ¹HNMR (400 MHz, CDCl₃)δ 7.76 (s, 1H), 7.57-7.47 (m, 2H), 7.33 (d, J=8.6 Hz, 1H), 6.62 (dd, J=16.6, 10.5 Hz, 1H), 6.38 (d, J=16.7 Hz, 1H), 5.79 (d, J=10.5 Hz, 1H), 5.73 (d, J=5.9 Hz, 1H), 4.14 (p, J=4.5 Hz, 1H), 3.88 (q, J=13.9, 9.9 Hz, 8H), 3.58 (s, 1H), 3.24 (d, J=12.2 Hz, 2H), 2.86 (s, 3H), 2.50 (dq, J=33.2, 10.1, 8.7 Hz, 2H), 2.19 (s, 3H), 1.26 (s, 1H), 0.27-0.08 (m, 4H). ES-API: [M+1]⁺=588.2.

### Example 33 Preparation of Z33

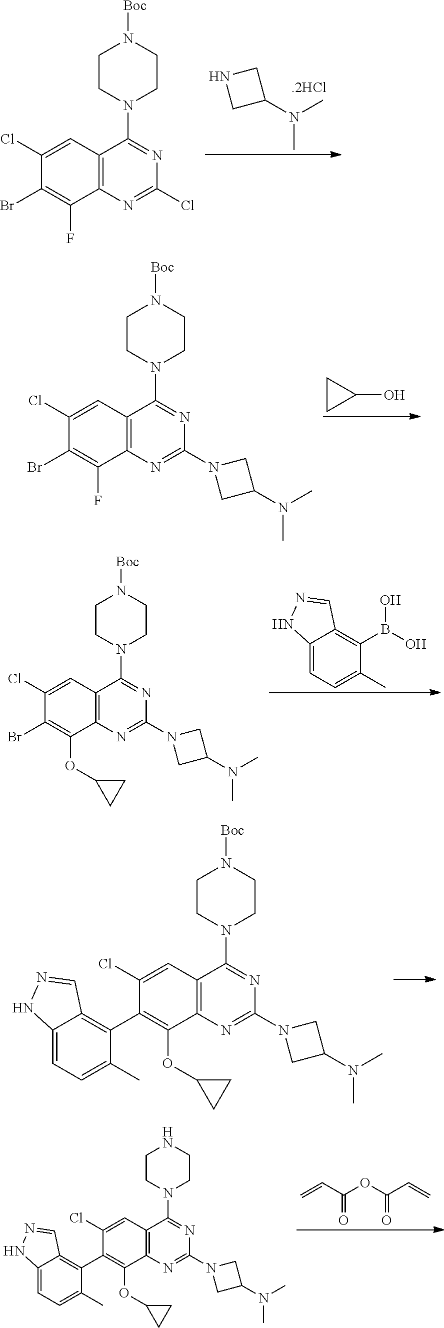

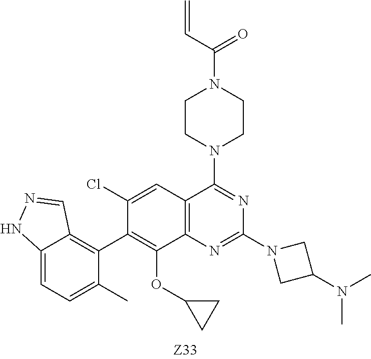 [SMILES: C=CC(=O)N1CCN(c2nc(N3CC(N(C)C)C3)nc3c(OC4CC4)c(-c4c(C)ccc5[nH]ncc45)c(Cl)cc23)CC1]

step 1: tert-butyl 4-(7-bromo-2,6-dichloro-8-fluoroquinazolin-4-yl)piperazin-1-carboxylate (5 g, 10.46 mmol) was dissolved in N,N-dimethylformamide (50 mL), then N,N-dimethylazacyclo-3-amine hydrochloride (5.43 g, 31.38 mmol), triethylene diamine (0.176 g, 1.57 mmol) and cesium carbonate (10.23 g, 31.38 mmol) were added. The mixture was heated to 80° C. and the reaction was conducted for 2 h. After cooling to room temperature, the mixture was separated by adding water and ethyl acetate. The organic phase was washed with water and washed with brine, and dried over anhydrous sodium sulfate. The solvent was concentrated under reduced pressure to obtain crude tert-butyl 4-(7-bromo-6-chloro-2-(3-(dimethylamino)azetidine-1-yl)-8-fluoroquinazolin-4-yl)piperazin-1-carboxylate (5.5 g, Y:: 97%). ES-API: [M+1]⁺=543.1

step 2: tert-butyl 4-(7-bromo-6-chloro-2-(3-(dimethylamino)azetidine-1-yl)-8-fluoroquinazolin-4-yl)piperazin-1-carboxylate (5.5 g, 10.15 mmol), cyclopropanol (2.94 g, 50.7 mmol) were dissolved in tetrahydrofuran (50 mL), and at 0° C. sodium hydrogen (4.06 g, 101.5 mmol) was added in batches. The reaction was stirred for 2 h while keeping the temperature. The reaction was quenched by adding water, and the mixture was extracted with ethyl acetate. The organic phase was washed with brine, dried over anhydrous sodium sulfate, and dried by rotary evaporation under reduced pressure to obtain crude tert-butyl 4-(7-bromo-6-chloro-8-cyclopropoxy-2-(3-(dimethylamino)azetidine-1-yl)quinazolin-4-yl) piperazin-1-carboxylate (6.2 g, Y: 100%). ES-API: [M+1]⁺=581.2

step 3: tert-butyl 4-(7-bromo-6-chloro-8-cyclopropoxy-2-(3-(dimethylamino)azetidine-1-yl)quinazolin-4-yl)piperazin-1-carboxylate (4 g, crude, 7.36 mmol), (5-methyl-1H-indazol-4-yl) boronic acid (2.59 g, 14.7 mmol), potassium phosphate (3.12 g, 14.7 mmol), Sphos (604 mg, 1.47 mmol) and Pd₂(dba)₃ (674 mg, 0.736 mmol) were added in sequence to dioxane (60 mL) and water (10 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once, it was dried over anhydrous sodium sulfate and dried by rotary evaporation under reduced pressure to give a crude which was purified by column (dichloromethane/methanol=100/1) to obtain tert-butyl 4-(6-chloro-8-cyclopropoxy-2-(3-(dimethylamino)azetidine-1-yl)-7-(5-methyl-1H-indazol-4-yl)quinazolin-4-yl)1-carboxylate piperazine (500 mg, Y: 10.8%). ES-API: [M+1]⁺=633.2

step 4: tert-butyl 4-(6-chloro-8-cyclopropoxy-2-(3-(dimethylamino)azetidine-1-yl)-7-(5-methyl-1H-indazol-4-yl)quinazolin-4-yl)1-carboxylate piperazine (500 mg, 0.79 mmol) was dissolved in dichloromethane (5 mL) and then trifluoroacetic acid (5 mL) was added. The reaction was conducted at room temperature for 0.5 h and the reaction mixture was dried by rotary evaporation to obtain crude 1-(6-chloro-8-cyclopropoxy-7-(5-methyl-1H-indazol-4-yl)-4-(piperazin-1-yl)quinazolin-2-yl)-N,N-dimethylazetidine-3-amine (600 mg, Y: 100%). ES-API: [M+1]⁺=533.2

step 5: 1-(6-chloro-8-cyclopropoxy-7-(5-methyl-1H-indazol-4-yl)-4-(piperazin-1-yl) quinazolin-2-yl)-N,N-dimethylazetidine-3-amine (600 mg, 0.79 mmol) and triethylamine (3 mL) were dissolved in dichloromethane (10 mL). At 0° C. acrylic anhydride (80 mg, 0.632 mmol) was added, and the reaction was conducted at 0° C. for 0.5 h. The reaction solution was extracted with water and dichloromethane. The organic phase was washed with brine, dried over anhydrous sodium sulfate, dried by rotary evaporation under reduced pressure and purified by preparation (column: Ultimate XB-C18, 50*250 mm, 10 um, mobile phase: A: purified water B: purified acetonitrile, flow rate: 80 ml/min, gradient: within 50 min, B/A=20%-90%, wavelength: 214 nm, column temperature: room temperature) to obtain 1-(4-(6-chloro-8-cyclopropoxy-2-(3-(dimethylamino)azetidine-1-yl)-7-(5-methyl-1H-indazol-4-yl)quinazolin-4-yl)piperazin-1-yl)prop-2-en-1-one (93.5 mg, Y: 20.1%). ES-API: [M+1]⁺=587.3. ¹HNMR (400 MHz, CDCl₃)δ10.20 (s, 1H), 7.63 (d, J=10.1 Hz, 2H), 7.44 (d, J=8.5 Hz, 1H), 7.31 (s, 1H), 6.62 (dd, J=16.8, 10.5 Hz, 1H), 6.36 (d, J=16.8 Hz, 1H), 5.77 (d, J=10.5 Hz, 1H), 4.36-4.16 (m, 3H), 4.04 (t, J=7.7 Hz, 2H), 3.92 (s, 2H), 3.85-3.68 (m, 6H), 3.25-3.17 (m, 1H), 2.22 (d, J=10.7 Hz, 9H), 0.36-0.05 (m, 4H).

### Example 34 Preparation of Z34

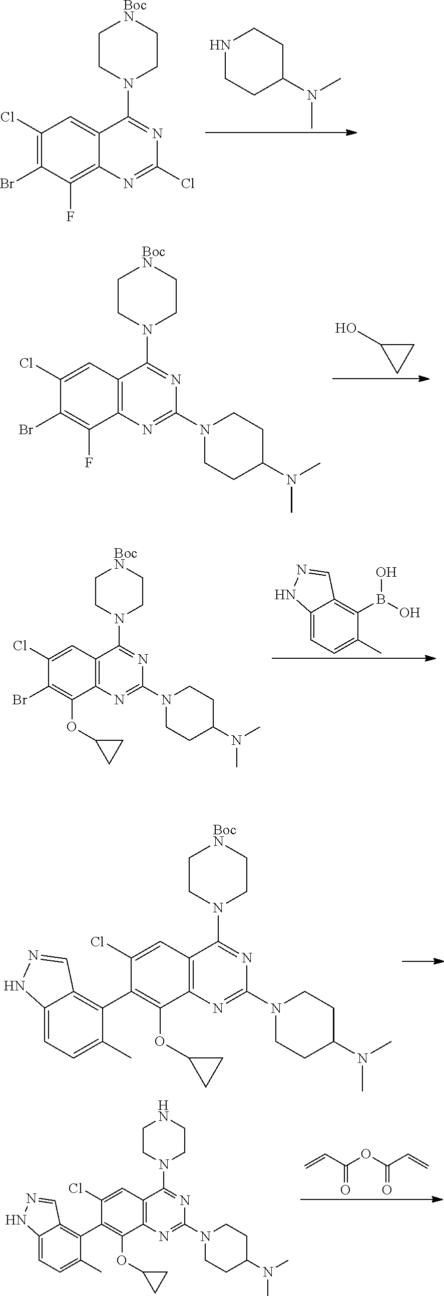

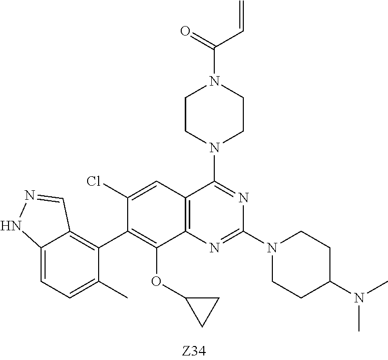 [SMILES: C=CC(=O)N1CCN(c2nc(N3CCC(N(C)C)CC3)nc3c(OC4CC4)c(-c4c(C)ccc5[nH]ncc45)c(Cl)cc23)CC1]

step 1: tert-butyl 4-(7-bromo-2,6-dichloro-8-fluoroquinazolin-4-yl)piperazin-1-carboxylate (6 g, 12.5 mmol) and N,N-dimethylpiperidin-4-amine (1.9 g, 15.0 mmol) were dissolved in anhydrous N,N-dimethylformamide (60 mL), then N,N-diisopropylethylamine (4.8 g, 37.5 mmol) and potassium iodide (0.21 g, 1.25 mmol) were added. After warming up to 60° C. the reaction was stirred for 2 h. After the reaction solution was cooled to room temperature, it was poured into ice water slowly and filtered. The filter cake was washed with water and little acetonitrile. The filtration was dried and dried by rotary evaporation to obtain tert-butyl 4-(7-bromo-6-chloro-2-(4-(dimethylamino)piperidin-1-yl)-8-fluoroquinazolin-4-yl)piperazin-1-carboxylate (4.8 g, Y: 67.1%, as a yellow solid). ES-API: [M+1]⁺=571.2

step 2: sodium hydride (1.4 g, 35.0 mmol) was suspended in dry DMF (20 mL) and after cooling to 0° C. cyclopropanol (305 mg, 5.2 mmol) was added. The reaction was stirred at 0° C. for 30 min, then a solution of tert-butyl 4-(7-bromo-6-chloro-2-(4-(dimethylamino)piperidin-1-yl)-8-fluoroquinazolin-4-yl)piperazin-1-carboxylate (2.0 g, 3.5 mmol) in N,N-dimethylformamide (2 mL) was added dropwise. After slowly warming up to 50° C., the reaction was stirred for 2.5 h. The reaction solution was quenched with water, extracted with ethyl acetate and concentrated under reduced pressure to give a crude which was purified by column chromatography (petroleum ether/ethyl acetate=3/7) to obtain tert-butyl 4-(7-bromo-6-chloro-8-cyclopropoxy-2-(4-(dimethylamino)piperidin-1-yl)quinazolin-4-yl)piperazin-1-carboxylate (1.3 g, Y: 61.0%, as a yellow solid). ES-API: [M+1]⁺=609.2

step 3: tert-butyl 4-(7-bromo-6-chloro-8-cyclopropoxy-2-(4-(dimethylamino)piperidin-1-yl) quinazolin-4-yl)piperazin-1-carboxylate (650 mg, 1.07 mmol), (5-methyl-1H-indazol-4-yl) boronic acid (281 mg, 1.60 mmol), tris (dibenzylideneindenacetone)dipalladium (97.5 mg, 0.11 mmol), potassium phosphate (1.1 g, 5.36 mmol) and 2-dicyclohexylphosphorus-2′,6′-diisopropoxy-1,1′-biphenyl (100 mg, 0.22 mmol) were dissolved in dioxane/water (8 mL/2 mL). Under nitrogen protection the reaction was stirred at 100° C. for 16 h. The reaction solution was extracted with ethyl acetate and concentrated under reduced pressure to give a crude which was purified by column chromatography (ethyl acetate/methanol=9/1) to obtain tert-butyl 4-(6-chloro-8-cyclopropoxy-2-(4-(dimethylamino)piperidin-1-yl)-7-(5-methyl-1H-indazol-4-yl)quinazolin-4-yl) piperazin-1-carboxylate (380 mg, Y: 54.0%, as a pale yellow oil). ES-API: [M+1]⁺=661.2

step 4: tert-butyl 4-(6-chloro-8-cyclopropoxy-2-(4-(dimethylamino)piperidin-1-yl)-7-(5-methyl-1H-indazol-4-yl)quinazolin-4-yl) piperazin-1-carboxylate (380 mg, 0.57 mmol) was dissolved in dichloromethane (5 mL) and then trifluoroacetic acid (1 mL) was added. The reaction was stirred at room temperature for 2 h. The reaction solution was directly concentrated under reduced pressure to obtain crude 1-(6-chloro-8-cyclopropoxy-7-(5-methyl-1H-indazol-4-yl)-4-(piperazin-1-yl)quinazolin-2-yl)-N,N-dimethylpiperidin-4-amine (322 mg, Y: crude, as a yellow solid), which was used directly in the next step. ES-API: [M+1]⁺=561.3

step 5: 1-(6-chloro-8-cyclopropoxy-7-(5-methyl-1H-indazol-4-yl)-4-(piperazin-1-yl) quinazolin-2-yl)-N,N-dimethylpiperidin-4-amine (322 mg, 0.57 mmol) was dissolved in dichloromethane (10 mL) and after cooling to 0° C. triethylamine (5 mL) and acrylic anhydride (65 mg, 0.52 mmol) were added dropwise. The reaction was stirred at 0° C. for 30 min. The reaction solution was extracted with dichloromethane and concentrated under reduced pressure to give a crude which was purified by reverse-phase preparation to obtain 1-(4-(6-chloro-8-cyclopropoxy-2-(4-(dimethylamino)piperidin-1-yl)-7-(5-methyl-1H-indazol-4-yl)quinazolin-4-yl)piperazin-1-yl)prop-2-en-1-one (49.5 mg, Y: 13.9%, a white solid). The preparation and separation conditions were the same as in Example 34. ES-API: [M+1]⁺=615.2. ¹HNMR (400 MHz, CDCl₃)δ 10.01 (s, 1H), 7.63 (d, J=6.3 Hz, 2H), 7.45 (d, J=8.6 Hz, 1H), 7.34 (d, J=8.5 Hz, 1H), 6.63 (dd, J=16.8, 10.5 Hz, 1H), 6.37 (d, J=16.7 Hz, 1H), 5.77 (d, J=10.5 Hz, 1H), 4.95 (d, J=13.4 Hz, 2H), 4.28 (s, 1H), 3.94 (s, 2H), 3.83 (s, 2H), 3.73 (d, J=5.1 Hz, 4H), 2.93 (t, J=12.8 Hz, 2H), 2.36 (s, 6H), 2.22 (s, 3H), 1.51 (s, 4H), 0.22 (t, J=24.2 Hz, 4H).

### Example 35 Preparation of Z35

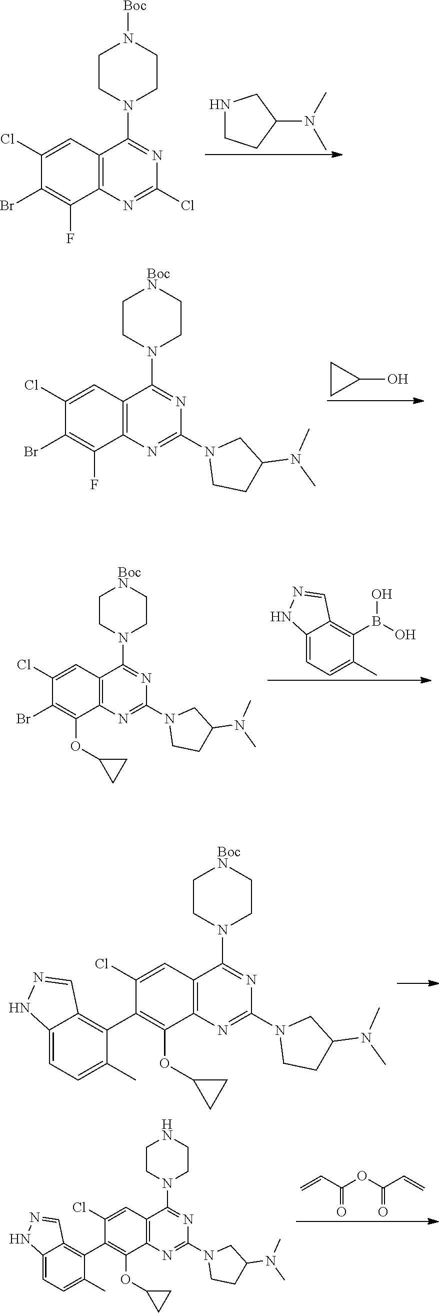

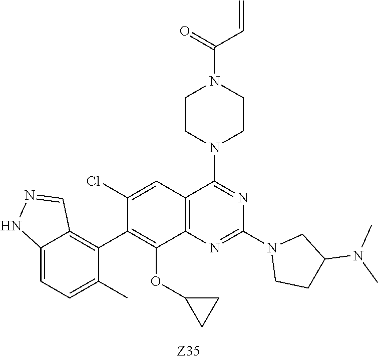 [SMILES: C=CC(=O)N1CCN(c2nc(N3CCC(N(C)C)C3)nc3c(OC4CC4)c(-c4c(C)ccc5[nH]ncc45)c(Cl)cc23)CC1]

step 1: tert-butyl 4-(7-bromo-2,6-dichloro-8-fluoroquinazolin-4-yl)piperazin-1-carboxylate (1 g, 2.0 mmol) was added to DMF (20 mE) and then N,N-dimethylpyrrolidin-3-amine (342 mg, 3.0 mmol) and cesium carbonate (1.8 g, 6.0 mmol) were added. The mixture was heated to 80° C. and the reaction was conducted for 2 h. It was detected by LCMS till the reaction was complete. After the reaction solution was cooled to room temperature, 50 mL of water was added, and the mixture was extracted with ethyl acetate (50 mL*3). The organic phase was dried and dried by rotary evaporation to obtain a crude which was purified by flash column on silica gel (DCM/MeOH=100/1) to obtain 1.1 g of product tert-butyl 4-(7-bromo-6-chloro-2-(3-(dimethylamino)pyrrolidin-1-yl)-8-fluoroquinazolin-4-yl)piperazin-1-carboxylate (Y: 88%). ES-API: [M+1]⁺=557.1

step 2: cyclopropanol (240 mg, 4.0 mmol) was dissolved in tetrahydrofuran (20 mL) and at 0° C. sodium hydrogen (650 mg, 26.9 mmol) was added. The reaction was stirred for 0.5 h while keeping the temperature. tert-butyl 4-(7-bromo-6-chloro-2-(3-(dimethylamino)pyrrolidin-1-yl)-8-fluoroquinazolin-4-yl)piperazin-1-carboxylate (1.5 g, 2.69 mmol) was then added into the reaction solution. The reaction was conducted for 1 h while keeping the temperature. The reaction solution was poured into ethyl acetate (50 mL), and the organic phase was washed with water (50 mL×2) and washed with brine (50 mL), dried over anhydrous sodium sulfate and filtered. The filtration was dried by rotary evaporation to obtain a yellow oil. The crude was purified by flash column on silica gel (DCM/MeOH=100/1) to obtain tert-butyl 4-(7-bromo-6-chloro-8-cyclopropoxy-2-(3-(dimethylamino)pyrrolidin-1-yl) quinazolin-4-yl)piperazin-1-carboxylate (1.1 g, Y: 63%). ES-API: [M+1]⁺=595.1

step 3: tert-butyl (4-(7-bromo-6-chloro-8-cyclopropoxy-2-(3-(dimethylamino)pyrrolidin-1-yl)quinazolin-4-yl)piperazin-1-carboxylate (1.1 g, 1.85 mmol), (5-methyl-1H-indazol-4-yl) boronic acid (488 mg, 2.78 mmol), tripotassium phosphate (1.96 g, 9.25 mmol), tris (dibenzylideneindenacetone)dipalladium (170 mg, 0.185 mmol), 2-dicyclohexylphosphine-2′,4′,6′-triisopropylbiphenyl (90 mg, 0.185 mmol) were added in sequence into dioxane (8 mL) and water (2 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once it was purified by column on silica gel (methanol:dichloromethane=0˜1:20) to obtain tert-butyl 4-(6-chloro-8-cyclopropoxy-2-(3-(dimethylamino)pyrrolidin-1-yl)-7-(5-methyl-TH-indazol-4-yl)quinazolin-4-yl) piperazin-1-carboxylate (800 mg, Y: 66%) as a yellow solid. ES-API [M+1]⁺=647.1

step 4: tert-butyl 4-(6-chloro-8-cyclopropoxy-2-(3-(dimethylamino)pyrrolidin-1-yl)-7-(5-methyl-TH-indazol-4-yl)quinazolin-4-yl)piperazin-1-carboxylate (800 mg, 1.24 mmol) was dissolved in dichloromethane (8 mL) and then trifluoroacetic acid (3 mL) was added. The reaction was conducted at room temperature for 1 h and the reaction mixture was dried by rotary evaporation to obtain crude 1-(6-chloro-8-cyclopropoxy-7-(5-methyl-1H-indazol-4-yl)-4-(piperazin-1-yl)quinazolin-2-yl)-N,N-dimethylpyrrolidin-3-amine (800 mg, Y: 100%) as a yellow oil.

step 5: 1-(6-chloro-8-cyclopropoxy-7-(5-methyl-1H-indazol-4-yl)-4-(piperazin-1-yl) quinazolin-2-yl)-N,N-dimethylpyrrolidin-3-amine (800 mg, 1.24 mmol) and triethylamine (3 mL) were dissolved in dichloromethane (8 mL). At 0° C. acrylic anhydride (140 mg, 1.1 mmol) was added, and the reaction was conducted at 0° C. for 0.5 h. 20 mL of water and DCM (20 mL*3) were added to the reaction solution, which was extracted and the organic phase was dried by rotary evaporation to obtain 780 mg of crude, which was purified to obtain 1-(4-(6-chloro-8-cyclopropoxy-2-(3-(dimethylamino)pyrrolidin-1-yl)-7-(5-methyl-1H-indazol-4-yl) quinazolin-4-yl)piperazin-1-yl)prop-2-en-1-one (21 mg, Y: 2.7%). The preparation and separation conditions were the same as in Example 10. ES-API: [M+1]⁺=601.2. ¹HNMR (400 MHz, CDCl₃)δ 10.17 (s, 0H), 7.63 (d, J=7.3 Hz, 2H), 7.44 (d, J=8.4 Hz, 1H), 7.33 (d, J=8.6 Hz, 1H), 6.62 (dd, J=16.6, 10.5 Hz, 1H), 6.36 (d, J=16.8 Hz, 1H), 5.77 (d, J=10.6 Hz, 1H), 4.35 (s, 1H), 4.10-3.67 (m, 11H), 3.59 (d, J=12.6 Hz, 1H), 3.51-3.34 (m, 1H), 2.87 (s, 1H), 2.35 (s, 6H), 2.22 (s, 4H), 0.37-0.05 (m, 4H).

### Example 36 Preparation of Z36

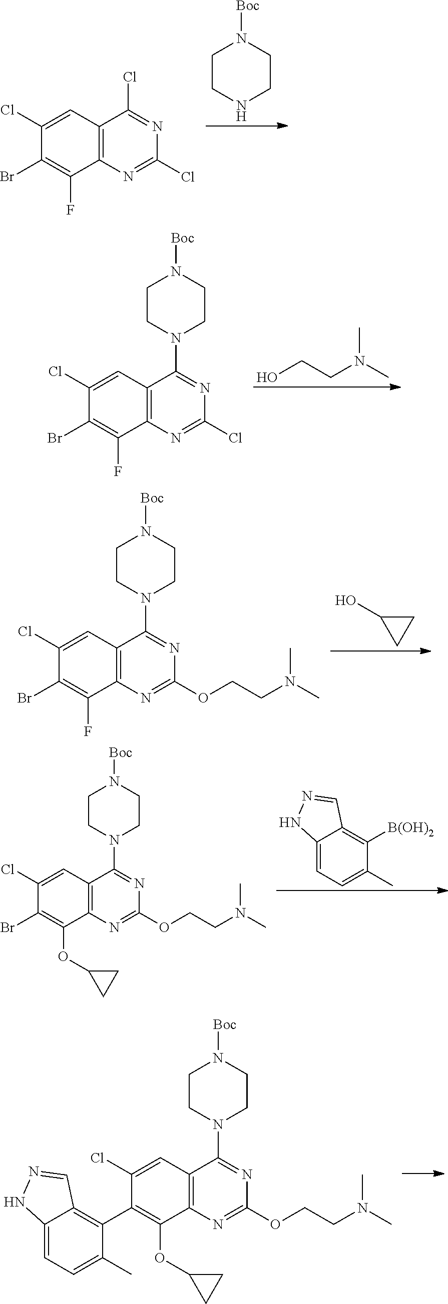

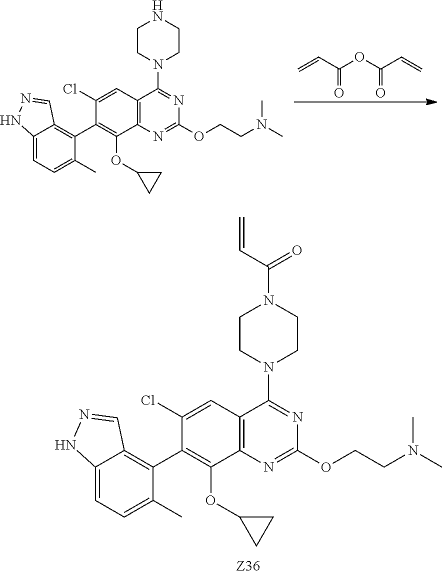

step 1: 7-bromo-2,4,6-trichloro-8-fluoroquinazoline (10 g, 30.3 mmol) was added to DCM/ACN(75/75 mL). The reaction solution was cooled to 0° C. and then TEA (15.3 g, 227.5 mmol) was added slowly. After that tert-butyl piperazin-1-carboxylate (6.2 g, 33.3 mmol) was added. The reaction solution was slowly warmed up to room temperature and then stirred for 2 h. It was detected by LCMS till the reaction was complete. DCM (100 mL*3) and water (200 mL) were added, which was extracted and the organic phase was dried and dried by rotary evaporation to obtain a brown solid, which was beat with AcN(50 mL) to obtain 12 g of tert-butyl 4-(7-bromo-2,6-dichloro-8-fluoroquinazolin-4-yl)piperazin-1-carboxylate (Y: 82.7%) as a white solid. ES-API: [M+1]⁺=481.0.

step 2: to a mixed solution of tert-butyl 4-(7-bromo-2,6-dichloro-8-fluoroquinazolin-4-yl) piperazin-1-carboxylate (6 g, 12.5 mmol) in N,N′-dimethylformamide (60 mL) cesium carbonate (12 g, 37.5 mmol) and N, N-dimethylethanolamine (1.3 g, 15 mmol) were added. Then the mixture was stirred at 80° C. for 2 h. The reaction mixture was poured into ice water (100 mL) and extracted with ethyl acetate (3×100 mL). The combined organic phase was washed with water (100 mL) and washed with saturated brine (100 mL) and dried over anhydrous sodium sulfate. The solvent was removed by rotary evaporation. The residue was purified by flash chromatography (eluting with dichloromethane/methanol=50/1) to obtain tert-butyl 4-(7-bromo-6-chloro-2-(2-(dimethylamino)ethoxy)-8-fluoroquinazolin-4-yl)piperazin-1-carboxylate (5.6 g, 71.8%) as a yellow solid. MS: m/z=532.1 (M+1, ESI).

step 3: at 0° C., to a mixed solution of cyclopropanol (912 mg, 15.7 mmol) in tetrahydrofuran (50 mL) sodium hydride (2.5 g, 105 mmol) was added. The obtained mixture was stirred at 0° C. for 0.5 h. tert-butyl 4-(7-bromo-6-chloro-2-(2-(dimethylamino)ethoxy)-8-fluoroquinazolin-4-yl) piperazin-1-carboxylate (5.6 g, 10.5 mmol) was added to the reaction mixture. Then the reaction was conducted at room temperature for 1 h. The reaction mixture was poured into ice water (100 mL) and extracted with dichloromethane (3×100 mL). The combined organic phase was washed with saturated ammonium chloride (100 mL) and washed with saturated brine (100 mL). The solvent was removed by rotary evaporation. The residue was purified by flash chromatography (eluting with dichloromethane/methanol=50/1) to obtain tert-butyl 4-(7-bromo-6-chloro-8-cyclopropoxy-2-(2-(dimethylamino)ethoxy)quinazolin-4-yl)piperazin-1-carboxylate (4.1 g, 68%) as a yellow solid. MS: m/z=570.2 (M+1, ESI).

step 4: to a suspension of tert-butyl 4-(7-bromo-6-chloro-8-cyclopropoxy-2-(2-(dimethylamino)ethoxy)quinazolin-4-yl)piperazin-1-carboxylate (4.1 g, 7.2 mmol), (5-methyl-TH-indazol-4-yl) boronic acid (2.5 mg, 14.4 mmol), potassium phosphate (4.6 g, 21.6 mmol) in 1,4-dioxane (30 mL) and deionized water (8 mL) tris (dibenzylideneindenacetone)dipalladium (700 mg, 0.72 mmol) and dicyclohexylphosphorus-2,6-diisopropoxy 1,1″-biphenyl (350 mg, 0.72 mmol) were added. The resulting mixture was heated to 120° C. and the reaction was conducted for 16 h. After cooling to room temperature, the mixture was extracted with ethyl acetate (3×100 mL). The combined organic phase was washed with saturated brine (100 mL) and dried over anhydrous sodium sulfate. The solvent was removed by rotary evaporation. The residue was purified by flash chromatography (eluting with dichloromethane/methanol=50/1) to obtain tert-butyl 4-(6-chloro-8-cyclopropoxy-2-(2-(dimethylamino)ethoxy)-7-(5-methyl-TH-indazol-4-yl)quinazolin-4-yl)piperazin-1-carboxylate (1.9 g, 43%) as a yellow solid. MS: m/z=608.3 (M+1, ESI).

step 5: to a solution of tert-butyl 4-(6-chloro-8-cyclopropoxy-2-(2-(dimethylamino)ethoxy)-7-(5-methyl-1H-indazol-4-yl)quinazolin-4-yl)piperazin-1-carboxylate (1.9 g, 3.0 mmol) in dichloromethane (15 mL) was added trifluoroacetic acid (4 mL). The obtained mixture was stirred at room temperature for 30 min. The solvent was removed by rotary evaporation to obtain 2-((6-chloro-8-cyclopropoxy-7-(5-methyl-TH-indazol-4-yl)-4-(piperazin-1-yl)quinazolin-2-yl)oxy)-N,N-dimethylethylamine (1.5 g, 100%) as a yellow oil, which was used directly in the next step without further purification. MS: m/z=508.3 (M+1, ESI).

step 6: 2-((6-chloro-8-cyclopropoxy-7-(5-methyl-TH-indazol-4-yl)-4-(piperazin-1-yl) quinazolin-2-yl)oxy)-N,N-dimethylethylamine (1.5 g, 3.0 mmol) was dissolved in dichloromethane (15 mL). After the mixture was cooled to 0° C., triethylamine (5 mL) was added dropwise slowly. At 0° C. acrylic anhydride (310 mg, 2.4 mmol) was added and stirred for 1 h. Then the solvent was removed under reduced pressure. The residue was purified by Prep-HPLC to obtain a white solid (330 mg, 20%). The preparation and separation conditions were the same as in Example 10. MS: m/z=576.2 (M+1, ESI). ¹HNMR (400 MHz, CDCl₃)δ 10.47 (s, 1H), 7.75 (s, 1H), 7.58 (s, 1H), 7.47 (d, J=8.5 Hz, 1H), 7.33 (d, J=8.6 Hz, 1H), 6.61 (dd, J=16.7, 10.6 Hz, 1H), 6.37 (d, J=16.9 Hz, 1H), 5.77 (d, J=10.6 Hz, 1H), 4.63 (t, J=5.9 Hz, 2H), 4.31 (s, 1H), 3.89 (d, J=29.4 Hz, 8H), 2.91 (d, J=6.1 Hz, 2H), 2.42 (s, 6H), 2.20 (s, 3H), 0.32-0.13 (m, 4H).

### Example 37 Preparation of Z37

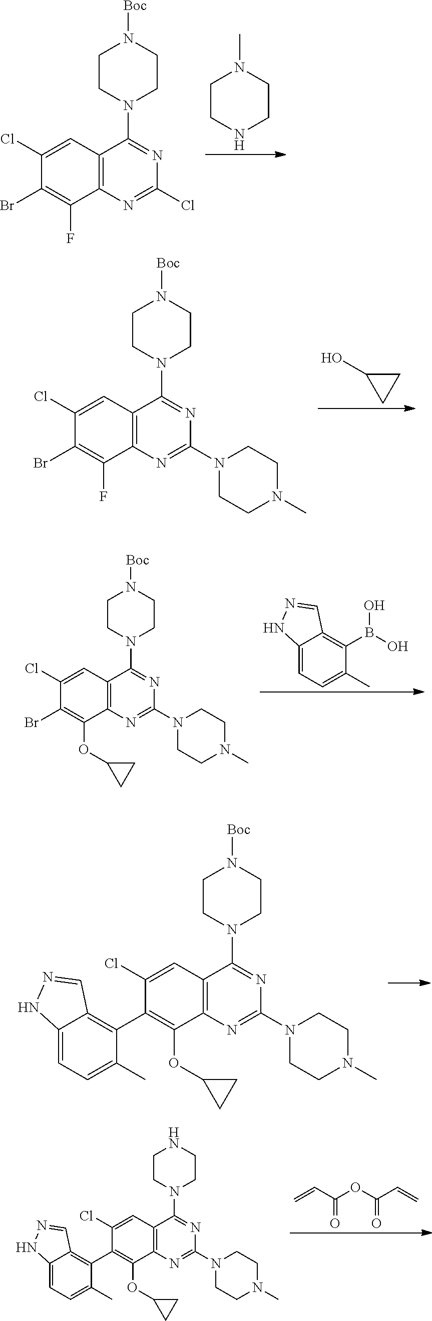

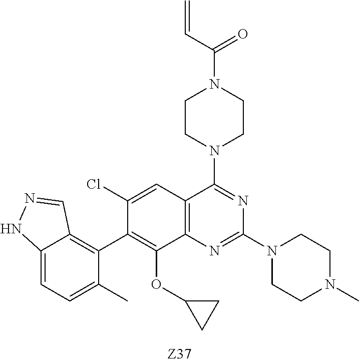 [SMILES: C=CC(=O)N1CCN(c2nc(N3CCN(C)CC3)nc3c(OC4CC4)c(-c4c(C)ccc5[nH]ncc45)c(Cl)cc23)CC1]

step 1: at 0° C., to a mixed solution of tert-butyl 4-(7-bromo-2,6-dichloro-8-fluoroquinazolin-4-yl)piperazin-1-carboxylate (3.0 g, 6.25 mmol) and N,N′-diisopropylethylamine (2.42 g, 18.74 mmol) in dichloromethane (30 mL) and acetonitrile (30 mL)N-methyl piperazine (626 mg, 6.25 mmol) was added. Then the mixture was stirred at room temperature overnight. Ice water (200 mL) was added and the mixture was extracted with dichloromethane (3×80 mL). The combined organic phase was washed with saturated brine (3×80 mL) and dried over anhydrous sodium sulfate. The solvent was removed by rotary evaporation. The residue was triturated with dichloromethane and petroleum ether and filtered with suction to obtain tert-butyl 4-(7-bromo-6-chloro-8-fluoro-2-(4-methylpiperazin-1-yl)quinazolin-4-yl)piperazin-1-carboxylate (2.7 g, Y: 79.46%) as a yellow solid, which was used directly in the next step without further purification. MS: m/z=543.1 (M+1, ESI).

step 2: at 0° C., to a mixed solution of cyclopropanol (481 mg, 8.27 mmol) in tetrahydrofuran (30 mL) sodium hydride (552 mg, 13.79 mmol, the purity is 60%) was added. The obtained mixture was stirred at 0° C. for 1 h. tert-butyl 4-(7-bromo-6-chloro-8-fluoro-2-(4-methylpiperazin-1-yl)quinazolin-4-yl)piperazin-1-carboxylate (1.5 g, 2.76 mmol) was added to the reaction mixture. Then the reaction was conducted at room temperature overnight.

The reaction mixture was poured into ice water (300 mL) and extracted with dichloromethane (3×200 mL). The combined organic phase was washed with saturated ammonium chloride (3×100 mL) and washed with saturated brine (3×100 mL). The solvent was removed by rotary evaporation. The residue was purified by flash chromatography, eluting with dichloromethane and methanol (3%), to obtain tert-butyl 4-(7-bromo-6-chloro-8-cyclopropoxy-2-(4-methylpiperazin-1-yl)quinazolin-4-yl)piperazin-1-carboxylate (1.0 g, Y: 62.31%), as yellow oil. MS: m/z=581.2 (M+1, ESI).

step 3: to a suspension of tert-butyl 4-(7-bromo-6-chloro-8-cyclopropoxy-2-(4-methylpiperazin-1-yl)quinazolin-4-yl)piperazin-1-carboxylate (0.8 g, 1.37 mmol), (5-methyl-1H-indazol-4-yl) boronic acid (484 mg, 2.75 mmol), potassium phosphate (584 mg, 2.75 mmol) in 1,4-dioxane (10 mL) and deionized water (2.5 mL) tris (dibenzylideneindenacetone)dipalladium (252 mg, 0.27 mmol) and dicyclohexylphosphorus-2,6-diisopropoxy 1,1″-biphenyl (128 mg, 0.27 mmol) were added. The resulting mixture was heated to 100° C. and stirred under an argon atmosphere for 6.5 h. After cooling to room temperature, the reaction mixture was extracted with dichloromethane (3×150 mL). The combined organic phase was washed with water (3×50 mL) and washed with saturated brine (3×100 mL) and dried over anhydrous sodium sulfate. The solvent was removed by rotary evaporation. The residue was purified by flash chromatography, eluting with dichloromethane and methanol (3%), to obtain tert-butyl 4-(6-chloro-8-cyclopropoxy-7-(5-methyl-1H-indazol-4-yl)-2-(4-methylpiperazin-1-yl)quinazolin-4-yl)piperazin-1-carboxylate (376 mg, Y: 43.20%), as a colorless oily liquid. MS: m/z=633.3 (M+1, ESI).

step 4: to a solution of tert-butyl 4-(6-chloro-8-cyclopropoxy-7-(5-methyl-1H-indazol-4-yl)-2-(4-methylpiperazin-1-yl)quinazolin-4-yl)piperazin-1-carboxylate (375 mg, 0.59 mmol) in dichloromethane (5 mL) trifluoroacetic acid (2 mL) was added. The obtained mixture was stirred at room temperature for 30 min. The pH of the reaction mixture was adjusted to 8 with 1M sodium bicarbonate. The reaction mixture was extracted with dichloromethane (3×30 mL). The combined organic phase was washed with saturated brine (3×30 mL) and dried over anhydrous sodium sulfate. The solvent was removed by rotary evaporation to obtain 6-chloro-8-cyclopropoxy-7-(5-methyl-1H-indazol-4-yl)-2-(4-methylpiperazin-1-yl)-4-(piperazin-1-yl)quinazoline (316 mg, Y: 100%) as a yellow oil, which was used directly in the next step without further purification. MS: m/z=533.3 (M+1, ESI).

step 5: to a solution of 6-chloro-8-cyclopropoxy-7-(5-methyl-1H-indazol-4-yl)-2-(4-methylpiperazin-1-yl)-4-(piperazin-1-yl)quinazoline (316 mg, 0.593 mmol) in dichloromethane (5 mL) was added triethylamine (180 mg, 1.78 mmol). The mixture was cooled to 0° C. At 0° C. acrylic anhydride (74 mg, 0.593 mmol) was added and stirred for 1 h. Then the solvent was removed under reduced pressure. The residue was purified by Thick-TLC to obtain Z37 (20.2 mg, Y: 4.83%) as a white solid. MS: m/z=587.2 (M+1, ESI). ¹H NMR (400 MHz, CDCl₃) δ 10.39 (s, 1H), 7.62 (d, J=14.8 Hz, 2H), 7.45 (d, J=8.4 Hz, 1H), 7.33 (d, J=8.7 Hz, 1H), 6.62 (dd, J=16.6, 10.4 Hz, 1H), 6.37 (d, J=16.8 Hz, 1H), 5.77 (d, J=10.5 Hz, 1H), 4.37-4.21 (m, 1H), 3.97 (d, J=28.8 Hz, 6H), 3.89-3.64 (m, 6H), 2.61 (t, J=5.0 Hz, 4H), 2.43 (d, J=7.6 Hz, 3H), 2.21 (s, 3H), 0.41-0.06 (m, 4H).

### Example 38 Preparation of Z38

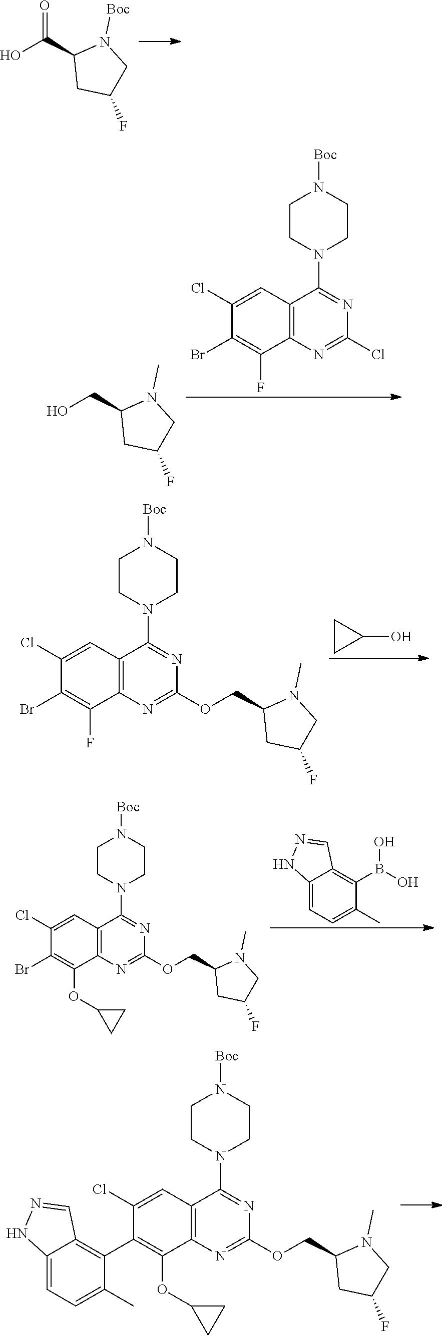

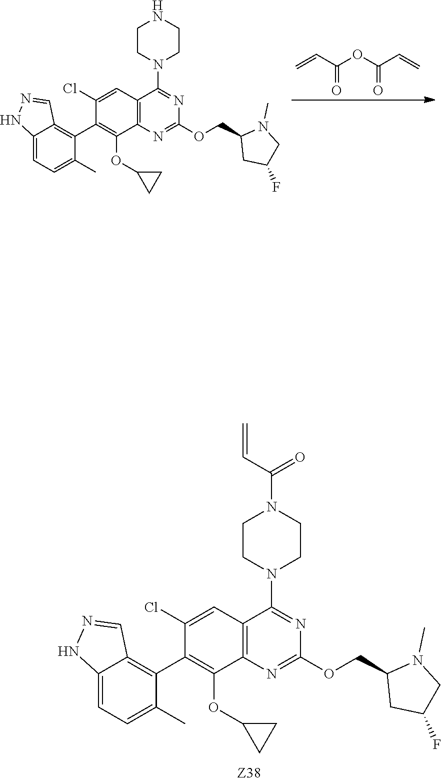

step 1: (2S, 4R)-1-(tert-butoxycarbonyl)-4-fluoropyrrolidin-2-carboxylic acid (4 g, 17.2 mmol) was dissolved in tetrahydrofuran (80 mL) and then lithium aluminum hydride (1.96 g, 51.6 mmol) was added. The reaction was stirred at room temperature for 16 h. The reaction was quenched by adding sodium sulfate decahydrate and filtered. The filtration was concentrated to dryness to obtain 2.1 g of crude ((2S, 4R)-4-fluoro-1-methylpyrrolidin-2-yl) methanol (Y: 90%), which was used directly in the next step. ES-API: [M+1]⁺=134.1

step 2: ((2S, 4R)-4-fluoro-1-methylpyrrolidin-2-yl) methanol (2 g, 4.16 mmol) was dissolved in N, N-dimethylformamide (10 mL), then tert-butyl 4-(7-bromo-2,6-dichloro-8-fluoroquinazolin-4-yl)piperazin-1-carboxylate (1.65 g, 12.48 mmol), triethylene diamine (0.126 g, 0.832 mmol) and cesium carbonate (2.7 g, 8.32 mmol) were added. The reaction was conducted at room temperature for 16 h. The mixture was separated by adding water and ethyl acetate. The organic phase was washed with water and washed with brine and dried over anhydrous sodium sulfate. The solvent was concentrated under reduced pressure to give a crude which was purified by column chromatography to obtain 1 g of tert-butyl 4-(7-bromo-6-chloro-8-fluoro-2-((((2S, 4R)-4-fluoro-1-methylpyrrolidin-2-yl)methoxy)quinazolin-4-yl)piperazin-1-carboxylate. (Y: 41.8%). ES-API: [M+1]⁺=576.1

step 3: tert-butyl 4-(7-bromo-6-chloro-8-fluoro-2-((((2S, 4R)-4-fluoro-1-methylpyrrolidin-2-yl)methoxy)quinazolin-4-yl)piperazin-1-carboxylate (1 g, 1.73 mmol), cyclopropanol (201 mg, 3.46 mmol) was dissolved in tetrahydrofuran (20 mL), and at 0° C. sodium hydrogen (692 mg, 17.3 mmol) was added in batches. The reaction was stirred for 2 h while keeping the temperature. The reaction was quenched by adding water, and the mixture was extracted with ethyl acetate. The organic phase was washed with brine, dried over anhydrous sodium sulfate and dried by rotary evaporation under reduced pressure to give a crude, which was purified by column chromatography (methanol:dichloromethane=1:10) to obtain tert-butyl 4-(7-bromo-6-chloro-8-cyclopropoxy-2-((((2S, 4R)-4-fluoro-1-methylpyrrolidin-2-yl)methoxy)quinazolin-4-yl)piperazin-1-carboxylate (790 mg, Y: 65%). ES-API: [M+1]⁺=614.1

step 4: tert-butyl 4-(7-bromo-6-chloro-8-cyclopropoxy-2-((((2S, 4R)-4-fluoro-1-methylpyrrolidin-2-yl)methoxy)quinazolin-4-yl)piperazin-1-carboxylate (690 mg, 1.12 mmol), (5-methyl-TH-indazol-4-yl) boronic acid (394 m g, 2.24 mmol), potassium phosphate (712 mg, 3.36 mmol), Ruphos (104 mg, 0.224 mmol) and Pd₂(dba)₃ (102 mg, 0.112 mmol) were added in sequence into dioxane (10 mL) and water (2 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once, it was dried over anhydrous sodium sulfate and dried by rotary evaporation under reduced pressure and purified by column to obtain tert-butyl 4-(6-chloro-8-cyclopropoxy-2-((((2S, 4R)-4-fluoro-1-methylpyrrolidin-2-yl)methoxy)-7-(5-methyl-TH-indazol-4-yl)quinazolin-4-yl)piperazin-1-carboxylate (450 mg, Y: 60.4%). ES-API: [M+1]⁺=666.2.

step 5: tert-butyl 4-(6-chloro-8-cyclopropoxy-2-((((2S, 4R)-4-fluoro-1-methylpyrrolidin-2-yl) methoxy)-7-(5-methyl-1H-indazol-4-yl)quinazolin-4-yl)piperazin-1-carboxylate (450 mg, 0.79 mmol) was dissolved in dichloromethane (5 mL) and then trifluoroacetic acid (2 mL) was added. The reaction was conducted at room temperature for 1 h and the reaction mixture was dried by rotary evaporation to obtain crude 6-chloro-8-cyclopropoxy-2-((((2S, 4R)-4-fluoro-1-methylpyrrolidin-2-yl)methoxy)-7-(5-methyl-1H-indazol-4-yl)-4-(piperazin-1-yl)quinazoline (500 mg, Y: 100%). ES-API: [M+1]⁺=566.2.

step 6: 6-chloro-8-cyclopropoxy-2-((((2S, 4R)-4-fluoro-1-methylpyrrolidin-2-yl)methoxy)-7-(5-methyl-1H-indazol-4-yl)-4-(piperazin-1-yl)quinazoline (500 mg, 0.67 mmol) and triethylamine (203 mg) were dissolved in dichloromethane (5 mL). At 0° C. acrylic anhydride (75 mg, 0.6 mmol) was added, and the reaction was conducted at 0° C. for 0.5 h. The reaction solution was extracted by adding water and DCM. The organic phase was washed with brine, dried over anhydrous sodium sulfate, dried by rotary evaporation under reduced pressure and purified by preparation to obtain 1-(4-(6-chloro-8-cyclopropoxy-2-((((2S, 4R)-4-fluoro-1-methylpyrrolidin-2-yl)methoxy)-7]-(5-methyl-1H-indazol-4-yl)quinazolin-4-yl)piperazin-1-yl)prop-2-en-1-one (112 mg, Y: 27%). The preparation and separation conditions were the same as in Example 10. ES-API: [M+1]⁺=620.2. ¹HNMR (400 MHz, CDCl₃)δ 10.52 (s, 1H), 7.76 (s, 1H), 7.59 (s, 1H), 7.47 (d, J=8.5 Hz, 1H), 7.35 (s, 1H), 6.62 (dd, J=16.8, 10.5 Hz, 1H), 6.38 (d, J=16.7 Hz, 1H), 5.78 (d, J=10.5 Hz, 1H), 5.24 (s, 1H), 4.66-4.57 (m, 1H), 4.47-4.30 (m, 2H), 3.90 (d, J=31.0 Hz, 8H), 3.56 (ddd, J=25.1, 12.0, 5.3 Hz, 1H), 3.11 (dt, J=11.0, 5.1 Hz, 1H), 2.61 (dd, J=32.5, 11.8 Hz, 2H), 2.52 (s, 3H), 2.32 (ddd, J=22.4, 14.3, 5.9 Hz, 1H), 2.20 (s, 3H), 2.02 (dd, J=28.2, 10.8 Hz, 2H), 0.20 (ddq, J=20.4, 14.2, 8.4, 7.3 Hz, 4H).

### Example 39 Preparation of Z39

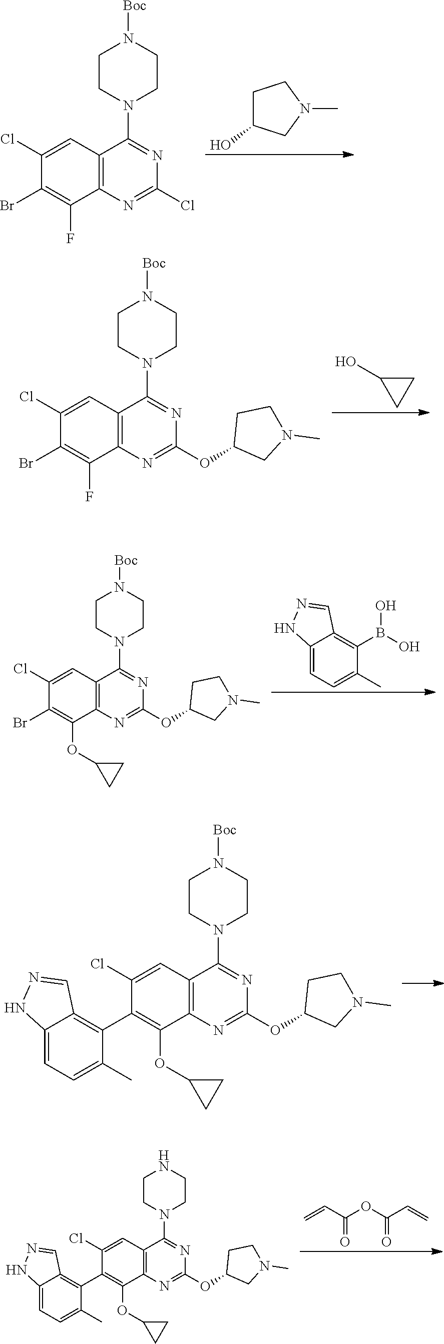

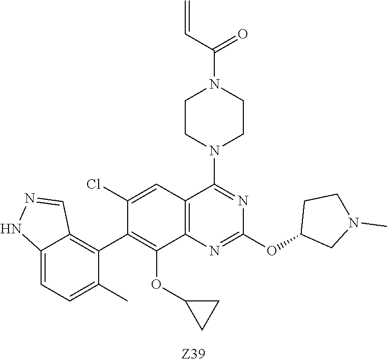 [SMILES: C=CC(=O)N1CCN(c2nc(O[C@@H]3CCN(C)C3)nc3c(OC4CC4)c(-c4c(C)ccc5[nH]ncc45)c(Cl)cc23)CC1]

step 1: to a mixed solution of tert-butyl 4-(7-bromo-2,6-dichloro-8-fluoroquinazolin-4-yl) piperazin-1-carboxylate (18.7 g, 38.95 mmol) in N,N′-dimethylformamide (150 mL) cesium carbonate (38.07 g, 116.84 mmol), triethylene diamine (873 mg, 7.79 mmol) and (R)3-hydroxy-1-methyltetrahydropyrrole (1.19 g, 11.75 mmol) were added. Then the reaction was conducted at room temperature for 4 h. The reaction mixture was poured into ice water (1500 mL) and extracted with ethyl acetate (3×500 mL). The combined organic phase was washed with water (3×300 mL) and washed with saturated brine (3×200 mL) and dried over anhydrous sodium sulfate. The solvent was removed by rotary evaporation. The residue was purified by flash chromatography, eluting with dichloromethane and methanol (6%), to obtain tert-butyl (R)-4-(7-bromo-6-chloro-8-fluoro-2-((1-methylpyrrolidin-3-yl)oxy)quinazolin-4-yl)piperazin-1-carboxylate (9.6 g, Y: 45%) as a yellow oil. MS: m/z=544.1 (M+1, ESI).

step 2: at 0° C., to a mixed solution of cyclopropanol (3.07 g, 17.62 mmol) in tetrahydrofuran (30 mL) sodium hydride (3.52 g, 88.10 mmol, the purity is 60%) was added. The obtained mixture was stirred at 0° C. for 1 h. tert-butyl (R)-4-(7-bromo-6-chloro-8-fluoro-2-((1-methylpyrrolidin-3-yl)oxy)quinazolin-4-yl)piperazin-1-carboxylate (9.6 g, 17.62 mmol) was added to the reaction mixture. Then the reaction was conducted at room temperature for 4 h. The reaction mixture was poured into ice water (500 mL) and extracted with dichloromethane (3×200 mL). The combined organic phase was washed with saturated ammonium chloride (3×100 mL) and washed with saturated brine (3×100 mL). The solvent was removed by rotary evaporation. The residue was purified by flash chromatography, eluting with dichloromethane and methanol (3%), to obtain tert-butyl (R)-4-(7-bromo-6-chloro-8-cyclopropoxy-2-((1-methylpyrrolidin-3-yl)oxy)quinazolin-4-yl)piperazin-1-carboxylate (7.1 g, Y: 69.13%) as a white solid. MS: m/z=582.1 (M+1, ESI).

step 3: to a suspension of tert-butyl (R)-4-(7-bromo-6-chloro-8-cyclopropoxy-2-((1-methylpyrrolidin-3-yl)oxy)quinazolin-4-yl)piperazin-1-carboxylate (7.1 g, 12.18 mmol), (5-methyl-1H-indazol-4-yl) boronic acid (4.29 g, 24.36 mmol) and potassium phosphate (5.17 g, 24.36 mmol) in 1,4-dioxane (70 mL) and deionized water (17.5 mL) tris (dibenzylideneindenacetone)dipalladium (2.23 g, 2.44 mmol) and dicyclohexylphosphorus-2,6-diisopropoxy 1,1″-biphenyl (1.14 g, 2.44 mmol) were added. The obtained mixture was heated to 100° C. and stirred overnight under an argon atmosphere. After cooling to room temperature, the reaction mixture was extracted with dichloromethane (3×200 mL). The combined organic phase was washed with water (3×100 mL) and washed with saturated brine (3×100 mL) and dried over anhydrous sodium sulfate. The solvent was removed by rotary evaporation. The residue was purified by flash chromatography, eluting with dichloromethane and methanol (3%), to obtain tert-butyl (R)-4-(6-chloro-8-cyclopropoxy-7-(5-methyl-1H-indazol-4-yl)-2-((1-methylpyrrolidin-3-yl)oxy)quinazolin-4-yl-1-carboxylate piperazine (2.3 g, Y: 29.78%) as a yellow solid. MS: m/z=634.3 (M+1, ESI).

step 4: to a solution of tert-butyl (R)-4-(6-chloro-8-cyclopropoxy-7-(5-methyl-1H-indazol-4-yl)-2-((1-methylpyrrolidin-3-yl)oxy)quinazolin-4-yl-1-carboxylate piperazine (1.1 g, 1.73 mmol) in dichloromethane (10 mL) trifluoroacetic acid (3.87 mL) was added. The obtained mixture was stirred at room temperature for 30 min. The pH of the reaction mixture was adjusted to 8 with 1M sodium bicarbonate. The reaction mixture was extracted with dichloromethane (3×50 mL). The combined organic phase was washed with saturated brine (3×30 mL), and dried over anhydrous sodium sulfate. The solvent was removed by rotary evaporation to obtain (R)-6-chloro-8-cyclopropoxy-7-(5-methyl-1H-indazol-4-yl)-2-((1-methylpyrrolidin-3-yl) oxy)-4-(piperazin-1-yl)quinazoline (0.7 g, Y: 75.57%) as a yellow oil, which was used directly in the next step without further purification. MS: m/z=534.2 (M+1, ESI).

step 5: to a solution of (R)-6-chloro-8-cyclopropoxy-7-(5-methyl-1H-indazol-4-yl)-2-((1-methylpyrrolidin-3-yl)oxy)-4-(piperazin-1-yl)quinazoline (0.7 g, 1.31 mmol) in dichloromethane (10 mL) triethylamine (398 mg, 3.93 mmol) was added. The mixture was cooled to 0° C. At 0° C. acrylic anhydride (165 mg, 1.31 mmol) was added and stirred for 1 h. Then the solvent was removed under reduced pressure. The residue was purified by flash chromatography, eluting with dichloromethane and methanol (3%), to obtain 1-(4-(6-chloro-8-cyclopropoxy-7-(5-methyl-1H-indazol-4-yl)-2-(((R)-1-methylpyrrolidin-3-yl)oxy) quinazolin-1-one (250 m g, Y: 32.43%) as a white solid. MS: m/z=588.2 (M+1, ESI). ¹HNMR (400 MHz, CDCl₃)δ 7.76 (s, 1H), 7.58 (s, 1H), 7.48 (d, J=8.5 Hz, 1H), 7.35 (d, J=8.5 Hz, 1H), 6.62 (dd, J=16.8, 10.5 Hz, 1H), 6.38 (dd, J=16.7, 1.8 Hz, 1H), 5.79 (dd, J=10.5, 1.8 Hz, 1H), 5.63 (s, 1H), 4.26 (d, J=4.0 Hz, 1H), 3.90 (d, J=28.6 Hz, 9H), 2.95 (s, 2H), 2.57 (s, 3H), 2.20 (s, 3H), 1.27 (d, J=11.0 Hz, 2H), 0.21 (ddd, J=14.6, 11.2, 7.0 Hz, 4H).

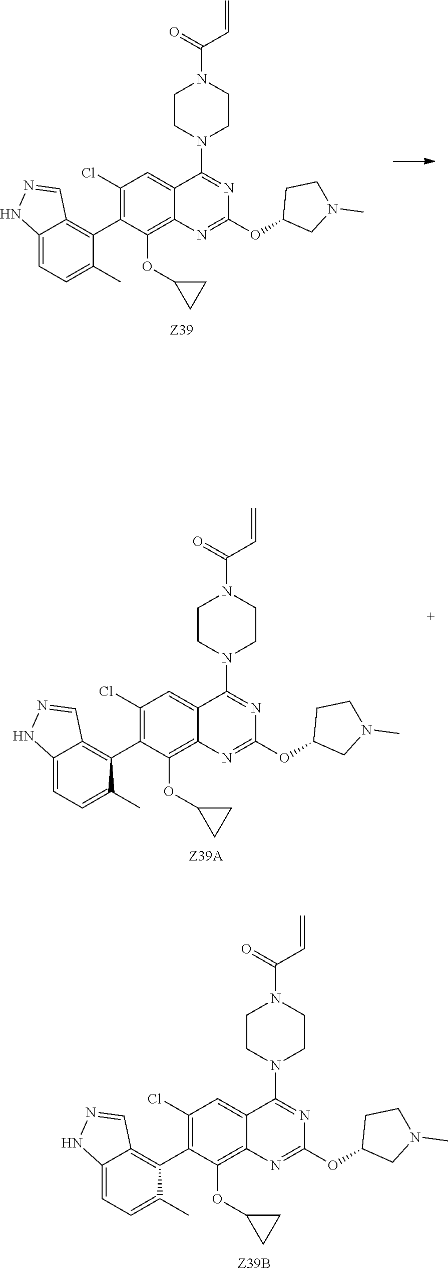

Compound Z39 (240 mg) was separated through chiral resolution (mobile phase:EtOH (0.2% DEA)); chromatography column: IG 250 mm*4.6 mm 5 um); flow rate: 1.0 ml/min; column temperature: 30.0° C.) to obtain one isomer, arbitrarily designated as Z39A: 1-(4-(6-chloro-8-cyclopropoxy-7-((R)-5-methyl-1H-indazol-4-yl)-2-((((R)-1-methylpyrrolidin-3-yl) oxy]]quinazolin-pyridin-4-yl)piperazin-1-yl)prop-2-en-1-one (retention time: 7.68 min; 84 mg; purity: 99%; de value: 99.2%); ES-API: [M+H]⁺=588.2; and the other isomer, arbitrarily designated as Z39B: 1-(4-(6-chloro-8-cyclopropoxy-7-((S)-5-methyl-1H-indazol-4-yl)-2-((((R)-1-methylpyrrolidin-3-yl)oxy]]quinazolin-pyridin-4-yl)piperazin-1-yl)prop-2-en-1-one (retention time: 10.73 min; 120 mg; purity: 95.2%; de value: 93.2%); ES-API: [M+H]⁺=588.2.

### Example 40 Preparation of Z40

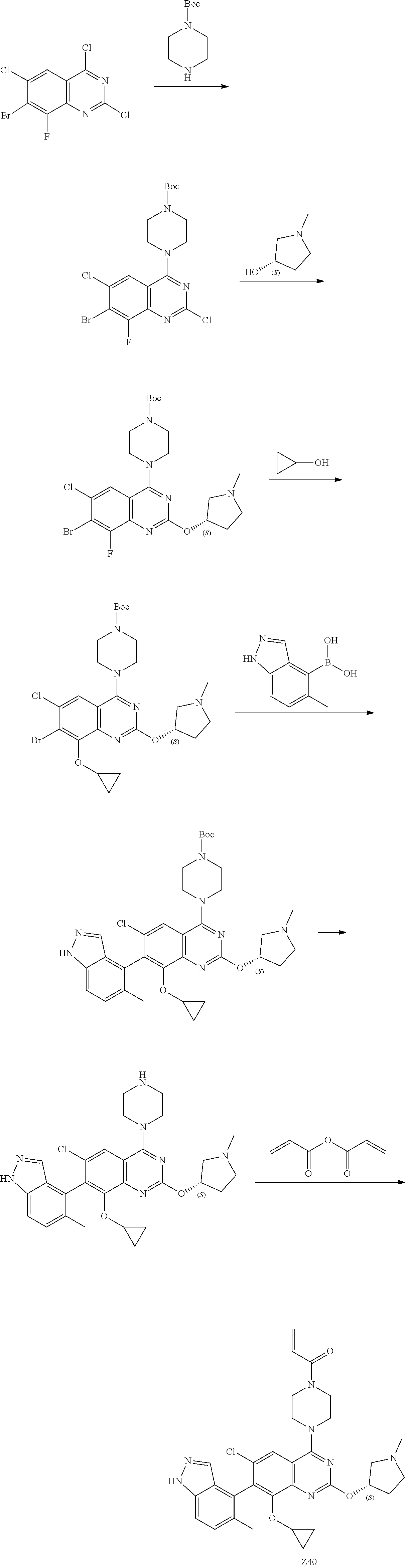

step 1: 7-bromo-2,4,6-trichloro-8-fluoroquinazoline (15 g, 45.4 mmol) was added to dichloromethane/acetonitrile (50/50 mL). The reaction solution was cooled to 0° C. and then triethylamine (23 g, 227.3 mmol) was added slowly. After that tert-butyl piperazin-1-carboxylate (8.9 g, 47.7 mmol) was added. The reaction solution was slowly warmed up to room temperature and then stirred for 2 h. Dichloromethane (70 mL*3) and water (70 mL) were added, which was extracted and the organic phase was dried and dried by rotary evaporation to give a crude, which was beat with acetonitrile (40 mL) to obtain the product (17.6 g, Y: 81%). ES-API: [M+1]⁺=481.0

step 2: tert-butyl 4-(7-bromo-2,6-dichloro-8-fluoroquinazolin-4-yl)piperazin-1-carboxylate (10 g, 20.8 mmol) was dissolved in dimethylformamide (60 mL), then (S)-1-methylpyrrolidin-3-ol (4.2 g, 41.7 mmol), cesium carbonate (20.4 g, 62.5 mmol) and triethylene diamine (467 mg, 4.2 mmol) were added. The reaction was conducted at room temperature for 2 h. 100 mL of water was added and the mixture was extracted with ethyl acetate (100 mL*3). The organic phase was dried and dried by rotary evaporation to obtain a crude which was purified by flash column on silica gel (dichloromethane/methanol=30/1) to obtain the product (10 g, Y: 88%). (Rf=0.2, dichloromethane/methanol=10/1). ES-API: [M+1]⁺=546.1

step 3: cyclopropanol (1.6 g, 27.5 mmol) was dissolved in tetrahydrofuran (50 mL) and at 0° C. sodium hydrogen (7.3 g, 183.5 mmol) was added. The reaction was stirred for 0.5 h while keeping the temperature. A solution of tert-butyl (S)₄-(7-bromo-6-chloro-8-fluoro-2-((1-methylpyrrolidin-3-yl)oxy)quinazolin-4-yl)piperazin-1-carboxylate (10 g, 18.3 mmol) in tetrahydrofuran (50 mL) was then added into the reaction solution. The reaction was conducted at room temperature for 6 h. The reaction solution was quenched with water, extracted with ethyl acetate, dried over anhydrous sodium sulfate and filtered. The filtration was dried by rotary evaporation to obtain a yellow oil. The crude was purified by flash column on silica gel (dichloromethane/methanol=30/1) to obtain the product (9.2 g, Y: 86%). (Rf=0.3, dichloromethane/methanol=20/1). ES-API: [M+1]⁺=584.1

step 4: tert-butyl (S)-4-(7-bromo-6-chloro-8-cyclopropoxy-2-((1-methylpyrrolidin-3-yl) oxy)quinazolin-4-yl)piperazin-1-carboxylate (5 g, 8.58 mmol), (5-methyl-1H-indazol-4-yl) boronic acid (3 g, 17.1 mmol), tripotassium phosphate (5.4 g, 25.7 mmol), tris (dibenzylideneindenacetone)dipalladium (785 mg, 0.86 mmol), 2-dicyclohexylphosphine-2′,4′,6′-triisopropylbiphenyl (400 mg, 0.86 mmol) were added in sequence into dioxane (40 mL) and water (10 mL). The system atmosphere was replaced with nitrogen three times, then the temperature was warmed up to 100° C. and the reaction was conducted for 16 h. After cooling to room temperature, the reaction solution was poured into ethyl acetate (100 mL) and after being washed with brine once it was purified by column on silica gel (dichloromethane/methanol=20/1) to obtain (2.1 g, Y: 39%). (Rf=0.2, dichloromethane/methanol=15/1). ES-API [M+1]⁺=634.3

step 5: tert-butyl 4-(6-chloro-8-cyclopropoxy-7-(5-methyl-1H-indazol-4-yl)-2-((((S)-1-methylpyrrolidin-3-yl)oxy)quinazolin-4-yl)piperazin-1-carboxylate (2.1 g, 3.31 mmol) was dissolved in dichloromethane (10 mL) and then trifluoroacetic acid (4 mL) was added. The reaction was conducted at room temperature for 1 h and the reaction mixture was dried by rotary evaporation to obtain the crude product, which was used in the next step directly.

step 6: 6-chloro-8-cyclopropoxy-7-(5-methyl-1H-indazol-4-yl)-2-((((S)-1-methylpyrrolidin-3-yl)oxy]-4-(piperazin-1-yl)quinazoline (crude) was dissolved in dichloromethane (15 mL). At 0° C. triethylamine (7 mL) and acrylic anhydride (376 mg, 2.98 mmol) were added, and the reaction was conducted at 0° C. for 1 h. The reaction solution was extracted by adding 30 mL of water and dichloromethane (30 mL*3) and the organic phase was dried by rotary evaporation to obtain a crude, which was purified firstly by flash column on silica gel (dichloromethane/methanol=20/1) and then by preparation to obtain the product (265 mg, Y: 14%). ES-API: [M+1]⁺=588.2. The preparation and separation conditions were the same as in Example 34. ¹HNMR (400 MHz, CDCl₃)δ 10.36 (s, 1H), 7.75 (d, J=2.0 Hz, 1H), 7.58 (s, 1H), 7.47 (d, J=8.5 Hz, 1H), 7.34 (d, J=8.3 Hz, 1H), 6.68-6.57 (m, 1H), 6.37 (d, J=16.7 Hz, 1H), 5.78 (d, J=10.4 Hz, 1H), 5.59 (s, 1H), 4.29 (s, 1H), 3.95-3.75 (m, 9H), 3.16 (s, 1H), 2.88-2.81 (m, 2H), 2.68 (s, 1H), 2.52-2.36 (m, 4H), 2.22-2.12 (m, 4H), 0.27-0.14 (m, 4H).

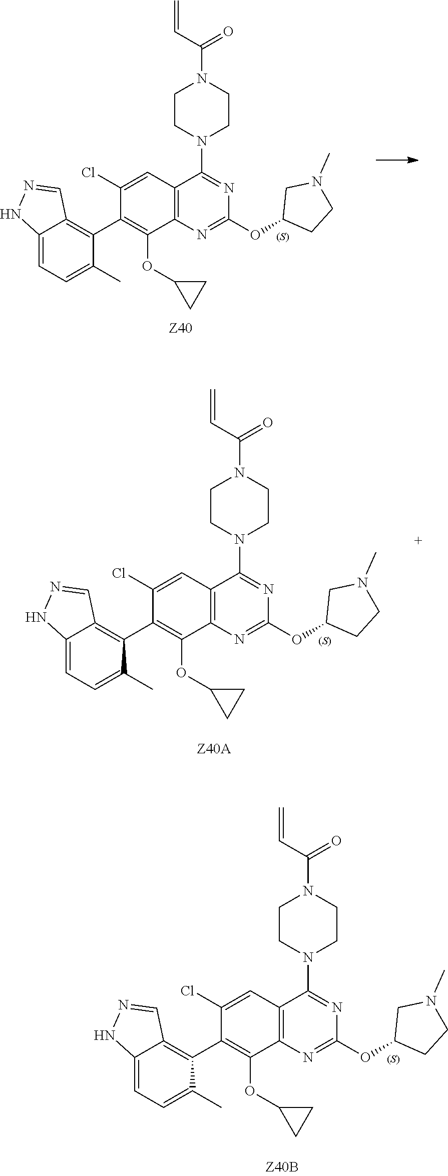

Z40 (220 mg) was separated through chiral resolution (mobile phase:MeOH:ACN(0.2% MA)=45:55; column: OZ PDA 293.0 nm; flow rate: 1 ml/min; column temperature: 25.0° C.) to obtain one isomer, arbitrarily designated as Z40A: 1-(4-((S)-6-chloro-8-cyclopropoxy-7-(5-methyl-1H-indazol-4-yl)-2-(((S)-1-methylpyrrolidin-3-yl) oxy)quinazolin-pyridin-4-yl)piperazin-1-yl)prop-2-en-1-one (retention time: 3.038 min; 130 mg, purity: 99.6%, de value: 100%). ES-API: [M+H]⁺=588.2; and the other isomer, arbitrarily designated as Z40B: 1-(4-((R)-6-chloro-8-cyclopropoxy-7-(5-methyl-1H-indazol-4-yl)-2-(((S)-1-methylpyrrolidin-3-yl)oxy)quinazolin-pyridin-4-yl)piperazin-1-yl)prop-2-en-1-one (retention time: 4.242 min; 80 mg, purity: 100%, de value: 100%). ES-API: [M+H]⁺=588.2.

### Example 41 Preparation of Z41

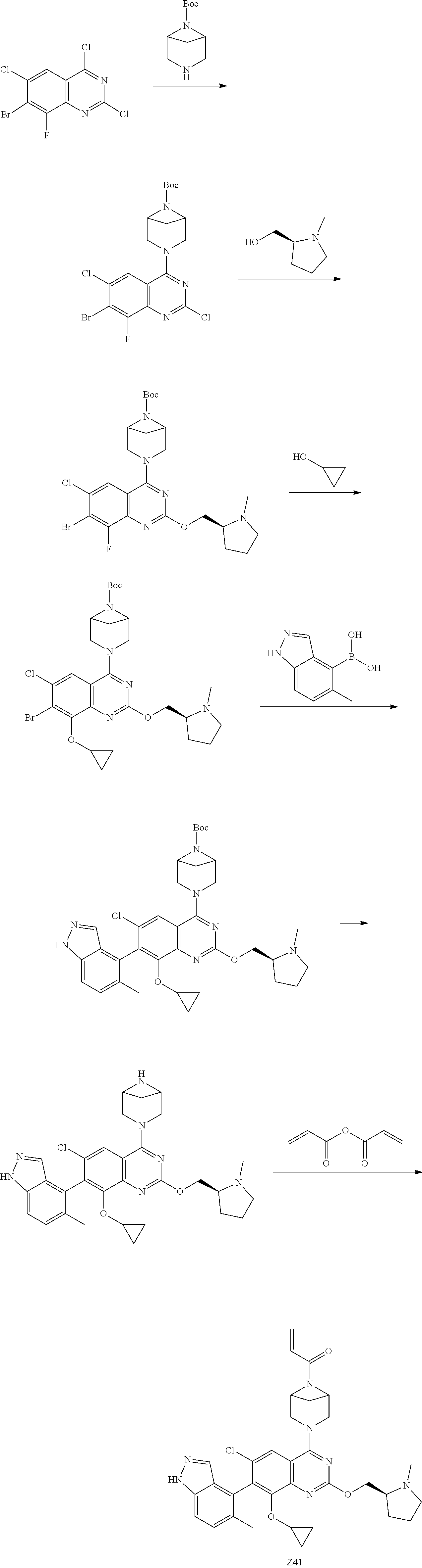

step 1: 7-bromo-2,4,6-trichloro-8-fluoroquinazoline (828 mg, 2.525 mmol), tert-butyl 3,6-diazabicyclo[3.1.1]heptane-6-carboxylate (500 mg, 2.525 mmol) were added to dichloromethane (8 mL). The reaction solution was cooled to 0° C. and then triethylamine (510 mg, 5.05 mmol) was added slowly. The reaction was stirred for 0.5 h. The mixture was separated by adding water and dichloromethane and the organic phase was washed with brine, dried over sodium sulfate, and dried by rotary evaporation to obtain crude tert-butyl 3-(7-bromo-2,6-dichloro-8-fluoroquinazolin-4-yl)-3,6-diazabicyclo[3.1.1]heptane-6-carboxylate (1.1 g, Y: 88.7%). ES-API: [M+1]⁺=491.0

step 2: tert-butyl 3-(7-bromo-2,6-dichloro-8-fluoroquinazolin-4-yl)-3,6-diazabicyclo[3.1.1]heptane-6-carboxylate (1.1 g, 2.24 mmol) was added to (S)-(1-methylpyrrolidin-2-yl) methanol (6 mL) and then potassium fluoride (391 mg, 6.73 mmol) was added. The mixture was heated to 100° C. and the reaction was conducted for 2 h. It was detected by LCMS till the reaction was complete. After the reaction solution was cooled to room temperature 30 mL of water was added and the mixture was extracted with ethyl acetate (30 mL*3). The organic phase was dried and dried by rotary evaporation to obtain a crude which was purified by flash column on silica gel to obtain tert-butyl 3-(7-bromo-6-chloro-8-fluoro-2-((((S)-1-methylpyrrolidin-2-yl)methoxy)quinazolin-4-yl)-3,6-diazabicyclo [3.1.1]heptane-6-carboxylate (930 mg, Y: 72.9%). ES-API: [M+1]⁺=570.1

step 3: tert-butyl 3-(7-bromo-6-chloro-8-fluoro-2-((((S)-1-methylpyrrolidin-2-yl) methoxy)quinazolin-4-yl)-3,6-diazabicyclo[3.1.1]heptane-6-carboxylate (930 mg, 1.63 mmol), cyclopropanol (474 mg, 8.17 mmol) were dissolved in tetrahydrofuran (10 mL), and at 0° C. sodium hydrogen (652 mg, 16.3 mmol) was added in batches. The reaction was stirred for 0.5 h while keeping the temperature. The reaction was quenched by adding water, and the mixture was extracted with ethyl acetate. The organic phase was washed with brine, dried over anhydrous sodium sulfate and dried by rotary evaporation under reduced pressure to obtain crude tert-butyl 3-(7-bromo-6-chloro-8-cyclopropoxy-2-(((S)-1-methylpyrrolidin-2-yl)methoxy)quinazolin-4-yl)-3,6-diazabicyclo[3.1.1]heptane-6-carboxylate (1200 mg, Y: 100%). ES-API: [M+1]⁺=608.2

step 4: tert-butyl 3-(7-bromo-6-chloro-8-cyclopropoxy-2-(((S)-1-methylpyrrolidin-2-yl) methoxy)quinazolin-4-yl)-3,6-diazabicyclo[3.1.1]heptane-6-carboxylate (1200 mg, crude, 1.63 mmol), (5-methyl-1H-indazol-4-yl) boronic acid (574 mg, 3.26 mmol), potassium phosphate (691 mg, 3.26 mmol), Ruphos (152 mg, 0.326 mmol) and Pd₂(dba)₃ (149 mg, 0.163 mmol) were added in sequence to dioxane (10 mL) and water (2 mL). The system atmosphere was replaced with nitrogen three times, then the temperature was warmed up to 100° C. and the reaction was conducted for 6 h. After cooling to room temperature, the reaction solution was poured into ethyl acetate (30 mL) and after being washed with brine once, it was dried over anhydrous sodium sulfate, dried by rotary evaporation under reduced pressure, and purified by column (dichloromethane/methanol=100/1) to obtain tert-butyl 3-(6-chloro-8-cyclopropoxy-7-(5-methyl-1H-indazol-4-yl)-2-(((S)-1-methylpyrrolidin-2-yl)methoxy) quinazolin-4-yl)-3,6-diazabicyclo[3.1.1]heptane-6-carboxylate (560 mg, Y: 52.1%). ES-API: [M+1]⁺=660.3

step 5: tert-butyl 3-(6-chloro-8-cyclopropoxy-7-(5-methyl-1H-indazol-4-yl)-2-(((S)-1-methylpyrrolidin-2-yl)methoxy)quinazolin-4-yl)-3,6-diazabicyclo[3.1.1]heptane-6-carboxylate (560 mg, 0.85 mmol) was dissolved in dichloromethane (3 mL) and then trifluoroacetic acid (3 mL) was added. The reaction was conducted at room temperature for 0.5 h and the reaction mixture was dried by rotary evaporation to obtain crude 3-(6-chloro-8-cyclopropoxy-7-(5-methyl-1H-indazol-4-yl)-2-((((S)-1-methylpyrrolidin-2-yl)methoxy) quinazolin-4-yl)-3,6-diazabicyclo[3.1.1]heptane (700 mg, Y: 100%). ES-API: [M+1]⁺=560.3

step 6: 3-(6-chloro-8-cyclopropoxy-7-(5-methyl-1H-indazol-4-yl)-2-((((S)-1-methylpyrrolidin-2-yl)methoxy)quinazolin-4-yl)-3,6-diazabicyclo[3.1.1]heptane (700 mg, crude, 0.85 mmol) and triethylamine (3 mL) were dissolved in dichloromethane (3 mL). At 0° C. acrylic anhydride (86 mg, 0.68 mmol) was added, and the reaction was conducted at 0° C. for 0.5 h. The reaction solution was extracted by adding 20 mL of water and dichloromethane (20 mL*3). The organic phase was washed with brine, dried over anhydrous sodium sulfate, dried by rotary evaporation under reduced pressure and purified by preparation (column: Ultimate XB-C18, 50*250 mm, 10 um, mobile phase: A: purified water B: purified acetonitrile, flow rate: 80 ml/min, gradient: within 50 min, B/A=20%-90%, wavelength: 214 nm, column temperature: room temperature) to obtain 1-(3-(6-chloro-8-cyclopropoxy-7-(5-methyl-1H-indazol-4-yl)-2-(((S)-1-methylpyrrolidin-2-yl)methoxy)quinazolin-4-yl)-3,6-diazabicyclo[3.1.1]heptan-6-yl)prop-2-en-1-one (45 mg, Y: 8.64%). ES-API: [M+1]⁺=614.3. ¹HNMR (400 MHz, CDCl₃)δ8.14 (s, 1H), 7.58 (s, 1H), 7.46 (d, J=8.6 Hz, 1H), 7.32 (d, J=8.6 Hz, 1H), 6.41 (d, J=17.0 Hz, 1H), 6.28 (dd, J=17.0, 10.2 Hz, 1H), 5.75 (d, J=10.2 Hz, 1H), 4.86-4.64 (m, 5H), 4.56-4.13 (m, 6H), 3.29 (s, 1H), 3.00 (s, 1H), 2.82 (d, J=8.5 Hz, 1H), 2.63 (s, 3H), 2.47 (s, 1H), 2.19 (s, 5H), 1.70 (d, J=9.0 Hz, 1H), 0.26-0.06 (m, 4H).

### Example 42 Preparation of Z42

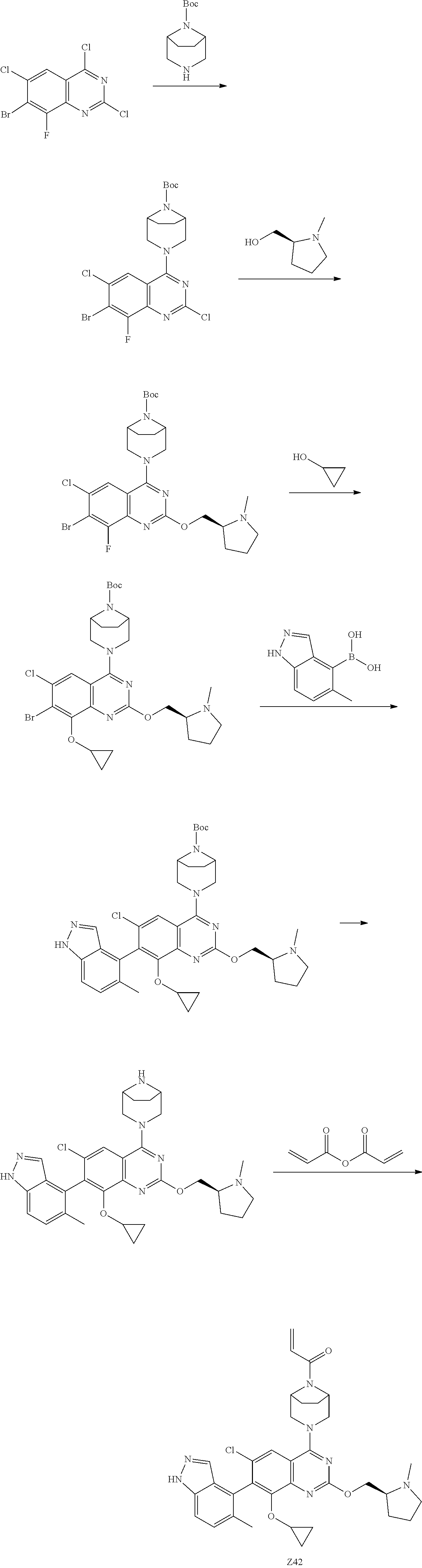

step 1: 7-bromo-2,4,6-trichloro-8-fluoroquinazoline (777 mg, 2.35 mmol), tert-butyl (1R, 5S)-3,8-diazabicyclo[3.2.1]octane-8-carboxylate (500 mg, 2.35 mmol) were added to DCM (5 mL). The reaction solution was cooled to 0° C. and then triethylamine (712 mg, 7.05 mmol) was added slowly. The reaction was stirred for 0.5 h. The mixture was separated by adding water and dichloromethane and the organic phase was washed with brine, dried over sodium sulfate, and dried by rotary evaporation to obtain crude tert-butyl (1R, 5S)-3-(7-bromo-2,6-dichloro-8-fluoroquinazolin-4-yl)-3,8-diazabicyclo[3.2.1]octane-8-carboxylate (1.1 g, Y: 92.5%). ES-API: [M+1]⁺=505.0

step 2: tert-butyl (1R, 5S)-3-(7-bromo-2,6-dichloro-8-fluoroquinazolin-4-yl)-3,8-diazabicyclo[3.2.1]octane-8-carboxylate (1.1 g, 2.24 mmol) was added to (S)-(1-methylpyrrolidin-2-yl) methanol (4 g) and then KF (364 mg, 6.51 mmol) was added. The mixture was heated to 100° C. and the reaction was conducted for 2 h. It was detected by LCMS till the reaction was complete. After the reaction solution was cooled to room temperature 30 mL of water was added, and the mixture was extracted with ethyl acetate (30 mL*3). The organic phase was dried and dried by rotary evaporation to obtain a crude which was purified by flash column on silica gel to obtain tert-butyl (1R, 5S)-3-(7-bromo-6-chloro-8-fluoro-2-((((S)-1-methylpyrrolidin-2-yl)methoxy)quinazolin-4-yl)-3,8-diazabicyclo[3.2.1]octane-8-carboxylate (1 g, Y: 78.7%). ES-API: [M+1]⁺=570.1

step 3: tert-butyl (1R, 5S)-3-(7-bromo-6-chloro-8-fluoro-2-((((S)-1-methylpyrrolidin-2-yl)methoxy)quinazolin-4-yl)-3,8-diazabicyclo[3.2.1]octane-8-carboxylate (1 g, 1.71 mmol) and cyclopropanol (298 mg, 5.13 mmol) were dissolved in tetrahydrofuran (20 mL), and at 0° C. sodium hydrogen (684 mg, 17.1 mmol) was added in batches. The reaction was stirred for 0.5 h while keeping the temperature. The reaction was quenched by adding water, and the mixture was extracted with ethyl acetate. The organic phase was washed with brine, dried over anhydrous sodium sulfate, dried by rotary evaporation under reduced pressure to obtain crude tert-butyl (1R, 5S)-3-(7-bromo-6-chloro-8-cyclopropoxy-2-((((S)-1-methylpyrrolidin-2-yl)methoxy)quinazolin-4-yl)-3,8-diazabicyclo[3.2.1]octane-8-carboxylate (1.1 g, Y: 100%). ES-API: [M+1]⁺=622.2

step 4: tert-butyl (1R, 5S)-3-(7-bromo-6-chloro-8-cyclopropoxy-2-((((S)-1-methylpyrrolidin-2-yl)methoxy)quinazolin-4-yl)-3,8-diazabicyclo[3.2.1]octane-8-carboxylate (1.1 g, crude, 1.71 mmol), (5-methyl-1H-indazol-4-yl) boronic acid (601 mg, 3.42 mmol), potassium phosphate (1.08 g, 5.13 mmol), Ruphos (156 mg, 0.342 mmol) and Pd₂(dba)₃ (156 mg, 0.171 mmol) were added in sequence to dioxane (10 mL) and water (2 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once, it was dried over anhydrous sodium sulfate, dried by rotary evaporation under reduced pressure and purified by column to obtain tert-butyl (1R, 5S)-3-(6-chloro-8-cyclopropoxy-7-(5-methyl-1H-indazol-4-yl)-2-((((S)-1-methylpyrrolidin-2-yl)methoxy)quinazolin-4-yl)-3,8-diazabicyclo[3.2.1]octane-8-carboxy late (380 mg, Y: 33%). ES-API: [M+1]⁺=674.3

step 5: tert-butyl (1R, 5S)-3-(6-chloro-8-cyclopropoxy-7-(5-methyl-1H-indazol-4-yl)-2-((((S)-1-methylpyrrolidin-2-yl)methoxy)quinazolin-4-yl)-3,8-diazabicyclo[3.2.1]octane-8-carboxylate (380 mg, 0.56 mmol) was dissolved in dichloromethane (3 mL) and then trifluoroacetic acid (3 mL) was added. The reaction was conducted at room temperature for 0.5 h and the reaction mixture was dried by rotary evaporation to obtain crude 4-((1R, 5S)-3,8-diazabicyclo[3.2.1]octane-3-yl)-6-chloro-8-cyclopropoxy-7-(5-methyl-1H-indazol-4-yl)-2-(((S)-1-methylpyrrolidin-2-yl)methoxy) quinazoline (450 mg, Y: 100%). ES-API: [M+1]⁺=574.3

step 6: 4-((1R, 5S)-3,8-diazabicyclo[3.2.1]octane-3-yl)-6-chloro-8-cyclopropoxy-7-(5-methyl-1H-indazol-4-yl)-2-(((S)-1-methylpyrrolidin-2-yl)methoxy)quinazoline (450 mg, crude, 0.56 mmol) and triethylamine (3 mL) were dissolved in dichloromethane (3 mL). At 0° C. acrylic anhydride (63 mg, 0.50 mmol) was added, and the reaction was conducted at 0° C. for 0.5 h. The reaction solution was extracted by adding 20 mL of water and DCM (20 mL*3). The organic phase was washed with brine, dried over anhydrous sodium sulfate, dried by rotary evaporation under reduced pressure and purified by preparation to obtain 1-((1R, 5S)-3-(6-chloro-8-cyclopropoxy-7-(5-methyl-1H-indazol-4-yl)-2-(((S)-1-methylpyrrolidin-2-yl)methoxy)quinazolin-4-yl)-3,8-diazabicyclo[3.2.1]octane-8-yl)prop-2-en-1-one (3.8 mg, Y: 2%). The preparation and separation conditions were the same as in Example 10. ES-API: [M+1]+=628.1. ¹HNMR (400 MHz, CDCl₃)δ 7.72 (s, 1H), 7.58 (s, 1H), 7.46 (d, J=8.4 Hz, 1H), 7.33 (d, J=8.4 Hz, 1H), 6.62-6.41 (m, 2H), 5.79 (d, J=10.2 Hz, 1H), 4.92 (s, 1H), 4.74 (s, 1H), 4.39 (d, J=56.6 Hz, 5H), 3.68 (d, J=27.9 Hz, 1H), 3.42 (d, J=54.6 Hz, 2H), 3.07 (s, 1H), 2.68 (s, 3H), 2.19 (s, 3H), 1.98 (s, 1OH), 0.19 (d, J=23.0 Hz, 4H).

### Example 43 Preparation of Z43

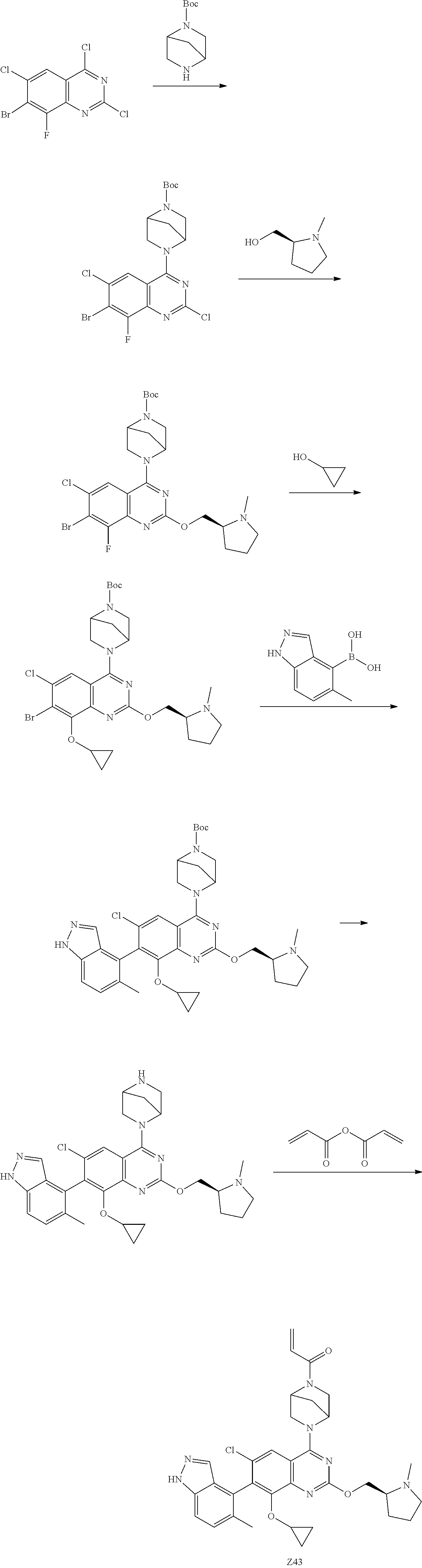

step 1: 7-bromo-2,4,6-trichloro-8-fluoroquinazoline (833 mg, 2.52 mmol) and triethylamine (510 mg, 5.04 mmol) were dissolved in dichloromethane (10 mL), and under ice bath tert-butyl 2,5-diazabicyclo[2.2.1]heptane-2-carboxylate (500 mg, 2.52 mmol) was added. After the addition, the reaction was conducted at room temperature for 0.5 h, The reaction solution was poured into water (50 mL), extracted with dichloromethane (50 mL×3). The organic phase was washed with water and washed with brine and then dried by rotary evaporation to obtain crude tert-butyl 5-(7-bromo-2,6-dichloro-8-fluoroquinazolin-4-yl)-2,5-diazabicyclo[2.2.1]heptane-2-carboxylate (1.3 g, Y: 100%) The crude product was used in the next step.

step 2: tert-butyl 5-(7-bromo-2,6-dichloro-8-fluoroquinazolin-4-yl)-2,5-diazabicyclo[2.2.1]heptane-2-carboxylate (1.2 g, 2.52 mmol) was added to potassium fluoride (438 mg, 7.56 mmol) and (S)-(1-methylpyrrolidin-2-yl) methanol (10 mL, 10V). The reaction was conducted at 100° C. for 2 h. After cooling, the reaction solution was poured into water (100 mL) and extracted with ethyl acetate (50 mL×3). The organic phase was washed with water and washed with brine and then dried by rotary evaporation, and purified by column chromatography (methanol:dichloromethane=0˜10%) to obtain tert-butyl (5-(7-bromo-6-chloro-8-fluoro-2-((((S)-1-methylpyrrolidin-2-yl)methoxy)quinazolin-4-yl)-2,5-diazabicyclo [2.2.1]heptane-2-carboxylate (0.9 g, Y: 62%) as a yellow oil. ES-API: [M+1]⁺=570.1 572.1

step 3: cyclopropanol (275 mg, 4.74 mmol) was dissolved in tetrahydrofuran (10 mL) and at 0° C. sodium hydrogen (190 mg, 4.74 mmol) was added. The reaction was stirred for 0.5 h while keeping the temperature. tert-butyl 5-(7-bromo-6-chloro-8-fluoro-2-((((S)-1-methylpyrrolidin-2-yl) methoxy)quinazolin-4-yl)-2,5-diazabicyclo[2.2.1]heptane-2-carboxylate (0.9 g, 1.58 mmol) was then added into the reaction solution. The reaction was conducted for 1 h while keeping the temperature. The reaction solution was poured into ethyl acetate (100 mL), the organic phase was washed with water (20 mL×2) and brine (20 mL), dried over anhydrous sodium sulfate and filtered. The filtration was dried by rotary evaporation to obtain tert-butyl 5-(7-bromo-6-chloro-8-cyclopropoxy-2-((((S)-1-methylpyrrolidin-2-yl)methoxy)quinazolin-4-yl)-2,5-diazabicyclo[2.2.1]heptane-2-carboxylate (1.2 g, Y: 100%) as a yellow oil. ES-API: [M/2+1]+=608.1 610.1

step 4: tert-butyl 5-(7-bromo-6-chloro-8-cyclopropoxy-2-((((S)-1-methylpyrrolidin-2-yl) methoxy)quinazolin-4-yl)-2,5-diazabicyclo[2.2.1]heptane-2-carboxylate (961 mg, 1.58 mmol), (5-methyl-1H-indazol-4-yl) boronic acid (417 mg, 2.37 mmol), tripotassium phosphate (670 mg, 3.16 mmol), tris (dibenzylideneindenacetone)dipalladium (100 mg), 2-dicyclohexylphosphine-2′,4′,6′-triisopropylbiphenyl (200 mg) were added in sequence into dioxane (10 mL) and water (2 mL). The system atmosphere was replaced with nitrogen three times, then the temperature was warmed up to 105° C. and the reaction was conducted for 6 h. After cooling to room temperature, the reaction solution was poured into ethyl acetate (50 mL) and after being washed with brine once it was purified by column on silica gel (methanol:dichloromethane=0˜1:10) to obtain tert-butyl 5-(6-chloro-8-cyclopropoxy-7-(5-methyl-1H-indazol-4-yl)-2-((((S)-1-methylpyrrolidin-2-yl)methoxy)quinazolin-4-yl)-2,5-diazabicyclo [2.2.1]heptane-2-carboxylate (430 mg, Y: 41%) as a yellow solid. ES-API: [M/2+1]⁺=660.3

step 5: tert-butyl 5-(6-chloro-8-cyclopropoxy-7-(5-methyl-1H-indazol-4-yl)-2-((((S)-1-methylpyrrolidin-2-yl)methoxy)quinazolin-4-yl)-2,5-diazabicyclo[2.2.1]heptane-2-carboxylate (430 mg, 0.65 mmol) was dissolved in dichloromethane (2 mL) and then trifluoroacetic acid (2 mL) was added. The reaction was conducted at room temperature for 1 h and the reaction mixture was dried by rotary evaporation to obtain crude 4-(2,5-diazabicyclo[2.2.1]heptane-2-yl)-6-chloro-8-cyclopropoxy-7-(5-methyl-1H-indazol-4-yl)-2-(((S)-1-methylpyrrolidin-2-yl)methoxy)quinazoline (363 mg, Y: 100%) as a yellow oil.

step 6: 4-(2,5-diazabicyclo[2.2.1]heptane-2-yl)-6-chloro-8-cyclopropoxy-7-(5-methyl-1H-indazol-4-yl)-2-(((S)-1-methylpyrrolidin-2-yl)methoxy)quinazoline (363 mg, 0.65 mmol) and triethylamine (197 mg, 1.95 mmol) were dissolved in dichloromethane (5 mL). At 0° C. acrylic anhydride (65 mg, 0.52 mmol) was added, and the reaction was conducted at 0° C. for 1 h. The reaction solution was dried by rotary evaporation and purified by preparative plate (dichloromethane:methanol:ammonium hydroxide=90:10) to obtain 1-(5-(6-chloro-8-cyclopropoxy-7-(5-methyl-1H-indazol-4-yl)-2-(((S)-1-methylpyrrolidin-2-yl)methoxy) quinazolin-4-yl)-2,5-diazabicyclo[2.2.1]heptane-2-yl)prop-2-en-1-one (15 mg, Y: 3.7%). ES-API: [M/2+1]⁺=614.2. ¹HNMR (400 MHz, CDCl₃)δ 7.86 (s, 1H), 7.52 (s, 1H), 7.48 (d, J=8.4 Hz, 1H), 7.32 (d, J=8.4 Hz, 1H), 6.40 (d, J=15.8 Hz, 1H), 6.35-6.27 (m, 1H), 5.71 (d, J=10.2 Hz, 1H), 5.45 (s, 1H), 5.16 (s, 1H), 5.02 (s, 1H), 4.81 (s, 1H), 4.24 (d, J=8.8 Hz, 1H), 4.13 (s, 1H), 4.06 (d, J=9.1 Hz, 1H), 3.92 (d, J=10.1 Hz, 1H), 3.78 (d, J=9.3 Hz, 1H), 3.64 (s, 1H), 3.47 (s, 3H), 2.97 (s, 4H), 2.20 (s, 3H), 2.07 (dd, J=33.7, 10.5 Hz, 6H), 0.19 (s, 4H).

### Example 44 Preparation of Z44

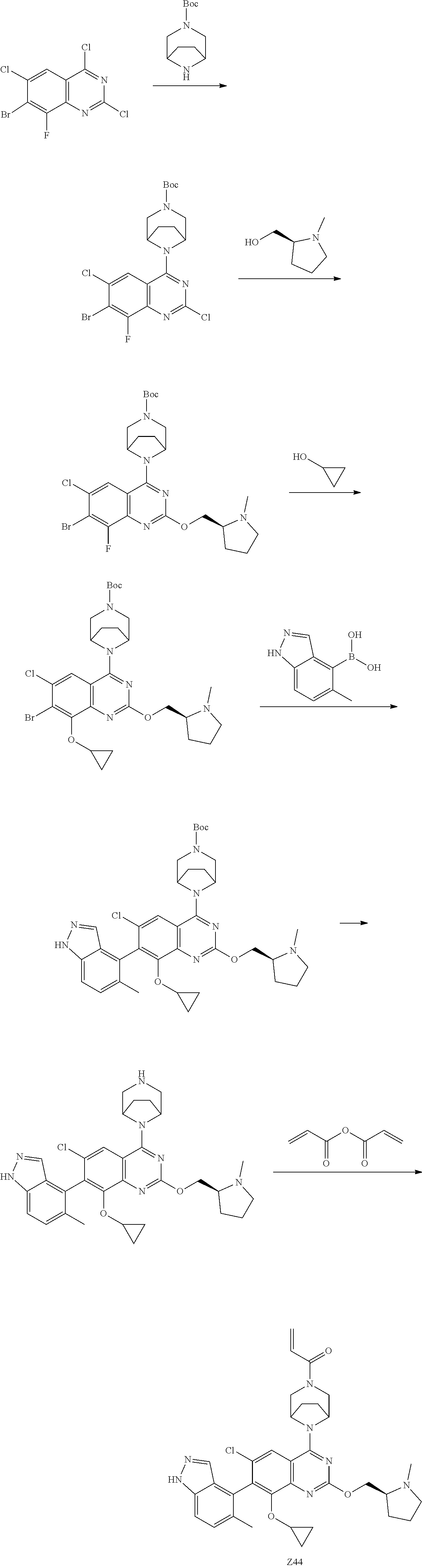

step 1: 7-bromo-2,4,6-trichloro-8-fluoroquinazoline (778 mg, 2.36 mmol) and triethylamine (477 mg, 4.71 mmol) were dissolved in dichloromethane (10 mL), and under ice bath tert-butyl (1R, 5S)3,8-diazabicyclo[3.2.1]octane-3-carboxylate (500 mg, 2.36 mmol) was added. After the addition, the reaction was conducted at room temperature for 0.5 h, The reaction solution was poured into water (50 mL), and extracted with dichloromethane (50 mL×3). The organic phase was washed with water and washed with brine and then dried by rotary evaporation to obtain crude tert-butyl (1R, 5S)-8-(7-bromo-2,6-dichloro-8-fluoroquinazolin-4-yl)-3,8-diazabicyclo[3.2.1]octane-3-carboxylate (1.3 g, Y: 100%), which was used in the next step.

step 2: tert-butyl (1R, 5S)-8-(7-bromo-2,6-dichloro-8-fluoroquinazolin-4-yl)-3,8-diazabicyclo[3.2.1]octane-3-carboxylate (1.2 g, 2.36 mmol) was added to potassium fluoride (411 mg, 7.08 mmol) and (S)-(1-methylpyrrolidin-2-yl) methanol (10 mL, 10V). The reaction was conducted at 100° C. for 2 h. After cooling, the reaction solution was poured into water (100 mL) and extracted with ethyl acetate (50 mL×3). The organic phase was washed with water and washed with brine and then dried by rotary evaporation, and purified by column chromatography (methanol:dichloromethane=0˜10%) to obtain tert-butyl (1R, 5S)-8-(7-bromo-6-chloro-8-fluoro-2-((((S)-1-methylpyrrolidin-2-yl)methoxy)quinazolin-4-yl)-3,8-diazabicyclo[3.2.1]octane-3-carboxylate (1 g, Y: 72%) as a yellow oil. ES-API: [M+1]⁺=584.1 586.1

step 3: cyclopropanol (298 mg, 5.14 mmol) was dissolved in tetrahydrofuran (10 mL) and at 0° C. sodium hydrogen (206 mg, 5.14 mmol) was added. The reaction was stirred for 0.5 h while keeping the temperature. tert-butyl (1R, 5S)-8-(7-bromo-6-chloro-8-fluoro-2-((((S)-1-methylpyrrolidin-2-yl)methoxy)quinazolin-4-yl)-3,8-diazabicyclo[3.2.1]octane-3-carboxy late (1 g, 1.71 mmol) was then added into the reaction solution. The reaction was conducted for 1 h while keeping the temperature. The reaction solution was poured into ethyl acetate (100 mL), the organic phase was washed with water (20 mL×2) and brine (20 mL), dried over anhydrous sodium sulfate and filtered. The filtration was dried by rotary evaporation to obtain tert-butyl (1R, 5S)-8-(7-bromo-6-chloro-8-cyclopropoxy-2-((((S)-1-methylpyrrolidin-2-yl)methoxy)quinazol in-4-yl)-3,8-diazabicyclo[3.2.1]octane-3-carboxylate (1.2 g, Y: 100%) as a yellow oil. ES-API: [M/2+1]⁺=622.1 624.1

step 4: tert-butyl (1R, 5S)-8-(7-bromo-6-chloro-8-cyclopropoxy-2-((((S)-1-methylpyrrolidin-2-yl)methoxy)quinazolin-4-yl)-3,8-diazabicyclo[3.2.1]octane-3-carboxylate (1.06 g, 1.71 mmol), (5-methyl-1H-indazol-4-yl) boronic acid (451 mg, 2.57 mmol), tripotassium phosphate (725 mg, 3.42 mmol), tris (dibenzylideneindenacetone)dipalladium (100 mg), 2-dicyclohexylphosphine-2′,4′,6′-triisopropylbiphenyl (200 mg) were added in sequence into dioxane (10 mL) and water (2 mL). The system atmosphere was replaced with nitrogen three times, then the temperature was warmed up to 105° C. and the reaction was conducted for 6 h. After cooling to room temperature, the reaction solution was poured into ethyl acetate (50 mL) and after being washed with brine once it was purified by column on silica gel (methanol:dichloromethane=0˜1:10) to obtain tert-butyl (1R, 5S)-8-(6-chloro-8-cyclopropoxy-7-(5-methyl-1H-indazol-4-yl)-2-((((S)-1-methylpyrrolidin-2-yl)methoxy)quinazolin-4-yl)-3,8-diazabicyclo[3.2.1]octane-3-carboxylate (370 mg, Y: 32%) as a yellow solid. ES-API: [M/2+1]⁺=674.3

step 5: tert-butyl (1R, 5S)-8-(6-chloro-8-cyclopropoxy-7-(5-methyl-1H-indazol-4-yl)-2-((((S)-1-methylpyrrolidin-2-yl)methoxy)quinazolin-4-yl)-3,8-diazabicyclo[3.2.1]octane-3-carboxy late (370 mg, 0.55 mmol) was dissolved in dichloromethane (2 mL) and then trifluoroacetic acid (2 mL) was added. The reaction was conducted at room temperature for 1 h and the reaction mixture was dried by rotary evaporation to obtain crude 4-(((1R, 5S)-3,8-azabicyclo[3.2.1]octane-8-yl)-6-chloro-8-cyclopropoxy-7-(5-methyl-1H-indazol-4-yl)-2-((((S)-1-methylpyrrolidin-2-yl)methoxy)quinazoline (315 mg, Y: 100%) as a yellow oil.

step 6: 4-(((1R, 5S)-3,8-azabicyclo[3.2.1]octane-8-yl)-6-chloro-8-cyclopropoxy-7-(5-methyl-1H-indazol-4-yl)-2-((((S)-1-methylpyrrolidin-2-yl)methoxy)quinazoline (315 mg, 0.55 mmol) and triethylamine (167 mg, 1.65 mmol) were dissolved in dichloromethane (5 mL). At 0° C. acrylic anhydride (55 mg, 0.44 mmol) was added, and the reaction was conducted at 0° C. for 1 h. The reaction solution was dried by rotary evaporation and purified by preparative plate (dichloromethane:methanol:ammonium hydroxide=90:10) to obtain 1-(((1R, 5S)-8-(6-chloro-8-cyclopropoxy-7-(5-methyl-1H-indazol-4-yl)-2-((((S)-1-methylpyrrolidin-2-yl)methoxy)quinazolin-4-yl)-3,8-azabicyclo[3.2.1]octane-3-yl)prop-2-en-1-one (25 mg, Y: 7%). ES-API: [M+1]⁺=628.2. ¹HNMR (400 MHz, CDCl₃)δ 10.58 (s, 1H), 7.79 (s, 1H), 7.58 (s, 1H), 7.47 (d, J=8.5 Hz, 1H), 7.32 (d, J=8.5 Hz, 1H), 6.59 (dd, J=16.6, 10.6 Hz, 1H), 6.36 (d, J=16.6 Hz, 1H), 5.76 (d, J=10.4 Hz, 1H), 4.94 (s, 2H), 4.70 (s, 1H), 4.60 (d, J=12.9 Hz, 1H), 4.47 (s, 1H), 4.34 (s, 1H), 3.88 (s, 1H), 3.82-3.70 (m, 1H), 3.28 (s, 2H), 2.99 (s, 1H), 2.63 (s, 3H), 2.48 (s, 1H), 2.19 (s, 3H), 2.10 (d, J=11.0 Hz, 3H), 1.95-1.73 (m, 5H), 0.18 (dd, J=14.5, 4.7 Hz, 4H).

### Example 45 Preparation of Z45

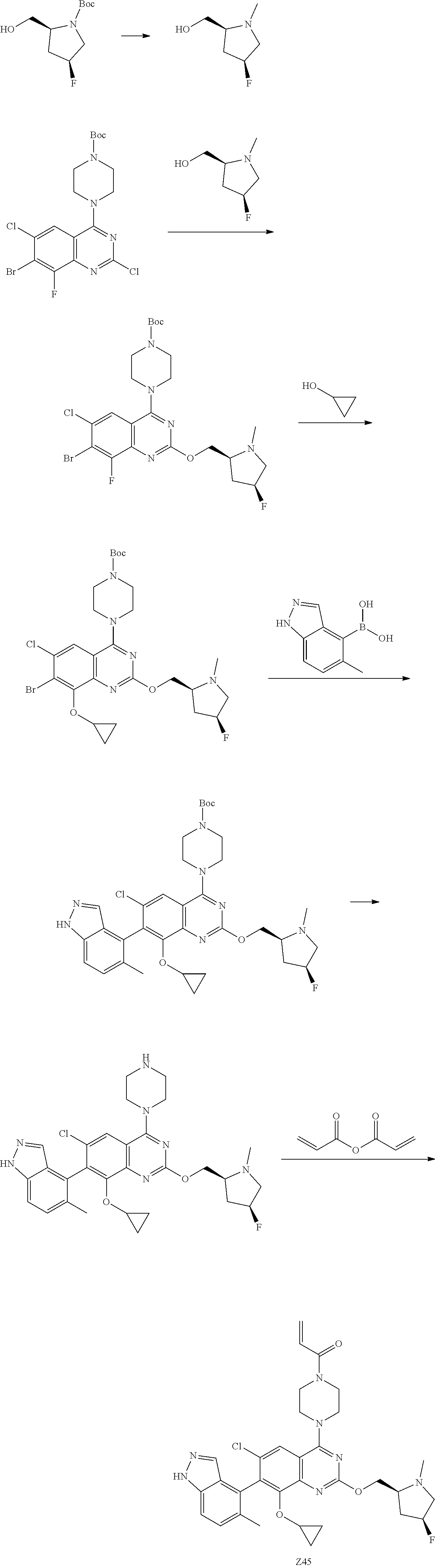

step 1: lithium aluminum tetrahydrogen (2.9 g, 0.077 mol) was added to tetrahydrofuran (50 mL), and then a solution of (2S, 4S)-1-(tert-butoxycarbonyl)-4-fluoropyrrolidin-2-carboxylic acid (6 g, 0.026 mol) in tetrahydrofuran (50 mL) was added dropwise while keeping stirring. After the addition, the reaction was conducted at room temperature for 18 h. Water (3 mL), sodium hydroxide solution (15%, 3 mL) and water (9 mL) were added dropwise to the reaction solution under ice bath, and stirred for 1 h after the addition. Then ethyl acetate (50 mL) was added and the organic phase was dried over anhydrous sodium sulfate and filtered. The filtrate was dried by rotary evaporation to obtain ((2S, 4S)-4-fluoro-1-methylpyrrolidin-2-yl) methanol (3 g, Y: 86.6%), which was used in the next step. ES-API: [M+1]⁺=134.1

step 2: tert-butyl 4-(7-bromo-2,6-dichloro-8-fluoroquinazolin-4-yl)piperazin-1-carboxylate (3.0 g, 6.25 mmol) was dissolved in N,N-dimethylformamide (30 mL) and then ((2S, 4S)-4-fluoro-1-methylpyrrolidin-2-yl) methanol (1.7 g, 12.29 mmol), cesium carbonate (6.1 g, 18.74 mmol) and triethylene diamine (71 mg, 0.63 mmol) were added. After the addition, the reaction was conducted at room temperature for 16 h. Ethyl acetate (100 mL) was added. The organic phase was washed with water and washed with brine and then dried by rotary evaporation, and purified by column chromatography (dichloromethane:methanol=10:1) to obtain tert-butyl 4-(7-bromo-6-chloro-8-fluoro-2-((((2S, 4S)-4-fluoro-1-methylpyrrolidin-2-yl)methoxy)quinazolin-4-yl)piperazin-1-carboxylate (1.8 g,Y: 50%). ES-API: [M+1]⁺=576.1

step 3: cyclopropanol (544 mg, 9.36 mmol) was dissolved in tetrahydrofuran (20 mL) and at 0° C. sodium hydrogen (375 mg, 9.36 mmol) was added. The reaction was stirred for 0.5 h while keeping the temperature. tert-butyl 4-(7-bromo-6-chloro-8-fluoro-2-((((2S, 4S)-4-fluoro-1-methylpyrrolidin-2-yl)methoxy)quinazolin-4-yl)piperazin-1-carboxylate (1.8 g, 3.12 mmol) was then added into the reaction solution. The reaction was conducted for 1 h while keeping the temperature. The reaction solution was poured into ethyl acetate (100 mL), the organic phase was washed with water (50 mL×2) and washed with brine (50 mL), dried over anhydrous sodium sulfate and filtered. The filtration was dried by rotary evaporation to obtain tert-butyl 4-(7-bromo-6-chloro-8-cyclopropoxy-2-((((2S, 4S)-4-fluoro-1-methylpyrrolidin-2-yl)methoxy)quinazolin-4-yl)piperazin-1-carboxylate (1.9 g, Y: 100%) as a yellow oil. ES-API: [M+1]⁺=614.1

step 4: 4-(7-bromo-6-chloro-8-cyclopropoxy-2-((((2S, 4S)-4-fluoro-1-methylpyrrolidin-2-yl)methoxy)q tert-butyl uinazolin-4-yl)piperazin-1-carboxylate (1.9 g, 3.09 mmol), (5-methyl-1H-indazol-4-yl) boronic acid (653 mg, 3.7 mmol), tripotassium phosphate (1.3 g, 6.18 mmol), tris (dibenzylideneindenacetone)dipalladium (200 mg) and 2-dicyclohexylphosphine-2′,4′,6′-triisopropylbiphenyl (400 mg) were added in sequence into dioxane (15 mL) and water (3 mL). The system atmosphere was replaced with nitrogen three times, then the temperature was warmed up to 105° C. and the reaction was conducted for 16 h. After cooling to room temperature, the reaction solution was poured into ethyl acetate (50 mL) and after being washed with brine once it was purified by column on silica gel (methanol:dichloromethane=0˜1:10) to obtain tert-butyl 4-(6-chloro-8-cyclopropoxy-2-((((2S, 4S)-4-fluoro-1-methylpyrrolidin-2-yl)methoxy)-7-(5-methyl-1H-indazol-4-yl)quinazolin-4-yl)piperazin-1-carboxylate (900 mg, Y: 43%) as a yellow solid. ES-API: [M+1]⁺=666.3

step 5: tert-butyl 4-(6-chloro-8-cyclopropoxy-2-((((2S, 4S)-4-fluoro-1-methylpyrrolidin-2-yl) methoxy)-7-(5-methyl-1H-indazol-4-yl)quinazolin-4-yl)piperazin-1-carboxylate (900 mg, 1.35 mmol) was dissolved in dichloromethane (2 mL) and then trifluoroacetic acid (2 mL) was added. The reaction was conducted at room temperature for 1 h and the reaction mixture was dried by rotary evaporation to obtain 6-chloro-8-cyclopropoxy-2-((((2S, 4S)-4-fluoro-1-methylpyrrolidin-2-yl)methoxy)-7-(5-methyl-1H-indazol-4-yl)-4-(piperazin-1-yl)quinazoline (780 mg, Y: 100%) as a yellow oil.

step 6: 6-chloro-8-cyclopropoxy-2-((((2S, 4S)-4-fluoro-1-methylpyrrolidin-2-yl)methoxy)-7-(5-methyl-1H-indazol-4-yl)-4-(piperazin-1-yl)quinazoline (764 mg, 1.35 mmol) and triethylamine (410 mg, 4.05 mmol) were dissolved in dichloromethane (10 mL). At 0° C. acrylic anhydride (153 mg, 1.22 mmol) was added, and the reaction was conducted at 0° C. for 1 h. After the reaction solution was dried by rotary evaporation, it was purified by preparation to obtain 1-(4-(6-chloro-8-cyclopropoxy-2-((((2S, 4S)-4-fluoro-1-methylpyrrolidin-2-yl)methoxy)-7]-(5-methyl-1H-indazol-4-yl)quinazolin-4-yl)piperazin-1-yl)prop-2-en-1-one (160 mg, Y: 19%). ES-API: [M+1]⁺=620.2. ¹HNMR (400 MHz, CDCl₃)δ 10.76 (s, 1H), 7.74 (s, 1H), 7.57 (s, 1H), 7.46 (d, J=8.3 Hz, 1H), 7.31 (d, J=8.4 Hz, 1H), 6.61 (dd, J=16.6, 10.6 Hz, 1H), 6.37 (d, J=16.7 Hz, 1H), 5.77 (d, J=10.4 Hz, 1H), 5.13 (d, J=54.5 Hz, 1H), 4.67 (d, J=10.0 Hz, 1H), 4.44 (d, J=4.3 Hz, 1H), 4.34 (s, 1H), 3.89 (d, J=26.8 Hz, 8H), 3.37 (dd, J=17.7, 12.2 Hz, 1H), 2.89 (s, 1H), 2.59-2.40 (m, 5H), 2.19 (s, 3H), 2.08 (s, 1H), 0.28-0.10 (m, 4H).

### Example 46 Preparation of Z46

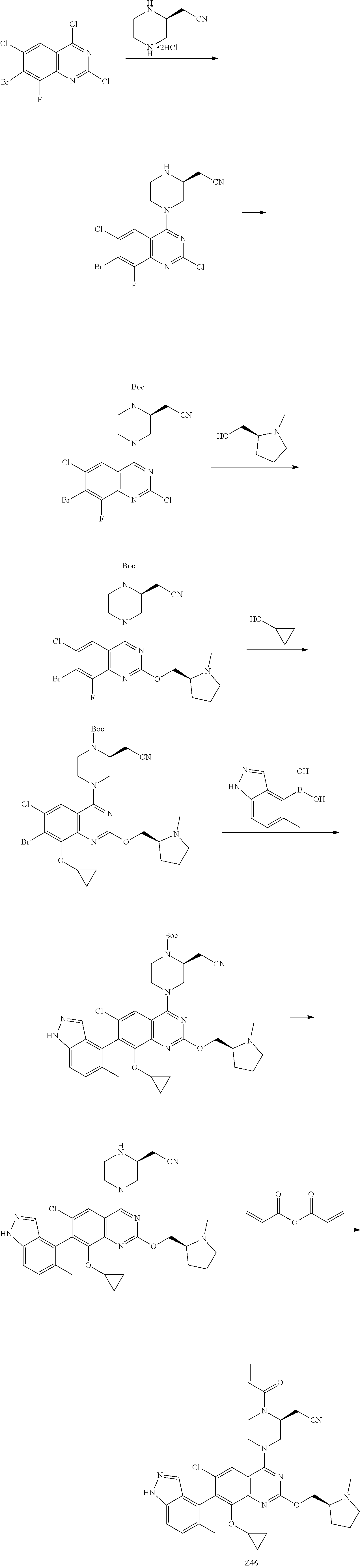

step 1: 7-bromo2,4,6-trichloro-8-fluoroquinazoline (4 g, 12.1 mmol) was added to DCM/ACN(30/30 mL). After the reaction solution was cooled to 0° C. and then TEA (6.1 g, 60.5 mmol) was added slowly. After that (S)-2-(piperazin-2-yl) acetonitrile hydrochloride (3.6 g, 18.2 mmol) was added. The reaction solution was slowly warmed up to room temperature and then stirred for 2 h. It was detected by LCMS till the reaction was complete. The reaction solution was not treated and directly proceeded to the next step. ES-API: [M+1]⁺=418.0

step 2: to the reaction solution obtain in step 1 DMAP (150 mg, 1.21 mmol) and Boc₂O (3.2 g, 14.5 mmol) were added. After the reaction solution was stirred at room temperature for 16 h, it was sent to LCMS to detect that the reaction was complete. Water (100 mL) was added to the reaction solution, which was extracted with ethyl acetate (2*100 mL). The organic phase was dried over anhydrous sodium sulfate and then dried by rotary evaporation to obtain a crude which was purified by flash column on silica gel (petroleum ether/ethyl acetate=5/1) to obtain 2.1 g of tert-butyl (S)-4-(7-bromo-2,6-dichloro-8-fluoroquinazolin-4-yl)-2-(cyanomethyl)piperazin-1-carboxylate (Y: 33%) as a pale yellow solid. ES-API: [M+1]⁺=518.0

step 3: tert-butyl (S)-4-(7-bromo-2,6-dichloro-8-fluoroquinazolin-4-yl)-2-(cyanomethyl)piperazin-1-carboxylate (2.1 g, 4.0 mmol) was added to (S)-(1-methylpyrrolidin-2-yl) methanol (9.3 g, 80 mmol) and then KF (700 mg, 12 mmol) was added. The mixture was heated to 100° C. and the reaction was conducted for 2 h. It was detected by LCMS till the reaction was complete. After the reaction solution was cooled to room temperature, 100 mL of water was added, and the mixture was extracted with ethyl acetate (50 mL*3). The organic phase was dried and dried by rotary evaporation to obtain a crude, which was purified by flash column on silica gel (dichloromethane/methanol=50/1) to obtain 1.7 g of product tert-butyl (S)-4-(7-bromo-6-chloro-8-fluoro-2-((((S)-1-methylpyrrolidin-2-yl) methoxy)quinazolin-4-yl)-2-cyanomethyl piperazin-1-carboxylate (Y: 70%). ES-API: [M+1]⁺=597.1

step 4: cyclopropanol (250 mg, 4.26 mmol) was dissolved in tetrahydrofuran (15 mL) and at 0° C. sodium hydrogen (681 mg, 28.4 mmol) was added. The reaction was stirred for 0.5 h while keeping the temperature. tert-butyl (S)-4-(7-bromo-6-chloro-8-fluoro-2-((((S)-1-methylpyrrolidin-2-yl)methoxy)quinazolin-4-yl)-2-cyanomethyl piperazin-1-carboxylate (1.7 g, 2.84 mmol) was then added into the reaction solution. The reaction was conducted for 1 h while keeping the temperature. The reaction solution was poured into ethyl acetate (30 mL), the organic phase was washed with water (30 mL×2) and washed with brine (30 mL), dried over anhydrous sodium sulfate and filtered. The filtration was dried by rotary evaporation to obtain a crude as a yellow oil, which was purified by flash column on silica gel (dichloromethane/methanol=100/1) to obtain tert-butyl (S)-4-(7-bromo-6-chloro-8-cyclopropoxy-2-((((S)-1-methylpyrrolidin-2-yl)methoxy)quinazolin-4-yl)-2-cyanomethyl piperazin-1-carboxylate (1 g, Y: 55%). ES-API: [M+1]⁺=635.1

step 5: tert-butyl (S)-4-(7-bromo-6-chloro-8-cyclopropoxy-2-((((S)-1-methylpyrrolidin-2-yl)methoxy)quinazolin-4-yl)-2-cyanomethyl piperazin-1-carboxylate (1 g, 1.58 mmol), (5-methyl-1H-indazol-4-yl) boronic acid (555 mg, 3.16 mmol), tripotassium phosphate (1 g, 4.74 mmol), tris (dibenzylideneindenacetone)dipalladium (150 mg, 0.16 mmol), 2-dicyclohexylphosphine-2′,4′,6′-triisopropylbiphenyl (75 mg, 0.16 mmol) were added in sequence into dioxane (8 mL) and water (2 mL). The system atmosphere was replaced with nitrogen three times, then the temperature was warmed up to 100° C. and the reaction was conducted for 16 h. After cooling to room temperature, the reaction solution was poured into ethyl acetate (30 mL) and after being washed with brine once it was purified by column on silica gel (dichloromethane/methanol=20/1) to obtain tert-butyl (2S)-4-(6-chloro-8-cyclopropoxy-7-(5-methyl-1H-indazol-4-yl)-2-((((S)-1-methylpyrrolidin-2-yl)) methoxy)quinazolin-4-yl)-2-(cyanomethyl)piperazin-1-carboxylate (600 mg, Y: 55%) as a yellow solid. ES-API: [M+1]⁺=687.3

step 6: tert-butyl (2S)-4-(6-chloro-8-cyclopropoxy-7-(5-methyl-1H-indazol-4-yl)-2-((((S)-1-methylpyrrolidin-2-yl))methoxy)quinazolin-4-yl)-2-(cyanomethyl)piperazin-1-carboxylate (600 mg, 0.87 mmol) was dissolved in dichloromethane (5 mL) and then trifluoroacetic acid (2 mL) was added. The reaction was conducted at room temperature for 1 h and the reaction mixture was dried by rotary evaporation to obtain crude 2-((2S)-4-(6-chloro-8-cyclopropoxy-7-(5-methyl-1H-indazol-4-yl)-2-((((S)-1-methylpyrrolidin-2-yl))methoxy)quinazolin-4-yl)piperazin-2-yl) acetonitrile (500 mg, Y: 100%) as a yellow oil.

step 7: 2-((2S)-4-(6-chloro-8-cyclopropoxy-7-(5-methyl-1H-indazol-4-yl)-2-((((S)-1-methylpyrrolidin-2-yl))methoxy)quinazolin-4-yl)piperazin-2-yl) acetonitrile (500 mg, 0.87 mmol) was dissolved in dichloromethane (5 mL). At 0° C. triethylamine (2 mL) and acrylic anhydride (100 mg, 0.78 mmol) were added. The reaction was conducted at 0° C. for 0.5 h. The reaction solution was extracted by adding 20 mL of water and DCM (20 mL*3) and the organic phase was dried by rotary evaporation to obtain 780 mg of a crude, which was purified by preparation to obtain 2-((2S)-1-acryloyl-4-(6-chloro-8-cyclopropoxy-7-(5-methyl-1H-indazol-4-yl)-2-((((S)-1-methylpyrrolidone-2-yl)methoxy)quinazolin-4-ylpiperazin-2-yl) acetonitrile (80 mg, Y: 14.6%). The preparation and separation conditions were the same as in Example 34. ES-API: [M+1]⁺=641.3. ¹HNMR (400 MHz, CDCl₃)δ 10.37 (s, 1H), 7.75 (s, 1H), 7.57 (d, J=3.0 Hz, 1H), 7.47 (d, J=8.5 Hz, 1H), 7.33 (d, J=8.6 Hz, 1H), 6.59 (d, J=13.1 Hz, 1H), 6.41 (d, J=16.6 Hz, 1H), 5.84 (d, J=10.5 Hz, 1H), 5.23 (d, J=82.3 Hz, 1H), 4.62 (s, 1H), 4.44-4.29 (m, 4H), 3.73 (d, J=40.1 Hz, 2H), 3.44 (s, 1H), 3.18 (s, 1H), 3.02-2.95 (m, 1H), 2.84 (s, 2H), 2.55 (s, 3H), 2.37 (d, J=16.7 Hz, 1H), 2.19 (d, J=2.6 Hz, 3H), 2.10 (s, 1H), 1.81 (s, 4H), 0.30-0.10 (m, 4H).

### Example 47 Preparation of Z47

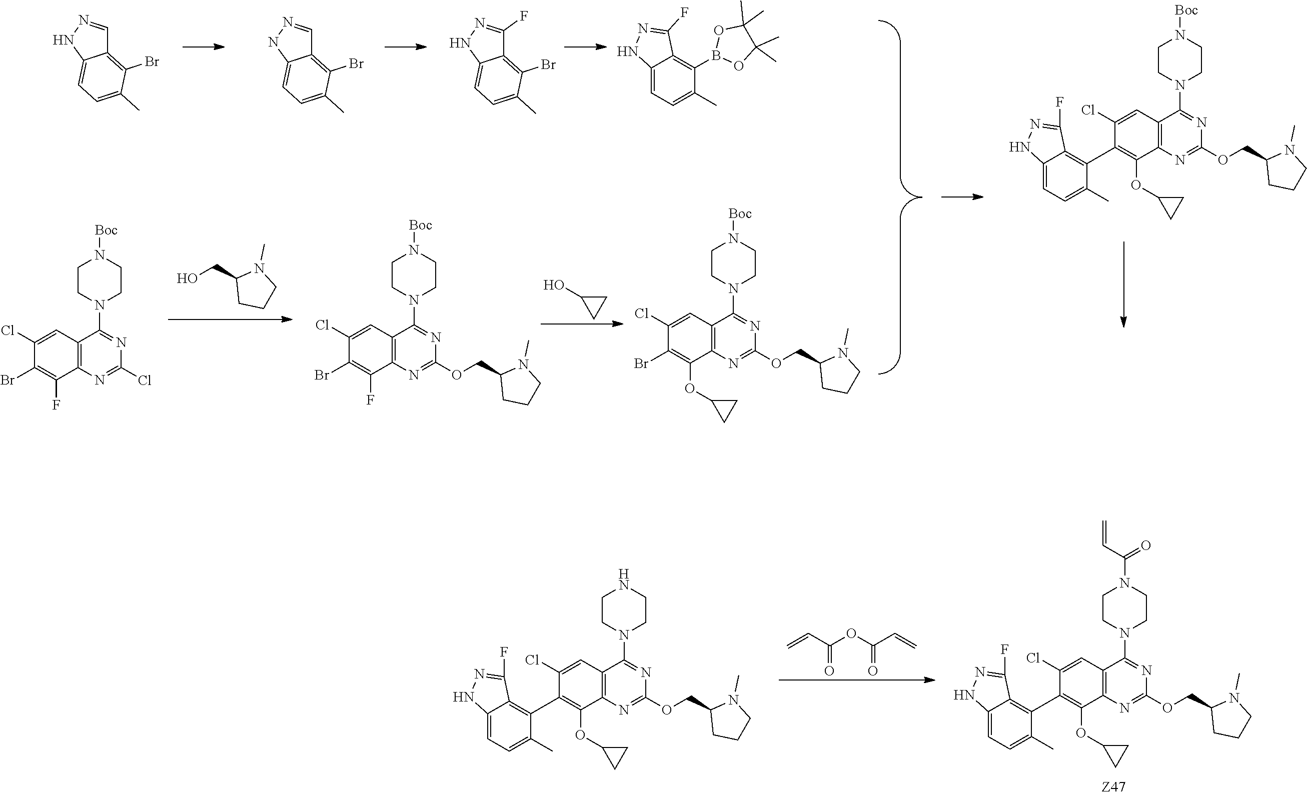

step 1: 4-bromo-5-methyl-1H-indazole (5 g, 23.8 mmol) was dissolved in dichloromethane (50 mL), and after cooling by ice water 3,4-dihydro-2H-pyran (4 g, 47.6 mmol) was added and then p-toluenesulfonic acid monohydrate (452 mg, 2.38 mmol) was added. The reaction was conducted at 10° C. for 1 h. The reaction solution was quenched by adding water, and the organic phase was washed with saturated sodium bicarbonate and brine and dried over sodium sulfate. The solvent was concentrated under reduced pressure to dryness to obtain crude product 4-bromo-5-methyl-1-(tetrahydro-2H-pyran-2-yl)-1H-indazole (8.5 g, Y: 100%). ES-API: [M+1]⁺=295.0

step 2: 4-bromo-5-methyl-1-(tetrahydro-2H-pyran-2-yl)-1H-indazole (8.5 g, 23.8 mmol) was dissolved in N,N-dimethylformamide (300 mL), and 1-chloromethyl-4-fluoro-1,4-diazabicyclo[2.2.2]octane ditetrafluoro boronic acid (29.5 g, 83.3 mmol) was added. The temperature was warmed up to 70° C. and the reaction was conducted for 2 h. The reaction solution was cooled, quenched by adding water, and extracted with ethyl acetate. The organic phase was washed with saturated brine and dried over sodium sulfate. The solvent was dried by rotary evaporation under reduced pressure, and purified by column (petroleum ether/ethyl acetate=3/1) to obtain 4-bromo-3-fluoro-5-methyl-1H-indazole (1.4 g, Y: 25.8%). ES-API: [M+MeOH]⁺=229.0

step 3: 4-bromo-3-fluoro-5-methyl-1H-indazole (1.4 g, 6.14 mmol), bis(pinacolato)diboron (2.34 g, 9.21 mmol), potassium acetate (1806 mg, 18.44 mmol) and Pd (dppf)Cl₂ (449 mg, 0.614 mmol) were added in sequence into N,N-dimethylformamide (15 mL). The system atmosphere was replaced with nitrogen, then the reaction was conducted at 100° C. for 4 h. The reaction solution was cooled, and separated by adding water and ethyl acetate. The organic phase was washed with brine, dried over sodium sulfate, dried by rotary evaporation under reduced pressure, and purified by column (petroleum ether/ethyl acetate=3/1), to obtain 3-fluoro-5-methyl-4-(4,4,5,5-tetramethyl-1,3,2-dioxaborane-2-yl)-1H-indazole (720 mg,Y: 42.5%). ES-API: [M+1]⁺=277.1

step 4: 3-fluoro-5-methyl-4-(4,4,5,5-tetramethyl-1,3,2-dioxaborane-2-yl)-1H-indazole (1 g, 2.1 mmol) was added to (S)-(1-methylpyrrolidin-2-yl) methanol (6 mL) and then potassium fluoride (364 mg, 6.3 mmol) was added. The mixture was heated to 100° C. and the reaction was conducted for 1 h, After the reaction solution was cooled to room temperature, water was added, and the mixture was extracted with ethyl acetate. The organic phase was dried and dried by rotary evaporation to obtain a crude which was purified by column (dichloromethane/methanol=100/1) to obtain tert-butyl (S)4-(7-bromo-6-chloro-8-fluoro-2-((1-methylpyrrolidin-2-yl) methoxy)quinazolin-4-yl)piperazin-1-carboxylate (1.1 g, Y: 94%). ES-API: [M+1]⁺=558.1

step 5: tert-butyl (S)4-(7-bromo-6-chloro-8-fluoro-2-((1-methylpyrrolidin-2-yl)methoxy) quinazolin-4-yl)piperazin-1-carboxylate (1.1 g, 1.97 mmol) and cyclopropanol (344 mg, 5.92 mmol) were dissolved in tetrahydrofuran (10 mL), and at 0° C. sodium hydrogen (788 mg, 19.7 mmol) was added in batches. The reaction was stirred for 0.5 h while keeping the temperature. The reaction was quenched by adding water, and the mixture was extracted with ethyl acetate. The organic phase was washed with brine, dried over anhydrous sodium sulfate and dried by rotary evaporation under reduced pressure, to obtain crude tert-butyl (S)4-(7-bromo-6-chloro-8-cyclopropoxy-2-((1-methylpyrrolidin-2-yl)methoxy)quinazolin-4-yl)piperazin-1-carboxylate (1.5 g, Y: 100%). ES-API: [M+1]⁺=596.2

step 6: tert-butyl (S)4-(7-bromo-6-chloro-8-cyclopropoxy-2-((1-methylpyrrolidin-2-yl) methoxy)quinazolin-4-yl)piperazin-1-carboxylate (650 mg, 1.09 mmol), 3-fluoro-5-methyl-4-(4,4,5,5-tetramethyl-1,3,2-dioxaborane-2-yl)-1H-indazole (603 mg, 2.18 mmol), potassium phosphate (462 mg, 2.18 mmol) and Sphos Pd G2 (78 mg, 0.109 mmol) were added in sequence into dioxane (20 mL) and water (5 mL). The system atmosphere was replaced with nitrogen three times, then the temperature was warmed up to 100° C. and the reaction was conducted for 3 h. After cooling to room temperature, the reaction solution was poured into ethyl acetate (30 mL) and after being washed with brine once, it was dried over anhydrous sodium sulfate, dried by rotary evaporation under reduced pressure and purified by column (dichloromethane/methanol=100/1), to obtain crude product tert-butyl 4-(6-chloro-8-cyclopropoxy-7-(3-fluoro-5-methyl-1H-indazol-4-yl)-2-((((S)-1-methylpyrrolidin-2-yl))methoxy)quinazolin-4-yl)piperazin-1-carboxylate (200 mg, Y: 27.6%). ES-API: [M+1]⁺=666.3

step 7: tert-butyl 4-(6-chloro-8-cyclopropoxy-7-(3-fluoro-5-methyl-1H-indazol-4-yl)-2-((((S)-1-methylpyrrolidin-2-yl))methoxy)quinazolin-4-yl)piperazin-1-carboxylate (200 mg, 0.3 mmol) was dissolved in dichloromethane (1 mL) and then trifluoroacetic acid (1 mL) was added. The reaction was conducted at room temperature for 0.5 h and the reaction mixture was dried by rotary evaporation to obtain crude 6-chloro-8-cyclopropoxy-7-(3-fluoro-5-methyl-1H-indazol-4-yl)-2-((((S)-1-methylpyrrolidin-2-yl)methoxy)-4-(piperazin-1-yl)quinazoline (250 mg, Y: 100%). ES-API: [M+1]⁺=566.3

step 8: 6-chloro-8-cyclopropoxy-7-(3-fluoro-5-methyl-1H-indazol-4-yl)-2-((((S)-1-methylpyrrolidin-2-yl)methoxy)-4-(piperazin-1-yl)quinazoline (250 mg, 0.3 mmol) was dissolved in dichloromethane (3 mL). After cooling by ice water, triethylamine (1 mL) was added, and then acrylic anhydride (30 mg, 0.24 mmol) was added. The reaction was conducted at 10° C. for 0.5 h. The reaction solution was separated by adding 20 mL of water and DCM (20 mL). The organic phase was washed with brine, dried over anhydrous sodium sulfate, dried by rotary evaporation under reduced pressure, and purified by chromatographic preparation (column: Ultimate XB-C18, 50*250 mm, 10 um, mobile phase: A: purified water B: purified acetonitrile, flow rate: 80 ml/min, gradient: within 50 min, B/A=20%-90%, wavelength: 214 nm, column temperature: room temperature) to obtain 1-(4-(6-chloro-8-cyclopropoxy-7-(3-fluoro-5-methyl-1H-indazol-4-yl)-2-((((S)-1-methylpyrrolidin-2-yl))methoxy)quinazolin-4-yl)piperazin-1-yl)prop-2-en-1-one (13.7 mg, Y: 7.36%). ES-API: [M+1]⁺=620.3. ¹HNMR (400 MHz, CDCl₃)δ9.54 (s, 0H), 7.72 (s, 1H), 7.35 (s, 2H), 6.61 (dd, J=16.9, 10.6 Hz, 1H), 6.37 (d, J=17.0 Hz, 1H), 5.77 (d, J=10.7 Hz, 1H), 4.73 (s, 0H), 4.51 (s, 1H), 4.41 (s, 1H), 3.89 (d, J=30.3 Hz, 8H), 2.69 (s, 3H), 2.49 (s, 1H), 2.17 (s, 3H), 1.90 (s, 7H), 0.21 (s, 3H).

### Example 48 Preparation of Z48

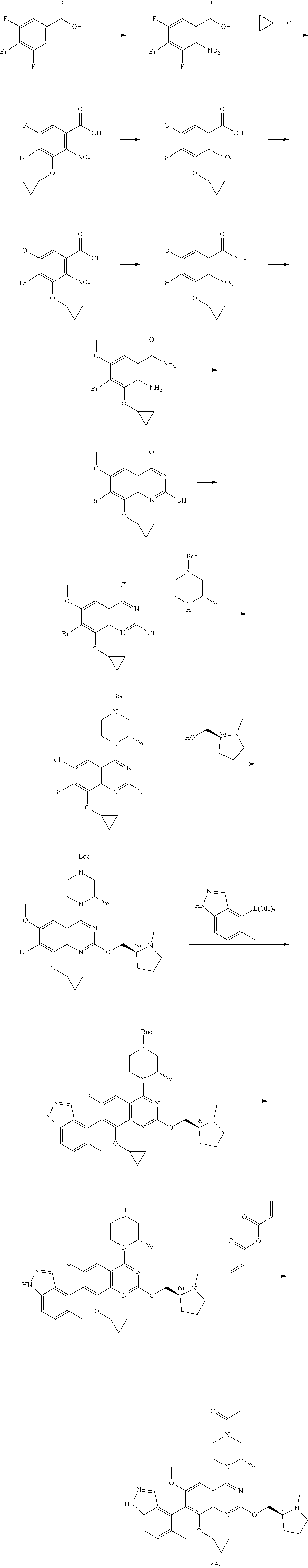

step 1: 4-bromo-3,5-difluorobenzoic acid (20 g, 0.084 mol) was added to concentrated sulfuric acid (100 mL), and then potassium nitrate (8.5 g, 0.084 mol) was added. After the mixture was stirred evenly, it was heated to 80° C. and then the reaction was conducted for 3 h. After cooling, the reaction solution was poured into water (500 mL) and extracted with ethyl acetate (300 mL×3). The organic phase was washed with water and washed wieh brine, dried by rotary evaporation to obtain the product 4-bromo-3,5-difluoro-2-nitrobenzoic acid (18 g,Y: 75%) as a yellow solid. ¹HNMR (400 MHz, DMSO-d6)δ 7.84 (d, J=8.1 Hz, 1H).

step 2: cyclopropanol (2.68 g, 0.046 mol) was added to tetrahydrofuran (400 mL) and after the mixture was cooled to 0° C. sodium hydrogen (5.5 g, 0.14 mol) was added. After the mixture was stirred evenly 4-bromo-3,5-difluoro-2-nitrobenzoic acid (13 g, 0.046 mol) was added. After the addition, the reaction was conducted at room temperature overnight. The reaction solution was quenched by adding water (20 mL) and tetrahydrofuran was removed by rotary evaporation. The pH of the mixture was adjusted by dilute hydrochloric acid to 4-5, and the mixture was extracted with ethyl acetate (100 mL×3), washed with water and washed with brine, dried by rotary evaporation to obtain the product 4-bromo-3-cyclopropoxy-5-fluoro-2-nitrobenzoic acid (13 g,Y: 80%) as a yellow solid. ¹HNMR (400 MHz, DMSO-d6)δ 7.65 (s, 1H), 4.47-4.38 (m, 1H), 0.69 (d, J=12.1 Hz, 2H), 0.65-0.57 (m, 2H).

step 3: 4-bromo-3-cyclopropoxy-5-fluoro-2-nitrobenzoic acid (13 g, 0.04 mol) was dissolved in methanol (80 mL) and then a solution of sodium methoxide in methanol (16 mL, 5M) was added. After the mixture was heated to 50° C. and the reaction was conducted for 2 h. The reaction solution was quenched by adding water (10 mL) and methanol was removed by rotary evaporation. The pH of the mixture was adjusted by dilute hydrochloric acid to 4-5, and the mixture was extracted with ethyl acetate (100 mL×3), washed with water and washed with brine, dried by rotary evaporation to obtain the product 4-bromo-3-cyclopropoxy-5-methoxy-2-nitrobenzoic acid (10 g, Y: 75%) as a yellow solid. ¹HNMR (400 MHz, DMSO-d6)δ 7.34 (s, 1H), 4.40-4.35 (m, 1H), 3.98 (s, 3H), 0.72-0.67 (m, 2H), 0.59 (d, J=6.8 Hz, 2H).

step 4: 4-bromo-3-cyclopropoxy-5-methoxy-2-nitrobenzoic acid (9.6 g, 28.91 mmol) was added to thionyl chloride (100 mL). After the mixture heated to 80° C. and the reaction was conducted for 3 h. Then the mixture was dried by rotary evaporation to give a crude which was used directly in the next step.

step 5: 4-bromo-3-cyclopropoxy-5-methoxy-2-nitrobenzoyl chloride (10.1 g, 28.91 mmol) was dissolved in ultradry tetrahydrofuran (100 mL), which was added dropwise into a 0° C. ammonium hydroxide (100 mL). After the addition, the reaction was conducted at room temperature for 3 h. The reaction mixture was extracted with ethyl acetate (100 mL×3), washed with water and washed with brine, dried by rotary evaporation to obtain 4-bromo-3-cyclopropoxy-5-methoxy-2-nitrobenzamide (9 g, Y: 90%). ES-API: [M+H]⁺=331.0.

step 6: 4-bromo-3-cyclopropoxy-5-methoxy-2-nitrobenzamide (8.9 g, 0.027 mol) was added to a mixed solvent of acetic acid (140 ml) and water (70 mL). After the mixture was heated to 60° C. iron powder (15.1 g, 0.27 mol) was added in batches. After the addition, the reaction was conducted for 2 h while keeping the temperature. After the reaction solution was cooled, ethyl acetate (500 mL) was added. The mixture was filtered and the filtration was dried by rotary evaporation to obtain a crude, which was beat (10% ethyl acetate/petroleum ether 50 mL) to obtain 2-amino-4-bromo-3-cyclopropoxy-5-methoxybenzamide (4 g, Y: 49%) as a white solid. ES-API: [M+H]⁺=301.0

step 7: 2-amino-4-bromo-3-cyclopropoxy-5-methoxybenzamide (4 g, 0.013 mol) was dissolved in tetrahydrofuran (65 ml). After the mixture was cooled to 0° C., sodium hydrogen (2.7 g, 0.066 mol) was added. After stirring for 0.5 h, carbonyldiimidazole (3.2 g, 0.02 mol) was slowly added. After the addition the reaction was conducted at room temperature for 1 h. After cooling to 0° C. the reaction was quenched by adding water (5 mL) and the mixture was dried by rotary evaporation. The residue was beat (10% ethyl acetate/petroleum ether 50 mL) and dried to obtain 7-bromo-8-cyclopropoxy-6-methoxyquinazolin-2,4-diol (3.5 g, Y: 82%). ¹HNMR (400 MHz, DMSO-d6)δ 11.42 (s, 1H), 10.58 (s, 1H), 7.25 (s, 1H), 4.40-4.33 (m, 1H), 3.87 (s, 3H), 0.99 (p, J=5.2 Hz, 2H), 0.55-0.46 (m, 2H). ES-API: [M+H]⁺=327.0.

step 8: 7-bromo-8-cyclopropoxy-6-methoxyquinazolin-2,4-diol (2 g, 6.1 mol) was added to phosphorus oxychloride (30 ml). After the mixture was heated to 100° C., N,N-diisopropylethylamine was added slowly till the reaction was clear. After the mixture was heated to 130° C. the reaction was conducted for 2 h. After the reaction solution was cooled and phosphorus oxychloride was removed by rotary evaporation, the residue was poured into water (100 ml). The mixture was washed with water and washed with brine. The organic phase was dried and dried by rotary evaporation to obtain crude 7-bromo-2,4-dichloro-8-cyclopropoxy-6-methoxyquinazoline (2.2 g, Y: 95%).

step 9: 7-bromo-2,4-dichloro-8-cyclopropoxy-6-methoxyquinazoline (2 g, 5.49 mol) was dissolved in tetrahydrofuran (50 ml), and then tert-butyl (S)3-methylpiperazin-1-carboxylate (1.2 g, 6.04 mol) and triethylamine (1.7 g, 16.48 mol) were added. The mixture was heated to 60° C. and the reaction was conducted for 5 h, After the reaction solution was cooled and ethyl acetate (100 ml) was added, the mixture was washed with water, washed with ammonium chloride solution and washed with brine. The organic phase was dried and dried by rotary evaporation and purified by column (petroleum ether:ethyl acetate=3:1) to obtain tert-butyl (S)4-(7-bromo-2-chloro-8-cyclopropoxy-6-methoxyquinazolin-4-yl)-3-methylpiperazin-1-carboxy late (3 g, Y: 100%) as a yellow solid. ES-API: [M+H]⁺=527.1

step 10: after cooling to 0° C. (S)-(1-methylpyrrolidin-2-yl) methanol (20 mL) was added to sodium hydrogen (318 mg, 7.96 mmol), then tert-butyl (S)4-(7-bromo-2-chloro-8-cyclopropoxy-6-methoxyquinazolin-4-yl)-3-methylpiperazin-1-carboxylate (1.4 g, 2.65 mmol) was added. After the mixture was stirred evenly it was heated to 110° C. and the reaction was conducted for 1 h. After cooling, the reaction solution was poured into water (100 mL), extracted with ethyl acetate (100 mL×3), and purified by column (methanol:dichloromethane=0˜1:10) to obtain tert-butyl (S)-4-(7-bromo-8-cyclopropoxy-6-methoxy-2-((((S)-1-methylpyrrolidin-2-yl)methoxy)quinazolin-4-yl)-3-methylpiperazin-1-carboxylate (1 g, Y: 58%) as a yellow solid. ES-API: [M+H]⁺=606.2

step 11: tert-butyl (S)-4-(7-bromo-8-cyclopropoxy-6-methoxy-2-((((S)-1-methylpyrrolidin-2-yl)methoxy)quinazolin-4-yl)-3-methylpiperazin-1-carboxylate (1 g, 1.65 mmol), (5-methyl-1H-indazol-4-yl) boronic acid (435 mg, 2.47 mmol), tripotassium phosphate (1.05 g, 4.95 mmol), tris (dibenzylideneindenacetone)dipalladium (100 mg), 2-dicyclohexylphosphine-2′,4′,6′-triisopropylbiphenyl (200 mg) were added in sequence into dioxane (10 mL) and water (2 mL). The system atmosphere was replaced with nitrogen three times, then the temperature was warmed up to 110° C. and the reaction was conducted for 16 h. After cooling to room temperature, the reaction solution was poured into ethyl acetate (50 mL) and after being washed with brine once it was purified by column on silica gel (methanol:dichloromethane=0˜1:10) to obtain tert-butyl (3S)-4-(8-cyclopropoxy-6-methoxy-7-(5-methyl-1H-indazol-4-yl)-2-((((S)-1-methylpyrrolidin-2-yl))methoxy)quinazolin-4-yl)-3-methylpiperazin-1-carboxylate (550 mg, Y: 50%) as a yellow solid. ES-API: [M+H]⁺=658.4

step 12: tert-butyl (3S)-4-(8-cyclopropoxy-6-methoxy-7-(5-methyl-1H-indazol-4-yl)-2-((((S)-1-methylpyrrolidin-2-yl))methoxy)quinazolin-4-yl)-3-methylpiperazin-1-carboxylate (550 mg, 0.84 mmol) was dissolved in dichloromethane (2 mL) and then trifluoroacetic acid (1 mL) was added. The reaction was conducted at room temperature for 1 h and the reaction mixture was dried by rotary evaporation to obtain crude 8-cyclopropoxy-6-methoxy-7-(5-methyl-1H-indazol-4-yl)-4-((S)-2-methylpiperazin-1-yl)-2-((((S)-1-methylpyrrolidin-2-yl)methoxy) quinazoline (468 mg, Y: 100%) as a yellow oil.

step 13: 8-cyclopropoxy-6-methoxy-7-(5-methyl-1H-indazol-4-yl)-4-((S)-2-methylpiperazin-1-yl)-2-((((S)-1-methylpyrrolidin-2-yl)methoxy)quinazoline (468 mg, 0.84 mmol) and triethylamine (255 mg, 2.52 mmol) were dissolved in dichloromethane (4 mL). After cooling to 0° C. acrylic anhydride (95 mg, 0.76 mmol) was added, and then the reaction was conducted at 0° C. for 1 h. Water (10 mL) was added to the reaction solution, which was extracted with dichloromethane (10 mL×3). The organic phase was dried by rotary evaporation and purified by preparation to obtain a white solid (60 mg, Y: 11%) (Preparation and separation conditions:column:Ultimate XB-C18, 50*250 mm, 10 um, mobile phase: A: purified water B: purified acetonitrile, flow rate: 80 ml/min, gradient: within 50 min, B/A=20%-90%, wavelength: 214 nm, column temperature: room temperature) ES-API: [M+H]⁺=612.3. ¹HNMR (400 MHz, CDCl₃)δ 10.16 (s, 1H), 7.59 (s, 1H), 7.43 (d, J=8.6 Hz, 1H), 7.33 (d, J=8.6 Hz, 1H), 6.97 (s, 1H), 6.61 (dd, J=30.7, 11.0 Hz, 1H), 6.38 (d, J=16.4 Hz, 1H), 5.77 (d, J=10.9 Hz, 1H), 4.75-4.33 (m, 5H), 4.11 (s, 1H), 3.70 (d, J=25.7 Hz, 5H), 3.28 (d, J=13.7 Hz, 1H), 3.11 (s, 1H), 2.75 (s, 1H), 2.50 (s, 3H), 2.28 (s, 1H), 2.21 (s, 3H), 2.07 (s, 1H), 1.79 (d, J=21.6 Hz, 4H), 1.43 (d, J=6.7 Hz, 3H), 0.26-0.10 (m, 4H).

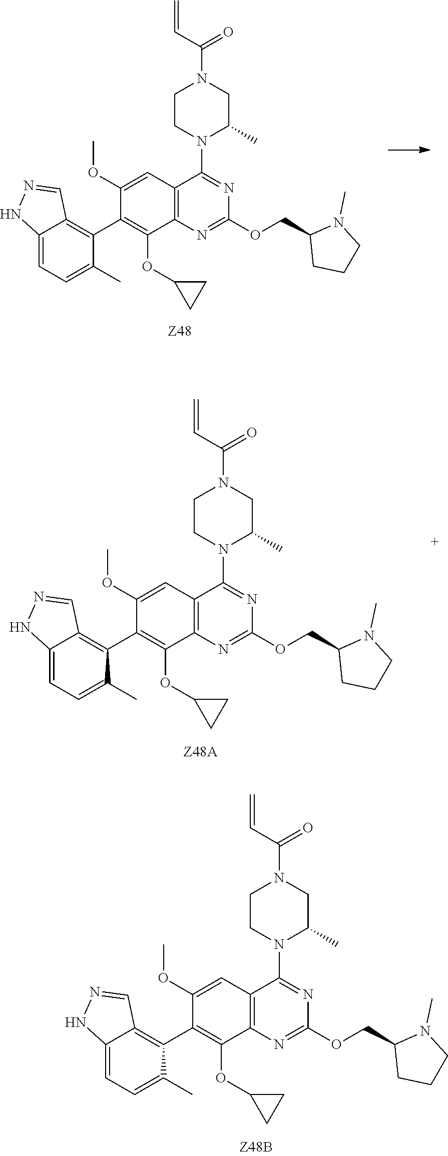

The above compound Z48 (50 mg) was separated through chiral resolution (mobile phase: HEX:ETOH:DEA=80:20:0.2; column: IG 254 nanometer@4.8 nanometer; flow rate: 1.0 ml/min; column temperature: 30.0° C.) to obtain one isomer, arbitrarily designated as Z48A: 1-((S)-4-((R)-8-cyclopropoxy-6-methoxy-7-(5-methyl-TH-indazol-4-yl)-2-((((S)-1-methylpyrrolid one-pyridin-2-yl)methoxy)quinazolin-4-yl)-3-methylpiperazin-1-yl)prop-2-en-1-one (retention time: 6.171 min; 19 mg, purity: 100%, de value: 100%). ES-API: [M+H]⁺=612.3; and the he other isomer, arbitrarily designated as Z48B: 1-((S)-4-((S)-8-cyclopropoxy-6-methoxy-7-(5-methyl-TH-indazol-4-yl)-2-((((S)-1-methylpyrrolid one-pyridin-2-yl)methoxy)quinazolin-4-yl)-3-methylpiperazin-1-yl)prop-2-en-1-one (retention time: 7.660 min; 23 mg; purity: 100%, de value: 100%). ES-API: [M+H]⁺=612.3.

### Example 49 Preparation of Z49

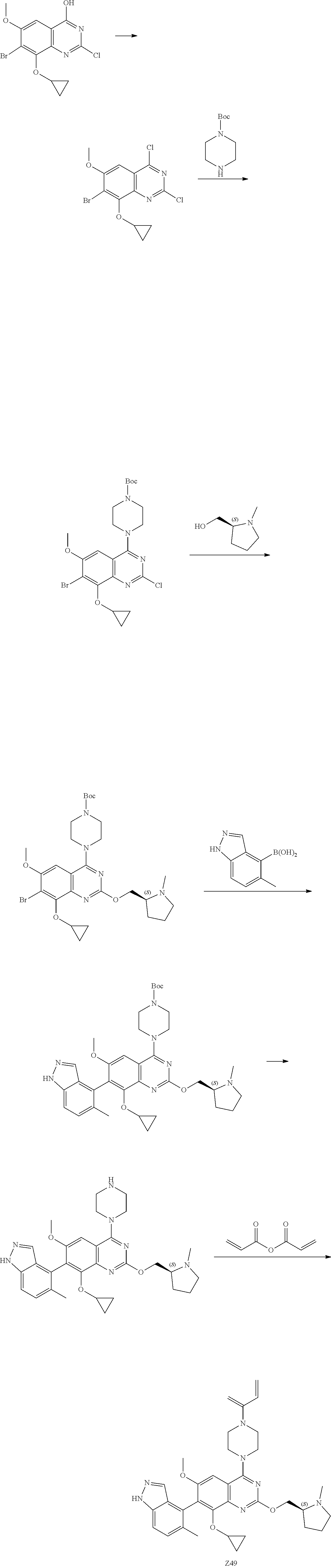

step 1: 7-bromo-8-cyclopropoxy-6-methoxyquinazolin-2,4-diol (1.5 g, 4.59 mol) was added to phosphorus oxychloride (20 ml). After the mixture was heated to 100° C., N,N-diisopropylethylamine was added slowly till the reaction solution was clear. After the mixture was heated to 130° C. the reaction was conducted for 2 h. After the reaction solution was cooled and phosphorus oxychloride was removed by rotary evaporation, the residue was poured into water (100 ml). The organic phase was washed with water and washed with brine, dried and dried by rotary evaporation to obtain crude 7-bromo-2,4-dichloro-8-cyclopropoxy-6-methoxyquinazoline (2.1 g, Y: 100%). ES-API: [M+H]⁺=362.9

step 2: 7-bromo-2,4-dichloro-8-cyclopropoxy-6-methoxyquinazoline (2.1 g, 5.78 mol) was dissolved in tetrahydrofuran (50 ml) and then tert-butyl piperazin-1-carboxylate (1.6 g, 8.68 mol) and triethylamine (1.75 g, 17.34 mol) were added. The mixture was heated to 60° C. and the reaction was conducted for 5 h, After the reaction solution was cooled, ethyl acetate (100 ml) was added. The organic phase was washed with water, washed with ammonium chloride solution and washed with brine, dried by rotary evaporation and purified by column (petroleum ether:ethyl acetate=3:1) to obtain tert-butyl 4-(7-bromo-2-chloro-8-cyclopropoxy-6-methoxyquinazolin-4-yl)piperazin-1-carboxylate (1.1 g, Y: 50%) as a yellow solid. ES-API: [M+H]⁺=513.1

step 3: at 0° C., (S)-(1-methylpyrrolidin-2-yl) methanol (15 mL) was added to sodium hydrogen (390 mg, 9.73 mmol) and tert-butyl 4-(7-bromo-2-chloro-8-cyclopropoxy-6-methoxyquinazolin-4-yl)piperazin-1-carboxylate (1 g, 1.95 mmol). After the mixture was stirred evenly it was heated to 110° C. and the reaction was conducted for 1 h. After cooling, the reaction solution was poured into water (100 mL), extracted with ethyl acetate (100 mL×3), and purified by column (methanol:dichloromethane=0˜1:10) to obtain tert-butyl ((S)-4-(7-bromo-8-cyclopropoxy-6-methoxy-2-((1-methylpyrrolidin-2-yl)methoxy)quinazolin-4-yl) piperazin-1-carboxylate (750 mg, Y: 68%) as a yellow solid. ES-API: [M+H]⁺=592.2

step 4: tert-butyl ((S)-4-(7-bromo-8-cyclopropoxy-6-methoxy-2-((1-methylpyrrolidin-2-yl)methoxy)quinazolin-4-yl)piperazin-1-carboxylate (750 mg, 1.26 mmol), (5-methyl-1H-indazol-4-yl) boronic acid (450 mg, 2.52 mmol), tripotassium phosphate (850 mg, 3.78 mmol), tris (dibenzylideneindenacetone)dipalladium (100 mg) and 2-dicyclohexylphosphine-2′,4′,6′-triisopropylbiphenyl (60 mg) were added in sequence to dioxane (8 mL) and water (2 mL). The system atmosphere was replaced with nitrogen three times, then the reaction was conducted at 110° C. for 16 h. After cooling to room temperature, the reaction solution was poured into ethyl acetate (50 mL) and after being washed with brine once it was purified by column on silica gel (methanol:dichloromethane=0˜1:10) to obtain crude tert-butyl 4-(8-cyclopropoxy-6-methoxy-7-(5-methyl-1H-indazol-4-yl)-2-((((S)-1-methylpyrrolidin-2-yl)methoxy)quinazolin-4-yl)piperazin-1-carboxylate (200 mg, Y: 30%) as a yellow solid. ES-API: [M+H]⁺=644.3

step 5: tert-butyl 4-(8-cyclopropoxy-6-methoxy-7-(5-methyl-1H-indazol-4-yl)-2-((((S)-1-methylpyrrolidin-2-yl)methoxy)quinazolin-4-yl)piperazin-1-carboxylate (200 mg, 0.3 mmol) was dissolved in dichloromethane (4 mL) and then trifluoroacetic acid (2 mL) was added. The reaction was conducted at room temperature for 1 h and the reaction mixture was dried by rotary evaporation to obtain 8-cyclopropoxy-6-methoxy-7-(5-methyl-1H-indazol-4-yl)-2-((((S)-1-methylpyrrolidin-2-yl)methoxy)-4-(piperazin-1-yl)quinazoline (169 mg, Y: 100%) as a yellow oil. ES-API: [M+H]⁺=544.3

step 6: 8-cyclopropoxy-6-methoxy-7-(5-methyl-1H-indazol-4-yl)-2-((((S)-1-methylpyrrolidin-2-yl)methoxy)-4-(piperazin-1-yl)quinazoline (169 mg, 0.3 mmol) was dissolved in dichloromethane (4 mL). At 0° C., triethylamine (2 mL) and acrylic anhydride (32 mg, 0.25 mmol) were added and then the reaction was conducted at 0° C. for 1 h. Water (10 mL) was added to the reaction solution, which was extracted with dichloromethane (10 mL×3). The organic phase was dried and dried by rotary evaporation, and purified by chromatography preparation, chromatography column: Ultimate XB-C18, 50*250 mm, 10 um, mobile phase:acetonitrile/water=10/90-90/10, to obtain 1-(4-(8-cyclopropoxy-6-methoxy-7-(5-methyl-1H-indazol-4-yl)-2-(((S)-1-methylpyrrolidin-2-yl)methoxy)quinazolin-4-yl)piperazin-1-yl)prop-2-en-1-one (8.5 mg, Y: 10%) as a white solid. ES-API: [M+H]⁺-598.3. ¹HNMR (400 MHz, CDCl₃)δ 12.57 (s, 1H), 7.55 (s, 1H), 7.46 (d, J=8.5 Hz, 1H), 7.32 (d, J=8.5 Hz, 1H), 7.01 (s, 1H), 6.63 (dd, J=16.8, 10.5 Hz, 1H), 6.37 (d, J=16.7 Hz, 1H), 5.78 (d, J=10.5 Hz, 1H), 5.20 (s, 1H), 4.85 (d, J=12.4 Hz, 1H), 4.15 (d, J=15.5 Hz, 2H), 3.90 (d, J=29.1 Hz, 9H), 3.74 (d, J=2.6 Hz, 3H), 3.07 (s, 3H), 3.01-2.81 (m, 2H), 2.34 (s, 2H), 2.20 (s, 3H), 2.09 (s, 1H), 0.15 (d, J=14.5 Hz, 4H).

### Example 50 Preparation of Z50

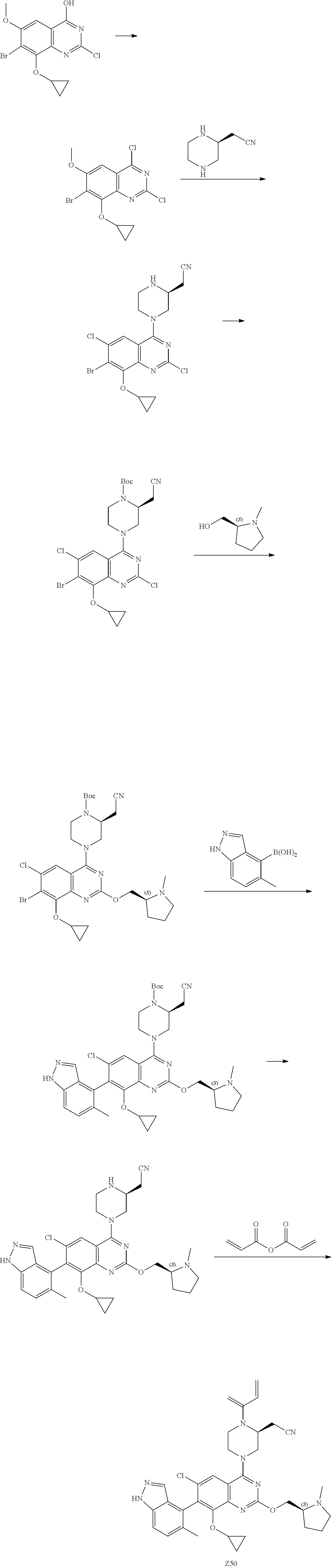

step 1: 7-bromo-8-cyclopropoxy-6-methoxyquinazolin-2,4-diol (3.8 g, 11.6 mol) was added to phosphorus oxychloride (40 ml). After the mixture was heated to 100° C., N,N-diisopropylethylamine was added slowly till the reaction solution was clear. After the mixture was heated to 130° C. the reaction was conducted for 2 h. After the reaction solution was cooled and phosphorus oxychloride was removed by rotary evaporation, the residue was poured into water (100 ml). The organic phase was washed with water and washed with brine, and dried by rotary evaporation to obtain crude 7-bromo-2,4-dichloro-8-cyclopropoxy-6-methoxyquinazoline (5 g, Y: 100%).

step 2: 7-bromo-2,4-dichloro-8-cyclopropoxy-6-methoxyquinazoline (2.1 g, 5.78 mol) was dissolved in tetrahydrofuran (50 ml), and then (S)-2-(piperazin-2-yl) acetonitrile (3.2 g, 16.5 mol) and triethylamine (3.3 g, 33 mol) were added. The mixture was heated to 60° C. and the reaction was conducted for 2 h. After the reaction solution was cooled, ethyl acetate (100 ml) was added. The organic phase was washed with water, washed with ammonium chloride solution and washed with brine, and dried by rotary evaporation to obtain (S)-2-(4-(7-bromo-2-chloro-8-cyclopropoxy-6-methoxyquinazolin-4-yl)piperazin-2-yl)acetonitrile (2.7 g, Y: 55%) as a yellow solid.

step 3: (S)-2-(4-(7-bromo-2-chloro-8-cyclopropoxy-6-methoxyquinazolin-4-yl)piperazin-2-yl) acetonitrile (3.7 g, 8.2 mol) was dissolved in tetrahydrofuran and water (40/20 ml) and then sodium hydroxide solid (984 mg, 24.6 mmol) and di-tert-butyl dicarbonate (9 g, 41 mmol) were added. The reaction was stirred at room temperature for 16 h. The reaction is monitored till it was complete. After the reaction solution was cooled, ethyl acetate (100 ml) was added. The organic phase was washed with water, washed with ammonium chloride solution and washed with brine, dried and dried by rotary evaporation and purified by column (petroleum ether/ethyl acetate=3/1) to obtain tert-butyl (S)-4-(7-bromo-2-chloro-8-cyclopropoxy-6-methoxyquinazolin-4-yl)-2-(cyanomethyl)piperazin-1-carboxylate (2.78 g, Y: 62%) as a pale yellow solid.

step 4: tert-butyl (S)-4-(7-bromo-2-chloro-8-cyclopropoxy-6-methoxyquinazolin-4-yl)-2-(cyanomethyl)piperazin-1-carboxylate (2.5 g, 4.5 mmol) was dissolved in (S)-(1-methylpyrrolidin-2-yl) methanol (20 mL) and then potassium fluoride (1.3 g, 22.7 mmol) was added. After the mixture was stirred evenly it was heated to 110° C. and the reaction was conducted for 2 h. After cooling, the reaction solution was poured into water (100 mL), extracted with ethyl acetate (100 mL×3) and purified by column (methanol:dichloromethane=0˜1:10) to obtain tert-butyl (S)-4-(7-bromo-8-cyclopropoxy-6-methoxy-2-((((S)-1-methylpyrrolidin-2-yl) methoxy)quinazolin-4-yl)-2-(cyanomethylpiperazin-1-carboxylate (2 g, Y: 71%) as a yellow solid.

step 5: tert-butyl (S)-4-(7-bromo-8-cyclopropoxy-6-methoxy-2-((((S)-1-methylpyrrolidin-2-yl)methoxy)quinazolin-4-yl)-2-cyanomethyl piperazin-1-carboxylate (2 g, 3.17 mmol), (5-methyl-1H-indazol-4-yl) boronic acid (1.13 g, 6.35 mmol), tripotassium phosphate (2 g, 9.51 mmol), tris (dibenzylideneindenacetone)dipalladium (290 mg), 2-dicyclohexylphosphine-2′,4′,6′-triisopropylbiphenyl (150 mg) were added in sequence into dioxane (16 mL) and water (4 mL). The system atmosphere was replaced with nitrogen three times, then the temperature was warmed up to 110° C. and the reaction was conducted for 16 h. After cooling to room temperature, the reaction solution was poured into ethyl acetate (100 mL) and after being washed with brine once it was purified by column on silica gel (methanol dichloromethane=0˜1:10) to obtain tert-butyl (2S)-2-(cyanomethyl)-4-(8-cyclopropoxy-6-methoxy-7-(5-methyl-1H-indazol-4-yl)-2-((((S)-1-methylpyrrolidin-2-yl)methoxy)quinazolin-4-ylpiperazin-1-carboxylate (910 mg, Y: 43%) as a yellow solid step 6: tert-butyl (2S)-2-(cyanomethyl)-4-(8-cyclopropoxy-6-methoxy-7-(5-methyl-1H-indazol-4-yl)-2-((((S)-1-methylpyrrolidin-2-yl)methoxy)quinazolin-4-ylpiperazin-1-carboxylate (910 mg, 1.33 mmol) was dissolved in dichloromethane (10 mL) and then trifluoroacetic acid (4 mL) was added. The reaction was conducted at room temperature for 1 h and the reaction mixture was dried by rotary evaporation to obtain 2-((2S)-4-(8-cyclopropoxy-6-methoxy-7-(5-methyl-1H-indazol-4-yl)-2-((((S)-1-methylpyrrolidin-2-yl))methoxy)quinazolin-4-ylpiperazin-2-yl acetonitrile (770 mg, Y: 100%) as a yellow oil step 7: 2-((2S)-4-(8-cyclopropoxy-6-methoxy-7-(5-methyl-1H-indazol-4-yl)-2-((((S)-1-methylpyrrolidin-2-yl))methoxy)quinazolin-4-ylpiperazin-2-yl acetonitrile (770 mg, 1.33 mmol) was dissolved in dichloromethane (5 mL), and after cooling to 0° C. triethylamine (3 mL) and acrylic anhydride (150 mg, 1.2 mmol) were added, and then the reaction was conducted at 0° C. for 1 h. Water (10 mL) was added to the reaction solution, which was extracted with dichloromethane (30 mL×3). The organic phase was dried and dried by rotary evaporation, and purified by preparation to obtain 2-((2S)-1-acryloyl-4-(8-cyclopropoxy-6-methoxy-7-(5-methyl-1H-indazol-4-yl)-2-((((S)-1-methylpyrrolidone-2-yl)methoxy)quinazolin-4-ylpiperazin-2-yl) acetonitrile (8.2 mg, Y: 10%) as a white solid. The preparation and separation conditions were the same as in Example 10. ¹HNMR (400 MHz, CDCl₃)δ 7.58 (d, J=3.8 Hz, 1H), 7.44 (d, J=8.5 Hz, 1H), 7.33 (d, J=8.5 Hz, 1H), 7.02 (d, J=17.0 Hz, 1H), 6.63 (s, 1H), 6.42 (d, J=16.6 Hz, 1H), 5.84 (d, J=10.5 Hz, 1H), 4.69 (s, 1H), 4.45 (s, 1H), 4.31 (d, J=11.7 Hz, 3H), 3.75 (s, 3H), 3.73 (s, 1H), 3.61 (d, J=12.4 Hz, 1H), 3.32 (d, J=39.7 Hz, 2H), 3.07-2.75 (m, 3H), 2.62 (s, 3H), 2.45 (s, 1H), 2.21 (d, J=3.3 Hz, 4H), 2.14 (s, 1H), 1.87 (s, 4H), 0.23-0.10 (m, 4H).

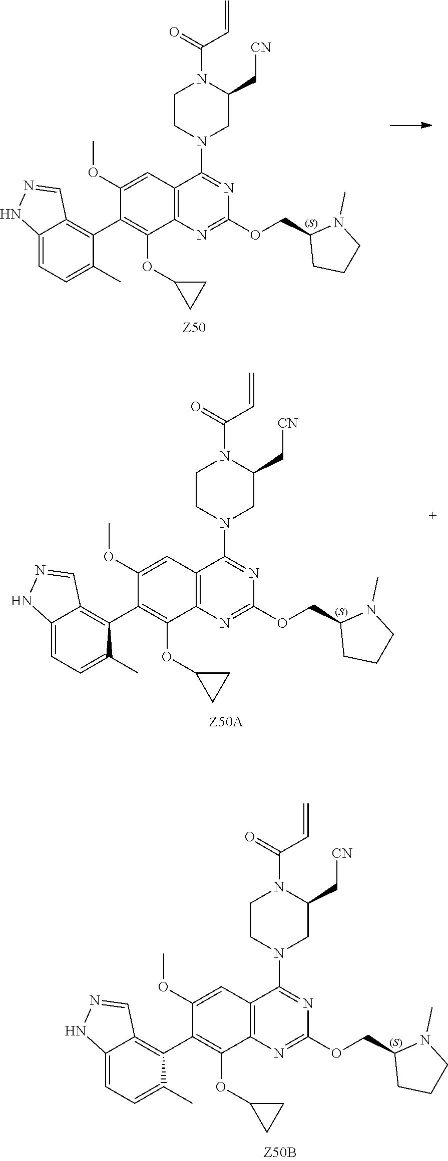

The above compound Z50 (42 mg) was separated through chiral resolution (mobile phase:acetonitrile:isopropanol:diethylamine=90:10:0.2; column: IC 254 nanometer@4.8 nanometer; flow rate: 1.0 ml/min; column temperature: 30.0° C.) to obtain: one isomer, arbitrarily designated as Z50A: 2-((S)-1-acryloyl-4-((R)-8-cyclopropoxy-6-methoxy-7-(5-methyl-1H-indazol-4-yl)-2-((((S-1-methylpyrrolidin-2-yl)methoxy)quinazolin-4-ylpiperazin-2-yl) acetonitrile (retention time: 4.966 min; 23 mg; purity: 94.58%, de value: 99.6%. ES-API: [M+H]⁺=637.3; and the other isomer, arbitrarily designated as Z50B: 2-((S)-1-acryloyl-4-((S)-8-cyclopropoxy-6-methoxy-7-(5-methyl-1H-indazol-4-yl)-2-((((S-1-methylpyrrolidin-2-yl)methoxy)quinazolin-4-ylpiperazin-2-yl) acetonitrile (retention time: 6.381 min; 19 mg; purity: 93.55%, de value: 100%). ES-API: [M+H]⁺=637.3.

### Test Example 1. Cell Proliferation Inhibition Experiment

NCI-H358 was a human non-small cell lung cancer cell line with Kras G12C mutation, cultured in 10% FBS RPMI-1640 medium; A549 was a human lung adenocarcinoma cell line with Kras G12S mutation, cultured in 10% FBS F-12K medium. The cells in the logarithmic growth phase was taken, digested with trypsin EDTA, collected and counted, and H358 was adjusted to 1.8E4 cells/ml using 2% FBS RPMI-1640 medium, and A549 was adjusted to 8.9E3 cells/ml using 2% FBS F-12K medium; 800 (45 μl) H358 or 400 (45 μl) A549 cells were seeded in a 384-well sphere plate respectively, cultured overnight to establish a 3D cell model. DMSO was used to prepare 1000× compound 3.16-fold gradient concentration stock solution, and 2% FBS medium was used to dilute 100-fold to 10× compound stock solution. On the second day after cell seeding, 5 μl 10× compound stock solution was added to each cell culture well, the final concentration was 1×, and DMSO content was 0.1%. DMSO was used as the experimental control (control) and 2% FBS medium as the blank control (blank). After the compound and cells were added and cultured for 5 days, 25 μl CellTiter-Glo working solution was added to each well, mixed at 400 rpm and incubated for 30 minutes. After standing 30 minutes at room temperature, 40 μl of the mixture was transfered to a 384-well plate with a non-transparent white bottom, the luminescence chemiluminescence value was read, and the cells proliferation inhibition rate IR (%)=(RLU control-RLU compound)/(RLU control-RLU blank)×100% was calculated, Prism 6 four-parameter method was used to fit the compound's gradient dilution concentration and the corresponding cell proliferation inhibition rate, and the IC₅₀ value was calculated. It can be seen from the results that the compounds of the present invention had high inhibitory activity against Kras G12C mutant NCI-H358 cells, with IC₅₀ of less than 1000 nM, or less than 500 nM, or even less than 200 nM, and some compound had the activity less than 100 nM, or even less than 50 nM; while their inhibitory activity against A549 cells was low, with IC₅₀ more than 3000 nM, or more than 5000 nM, and some compound had the activity more than 10000 nM, or even more than 14000 nM. The result of the exemplary compounds was shown in the following table 1.

TABLE 1
Inhibitory activity of compounds against H358 and A549 cells

| Compound | H358 | A549 |
|---|---|---|
| No. | IC₅₀ (μM) | IC₅₀ (μM) |
| Z1 | 0.108 | >1 |
| Z2 | 0.126 | >1 |
| Z3 | 0.060 | >1 |
| Z4 | 0.136 | >1 |
| Z5 | 0.311 | >10 |
| Z6 | 0.139 | >5 |
| Z7 | 0.016 | >1 |
| Z7B | 0.006 | >1 |
| Z8 | 0.070 | >10 |
| Z10A | 0.257 | >10 |
| Z12 | 0.018 | >10 |
| Z12A | 0.009 | >1 |
| Z12B | 0.390 | >5 |
| Z13 | 0.336 | >1 |
| Z27 | 0.324 | >1 |
| Z28 | 0.011 | >1 |
| Z28B | 0.004 | >1 |
| Z29 | 0.051 | >10 |
| Z30 | 0.026 | >5 |
| Z31 | 0.034 | >10 |
| Z32 | 0.020 | >1 |
| Z33 | 0.039 | >5 |
| Z34 | 0.038 | >30 |
| Z35 | 0.043 | >5 |
| Z36 | 0.016 | >30 |
| Z37 | 0.051 | >5 |
| Z38 | 0.019 | >30 |
| Z39 | 0.021 | >5 |
| Z39B | 0.012 | >30 |
| Z40 | 0.025 | >1 |
| Z40A | 0.012 | >30 |
| Z42 | 0.331 | >30 |
| Z45 | 0.018 | >30 |
| Z46 | 0.002 | >30 |
| Z47 | 0.038 | >5 |
| Z48B | 0.010 | >10 |
| Z49 | 0.083 | >30 |
| Z50A | 0.050 | >10 |

It can be seen from Table 1 that the exemplary compounds of the present invention have higher inhibitory activity against Kras G12C mutant NCI-H358 cells, but have lower inhibitory activity against A549 cells, and have obvious selective inhibitory activity.

All documents mentioned in the present invention are cited as references in this application, as if each document is individually cited as a reference. In addition, it should be understood that after reading the above teaching content of the present invention, those skilled in the art may make various changes or modifications to the present invention, and these equivalent forms also fall within the scope defined by the appended claims of the present application.

1. An oxygen-substituted six-membered cyclopyrimidine compound, or a pharmaceutically acceptable salt, stereoisomer, solvent or prodrug thereof, and the structure of the compound is as shown in formula (I): 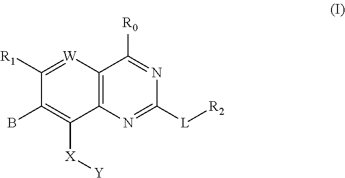
wherein,
R₀ is 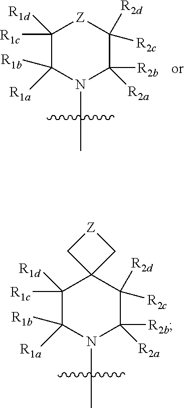
wherein, “ [symbol] ” represents that the nitrogen atom is connected with other parts of the molecule;
R₁ₐ, R_{1b}, R_{1c}, R_{1d}, R₂ₐ, R_{2b}, R_{2c}, R₂ₐ are the same or different, and are each independently hydrogen, halogen, C₁₋₃ alkyl, —C₁₋₃ alkyl-hydroxy, —C₁₋₃ alkyl-cyano, —C₁₋₃ alkyl-C₁₋₆ alkoxy, —C₁₋₃ alkyl-halo C₁₋₆ alkyl or —C₁₋₃ alkyl-halo C₁₋₆ alkoxy;
Z is N—C(O)—CR_{X3}═CR_{X1}R_{X2} or N—C(O)—C≡CR_{X4}; wherein, R_{X1}, R_{X2} are each independently hydrogen, halogen, cyano, NR^{a}R^{b}, C₁₋₃ alkyl, halo C₁₋₃ alkyl, —C₁₋₃ alkyl-hydroxy, —C₁₋₃ alkyl-cyano, —C₁₋₃ alkyl-C₁₋₃ alkoxy, —C₁₋₃ alkyl-NR^{a}R^{b}, —C₁₋₃ alkyl-3- to 6-membered heterocycloalkyl, —C₁₋₃ alkyl-5- or 6-membered monocyclic heteroaryl; wherein, R^{a}, R^{b} are each independently hydrogen or C₁₋₃ alkyl; R_{X3} is hydrogen, halogen, —O—C₁₋₃ alkyl or —O—C₃₋₆ cycloalkyl; R_{X4} is hydrogen, halo C₁₋₃ alkyl, —C₁₋₃ alkyl-hydroxy, —C₁₋₃ alkyl-cyano, —C₁₋₃ alkyl-C₁₋₃ alkoxy;
and
when R₀ is 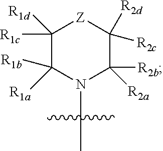
R₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁₁, —NH—Rn or —N(R₁₁)R₁₂; wherein, R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; or
when R₀ is 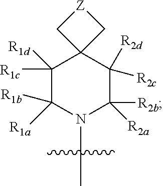
R₁ is halogen, cyano, substituted or unsubstituted C₁₋₆ alkyl, —O—Rn, —NH—Rn or —N(R₁)R₁₂; wherein R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms;
L is a bond, —CR_{L1}R_{L2}—, —O—(CR_{L1}R_{L2})ₜ₁— or —NH—(CR_{L3}R_{L4})ₜ₂—; wherein, R_{L1}, R_{L2}, R_{L3}, R_{L4} are the same or different, and are each independently hydrogen, halogen, hydroxyl, hydroxymethyl, hydroxyethyl, C₁₋₃ alkyl or oxo group; t1, t2 are each independently 0, 1, 2, 3 or 4; among R_{L1} and R_{L2} or among R_{L3} and R_{L4}, when one of them is oxo group, the other does not exist;
R₂ is halogen, hydroxy, —SO₂C₁₋₆ alkyl, substituted or unsubstituted 3- to 20-membered heterocycloalkyl, substituted or unsubstituted C₃₋₂₀ cycloalkyl, substituted or unsubstituted 5- or 6-membered monocyclic heteroaryl or NR₂₁R₂₂; wherein, R₂₁, R₂₂ are each independently hydrogen, substituted or unsubstituted C₁₋₆ alkyl, —SO₂C₁₋₆ alkyl, —SO₂C₃₋₆ cycloalkyl, —C(O)C₁₋₆ alkyl or —C(O)halo C₁₋₆ alkyl; or R₂₁ and R₂₂ together with the nitrogen atom connected form a substituted or unsubstituted 3- to 20-membered heterocycloalkyl; wherein, the 3- to 20-membered heterocycloalkyl, the 5- or 6-membered monocyclic heteroaryl each independently have 1, 2 or 3 heteroatoms selected from N, O and S as ring atoms;
X is O, NR₃, S, S(O) or S(O)₂; wherein, R₃ is hydrogen, C₁₋₆ alkyl, C₂₋₆ alkenyl, C₃₋₆ cycloalkyl or 3- to 6-membered heterocycloalkyl; wherein, the 3- to 6-membered heterocycloalkyl has 1, 2 or 3 heteroatoms selected from N, O and S as ring atoms;
Y is substituted or unsubstituted C₃₋₂₀ cycloalkyl or substituted or unsubstituted 3- to 20-membered heterocycloalkyl; wherein, the 3- to 20-membered heterocycloalkyl has 1, 2 or 3 heteroatoms selected from N, O and S as ring atoms;
W is CR₄ or N; wherein R₄ is hydrogen, halogen, cyano, hydroxyl, substituted or unsubstituted C₁₋₆ alkyl, substituted or unsubstituted C₁₋₆ alkoxy, substituted or unsubstituted C₃₋₂₀ cycloalkyl, substituted or unsubstituted C₃₋₂₀ cycloalkoxy, —NH—(C₁₋₄ alkyl) or —N(C₁₋₄ alkyl)₂;
the “substituted” mentioned above each independently means that 1, 2, 3, or 4 hydrogen atoms in the group are replaced by substituents independently selected from the group S; and the substituents in the group S are selected from the group consisting of hydroxy, halogen, nitro, oxo, C₁₋₆ alkyl, C₁₋₆ alkyl substituted with hydroxy, benzyl, —(CH₂)ᵤ-cyano, —(CH₂)ᵤ—C₁₋₆ alkoxy, —(CH₂)ᵤ-halo C₁₋₆ alkoxy, —(CH₂)ᵤ-halo C₁₋₆ alkyl, —(CH₂)ᵤ₋₃— to 6-membered heterocycloalkyl, —(CH₂)ᵤ₋₅— or 6-membered monocyclic heteroaryl, —(CH₂)ᵤ—C₃₋₈ cycloalkyl, —(CH₂)ᵤ—O—(CH₂)ᵥ—C₃₋₈ cycloalkyl, —(CH₂)ᵤ—O—(CH₂)ᵥ—C₁₋₆ alkoxy, —(CH₂)ᵤ—O—(CH₂)ᵥOH, —(CH₂)ᵤ—SO₂C₁₋₆ alkyl, —(CH₂)ᵤ—NRₐ₀R_{b0}, —(CH₂)ᵤ—C(O)NRₐ₀R_{b0}, —(CH₂)ᵤ—C(O)C₁₋₆ alkyl, —C(O)OC₁₋₆ alkyl, —NRₐ₀C(O)—(CH₂)ᵤ—NRₐ₀R_{b0}, —NRₐ₀C(O)—(CH₂)ᵤOH, —NRₐ₀C(O)-halo C₁₋₆ alkyl; wherein, the 3- to 6-membered heterocycloalkyl, the 5- or 6-membered monocyclic heteroaryl each independently have 1, 2 or 3 heteroatoms selected from N, O and S as ring atoms; the 3- to 6-membered heterocycloalkyl, the 5- or 6-membered monocyclic heteroaryl are optionally substituted with 1, 2 or 3 substituents selected from halogen, cyano, C₁₋₃ alkyl, C₁₋₃ alkoxy and C₃₋₆ cycloalky; u, v are each independently 0, 1, 2, 3 or 4; Rₐ₀, R_{b0} are each independently hydrogen or C₁₋₃ alkyl;
B is C₆₋₁₀ aryl, 5- or 6-membered monocyclic heteroaryl or 8- to 10-membered bicyclic heteroaryl; wherein the 5- or 6-membered monocyclic heteroaryl, the 8- to 10-membered bicyclic heteroaryl each independently have 1, 2 or 3 heteroatoms selected from N, O and S as ring atoms; the C₆₋₁₀ aryl, the 5- or 6-membered monocyclic heteroaryl, the 8- to 10-membered bicyclic heteroaryl are unsubstituted or substituted with 1, 2, 3 or 4 groups independently selected from Rₛ₁; or
B is the structure as shown in formula (B): 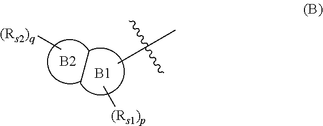
wherein, the B1 ring is a benzene ring or a 5- or 6-membered monocyclic heteroaryl ring; the B2 ring is a 5- or 6-membered heterocycloalkyl ring fused with the B1 ring or a 5- or 6-membered cycloalkyl ring fused with the B1 ring; wherein the 5- or 6-membered monocyclic heteroaryl ring, the 5- or 6-membered heterocycloalkyl ring each independently have 1, 2 or 3 heteroatoms selected from N, O and S as ring atoms;
(Rₛ₁)ₚ means that the hydrogens on the B1 ring are replaced by p of Rₛ₁, p is 0, 1, 2 or 3, and each Rₛ₁ is the same or different;
(Rₛ₂)_{q} means that the hydrogens on the B2 ring are replaced by q of Rₛ₂, q is 0, 1, 2 or 3, and each Rₛ₂ is the same or different;
Rₛ₁, Rₛ₂ are each independently hydroxy, halogen, nitro, oxo, C₁₋₆ alkyl, C₁₋₆ alkyl substituted with hydroxy, benzyl, —(CH₂)ᵤ₁-cyano, —(CH₂)ᵤ₁—C₁₋₆ alkoxy, —(CH₂)ᵤ₁-halo C₁₋₆ alkoxy, —(CH₂)ᵤ₁-halo C₁₋₆ alkyl, —(CH₂)ᵤ₁-3- to 6-membered heterocycloalkyl, —(CH₂)ᵤ₁-5- or 6-membered monocyclic heteroaryl, —(CH₂)ᵤ₁—C₃₋₈ cycloalkyl, —(CH₂)ᵤ₁—O—(CH₂)ᵥ₁—C₃₋₈ cycloalkyl, —(CH₂)ᵤ₁—O—(CH₂)ᵥ₁—C₁₋₆ alkoxy, —(CH₂)ᵤ₁—O—(CH₂)ᵥ1OH, —(CH₂)ᵤ₁—SO₂C₁₋₆ alkyl, —(CH₂)ᵤ₁—NRₐ₀R_{b0}, —(CH₂)ᵤ₁—C(O)NRₐ₀R_{b0}, —(CH₂)ᵤ₁—C(O)C₁₋₆ alkyl, —C(O)OC₁₋₆ alkyl, —NRₐ₀C(O)—(CH₂)ᵤ₁—NRₐ₀R_{b0}, —NRₐ₀C(O)—(CH₂)ᵤ₁OH, —NRₐ₀C(O)-halo C₁₋₆ alkyl; wherein, the 3- to 6-membered heterocycloalkyl, the 5- or 6-membered monocyclic heteroaryl each independently have 1, 2 or 3 heteroatoms selected from N, O and S as ring atoms; the 3- to 6-membered heterocycloalkyl, the 5- or 6-membered monocyclic heteroaryl are optionally substituted with 1, 2 or 3 substituents selected from halogen, cyano, C₁₋₃ alkyl, C₁₋₃ alkoxy and C₃₋₆ cycloalkyl; u1, v1 are each independently 0, 1, 2, 3 or 4; Rₐ₀, R_{b0} are each independently hydrogen or C₁₋₃ alkyl.

2. The compound according to claim 1, or a pharmaceutically acceptable salt, stereoisomer, solvent or prodrug thereof, wherein, the compound represented by formula (I) is the compound represented by formula (II-1) or formula (III-1): 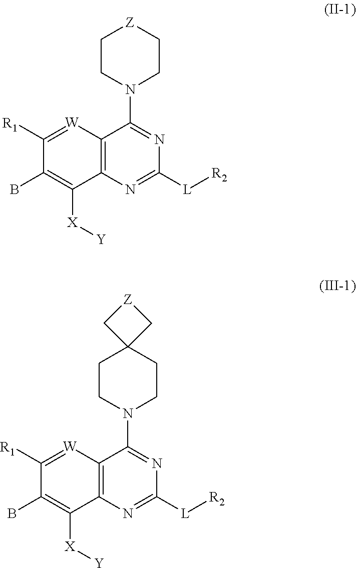
in each formula, R₂, X, Y, B, W, Z, L are defined as before; in formula (II-1), R₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁₁, —NH—Rn or —N(R₁₁)R₁₂; wherein, R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; in formula (III-1), R₁ is halogen, cyano, substituted or unsubstituted C₁₋₆ alkyl, —O—R₁₁, —NH—Rn or —N(R₁₁)R₁₂; wherein, R₁₁, R₁₂ are each independently substituted or unsubstituted C₁₋₆ alkyl, substituted or unsubstituted C₃₋₆ cycloalkyl, substituted or unsubstituted 3- to 6-membered heterocycloalkyl, substituted or unsubstituted C₆₋₁₀ aryl, substituted or unsubstituted 5- or 6-membered monocyclic heteroaryl or substituted or unsubstituted 8- to 10-membered bicyclic heteroaryl; or R₁₁, R₁₂ together with the nitrogen atom connected form a substituted or unsubstituted 3- to 6-membered heterocycloalkyl; wherein, the 3- to 6-membered heterocycloalkyl, the 5- or 6-membered monocyclic heteroaryl, the 8- to 10-membered bicyclic heteroaryl each independently have 1, 2 or 3 heteroatoms selected from N, O and S as ring atoms; the “substituted” mentioned above each independently means that 1, 2, 3, or 4 hydrogen atoms in the group are replaced by substituents independently selected from the group S.

3. The compound according to claim 1, or a pharmaceutically acceptable salt, stereoisomer, solvent or prodrug thereof, wherein, when R₀ is 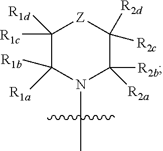
R₁ is halogen, cyano, substituted or unsubstituted C₁₋₆ alkyl, substituted or unsubstituted C₂₋₆ alkenyl, substituted or unsubstituted C₃₋₆ cycloalkyl, —O—R₁₁ or —NH—R₁₁; wherein, R₁₁ is substituted or unsubstituted C₁₋₆ alkyl or substituted or unsubstituted C₃₋₆ cycloalkyl; the “substituted” mentioned above each independently means that 1, 2, 3, or 4 hydrogen atoms in the group are replaced by substituents independently selected from the group S.

4. The compound according to claim 1, or a pharmaceutically acceptable salt, stereoisomer, solvent or prodrug thereof, wherein, when R₀ is 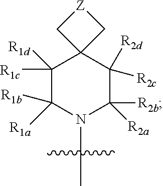
R₁ is halogen, cyano, substituted or unsubstituted C₁₋₆ alkyl, —O—R₁₁ or —NH—R₁₁; wherein, R₁₁ is substituted or unsubstituted C₁₋₆ alkyl or substituted or unsubstituted C₃₋₆ cycloalkyl; the “substituted” mentioned above each independently means that 1, 2, 3, or 4 hydrogen atoms in the group are replaced by substituents independently selected from the group S.

5. The compound according to claim 1, or a pharmaceutically acceptable salt, stereoisomer, solvent or prodrug thereof, wherein, X is O, NH, S, S(O) or S(O)₂.

6. The compound according to claim 1, or a pharmaceutically acceptable salt, stereoisomer, solvent or prodrug thereof, wherein, Y is substituted or unsubstituted C₃₋₆ cycloalkyl or substituted or unsubstituted 3- to 6-membered heterocycloalkyl; wherein, the 3- to 6-membered heterocycloalkyl has 1, 2 or 3 heteroatoms selected from N, O and S as ring atoms; the “substituted” mentioned above each independently means that 1, 2, 3, or 4 hydrogen atoms in the group are replaced by substituents independently selected from the group S.

7. The compound according to claim 1, or a pharmaceutically acceptable salt, stereoisomer, solvent or prodrug thereof, wherein, in R₁, B, the C₆₋₁₀ aryl is each independently phenyl or naphthyl.

8. The compound according to claim 1, or a pharmaceutically acceptable salt, stereoisomer, solvent or prodrug thereof, wherein, in R₁, R₂, B, the 5- or 6-membered monocyclic heteroaryl is each independently selected from thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

9. The compound according to claim 1, or a pharmaceutically acceptable salt, stereoisomer, solvent or prodrug thereof, wherein, in R₁, B, the 8- to 10-membered bicyclic heteroaryl is each independently selected from benzoxazole, benzisoxazole, benzimidazole, benzothiazole, benzisothiazole, benzotriazole, benzofuran, benzothiophene, indole, indazole, isoindole, quinoline, isoquinoline, quinazoline, quinoxaline, cinnoline, pyridopyrimidine and naphthyridine.

10. A pharmaceutical composition, comprising the compound according to claim 1, or a pharmaceutically acceptable salt, stereoisomer, solvate or prodrug thereof; and a pharmaceutically acceptable carrier.

11. A use of the compound according to claim 1, or a pharmaceutically acceptable salt, stereoisomer, solvate or prodrug thereof or a pharmaceutical composition comprising the compound according to claim 1, or a pharmaceutically acceptable salt, stereoisomer, solvate or prodrug thereof; and a pharmaceutically acceptable carrier, in the manufacture of a medicament for preventing and/or treating cancer or in the manufacture of an inhibitor against KRAS mutation.
